(19) United States
(12) Patent Application Publication
(10) Pub. No.: US 2026/0159836 A1
(43) Pub. Date: Jun. 11, 2026

(54) OLIGONUCLEOTIDE-MEDIATED KNOCKDOWN OF PLN

(71) Applicant: Soufflé Therapeutics, Inc., Watertown, MA (US)

(72) Inventors: Vadim Dudkin, Wellesley, MA (US); Timofey Zatsepin, Brookline, MA (US); Roman Bogorad, Newton, MA (US); Duncan Brown, Berkeley, CA (US); James McSwiggen, Arvada, CO (US); Jordan Chambers, Watertown, MA (US)

(21) Appl. No.: 19/411,019

(22) Filed: Dec. 5, 2025

Related U.S. Application Data
(60) Provisional application No. 63/728,317, filed on Dec. 5, 2024.

(51) Int. Cl. C12N 15/113; A61K 31/712; A61K 31/7125; A61K 31/713

(52) U.S. Cl. CPC C12N 15/113; A61K 31/712; A61K 31/7125; A61K 31/713; C12N 2310/11; C12N 2310/14; C12N 2310/315; C12N 2310/321; C12N 2310/322

(57) ABSTRACT

Provided herein are oligonucleotides for inhibiting the expression of phospholamban (PLN) and methods of using the oligonucleotides for reducing PLN expression in a subject.

### CROSS REFERENCE TO RELATED APPLICATIONS

This application claims the benefit of U.S. Provisional Application No. 63/728,317 filed on Dec. 5, 2024, the entirety of which is incorporated herein by reference.

### INCORPORATION BY REFERENCE OF SEQUENCE LISTING

The present application is being filed along with a Sequence Listing in electronic format. The Sequence Listing is provided as a file entitled 360770-0002_SeqList_ST26.xml, created Dec. 4, 2025, which is 17,820,546 bytes in size. The information in the electronic format of the Sequence Listing is incorporated herein by reference in its entirety.

### BACKGROUND

Phospholamban (PLN, also referred to as CMD1P, PLB, cardiac phospholamban, or CMH) denotes an essential regulator of cardiac contractility. PLN serves as a primary substrate for the cAMP-dependent protein kinase in cardiac muscle. The protein it encodes acts as an inhibitor of the cardiac muscle sarcoplasmic reticulum Ca(2+)-ATPase when in its unphosphorylated form, with the inhibition being lifted upon phosphorylation of the protein. This phosphorylation triggers the activation of the Ca(2+) pump, which enhances muscle relaxation rates and contributes to the inotropic response initiated by beta-agonists in the heart. PLN protein is a significant regulator of diastolic function in the heart. Mutations in this gene are linked to inherited human dilated cardiomyopathy resulting in refractory congestive heart failure, as well as familial hypertrophic cardiomyopathy.

Focusing on PLN for the prevention and/or treatment of heart failure with reduced ejection fraction (HF-rEF), arrhythmias, and/or cardiomyopathy is crucial, as IF-rEF is a leading cause of mortality and morbidity. The defining features of heart failure include impaired cardiac contraction and relaxation, coupled with irregularities in calcium handling and β-adrenergic signaling (Lou, Q, et al. Adv Exp Med Biol. 2012; 740: 1145-1174). In cardiomyocytes, cytosolic calcium is vital for regulating contraction and relaxation through the excitation-contraction coupling mechanism. Calcium influx via L-type calcium channels triggers calcium-induced calcium release from the sarco(endo)plasmic reticulum (SR) via ryanodine receptors, leading to cardiac contraction. The active relaxation process involves the transportation of calcium from the cytosol back into the SR, mediated by the SR Ca2+ ATPase (SERCA2a), whose activity is modulated by the small phosphoprotein PLN (Kranias, E G, et al., Circ Res. 2012; 110(12): 1646-1660).

### SUMMARY

In some embodiments, the present disclosure provides an oligonucleotide for inhibiting expression of phospholamban (PLN), wherein the oligonucleotide comprises an antisense strand comprising at least 14 contiguous nucleotides substantially complementary to a sequence of nucleotides encoding PLN, with no more than 4 mismatched nucleotides.

In some embodiments, the present disclosure provides a pharmaceutical composition comprising the oligonucleotide described herein, or a pharmaceutically acceptable salt thereof, and a pharmaceutically acceptable carrier or excipient.

In some embodiments, the present disclosure provides methods of using provided oligonucleotides. In some embodiments, the present disclosure provides a method for inhibiting PLN expression or treating a PLN related disorder in a subject, the method comprising administering an effective amount of an oligonucleotide described herein, or a pharmaceutically acceptable salt thereof, or a pharmaceutical composition comprising an oligonucleotide described herein to the subject.

### DETAILED DESCRIPTION

Provided herein are oligonucleotides for inhibiting the expression of phospholamban (“PLN”). In some embodiments, the oligonucleotide comprises an antisense strand substantially complementary to a sequence encoding PLN. In some embodiments, the antisense strand comprises at least 14 contiguous nucleotides (for example, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides) substantially complementary to the sequence of nucleotides encoding PLN, with no more than 4 mismatched nucleotides.

The term “antisense strand” refers to an oligonucleotide having a nucleotide sequence substantially complementary to a target sequence in a transcript, e.g., an mRNA encoding PLN. In embodiments wherein the oligonucleotide is an RNAi agent, the term antisense strand may be used interchangeably with the term “guide strand”. In some embodiments, an antisense strand disclosed herein is 14, 15, 16, 17, 18, 19, 20, 21, 22, 23, 24, 25, 26, 27, 28, 29, or 30 nucleotides in length.

The term “sense strand” refers to an oligonucleotide having a nucleotide sequence substantially complementary to an antisense strand, e.g., the antisense strand of an RNAi agent herein. The term “sense strand” may be used interchangeably with the term “passenger strand.” In some embodiments, a sense strand disclosed herein is 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. For example, each of the sense strands in Tables 1A-1C is 19 nucleotides in length and substantially complementary to the corresponding antisense strand shown in the same row.

As used herein, and unless otherwise indicated, the term “complementary,” when used to describe a first nucleotide sequence in relation to a second nucleotide sequence, refers to the ability of an oligonucleotide comprising the first nucleotide sequence to hybridize and form a duplex structure under certain conditions with an oligonucleotide comprising the second nucleotide sequence, as will be understood by the skilled person. In some embodiments, a duplex structure disclosed herein is 12, 13, 14, 15, 16, 17, 18, 19, 20, 21, 22, 23, 24, 25, 26, 27, 28, 29, or 30 base pairs in length.

The terms “complementary”, “completely complementary” and “substantially complementary” herein can be used with respect to the base matching between the antisense strand of an oligonucleotide, e.g., an RNAi agent, and a target sequence, or between the sense strand and the antisense strand of an RNAi agent, as will be understood from the context of their use. Complementary sequences, e.g., between an antisense strand and a target sequence in a target transcript, or between the sense and antisense strand of an siRNA, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “completely complementary” with respect to each other when there are 0 mismatched base pairs upon hybridization of the two sequences. However, where a first sequence is referred to as “substantially complementary” with respect to a second sequence herein, the two sequences can be completely complementary, or they can form one or more, but generally not more than 5, 4, 3, 2, or 1 mismatched base pairs upon hybridization for a duplex up to 30 base pairs, while retaining the ability to hybridize under the conditions most relevant to their ultimate application, e.g., inhibition of gene expression via a RNA-induced silencing complex (RISC) pathway. However, where two oligonucleotides are designed to form, upon hybridization, one or more single stranded overhangs, such overhangs shall not be regarded as mismatches with regard to the determination of complementarity. For example, an siRNA comprising one oligonucleotide 21 nucleotides in length and another oligonucleotide 19 nucleotides in length, wherein the longer oligonucleotide comprises a sequence of 19 nucleotides that is fully complementary to the shorter oligonucleotide, can yet be referred to as “completely complementary” for the purposes described herein. The terms “targets” or “targeting”, when used in phrases such as an oligonucleotide targets/targeting a sequence, the terms mean that the oligonucleotide (or a sense or antisense strand within the oligonucleotide) is substantially complementary or completely complementary to the sequence.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

If an RNA strand contains one or more thymidines (“T”s) in the sequence, the thymidines (“T”s) represent uridines (“U”s). For example, the nucleotide “T”, as used in the unmodified siRNAs sense and antisense strands in Table 1A, for example, SEQ ID NOs: 1-91, 600-719, and 1228-1256, represents RNA uridine, and the corresponding RNA sense and antisense strands are listed in Table 1C.

In some embodiments, the present disclosure provides an oligonucleotide or RNAi agent, as described herein, or a pharmaceutically acceptable salt thereof. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in, J. PHARMACEUTICAL SCIENCES, 1977, (66); 1-19, incorporated herein by reference. Pharmaceutically acceptable salts of the nucleic acids and analogues thereof of this disclosure include those derived from suitable inorganic and organic acids and bases. Salts derived from appropriate bases include alkali metal, alkaline earth metal, ammonium and N⁺(C₁₋₄alkyl)₄ salts. In some embodiments, a pharmaceutically acceptable salt is the sodium salt. In some embodiments, a pharmaceutically acceptable salt is the potassium salt.

In some embodiments, the oligonucleotide comprises an antisense strand comprising at least 14 contiguous nucleotides (for example, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides) substantially complementary to a sequence of nucleotides within a transcript encoding PLN. In some embodiments, the antisense strand has no more than 5 mismatched nucleotides to the sequence of nucleotides within the transcript encoding PLN. In some embodiments, the antisense strand has no more than 4 mismatched nucleotides to the sequence of nucleotides within the transcript encoding PLN. In some embodiments, the antisense strand has no more than 3 mismatched nucleotides to the sequence of nucleotides within the transcript encoding PLN. In some embodiments, the antisense strand has no more than 2 mismatched nucleotides to the sequence of nucleotides within the transcript encoding PLN. In some embodiments, the antisense strand has no more than 1 mismatched nucleotides to the sequence of nucleotides within the transcript encoding PLN. In some embodiments, the antisense strand is completely complementary to the sequence of nucleotides within the transcript encoding PLN (e.g., 0 mismatches).

PLN is a single protein. Exemplary sequences encoding PLN may be found, for example, at NCBI RefSeq ID NM_002667.5 (SEQ ID NO: 2523). In some embodiments, PLN is encoded by a nucleotide sequence corresponding to the transcript having the NCBI RefSeq ID NM_002667.5 (SEQ ID NO: 2523).

In some embodiments, the sequence of nucleotides encoding PLN comprises a nucleotide sequence having at least 75% nucleotide sequence identity to SEQ ID NO: 2523. In some embodiments, the sequence of nucleotides encoding PLN comprises a nucleotide sequence having at least 80% nucleotide sequence identity to SEQ ID NO: 2523. In some embodiments, the sequence of nucleotides encoding PLN comprises a nucleotide sequence having at least 85% nucleotide sequence identity to SEQ ID NO: 2523. In some embodiments, the sequence of nucleotides encoding PLN comprises a nucleotide sequence having at least 90% nucleotide sequence identity to SEQ ID NO: 2523. In some embodiments, the sequence of nucleotides encoding PLN comprises a nucleotide sequence having at least 95% nucleotide sequence identity to SEQ ID NO: 2523. In some embodiments, the sequence of nucleotides encoding PLN comprises a nucleotide sequence having at least 99% nucleotide sequence identity to SEQ ID NO: 2523.

In some embodiments, the antisense strand is substantially complementary to a sequence of nucleotides corresponding to an untranslated region of the PLN transcript. In some embodiments, the antisense strand is substantially complementary to the sequence of nucleotides corresponding to an untranslated region of the PLN transcript with no more than 5, 4, 3, 2, 1, or 0 mismatches. In some embodiments, the antisense strand is completely complementary to the sequence of nucleotides corresponding to an untranslated region of the PLN transcript.

### RNAi Agents

In some embodiments, the oligonucleotide is an RNAi agent or RNAi trigger for inhibiting expression of PLN. As used herein, an “RNAi agent” or “RNAi trigger” refers to an oligonucleotide molecule capable of inducing RNA interference (RNAi), which mediates the targeted cleavage of an RNA transcript via an RNA-induced silencing complex (RISC) pathway. The RNAi agents disclosed herein include but are not limited to: short (or small) interfering RNAs (siRNAs), double stranded RNAs (dsRNA), micro RNAs (miRNAs), short hairpin RNAs (shRNA), and dicer substrates (e.g., DsiRNAs).

In some embodiments, the RNAi agent is a double stranded RNA molecule comprising an antisense strand and a sense strand that are complementary to one another and hybridize to form a duplex or double stranded region. One strand of the RNAi agent, the antisense strand or guide strand, includes a region of complementarity to a target sequence in PLN. The other strand, the sense strand or passenger strand, includes a region that is complementary to the antisense strand, such that the two strands hybridize and form a duplex structure when combined under suitable conditions. In some embodiments, the double stranded RNA molecule may be formed by base pairing between two separate molecules of RNA (e.g., an antisense strand and a sense strand). In some embodiments, the double stranded RNA molecule is a self-complementary molecule formed by intramolecular base pairing between two separate regions of a single RNA molecule (e.g., an antisense region linked to a sense strand through an unpaired RNA linker forming a loop or hairpin loop).

Where the two strands are part of a self-complementary molecule, the connecting RNA chain is referred to as a “hairpin loop.” A hairpin loop can comprise at least one unpaired nucleotide. In some embodiments, the hairpin loop can comprise at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides. In some embodiments, the hairpin loop can contain 1-4 oligoethyne glycols, including ethylene glycol, di ethylene glycol, triethylene glycol, tetraethylene glycol, hexaethylene glycol, or any combination thereof.

Where the two substantially complementary strands of a double stranded RNA molecule comprise separate RNA molecules, those molecules need not, but can be covalently connected. In certain embodiments, where the two strands are connected covalently by means other than an uninterrupted chain of nucleotides between the 3′-end of one strand and the 5′-end of the respective other strand forming the duplex structure, the connecting structure is referred to as a “linker” (though it is noted that certain other structures defined elsewhere herein can also be referred to as a “linker”). The RNA strands may have the same or a different number of nucleotides. The maximum number of base pairs is the number of nucleotides in the shortest strand of the double stranded RNA molecule minus any overhangs that are present in the duplex.

In some embodiments, the RNAi agent is an siRNA.

In some embodiments, the RNAi agent is a shRNA.

In some embodiments, the RNAi agent is a dicer substrate (e.g., a Dicer-substrate siRNA).

In some embodiments, the sense and antisense strands of the dsRNA are each independently about 15 to about 30 nucleotides in length, or about 25 to about 30 nucleotides in length, e.g., each strand is independently between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length.

In some embodiments, the duplex structure is between 15 and 30 base pairs in length, e.g., between, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length.

An RNAi agent as described herein can further include one or more single-stranded nucleotide overhangs, e.g., an overhang of 1, 2, 3, or 4 nucleotides. RNAi agent having at least one nucleotide overhang can have unexpectedly superior inhibitory properties relative to their blunt-ended counterparts. A nucleotide overhang can comprise or consist of a nucleotide/nucleoside analog, including a deoxynucleotide, an inverted deoxynucleotide or an inverted abasic nucleotide. The overhang(s) can be on the sense strand, the antisense strand or any combination thereof. Furthermore, the nucleotide(s) of an overhang can be present on the 5′-end, 3′-end or both ends of either an antisense or sense strand of the RNAi agent. In certain embodiments, longer, extended overhangs are possible.

In some embodiments, the antisense strand is 21 nucleotides in length, and the sense strand is 19 nucleotides in length. In some embodiments, the antisense strand is 19 nucleotides in length, and the sense strand is 19 nucleotides in length.

The oligonucleotides disclosed herein may be unmodified or modified (e.g., chemically modified or conjugated). A modified oligonucleotide as disclosed herein comprises an identical nucleobase sequence as compared to a corresponding unmodified oligonucleotide, but further comprises one or more modifications as disclosed herein.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides from any one of the antisense strand sequences of Tables 1A-1C. In some embodiments, the antisense strand differs by no more than 3 nucleotides from any one of the antisense strand sequences of Tables 1A-1C. In some embodiments, the antisense strand differs by no more than 2 nucleotides from any one of the antisense strand sequences of Tables 1A-1C. In some embodiments, the antisense strand differs by no more than 1 nucleotide from any one of the antisense strand sequences of Tables 1A-1C. In some embodiments, the antisense strand comprises any one of the antisense strand sequences of Tables 1A-1C. In some embodiments, the antisense strand consists of any one of the antisense strand sequences of Tables 1A-1C.

In some embodiments, the present disclosure provides oligonucleotides for inhibiting expression of PLN, wherein the oligonucleotide comprises an antisense strand substantially complementary to a sequence encoding PLN. In some embodiments, the oligonucleotide targets a sequence corresponding to positions selected from Table 4.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides from the nucleotide sequence of any one of SEQ ID NOs: 629-1256 or 1885-2522. In some embodiments, the antisense strand differs by no more than 3 nucleotides from the nucleotide sequence of any one of SEQ ID NOs: 629-1256 or 1885-2522. In some embodiments, the antisense strand differs by no more than 2 nucleotides from the nucleotide sequence of any one of SEQ ID NOs: 629-1256 or 1885-2522. In some embodiments, the antisense strand differs by no more than 1 nucleotide from the nucleotide sequence of any one of SEQ ID NOs: 629-1256 or 1885-2522. In some embodiments, the antisense strand comprises the nucleotide sequence of any one of SEQ ID NOs: 629-1256 or 1885-2522. In some embodiments, the antisense strand consists of the nucleotide sequence of any one of SEQ ID NOs: 629-1256 or 1885-2522.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides from the nucleotide sequence of any one of SEQ ID NOs: 2644-2763. In some embodiments, the antisense strand differs by no more than 3 nucleotides from the nucleotide sequence of any one of SEQ ID NOs: 2644-2763. In some embodiments, the antisense strand differs by no more than 2 nucleotides from the nucleotide sequence of any one of SEQ ID NOs: 2644-2763. In some embodiments, the antisense strand differs by no more than 1 nucleotide from the nucleotide sequence of any one of SEQ ID NOs: 2644-2763. In some embodiments, the antisense strand comprises the nucleotide sequence of any one of SEQ ID NOs: 2644-2763. In some embodiments, the antisense strand consists of the nucleotide sequence of any one of SEQ ID NOs: 2644-2763.

In some embodiments, the antisense strand comprises a nucleotide sequence having at least 90% nucleotide sequence identity (for example, 90%, 91%, 92%, 93%, 94%, 95%, 96%, 97%, 98%, 99%, or 100% nucleotide sequence identity) to a portion of the nucleotide sequence of any one of SEQ ID NOs: 629-1256 or 1885-2522. In some embodiments, the antisense strand comprises a nucleotide sequence having at least 90% nucleotide sequence identity (for example, 90%, 91%, 92%, 93%, 94%, 95%, 96%, 97%, 98%, 99%, or 100% nucleotide sequence identity) to a portion of the nucleotide sequence of any one of SEQ ID NOs: 2644-2763.

In some embodiments, the antisense strand comprises a nucleotide sequence having at least 90% nucleotide sequence identity (for example, 90%, 91%, 92%, 93%, 94%, 95%, 96%, 97%, 98%, 99%, or 100% nucleotide sequence identity) to the nucleotide sequence of any one of SEQ ID NOs: 629-1256 or 1885-2522. In some embodiments, the antisense strand comprises a nucleotide sequence having at least 90% nucleotide sequence identity (for example, 90%, 91%, 92%, 93%, 94%, 95%, 96%, 97%, 98%, 99%, or 100% nucleotide sequence identity) to the nucleotide sequence of any one of SEQ ID NOs: 2644-2763.

In some embodiments, the antisense strand consists of a nucleotide sequence having the nucleotide sequence of any one of SEQ ID NOs: 629-1256 or 1885-2522. In some embodiments, the antisense strand consists of a nucleotide sequence having the nucleotide sequence of any one of SEQ ID NOs: 2644-2763.

In some embodiments, the sense strand comprises a nucleotide sequence comprising at least 15 contiguous nucleotides (for example, 15, 16, 17, 18, or 19 contiguous nucleotides) differing by no more than 4 nucleotides from any one of the sense strand sequences of Tables 1A-1C. In some embodiments, the sense strand differs by no more than 3 nucleotides from any one of the sense strand sequences of Tables 1A-1C. In some embodiments, the sense strand differs by no more than 2 nucleotides from any one of the sense strand sequences of Tables 1A-1C. In some embodiments, the sense strand differs by no more than 1 nucleotide from any one of the sense strand sequences of Tables 1A-1C. In some embodiments, the sense strand comprises a sequence of any one of the sense strands of Tables 1A-1C. In some embodiments, the sense strand consists of a sequence of any one of the sense strands of Tables 1A-1C.

In some embodiments, the sense strand comprises a nucleotide sequence comprising at least 15 contiguous nucleotides (for example, 15, 16, 17, 18, or 19 contiguous nucleotides) differing by no more than 4 nucleotides from the nucleotide sequence of any one of SEQ ID NOs: 1-628 or 1257-1884. In some embodiments, the sense strand differs by no more than 3 nucleotides from the nucleotide sequence of any one of SEQ ID NOs: 1-628 or 1257-1884. In some embodiments, the sense strand differs by no more than 2 nucleotides from the nucleotide sequence of any one of SEQ ID NOs: 1-628 or 1257-1884. In some embodiments, the sense strand differs by no more than 1 nucleotide from the nucleotide sequence of any one of SEQ ID NOs: 1-628 or 1257-1884. In some embodiments, the sense strand comprises the nucleotide sequence of any one of SEQ ID NOs: 1-628 or 1257-1884. In some embodiments, the sense strand consists of the nucleotide sequence of any one of SEQ ID NOs: 1-628 or 1257-1884.

In some embodiments, the sense strand comprises a nucleotide sequence comprising at least 15 contiguous nucleotides (for example, 15, 16, 17, 18, or 19 contiguous nucleotides) differing by no more than 4 nucleotides from the nucleotide sequence of any one of SEQ ID NOs: 2524-2643. In some embodiments, the sense strand differs by no more than 3 nucleotides from the nucleotide sequence of any one of SEQ ID NOs: 2524-2643. In some embodiments, the sense strand differs by no more than 2 nucleotides from the nucleotide sequence of any one of SEQ ID NOs: 2524-2643. In some embodiments, the sense strand differs by no more than 1 nucleotide from the nucleotide sequence of any one of SEQ ID NOs: 2524-2643. In some embodiments, the sense strand comprises the nucleotide sequence of any one of SEQ ID NOs: 2524-2643. In some embodiments, the sense strand consists of the nucleotide sequence of any one of SEQ ID NOs: 2524-2643.

In some embodiments, the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to a portion of the nucleotide sequence of any one of SEQ ID NOs: 1-628 or 1257-1884. In some embodiments, the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to the nucleotide sequence of any one of SEQ ID NOs: 1-628 or 1257-1884.

In some embodiments, the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to a portion of the nucleotide sequence of any one of SEQ ID NOs: 2524-2643. In some embodiments, the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to the nucleotide sequence of any one of SEQ ID NOs: 2524-2643.

In some embodiments, the sense strand consists of a nucleotide sequence having the nucleotide sequence of any one of SEQ ID NOs: 1-628 or 1257-1884. In some embodiments, the sense strand consists of a nucleotide sequence having the nucleotide sequence of any one of SEQ ID NOs: 2524-2643.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides from the nucleotide sequence of any one of SEQ ID NOs: 629-1256 or 1885-2522, and the sense strand comprises a nucleotide sequence at least 15 contiguous nucleotides (for example, 15, 16, 17, 18, or 19 contiguous nucleotides) differing by no more than 4 nucleotides from the nucleotide sequence of any one of SEQ ID NOs: 1-628 or 1257-1884.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides from the nucleotide sequence of any one of SEQ ID NOs: 2644-2763, and the sense strand comprises a nucleotide sequence at least 15 contiguous nucleotides (for example, 15, 16, 17, 18, or 19 contiguous nucleotides) differing by no more than 4 nucleotides from the nucleotide sequence of any one of SEQ ID NOs: 2524-2643.

In some embodiments, the antisense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to a portion of the nucleotide sequence of any one of SEQ ID NOs: 629-1256 or 1885-2522, and the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to a portion of the nucleotide sequence of any one of SEQ ID NOs: 1-628 or 1257-1884.

In some embodiments, the antisense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to a portion of the nucleotide sequence of any one of SEQ ID NOs: 2644-2763, and the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to a portion of the nucleotide sequence of any one of SEQ ID NOs: 2524-2643.

In some embodiments, the antisense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to the nucleotide sequence of any one of SEQ ID NOs: 629-1256 or 1885-2522, and the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to the nucleotide sequence of any one of SEQ ID NOs: 1-628 or 1257-1884.

In some embodiments, the antisense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to the nucleotide sequence of any one of SEQ ID NOs: 2644-2763, and the sense strand comprises a nucleotide sequence having at least 90% nucleotide sequence identity (for example, at least 90%, 91%, 92%, 93%, 94%, 95%, 96%, 97%, 98%, 99%, or 100% nucleotide sequence identity) to the nucleotide sequence of any one of SEQ ID NOs: 2524-2643.

In some embodiments, the antisense strand consists of a nucleotide sequence of any one of SEQ ID NOs: 629-1256 or 1885-2522, and the sense strand consists of a nucleotide sequence of any one of SEQ ID NOs: 1-628 or 1257-1884.

In some embodiments, the antisense strand consists of a nucleotide sequence of any one of SEQ ID NOs: 2644-2763, and the sense strand consists of a nucleotide sequence of any one of SEQ ID NOs: 2524-2643.

In some embodiments, the oligonucleotide of the present disclosure targets a sequence of nucleotides corresponding to any one of positions 202-220, 1209-1227, 1211-1229, 1481-1499, 2424-2442, 2430-2448, 42-60, 219-237, 329-347, 330-348, 1130-1148, 1202-1220, 1503-1521, 1504-1522, 1900-1918, 1906-1924, 1939-1957, 1940-1958, 1948-1966, 2301-2319, 2309-2327, 2425-2443, 2429-2447, 2431-2449, 226-244, 227-245, 228-246, 260-278, 276-294, 667-685, 668-686, 959-977, 1099-1117, 1100-1118, 1590-1608, 2522-2540, 2576-2594, 2837-2855, 225-243, 476-494, 477-495, 484-502, 809-827, 617-635, 666-684, 669-687, 274-292, 275-293, 1098-1116, 1101-1119, 1413-1431, 1483-1501, 1484-1502, 1485-1503, 1600-1618, 1876-1894, 1933-1951, 2158-2176, 2269-2287, 2349-2367, 2856-2874, 2857-2875, 278-296, 2692-2710, 757-775, 1379-1397, 2220-2238, 2102-2120, 2690-2708, 52-70, 1380-1398, 1132-1150, 1126-1144, 281-299, 2232-2250, 2219-2237, 199-217, 2093-2111, 1378-1396, 2840-2858, 1134-1152, 2222-2240, 2703-2721, 358-376, 285-303, 2184-2202, 1377-1395, 359-377, 194-212, 888-906, 193-211, 209-227, 210-228, 211-229, 248-266, 249-267, 250-268, 251-269, 252-270, 253-271, 606-624, 607-625, 608-626, 609-627, 203-221, 204-222, 206-224, 207-225, 223-241, 230-248, 300-318, 301-319, 302-320, 303-321, 306-324, 307-325, 308-326, 309-327, 310-328, 311-329, 312-330, 314-332, 315-333, 321-339, 322-340, 323-341, 324-342, 325-343, 326-344, 327-345, 328-346, 331-349, 334-352, 335-353, 336-354, 337-355, 338-356, 349-367, 350-368, 351-369, 356-374, 360-378, 399-417, 400-418, 451-469, 470-488, 471-489, 475-493, 478-496, 480-498, 481-499, 482-500, 483-501, 683-701, 684-702, 715-733, 716-734, 717-735, 786-804, 788-806, 789-807, 810-828, 811-829, 812-830, 264-282, 521-539, 522-540, 523-541, 524-542, 525-543, 593-611, 610-628, 618-636, 623-641, 624-642, 718-736, 776-794, 777-795, 778-796, 779-797, 780-798, 813-831, 839-857, 926-944, 49-67, 54-72, 55-73, 109-127, 110-128, 113-131, 115-133, 195-213, 231-249, 232-250, 233-251, 234-252, 237-255, 240-258, 244-262, 296-314, 304-322, 305-323, 316-334, 317-335, 318-336, 319-337, 320-338, 341-359, 342-360, 347-365, 352-370, 362-380, 402-420, 446-464, 450-468, 452-470, 479-497, 528-546, 595-613, 598-616, 600-618, 601-619, 602-620, 604-622, 605-623, 671-689, 672-690, 674-692, 675-693, 676-694, 679-697, 680-698, 681-699, 682-700, 690-708, 782-800, 783-801, 784-802, 785-803, 787-805, 790-808, 791-809, 792-810, 803-821, 838-856, 848-866, 849-867, 850-868, 872-890, 873-891, 876-894, 877-895, 878-896, 882-900, 883-901, 886-904, 889-907, 950-968, 951-969, 952-970, 953-971, 978-996, 979-997, 980-998, 983-1001, 984-1002, 1066-1084, 1068-1086, 1071-1089, 1072-1090, 1073-1091, 1077-1095, 1078-1096, 1079-1097, 1082-1100, 1083-1101, 1118-1136, 1119-1137, 1121-1139, 1205-1223, 1206-1224, 1207-1225, 1208-1226, 1210-1228, 1375-1393, 1417-1435, 1418-1436, 1419-1437, 1420-1438, 1422-1440, 1424-1442, 1956-1974, 1957-1975, 1958-1976, 2025-2043, 2030-2048, 2087-2105, 2088-2106, 2094-2112, 2099-2117, 2145-2163, 2146-2164, 2147-2165, 2148-2166, 2150-2168, 2151-2169, 2152-2170, 2153-2171, 2182-2200, 2183-2201, 2188-2206, 2216-2234, 2228-2246, 2281-2299, 2282-2300, 2284-2302, 2288-2306, 2293-2311, 2295-2313, 2297-2315, 2303-2321, 2306-2324, 2307-2325, 2308-2326, 2310-2328, 2311-2329, 2315-2333, 2317-2335, 2380-2398, 2389-2407, 2390-2408, 2420-2438, 2432-2450, 2434-2452, 2437-2455, 2443-2461, 2601-2619, 2681-2699, 2697-2715, 2700-2718, 2947-2965, 17-35, 18-36, 19-37, 20-38, 21-39, 45-63, 47-65, 48-66, 91-109, 94-112, 95-113, 96-114, 97-115, 98-116, 99-117, 100-118, 101-119, 102-120, 103-121, 104-122, 105-123, 106-124, 107-125, 108-126, 111-129, 112-130, 114-132, 161-179, 163-181, 181-199, 182-200, 183-201, 184-202, 185-203, 186-204, 187-205, 188-206, 189-207, 197-215, 198-216, 200-218, 201-219, 205-223, 212-230, 213-231, 214-232, 215-233, 217-235, 221-239, 222-240, 224-242, 229-247, 254-272, 255-273, 256-274, 257-275, 290-308, 291-309, 292-310, 293-311, 297-315, 298-316, 299-317, 313-331, 346-364, 348-366, 353-371, 354-372, 355-373, 357-375, 361-379, 363-381, 381-399, 382-400, 383-401, 387-405, 388-406, 389-407, 390-408, 391-409, 392-410, 393-411, 394-412, 438-456, 440-458, 441-459, 442-460, 443-461, 449-467, 469-487, 472-490, 473-491, 474-492, 526-544, 527-545, 531-549, 532-550, 533-551, 537-555, 538-556, 628-646, 630-648, 631-649, 639-657, 640-658, 641-659, 642-660, 643-661, 644-662, 645-663, 646-664, 670-688, 673-691, 677-695, 687-705, 688-706, 689-707, 711-729, 712-730, 713-731, 714-732, 781-799, 793-811, 797-815, 800-818, 842-860, 844-862, 845-863, 846-864, 928-946, 949-967, 957-975, 958-976, 960-978, 961-979, 962-980, 963-981, 964-982, 965-983, 966-984, 967-985, 968-986, 969-987, 972-990, 981-999, 985-1003, 1022-1040, 1023-1041, 1024-1042, 1031-1049, 1032-1050, 1061-1079, 1062-1080, 1063-1081, 1064-1082, 1067-1085, 1069-1087, 1070-1088, 1074-1092, 1075-1093, 1080-1098, 1081-1099, 1084-1102, 1086-1104, 1087-1105, 1120-1138, 1122-1140, 1136-1154, 1178-1196, 1179-1197, 1231-1249, 1232-1250, 1233-1251, 1234-1252, 1235-1253, 1236-1254, 1239-1257, 1240-1258, 1266-1284, 1267-1285, 1268-1286, 1286-1304, 1287-1305, 1288-1306, 1289-1307, 1290-1308, 1291-1309, 1292-1310, 1293-1311, 1294-1312, 1295-1313, 1296-1314, 1297-1315, 1304-1322, 1312-1330, 1313-1331, 1314-1332, 1315-1333, 1366-1384, 1367-1385, 1368-1386, 1369-1387, 1370-1388, 1371-1389, 1372-1390, 1373-1391, 1412-1430, 1414-1432, 1415-1433, 1416-1434, 1421-1439, 1423-1441, 1426-1444, 1430-1448, 1431-1449, 1432-1450, 1433-1451, 1434-1452, 1435-1453, 1436-1454, 1437-1455, 1438-1456, 1439-1457, 1440-1458, 1441-1459, 1442-1460, 1466-1484, 1467-1485, 1468-1486, 1469-1487, 1498-1516, 1501-1519, 1502-1520, 1505-1523, 1506-1524, 1508-1526, 1526-1544, 1527-1545, 1528-1546, 1529-1547, 1530-1548, 1531-1549, 1532-1550, 1533-1551, 1543-1561, 1674-1692, 1675-1693, 1706-1724, 1707-1725, 1708-1726, 1711-1729, 1782-1800, 1783-1801, 1817-1835, 1822-1840, 2000-2018, 2001-2019, 2002-2020, 116-134, 261-279, 263-281, 267-285, 268-286, 534-552, 535-553, 1089-1107, 1090-1108, 1091-1109, 1092-1110, 1093-1111, 1094-1112, 1095-1113, 1096-1114, 1097-1115, 1228-1246, 1229-1247, 1614-1632, 1615-1633, 1616-1634, 1673-1691, 1701-1719, 1702-1720, 1703-1721, 1704-1722, 1784-1802, 1818-1836, and 1821-1839 of SEQ ID NO: 2523 (NM_002667.5).

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from an antisense strand selected from Table 1B, and the sense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from a sense strand selected from Table 1B. In some embodiments, the antisense strand comprises a nucleotide sequence differing by no more than 4 nucleotides (for example, 1, 2, 3, or 4 nucleotides) from an antisense strand selected from Table 1B, and the sense strand comprises a nucleotide sequence differing by no more than 4 nucleotides (for example, 1, 2, 3, or 4 nucleotides) from a sense strand selected from Table 1B. In some embodiments, the antisense strand comprises an antisense strand selected from Table 1B, and the sense strand comprises a sense strand selected from Table 1B. In some embodiments, the antisense strand consists of an antisense strand selected from Table 1B, and the sense strand consists of a sense strand selected from Table 1B.

In some embodiments, the oligonucleotide of the present disclosure targets a sequence of nucleotides corresponding to any one of positions 470-488, 471-489, 475-493, 450-468, 452-470, 528-546, 381-399, 382-400, 383-401, and 472-490 of SEQ ID NO: 2523 (NM_002667.5).

In some embodiments, the antisense strand comprises a nucleotide sequence comprising at least 15 contiguous nucleotides (for example, 15, 16, 17, 18, 19, 20, or 21 contiguous nucleotides) differing by no more than 1, 2, 3, or 4 nucleotides from a sequence selected from SEQ ID NOs: 774, 775, 776, 843, 844, 846, 1042, 1043, 1044, and 1060. In some embodiments, the sense strand comprises a nucleotide sequence comprising at least 15 contiguous nucleotides (for example, 15, 16, 17, 18, or 19 contiguous nucleotides) differing by no more than 1, 2, 3, or 4 nucleotides from a nucleotide sequence selected from SEQ ID NOs: 146, 147, 148, 215, 216, 218, 414, 415, 416, and 432.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 774, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 146. In some embodiments, the antisense strand comprises a nucleotide sequence differing by no more than 4 nucleotides (for example, 1, 2, 3, or 4 nucleotides) from SEQ ID NO: 774, and the sense strand comprises a nucleotide sequence differing by no more than 4 nucleotides (for example, 1, 2, 3, or 4 nucleotides) from SEQ ID NO: 146. In some embodiments, the antisense strand comprises the nucleotide sequence SEQ ID NO: 774, and the sense strand comprises the nucleotide sequence SEQ ID NO: 146. In some embodiments, the antisense strand consists of the nucleotide sequence SEQ ID NO: 774, and the sense strand consists of the nucleotide sequence SEQ ID NO: 146.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 775, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 147. In some embodiments, the antisense strand comprises a nucleotide sequence differing by no more than 4 nucleotides (for example, 1, 2, 3, or 4 nucleotides) from SEQ ID NO: 775, and the sense strand comprises a nucleotide sequence differing by no more than 4 nucleotides (for example, 1, 2, 3, or 4 nucleotides) from SEQ ID NO: 147. In some embodiments, the antisense strand comprises the nucleotide sequence SEQ ID NO: 775, and the sense strand comprises the nucleotide sequence SEQ ID NO: 147. In some embodiments, the antisense strand consists of the nucleotide sequence SEQ ID NO: 775, and the sense strand consists of the nucleotide sequence SEQ ID NO: 147.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 776, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 148. In some embodiments, the antisense strand comprises a nucleotide sequence differing by no more than 4 nucleotides (for example, 1, 2, 3, or 4 nucleotides) from SEQ ID NO: 776, and the sense strand comprises a nucleotide sequence differing by no more than 4 nucleotides (for example, 1, 2, 3, or 4 nucleotides) from SEQ ID NO: 148. In some embodiments, the antisense strand comprises the nucleotide sequence SEQ ID NO: 776, and the sense strand comprises the nucleotide sequence SEQ ID NO: 148. In some embodiments, the antisense strand consists of the nucleotide sequence SEQ ID NO: 776, and the sense strand consists of the nucleotide sequence SEQ ID NO: 148.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 843, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 215. In some embodiments, the antisense strand comprises a nucleotide sequence differing by no more than 4 nucleotides (for example, 1, 2, 3, or 4 nucleotides) from SEQ ID NO: 843, and the sense strand comprises a nucleotide sequence differing by no more than 4 nucleotides (for example, 1, 2, 3, or 4 nucleotides) from SEQ ID NO: 215. In some embodiments, the antisense strand comprises the nucleotide sequence SEQ ID NO: 843, and the sense strand comprises the nucleotide sequence SEQ ID NO: 215. In some embodiments, the antisense strand consists of the nucleotide sequence SEQ ID NO: 843, and the sense strand consists of the nucleotide sequence SEQ ID NO: 215.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 844, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 216. In some embodiments, the antisense strand comprises a nucleotide sequence differing by no more than 4 nucleotides (for example, 1, 2, 3, or 4 nucleotides) from SEQ ID NO: 844, and the sense strand comprises a nucleotide sequence differing by no more than 4 nucleotides (for example, 1, 2, 3, or 4 nucleotides) from SEQ ID NO: 216. In some embodiments, the antisense strand comprises the nucleotide sequence SEQ ID NO: 844, and the sense strand comprises the nucleotide sequence SEQ ID NO: 216. In some embodiments, the antisense strand consists of the nucleotide sequence SEQ ID NO: 844, and the sense strand consists of the nucleotide sequence SEQ ID NO: 216.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 846, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 218. In some embodiments, the antisense strand comprises a nucleotide sequence differing by no more than 4 nucleotides (for example, 1, 2, 3, or 4 nucleotides) from SEQ ID NO: 846, and the sense strand comprises a nucleotide sequence differing by no more than 4 nucleotides (for example, 1, 2, 3, or 4 nucleotides) from SEQ ID NO: 218. In some embodiments, the antisense strand comprises the nucleotide sequence SEQ ID NO: 846, and the sense strand comprises the nucleotide sequence SEQ ID NO: 218. In some embodiments, the antisense strand consists of the nucleotide sequence SEQ ID NO: 846, and the sense strand consists of the nucleotide sequence SEQ ID NO: 218.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1042, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 414. In some embodiments, the antisense strand comprises a nucleotide sequence differing by no more than 4 nucleotides (for example, 1, 2, 3, or 4 nucleotides) from SEQ ID NO: 1042, and the sense strand comprises a nucleotide sequence differing by no more than 4 nucleotides (for example, 1, 2, 3, or 4 nucleotides) from SEQ ID NO: 414. In some embodiments, the antisense strand comprises the nucleotide sequence SEQ ID NO: 1042, and the sense strand comprises the nucleotide sequence SEQ ID NO: 414. In some embodiments, the antisense strand consists of the nucleotide sequence SEQ ID NO: 1042, and the sense strand consists of the nucleotide sequence SEQ ID NO: 414.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1043, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 415. In some embodiments, the antisense strand comprises a nucleotide sequence differing by no more than 4 nucleotides (for example, 1, 2, 3, or 4 nucleotides) from SEQ ID NO: 1043, and the sense strand comprises a nucleotide sequence differing by no more than 4 nucleotides (for example, 1, 2, 3, or 4 nucleotides) from SEQ ID NO: 415. In some embodiments, the antisense strand comprises the nucleotide sequence SEQ ID NO: 1043, and the sense strand comprises the nucleotide sequence SEQ ID NO: 415. In some embodiments, the antisense strand consists of the nucleotide sequence SEQ ID NO: 1043, and the sense strand consists of the nucleotide sequence SEQ ID NO: 415.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1044, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 416. In some embodiments, the antisense strand comprises a nucleotide sequence differing by no more than 4 nucleotides (for example, 1, 2, 3, or 4 nucleotides) from SEQ ID NO: 1044, and the sense strand comprises a nucleotide sequence differing by no more than 4 nucleotides (for example, 1, 2, 3, or 4 nucleotides) from SEQ ID NO: 416. In some embodiments, the antisense strand comprises the nucleotide sequence SEQ ID NO: 1044, and the sense strand comprises the nucleotide sequence SEQ ID NO: 416. In some embodiments, the antisense strand consists of the nucleotide sequence SEQ ID NO: 1044, and the sense strand consists of the nucleotide sequence SEQ ID NO: 416.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1060, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 432. In some embodiments, the antisense strand comprises a nucleotide sequence differing by no more than 4 nucleotides (for example, 1, 2, 3, or 4 nucleotides) from SEQ ID NO: 1060, and the sense strand comprises a nucleotide sequence differing by no more than 4 nucleotides (for example, 1, 2, 3, or 4 nucleotides) from SEQ ID NO: 432. In some embodiments, the antisense strand comprises the nucleotide sequence SEQ ID NO: 1060, and the sense strand comprises the nucleotide sequence SEQ ID NO: 432. In some embodiments, the antisense strand consists of the nucleotide sequence SEQ ID NO: 1060, and the sense strand consists of the nucleotide sequence SEQ ID NO: 432.

In some embodiments, the antisense strand comprises a nucleotide sequence comprising at least 15 contiguous nucleotides (for example, 15, 16, 17, 18, 19, 20, or 21 contiguous nucleotides) differing by no more than 1, 2, 3, or 4 nucleotides from a sequence selected from SEQ ID NOs: 2030, 2031, 2032, 2099, 2100, 2102, 2298, 2299, 2300, 2316, 2513, 2514, 2515, 2516, 2517, 2518, 2519, 2520, 2521, and 2522. In some embodiments, the sense strand comprises a nucleotide sequence comprising at least 15 contiguous nucleotides (for example, 15, 16, 17, 18, or 19 contiguous nucleotides) differing by no more than 1, 2, 3, or 4 nucleotides from a nucleotide sequence selected from SEQ ID NOs: 1402, 1403, 1404, 1471, 1472, 1474, 1670, 1671, 1672, and 1688. In some embodiments, the antisense strand comprises a nucleotide sequence comprising at least 15 contiguous nucleotides (for example, 15, 16, 17, 18, 19, 20, or 21 contiguous nucleotides) differing by no more than 1, 2, 3, or 4 nucleotides from a sequence selected from SEQ ID NOs: 2030, 2031, 2032, 2099, 2100, 2102, 2298, 2299, 2300, 2316, 2513, 2514, 2515, 2516, 2517, 2518, 2519, 2520, 2521, and 2522, and the sense strand comprises a nucleotide sequence comprising at least 15 contiguous nucleotides (for example, 15, 16, 17, 18, or 19 contiguous nucleotides) differing by no more than 1, 2, 3, or 4 nucleotides from a nucleotide sequence selected from SEQ ID NOs: 1402, 1403, 1404, 1471, 1472, 1474, 1670, 1671, 1672, and 1688.

In some embodiments, the antisense strand comprises a nucleotide sequence comprising at least 15 contiguous nucleotides (for example, 15, 16, 17, 18, 19, 20, or 21 contiguous nucleotides) differing by no more than 1, 2, 3, or 4 nucleotides from a sequence selected from SEQ ID NOs: 2513, 2514, 2515, 2516, 2517, 2518, 2519, 2520, 2521, and 2522. In some embodiments, the sense strand comprises a nucleotide sequence comprising at least 15 contiguous nucleotides (for example, 15, 16, 17, 18, or 19 contiguous nucleotides) differing by no more than 1, 2, 3, or 4 nucleotides from a nucleotide sequence selected from SEQ ID NOs: 1402, 1403, 1404, 1471, 1472, 1474, 1670, 1671, 1672, and 1688.

In some embodiments, the antisense strand comprises a nucleotide sequence comprising at least 15 contiguous nucleotides (for example, 15, 16, 17, 18, 19, 20, or 21 contiguous nucleotides) differing by no more than 1, 2, 3, or 4 nucleotides from a sequence selected from SEQ ID NOs: 2513, 2514, 2515, 2516, 2517, 2518, 2519, 2520, 2521, and 2522, and the sense strand comprises a nucleotide sequence comprising at least 15 contiguous nucleotides (for example, 15, 16, 17, 18, or 19 contiguous nucleotides) differing by no more than 1, 2, 3, or 4 nucleotides from a nucleotide sequence selected from SEQ ID NOs: 1402, 1403, 1404, 1471, 1472, 1474, 1670, 1671, 1672, and 1688.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1402, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2030. In some embodiments, the antisense strand comprises a nucleotide sequence differing by no more than 4 nucleotides (for example, 1, 2, 3, or 4 nucleotides) from SEQ ID NO: 1402, and the sense strand comprises a nucleotide sequence differing by no more than 4 nucleotides (for example, 1, 2, 3, or 4 nucleotides) from SEQ ID NO: 2030. In some embodiments, the antisense strand comprises the nucleotide sequence of SEQ ID NO: 1402, and the sense strand comprises the nucleotide sequence of SEQ ID NO: 2030. In some embodiments, the antisense strand consists of the nucleotide sequence of SEQ ID NO: 1402, and the sense strand consists of the nucleotide sequence of SEQ ID NO: 2030.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1403, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2031. In some embodiments, the antisense strand comprises a nucleotide sequence differing by no more than 4 nucleotides (for example, 1, 2, 3, or 4 nucleotides) from SEQ ID NO: 1403, and the sense strand comprises a nucleotide sequence differing by no more than 4 nucleotides (for example, 1, 2, 3, or 4 nucleotides) from SEQ ID NO: 2031. In some embodiments, the antisense strand comprises the nucleotide sequence of SEQ ID NO: 1403, and the sense strand comprises the nucleotide sequence of SEQ ID NO: 2031. In some embodiments, the antisense strand consists of the nucleotide sequence of SEQ ID NO: 1403, and the sense strand consists of the nucleotide sequence of SEQ ID NO: 2031.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1404, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2032. In some embodiments, the antisense strand comprises a nucleotide sequence differing by no more than 4 nucleotides (for example, 1, 2, 3, or 4 nucleotides) from SEQ ID NO: 1404, and the sense strand comprises a nucleotide sequence differing by no more than 4 nucleotides (for example, 1, 2, 3, or 4 nucleotides) from SEQ ID NO: 2032. In some embodiments, the antisense strand comprises the nucleotide sequence of SEQ ID NO: 1404, and the sense strand comprises the nucleotide sequence of SEQ ID NO: 2032. In some embodiments, the antisense strand consists of the nucleotide sequence of SEQ ID NO: 1404, and the sense strand consists of the nucleotide sequence of SEQ ID NO: 2032.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1471, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2099. In some embodiments, the antisense strand comprises a nucleotide sequence differing by no more than 4 nucleotides (for example, 1, 2, 3, or 4 nucleotides) from SEQ ID NO: 1471, and the sense strand comprises a nucleotide sequence differing by no more than 4 nucleotides (for example, 1, 2, 3, or 4 nucleotides) from SEQ ID NO: 2099. In some embodiments, the antisense strand comprises the nucleotide sequence of SEQ ID NO: 1471, and the sense strand comprises the nucleotide sequence of SEQ ID NO: 2099. In some embodiments, the antisense strand consists of the nucleotide sequence of SEQ ID NO: 1471, and the sense strand consists of the nucleotide sequence of SEQ ID NO: 2099.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1472, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2100. In some embodiments, the antisense strand comprises a nucleotide sequence differing by no more than 4 nucleotides (for example, 1, 2, 3, or 4 nucleotides) from SEQ ID NO: 1472, and the sense strand comprises a nucleotide sequence differing by no more than 4 nucleotides (for example, 1, 2, 3, or 4 nucleotides) from SEQ ID NO: 2100. In some embodiments, the antisense strand comprises the nucleotide sequence of SEQ ID NO: 1472, and the sense strand comprises the nucleotide sequence of SEQ ID NO: 2100. In some embodiments, the antisense strand consists of the nucleotide sequence of SEQ ID NO: 1472, and the sense strand consists of the nucleotide sequence of SEQ ID NO: 2100.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1474, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2102. In some embodiments, the antisense strand comprises a nucleotide sequence differing by no more than 4 nucleotides (for example, 1, 2, 3, or 4 nucleotides) from SEQ ID NO: 1474, and the sense strand comprises a nucleotide sequence differing by no more than 4 nucleotides (for example, 1, 2, 3, or 4 nucleotides) from SEQ ID NO: 2102. In some embodiments, the antisense strand comprises the nucleotide sequence of SEQ ID NO: 1474, and the sense strand comprises the nucleotide sequence of SEQ ID NO: 2102. In some embodiments, the antisense strand consists of the nucleotide sequence of SEQ ID NO: 1474, and the sense strand consists of the nucleotide sequence of SEQ ID NO: 2102.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1670, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2298. In some embodiments, the antisense strand comprises a nucleotide sequence differing by no more than 4 nucleotides (for example, 1, 2, 3, or 4 nucleotides) from SEQ ID NO: 1670, and the sense strand comprises a nucleotide sequence differing by no more than 4 nucleotides (for example, 1, 2, 3, or 4 nucleotides) from SEQ ID NO: 2298. In some embodiments, the antisense strand comprises the nucleotide sequence of SEQ ID NO: 1670, and the sense strand comprises the nucleotide sequence of SEQ ID NO: 2298. In some embodiments, the antisense strand consists of the nucleotide sequence of SEQ ID NO: 1670, and the sense strand consists of the nucleotide sequence of SEQ ID NO: 2298.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1671, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2299. In some embodiments, the antisense strand comprises a nucleotide sequence differing by no more than 4 nucleotides (for example, 1, 2, 3, or 4 nucleotides) from SEQ ID NO: 1671, and the sense strand comprises a nucleotide sequence differing by no more than 4 nucleotides (for example, 1, 2, 3, or 4 nucleotides) from SEQ ID NO: 2299. In some embodiments, the antisense strand comprises the nucleotide sequence of SEQ ID NO: 1671, and the sense strand comprises the nucleotide sequence of SEQ ID NO: 2299. In some embodiments, the antisense strand consists of the nucleotide sequence of SEQ ID NO: 1671, and the sense strand consists of the nucleotide sequence of SEQ ID NO: 2299.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1672, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2300. In some embodiments, the antisense strand comprises a nucleotide sequence differing by no more than 4 nucleotides (for example, 1, 2, 3, or 4 nucleotides) from SEQ ID NO: 1672, and the sense strand comprises a nucleotide sequence differing by no more than 4 nucleotides (for example, 1, 2, 3, or 4 nucleotides) from SEQ ID NO: 2300. In some embodiments, the antisense strand comprises the nucleotide sequence of SEQ ID NO: 1672, and the sense strand comprises the nucleotide sequence of SEQ ID NO: 2300. In some embodiments, the antisense strand consists of the nucleotide sequence of SEQ ID NO: 1672, and the sense strand consists of the nucleotide sequence of SEQ ID NO: 2300.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 1688, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 2316. In some embodiments, the antisense strand comprises a nucleotide sequence differing by no more than 4 nucleotides (for example, 1, 2, 3, or 4 nucleotides) from SEQ ID NO: 1688, and the sense strand comprises a nucleotide sequence differing by no more than 4 nucleotides (for example, 1, 2, 3, or 4 nucleotides) from SEQ ID NO: 2316. In some embodiments, the antisense strand comprises the nucleotide sequence of SEQ ID NO: 1688, and the sense strand comprises the nucleotide sequence of SEQ ID NO: 2316. In some embodiments, the antisense strand consists of the nucleotide sequence of SEQ ID NO: 1688, and the sense strand consists of the nucleotide sequence of SEQ ID NO: 2316.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2513, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1402. In some embodiments, the antisense strand comprises a nucleotide sequence differing by no more than 4 nucleotides (for example, 1, 2, 3, or 4 nucleotides) from SEQ ID NO: 2513, and the sense strand comprises a nucleotide sequence differing by no more than 4 nucleotides (for example, 1, 2, 3, or 4 nucleotides) from SEQ ID NO: 1402. In some embodiments, the antisense strand comprises the nucleotide sequence of SEQ ID NO: 2513, and the sense strand comprises the nucleotide sequence of SEQ ID NO: 1402. In some embodiments, the antisense strand consists of the nucleotide sequence of SEQ ID NO: 2513, and the sense strand consists of the nucleotide sequence of SEQ ID NO: 1402.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2514, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1403. In some embodiments, the antisense strand comprises a nucleotide sequence differing by no more than 4 nucleotides (for example, 1, 2, 3, or 4 nucleotides) from SEQ ID NO: 2514, and the sense strand comprises a nucleotide sequence differing by no more than 4 nucleotides (for example, 1, 2, 3, or 4 nucleotides) from SEQ ID NO: 1403. In some embodiments, the antisense strand comprises the nucleotide sequence of SEQ ID NO: 2514, and the sense strand comprises the nucleotide sequence of SEQ ID NO: 1403. In some embodiments, the antisense strand consists of the nucleotide sequence of SEQ ID NO: 2514, and the sense strand consists of the nucleotide sequence of SEQ ID NO: 1403.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2515, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1404. In some embodiments, the antisense strand comprises a nucleotide sequence differing by no more than 4 nucleotides (for example, 1, 2, 3, or 4 nucleotides) from SEQ ID NO: 2515, and the sense strand comprises a nucleotide sequence differing by no more than 4 nucleotides (for example, 1, 2, 3, or 4 nucleotides) from SEQ ID NO: 1404. In some embodiments, the antisense strand comprises the nucleotide sequence of SEQ ID NO: 2515, and the sense strand comprises the nucleotide sequence of SEQ ID NO: 1404. In some embodiments, the antisense strand consists of the nucleotide sequence of SEQ ID NO: 2515, and the sense strand consists of the nucleotide sequence of SEQ ID NO: 1404.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2516, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1471. In some embodiments, the antisense strand comprises a nucleotide sequence differing by no more than 4 nucleotides (for example, 1, 2, 3, or 4 nucleotides) from SEQ ID NO: 2516, and the sense strand comprises a nucleotide sequence differing by no more than 4 nucleotides (for example, 1, 2, 3, or 4 nucleotides) from SEQ ID NO: 1471. In some embodiments, the antisense strand comprises the nucleotide sequence of SEQ ID NO: 2516, and the sense strand comprises the nucleotide sequence of SEQ ID NO: 1471. In some embodiments, the antisense strand consists of the nucleotide sequence of SEQ ID NO: 2516, and the sense strand consists of the nucleotide sequence of SEQ ID NO: 1471.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2517, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1472. In some embodiments, the antisense strand comprises a nucleotide sequence differing by no more than 4 nucleotides (for example, 1, 2, 3, or 4 nucleotides) from SEQ ID NO: 2517, and the sense strand comprises a nucleotide sequence differing by no more than 4 nucleotides (for example, 1, 2, 3, or 4 nucleotides) from SEQ ID NO: 1472. In some embodiments, the antisense strand comprises the nucleotide sequence of SEQ ID NO: 2517, and the sense strand comprises the nucleotide sequence of SEQ ID NO: 1472. In some embodiments, the antisense strand consists of the nucleotide sequence of SEQ ID NO: 2517, and the sense strand consists of the nucleotide sequence of SEQ ID NO: 1472.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2518, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1474. In some embodiments, the antisense strand comprises a nucleotide sequence differing by no more than 4 nucleotides (for example, 1, 2, 3, or 4 nucleotides) from SEQ ID NO: 2518, and the sense strand comprises a nucleotide sequence differing by no more than 4 nucleotides (for example, 1, 2, 3, or 4 nucleotides) from SEQ ID NO: 1474. In some embodiments, the antisense strand comprises the nucleotide sequence of SEQ ID NO: 2518, and the sense strand comprises the nucleotide sequence of SEQ ID NO: 1474. In some embodiments, the antisense strand consists of the nucleotide sequence of SEQ ID NO: 2518, and the sense strand consists of the nucleotide sequence of SEQ ID NO: 1474.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2519, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1670. In some embodiments, the antisense strand comprises a nucleotide sequence differing by no more than 4 nucleotides (for example, 1, 2, 3, or 4 nucleotides) from SEQ ID NO: 2519, and the sense strand comprises a nucleotide sequence differing by no more than 4 nucleotides (for example, 1, 2, 3, or 4 nucleotides) from SEQ ID NO: 1670. In some embodiments, the antisense strand comprises the nucleotide sequence of SEQ ID NO: 2519, and the sense strand comprises the nucleotide sequence of SEQ ID NO: 1670. In some embodiments, the antisense strand consists of the nucleotide sequence of SEQ ID NO: 2519, and the sense strand consists of the nucleotide sequence of SEQ ID NO: 1670.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2520, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1671. In some embodiments, the antisense strand comprises a nucleotide sequence differing by no more than 4 nucleotides (for example, 1, 2, 3, or 4 nucleotides) from SEQ ID NO: 2520, and the sense strand comprises a nucleotide sequence differing by no more than 4 nucleotides (for example, 1, 2, 3, or 4 nucleotides) from SEQ ID NO: 1671. In some embodiments, the antisense strand comprises the nucleotide sequence of SEQ ID NO: 2520, and the sense strand comprises the nucleotide sequence of SEQ ID NO: 1671. In some embodiments, the antisense strand consists of the nucleotide sequence of SEQ ID NO: 2520, and the sense strand consists of the nucleotide sequence of SEQ ID NO: 1671.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2521, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1672. In some embodiments, the antisense strand comprises a nucleotide sequence differing by no more than 4 nucleotides (for example, 1, 2, 3, or 4 nucleotides) from SEQ ID NO: 2521, and the sense strand comprises a nucleotide sequence differing by no more than 4 nucleotides (for example, 1, 2, 3, or 4 nucleotides) from SEQ ID NO: 1672. In some embodiments, the antisense strand comprises the nucleotide sequence of SEQ ID NO: 2521, and the sense strand comprises the nucleotide sequence of SEQ ID NO: 1672. In some embodiments, the antisense strand consists of the nucleotide sequence of SEQ ID NO: 2521, and the sense strand consists of the nucleotide sequence of SEQ ID NO: 1672.

In some embodiments, the antisense strand comprises a nucleotide sequence comprising at least 15 contiguous nucleotides (for example, 15, 16, 17, 18, 19, 20, or 21 contiguous nucleotides) differing by no more than 4 nucleotides (for example, 1, 2, 3, or 4 nucleotides) from SEQ ID NO: 2522, and the sense strand comprises a nucleotide sequence comprising at least 15 contiguous nucleotides (for example, 15, 16, 17, 18, or 19 contiguous nucleotides) differing by no more than 4 nucleotides (for example, 1, 2, 3, or 4 nucleotides) from SEQ ID NO: 1688. In some embodiments, the antisense strand comprises a nucleotide sequence differing by no more than 4 nucleotides (for example, 1, 2, 3, or 4 nucleotides) from SEQ ID NO: 2522, and the sense strand comprises a nucleotide sequence differing by no more than 4 nucleotides (for example, 1, 2, 3, or 4 nucleotides) from SEQ ID NO: 1688. In some embodiments, the antisense strand comprises the nucleotide sequence of SEQ ID NO: 2522, and the sense strand comprises the nucleotide sequence of SEQ ID NO: 1688. In some embodiments, the antisense strand consists of the nucleotide sequence of SEQ ID NO: 2522, and the sense strand consists of the nucleotide sequence of SEQ ID NO: 1688.

A PLN RNAi agent described herein can contain one or more mismatches to the target sequence (e.g., a sequence of between 15 and 30 nucleotides in length). In some embodiments, a PLN RNAi agent as described herein contains no more than 4 mismatches (e.g., no more than 4, 3, 2, 1, or 0 mismatches to the target sequence). If the antisense strand of the RNAi agent contains mismatches to a target sequence, it may be preferable that the area of mismatch is not located in the center of the region of complementarity. If the antisense strand of the RNAi agent contains mismatches to the target sequence, it may be preferable that the mismatch be restricted to be within the last 5 nucleotides from either the 5′- or 3′-end of the region of complementarity. For example, for a 21 nucleotide RNAi agent, the strand which is complementary to a region of, e.g., PLN, generally does not contain any mismatch within the central 11 nucleotides. The methods described herein or methods known in the art can be used to determine whether an RNAi agent containing a mismatch to a target sequence is effective in inhibiting the expression of PLN. Consideration of the efficacy of RNAi agents with mismatches in inhibiting expression of a target gene is important, especially if the particular region of complementarity in a target gene is known to have polymorphic sequence variation within the population.

In some embodiments, reduction in expression levels is assayed using an RT-qPCR assay to perform relative quantification of PLN mRNA in iCell cardiomyocytes. The skilled artisan will appreciate that various approaches to quantify knockdown of PLN expression for assessing RNAi agent activity may be used in the art, including commercially available kits, e.g., from Thermo. In some embodiments, any one of the RNAi agents described herein can reduce expression levels of a PLN mRNA when assayed in cultured cells. Any suitable cells known in the art may be used to assess the RNAi agents. In some embodiments, the cultured cells are iCell cardiomyocytes. In some embodiments, administration of any one of the RNAi agents disclosed herein to a cultured cell results in a reduction in expression level of a PLN. In some embodiments, administration of the RNAi agent results in at least a 30% reduction in expression levels of the PLN mRNA. In some embodiments, administration of the RNAi agent results in at least a 35% reduction in expression levels of the PLN mRNA. In some embodiments, administration of the RNAi agent results in at least a 40% reduction in expression levels of the PLN mRNA. In some embodiments, administration of the RNAi agent results in at least a 45% reduction in expression levels of the PLN mRNA. In some embodiments, administration of the RNAi agent results in at least a 50% reduction in expression levels of the PLN mRNA. In some embodiments, administration of the RNAi agent results in at least a 55% reduction in expression levels of the PLN mRNA. In some embodiments, administration of the RNAi agent results in at least a 60% reduction in expression levels of the PLN mRNA. In some embodiments, administration of the RNAi agent results in at least a 65% reduction in expression levels of the PLN mRNA. In some embodiments, administration of the RNAi agent results in at least a 70% reduction in expression levels of the PLN mRNA. In some embodiments, administration of the RNAi agent results in at least a 75% reduction in expression levels of the PLN mRNA. In some embodiments, administration of the RNAi agent results in at least an 80% reduction in expression levels of the PLN mRNA. In some embodiments, administration of the RNAi agent results in at least an 85% reduction in expression levels of the PLN mRNA. In some embodiments, administration of the RNAi agent results in at least a 90% reduction in expression levels of the PLN mRNA. In some embodiments, administration of the RNAi agent results in at least a 95% reduction in expression levels of the PLN mRNA.

### Single-Stranded Antisense Oligonucleotides

In some embodiments, the oligonucleotide is a single-stranded antisense oligonucleotide, or “ASO.” ASOs comprise an antisense strand with at least partial complementary to a target sequence in an RNA. Upon binding to a target sequence, downregulation of the RNA may be achieved through various mechanisms, including, but not limited to, sterically blocking translation or recruitment of RNase H.

In some embodiments, the ASO comprises a nucleic acid sequence of at least 14 contiguous nucleotides (for example, 14, 15, 16, 17, 18, 19, 20, or 21 contiguous nucleotides) that differ by no more than 1, 2, 3, or 4 nucleotides from any one of SEQ ID NOs: 629-1256 or 1885-2522. In some embodiments, the ASO comprises a nucleotide sequence of any one of SEQ ID NOs: 629-1256 or 1885-2522. In some embodiments, the ASO comprises a nucleic acid sequence of at least 14 contiguous nucleotides (for example, 14, 15, 16, 17, 18, 19, 20, or 21 contiguous nucleotides) that differ by no more than 1, 2, 3, or 4 nucleotides from any one of SEQ ID NOs: 2644-2763. In some embodiments, the ASO comprises a nucleotide sequence of any one of SEQ ID NOs: 2644-2763.

In some embodiments, the single-stranded ASO comprises a nucleic acid sequence comprising at least 14, 15, 16, 17, 18, 19, 20, or 21 contiguous nucleotides that differ by no more than 1, 2, 3, or 4 nucleotides from any one of SEQ ID NOs: 2513, 2514, 2515, 2516, 2517, 2518, 2519, 2520, 2521, and 2522.

In some embodiments, the ASO comprises 2′-deoxy ribonucleotides and phosphorothioate internucleoside linkages.

In some embodiments, the ASO is a “gapmer” ASO comprising a 2′-deoxy “gap” region flanked by “wings” having nucleotides with 2′-modified ribonucleotides. In some embodiments, the ASO is an “MOE gapmer” in which the 2′-modified ribonucleotide is a 2′-O-methoxyethyl (2′-MOE or simply MOE) modification, and each of the internucleoside linkages is a phosphorothioate.

### Modified Nucleotides

The oligonucleotides disclosed herein may be modified or unmodified. In some embodiments, any one of the oligonucleotides contain one or more modifications. As used herein, a modification to a nucleotide or “modified nucleotide” refers to any nucleotide other than the canonical ribonucleotides adenine, guanine, cytosine, and uracil.

In some embodiments, the oligonucleotide comprises one or more modified nucleotides. In some embodiments, no more than 1, 2, 3, 4, or 5 of the nucleotides of the oligonucleotide are unmodified nucleotides. In some embodiments, all nucleotides of the oligonucleotide are modified nucleotides.

In some embodiments, wherein the oligonucleotide is an RNAi agent, the antisense strand comprises one or more modified nucleotides. In some embodiments, no more than 1, 2, 3, 4, or 5 of the nucleotides of the antisense strand are unmodified nucleotides. In some embodiments, all nucleotides of the antisense strand are modified nucleotides.

In some embodiments, wherein the oligonucleotide is an RNAi agent, the sense strand comprises one or more modified nucleotides. In some embodiments, no more than 1, 2, 3, 4, or 5 of the nucleotides of the sense strand are unmodified nucleotides. In some embodiments, all nucleotides of the sense strand are modified nucleotides.

Modified nucleotides include, but are not limited to 2′-modified nucleotides, 3′ to 3′ linkages (inverted) nucleotides, bridged nucleotides, 2′,3′-seco nucleotide mimics (e.g., unlocked nucleobase analogues (UNAs), locked nucleotides (LNAs), 5′-(S)-methyl-2′-deoxy-2′-fluoronucleotide (5′Me-Nf), vinyl phosphonate deoxyribonucleotides, vinyl phosphonate nucleotides, a nucleotide comprising a 5′-phosphorothioate group, a nucleotide comprising a 5′-methylphosphonate group, a nucleotide comprising a 5′ phosphate or 5′ phosphate mimic and N-(methane sulfonyl) phosphoramidate group. In some embodiments, 2′-modified nucleotides (e.g., a nucleotide with a group other than a hydroxyl group at the 2′ position of the five-membered sugar ring) include, but are not limited to, 2′-O-methyl nucleotides (represented herein as a lower case letter n in a nucleotide sequence), 2′-deoxy-2′-fluoro nucleotides (represented herein as Nf, also represented herein as 2′-fluoro nucleotide). In some embodiments, a modified nucleotide is a nucleotide comprising a 5′-methylphosphate group. In some embodiments, a modified nucleotide is a nucleotide comprising a 5′-C-methylphosphate group.

In some embodiments, modified nucleotides include, but are not limited to, deoxyribonucleotides, nucleotide mimics, abasic nucleotides, 5′ to 5′ linkages (inverted) nucleotides, non-natural base-comprising nucleotides, peptide nucleic acids (PNAs), 3′-O-methoxy (2′ internucleoside linked) nucleotides, 2′-deoxy-2′-fluoro-arabino nucleotides, cyclopropyl phosphonate nucleotides (cPrpN), vinyl phosphonate 2′-(methoxyethyl) unlocked nucleotides, a nucleotide comprising 2-hydroxymethyl-tetrahydrofurane-5-phosphate, 2′-deoxy nucleotides (represented herein as dN), 2′-methoxy ethyloxy (2′-O-(2-methoxylethyl)) nucleotides, 2′-amino nucleotides, and 2′-alkyl nucleotides. It is not necessary for all positions in a given compound to be uniformly modified. Conversely, more than one modification may be incorporated in a single oligonucleotide. Modification at one nucleotide is independent of modification at another nucleotide.

In some embodiments, the nucleotide modification comprises a deoxyribonucleotide, a 3′-terminal deoxythymidine (dT) nucleotide, an abasic nucleotide, a 2′-modified nucleotide, a 3′ to 3′ linkages (inverted) nucleotide, a 5′ to 5′ linkages (inverted) nucleotide, a non-natural base-comprising nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising 2-hydroxymethyl-tetrahydrofurane-5-phosphate, or a nucleotide comprising a N-(methane sulfonyl) phosphoramidate group. In some embodiments, the nucleotide modification comprises a nucleotide comprising a 5′-methylphosphate group. In some embodiments, a modified nucleotide is a nucleotide comprising a 5′-C-methylphosphate group. In some embodiments, the oligonucleotide comprises a modified nucleotide selected from the group consisting of a deoxy nucleotide, a 3′-terminal deoxythymidine (dT) nucleotide, a 3′-3′ inverted nucleotide linkage, a 5′-5′ inverted nucleotide linkage, a 5′-(E)-vinylphosphonate-2′-O-methyl-uridine-3′-phosphate, a 5′-(E)-vinylphosphonate-2′-O-methyl-uridine-3′-phosphorothioate, and a combination thereof.

In some embodiments, the one or more modifications is selected from a ribose modification, a backbone modification, a nucleobase modification, or a combination thereof. In some embodiments, the one or more modifications is a combination of a ribose modification, a backbone modification, and/or a nucleobase modification.

In some embodiments, the ribose modification comprises a locked nucleic acid (LNA), a tricyclo-DNA (tcDNA), 2′-deoxy-2′-fluoro, 2′-O-methyl, 2′-methoxyethyl (2′-MOE), 2′-deoxy-2′-arabino-fluoro, 2′-O-benzyl, 2′-O-methyl-4-pyridine, 2′ cyclic ethyl (cET), phosphorodiamidate morpholino (PMO), glycol nucleic acid (GNA), unlocked nucleic acid (UNA), a threose nucleic acid (TNA), or a combination thereof. In some embodiments, the ribose modification comprises a 2′-deoxy-2′-fluoro, 2′-O-methyl, 2′-methoxyethyl (2′-MOE), glycol nucleic acid (GNA), unlocked nucleic acid (UNA), a threose nucleic acid (TNA), or a combination thereof. In some embodiments, the ribose modification comprises a 2′-deoxy-2′-fluoro, 2′-O-methyl, glycol nucleic acid (GNA), unlocked nucleic acid (UNA), a threose nucleic acid (TNA), or a combination thereof. In some embodiments, the ribose modification is 2′-deoxy-2′-fluoro modification, a 2′-O-methyl modification, or a combination thereof.

In some embodiments, the oligonucleotide comprises one or more nucleobase modifications. Nucleobase modifications include, for example, synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine, or combinations thereof.

In some embodiments, the oligonucleotide comprises one or more backbone modification or non-standard linkages (e.g., modified internucleoside linkages). In some embodiments, a backbone modification is a non-phosphate-containing covalent internucleoside linkage. Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified backbone or modified internucleoside linkage lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioform acetyl backbones, alkene-containing backbones, sulfamate backbones, methyieneimino and methylenehydrazino backbones, sulfonate and sulfonamide backbones, amide backbones, and other backbones having mixed N, O, S, and CH₂ components.

In some embodiments, the backbone modification comprises phosphorothioate.

In some embodiments, any one of the oligonucleotides disclosed herein further comprise a sequence of 3′-terminal deoxythymidine (dT) nucleotides. In some embodiments, any one of the antisense strands described herein further comprises at least one terminal dT nucleotide. In some embodiments, any one of the antisense strands described herein further comprises 2 or more terminal dT nucleotides, e.g., 2, 3, 4, or more terminal dT nucleotides. In some embodiments, wherein the oligonucleotide is an RNAi agent, any one of the sense strands described herein further comprises at least one terminal dT nucleotide. In some embodiments, any one of the sense strands described herein further comprises 2 or more terminal dT nucleotides, e.g., 2, 3, 4, or more terminal dT nucleotides. In some embodiments, the antisense and sense strand of an RNAi agent herein each contain one or more terminal dT nucleotides.

In some embodiments, any one of the oligonucleotides disclosed herein further comprises at least one phosphorothioate internucleoside or phosphorodithioate internucleoside linkage. In some embodiments, any one of the oligonucleotides disclosed herein further comprises at least one phosphorothioate internucleoside linkage. In some embodiments, at least one phosphorothioate internucleoside linkage is at the 5′ end of the antisense strand. In some embodiments, the oligonucleotide comprises at least 2 phosphorothioate internucleoside linkages at the 5′ end of the antisense strand. In some embodiments, at least one phosphorothioate internucleoside linkage is at the 3′ end of the antisense strand. In some embodiments, the oligonucleotide comprises at least 2 phosphorothioate internucleoside linkages at the 3′ end of the antisense strand. In some embodiments, wherein the oligonucleotide is an RNAi agent, the at least one phosphorothioate internucleoside linkage is at the 5′ end of the sense strand. In some embodiments, the oligonucleotide comprises at least 2 phosphorothioate internucleoside linkages at the 5′ end of the sense strand. In some embodiments, the at least one phosphorothioate internucleoside linkage is at the 3′ end of the sense strand. In some embodiments, the oligonucleotide comprises at least 2 phosphorothioate internucleoside linkages at the 3′ end of the sense strand.

In some embodiments, any one of the oligonucleotides disclosed herein further comprises a terminal, chiral modification. In some embodiments, the terminal chiral modification is at the first internucleoside linkage at the 3′ end of the sense and/or antisense strand. In some embodiments, the terminal chiral modification is at the first and second internucleoside linkage at the 3′ end of the sense and/or antisense strand. In some embodiments, the chiral modification comprises a phosphorus atom of the internucleoside linkage being in either Rp or Sp configuration. In some embodiments, the siRNA agent further comprises a terminal, chiral modification occurring at the first internucleoside linkage at the 3′ end of the antisense strand, having the linkage phosphorus atom in Sp configuration, a terminal, chiral modification occurring at the first internucleoside linkage at the 5′ end of the antisense strand, having the linkage phosphorus atom in Rp configuration, and a terminal, chiral modification occurring at the first internucleoside linkage at the 5′ end of the sense strand, having the linkage phosphorus atom in either Rp configuration or Sp configuration. In some embodiments, the siRNA agent further comprises a terminal, chiral modification occurring at the first and second internucleoside linkages at the 3′ end of the antisense strand, having the linkage phosphorus atom in Sp configuration, a terminal, chiral modification occurring at the first internucleoside linkage at the 5′ end of the antisense strand, having the linkage phosphorus atom in Rp configuration, and a terminal, chiral modification occurring at the first internucleoside linkage at the 5′ end of the sense strand, having the linkage phosphorus atom in either Rp or Sp configuration. In some embodiments, the siRNA agent further comprises a terminal, chiral modification occurring at the first, second, and third internucleoside linkages at the 3′ end of the antisense strand, having the linkage phosphorus atom in Sp configuration, a terminal, chiral modification occurring at the first internucleoside linkage at the 5′ end of the antisense strand, having the linkage phosphorus atom in Rp configuration, and a terminal, chiral modification occurring at the first internucleoside linkage at the 5′ end of the sense strand, having the linkage phosphorus atom in either Rp or Sp configuration. In some embodiments, the siRNA agent further comprises a terminal, chiral modification occurring at the first and second internucleoside linkages at the 3′ end of the antisense strand, having the linkage phosphorus atom in Sp configuration, a terminal, chiral modification occurring at the third internucleoside linkages at the 3′ end of the antisense strand, having the linkage phosphorus atom in Rp configuration, a terminal, chiral modification occurring at the first internucleoside linkage at the 5′ end of the antisense strand, having the linkage phosphorus atom in Rp configuration, and a terminal, chiral modification occurring at the first internucleoside linkage at the 5′ end of the sense strand, having the linkage phosphorus atom in either Rp or Sp configuration. In some embodiments, the siRNA agent further comprises a terminal, chiral modification occurring at the first and second internucleoside linkages at the 3′ end of the antisense strand, having the linkage phosphorus atom in Sp configuration, a terminal, chiral modification occurring at the first and second internucleoside linkages at the 5′ end of the antisense strand, having the linkage phosphorus atom in Rp configuration, and a terminal, chiral modification occurring at the first internucleoside linkage at the 5′ end of the sense strand, having the linkage phosphorus atom in either Rp or Sp configuration.

In some embodiments, any one of the oligonucleotides disclosed herein further comprises a phosphate or phosphate mimic at the 5′-end of the antisense strand. In some embodiments, a phosphate or phosphate mimic at the 5′-end of the antisense strand is a 5′-vinyl phosphonate (VP). In some embodiments, a phosphate or phosphate mimic at the 5′-end of the antisense strand is a 5′-(E)-vinyl phosphonate (VP).

### RNAi Agent Modification Motifs

In some embodiments, any one of the antisense or sense strands disclosed herein are modified according to a modification motif or pattern.

In some embodiments, an antisense strand disclosed herein comprises a modification pattern nsNfsnnnNfnNfNfnnnnNfnNfnnnsnsn, wherein ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

In some embodiments, an antisense strand disclosed herein comprises a modification pattern vpUsNfsnnnNfnNfNfnnnnNfnNfnnnsnsn, wherein vpUs is a 5′-vinylphosphonate-2′-O-methyl-uridine-3′-phosphorothioate; ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

In some embodiments, a sense strand disclosed herein comprises a modification pattern nsnsnnNfnNfNfNfnnnnnnnnnn, wherein ns is a 2′-O-methyl-nucleoside-3′-phosphorothioate; n is a 2′-O-methyl-nucleoside-3′-phosphate; and Nf is a 2′-deoxy-2′-fluoro-nucleoside-3′-phosphate.

In some embodiments, a sense strand disclosed herein comprises a modification pattern NfsnsNfnNfnNfnNfnNfnNfnNfnNfnNf, wherein ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

In some embodiments, an RNAi agent disclosed herein comprises a sense strand and an antisense strand, wherein the sense strand comprises a modification pattern nsnsnnNfnNfNfNfnnnnnnnnnn, and the antisense strand comprises a modification pattern nsNfsnnnNfnNfNfnnnnNfnNfnnnsnsn, wherein ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

In some embodiments, an RNAi agent disclosed herein comprises a sense strand and an antisense strand, wherein the sense strand comprises a modification pattern nsnsnnNfnNfNfNfnnnnnnnnnn, and the antisense strand comprises a modification pattern vpUsNfsnnnNfnNfNfnnnnNfnNfnnnsnsn, wherein vpUs is a 5′-vinylphosphonate-2′-O-methyl-uridine-3′-phosphorothioate; ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

In some embodiments, a strand of an RNAi agent comprises a modification pattern according to Nfsₐ(nNf)_{b}ns_{c}n, wherein n is a 2′-O-methyl-nucleoside-3′-phosphate; Nfs is a 2′-deoxy-2′-fluoro-nucleoside-3′-phosphorothioate; Nf is a 2′-deoxy-2′-fluoro-nucleoside-3′-phosphate; and ns is a 2′-O-methyl-nucleoside-3′-phosphorothioate; and wherein a is at least 1, b is at least 5-10, and c is at least 1. In some embodiments, a is at least 2, b is at least 8, and c is at least 2. In some embodiments, a is 2, b is 8, and c is 2. In some embodiments, the strand of the RNAi agent comprises the modification pattern NfsNfsnNfnNfnNfnNfnNfnNfnNfnNfnsnsn. In some embodiments, the strand is the antisense strand. In some embodiments, the antisense strand comprises the sequence of any one of SEQ ID NOs: 629-1256 or 1885-2522. In some embodiments, the antisense strand comprises the sequence of any one of SEQ ID NOs: 2644-2763. In some embodiments, the strand is the sense strand. In some embodiments, the sense strand comprises the sequence of any one of SEQ ID NOs: 1-628 or 1257-1884. In some embodiments, the sense strand comprises the sequence of any one of SEQ ID NOs: 2524-2643.

In some embodiments, a strand of an RNAi agent comprises a modification pattern according ns_{d}(Nfn)ₑNf, wherein n is a 2′-O-methyl-nucleoside-3′-phosphate; Nf is a 2′-deoxy-2′-fluoro-nucleoside-3′-phosphate; and ns is a 2′-O-methyl-nucleoside-3′-phosphorothioate; and wherein d is at least 1, and e is 5-10. In some embodiments, d is at least 2 and e is at least 8. In some embodiments, the RNAi agent comprises the modification pattern nsnsNfnNfnNfnNfnNfnNfnNfnNfnNf. In some embodiments, the strand is the sense strand. In some embodiments, the sense strand comprises the sequence of any one of SEQ ID NOs: 1-628 or 1257-1884. In some embodiments, the sense strand comprises the sequence of any one of SEQ ID NOs: 2524-2643. In some embodiments, the RNAi agent comprises the modification pattern nsnsNfnNfnNfnNfnNfnNfnNfnNfnNfnsn. In some embodiments, the strand is the antisense strand. In some embodiments, the antisense strand comprises the sequence of any one of SEQ ID NOs: 629-1256 or 1885-2522. In some embodiments, the antisense strand comprises the sequence of any one of SEQ ID NOs: 2644-2763.

In some embodiments, the antisense strand comprises a modification pattern according to Nfsₐ(nNf)_{b}ns_{c}n, wherein n is a 2′-O-methyl-nucleoside-3′-phosphate; Nfs is a 2′-deoxy-2′-fluoro-nucleoside-3′-phosphorothioate; Nf is a 2′-deoxy-2′-fluoro-nucleoside-3′-phosphate; and ns is a 2′-O-methyl-nucleoside-3′-phosphorothioate; and wherein a is at least 1, b is at least 5-10, and c is at least 1, and the sense strand comprises a modification pattern according ns_{d}(Nfn)ₑNf, wherein n is a 2′-O-methyl-nucleoside-3′-phosphate; Nf is a 2′-deoxy-2′-fluoro-nucleoside-3′-phosphate; and ns is a 2′-O-methyl-nucleoside-3′-phosphorothioate; and wherein d is at least 1, and e is 5-10. In some embodiments, the antisense strand comprises a nucleotide sequence according to any one of SEQ ID NOs: 629-1256 or 1885-2522. In some embodiments, the sense strand comprises a nucleotide sequence according to any one of SEQ ID NOs: 1-628 or 1257-1884. In some embodiments, the antisense strand comprises the sequence of any one of SEQ ID NOs: 2644-2763. In some embodiments, the sense strand comprises the sequence of any one of SEQ ID NOs: 2524-2643.

In some embodiments, the antisense strand comprises, or consists of, SEQ ID NO: 2513, and the sense strand comprises, or consists of, SEQ ID NO: 1402. In some embodiments, the antisense strand comprises, or consists of, SEQ ID NO: 2514, and the sense strand comprises, or consists of, SEQ ID NO: 1403. In some embodiments, the antisense strand comprises, or consists of, SEQ ID NO: 2515, and the sense strand comprises, or consists of, SEQ ID NO: 1404. In some embodiments, the antisense strand comprises, or consists of, SEQ ID NO: 2516, and the sense strand comprises, or consists of, SEQ ID NO: 1471. In some embodiments, the antisense strand comprises, or consists of, SEQ ID NO: 2517, and the sense strand comprises, or consists of, SEQ ID NO: 1472. In some embodiments, the antisense strand comprises, or consists of, SEQ ID NO: 2518, and the sense strand comprises, or consists of, SEQ ID NO: 1474. In some embodiments, the antisense strand comprises, or consists of, SEQ ID NO: 2519, and the sense strand comprises, or consists of, SEQ ID NO: 1670. In some embodiments, the antisense strand comprises, or consists of, SEQ ID NO: 2520, and the sense strand comprises, or consists of, SEQ ID NO: 1671. In some embodiments, the antisense strand comprises, or consists of, SEQ ID NO: 2521, and the sense strand comprises, or consists of, SEQ ID NO: 1672. In some embodiments, the antisense strand comprises, or consists of, SEQ ID NO: 2522, and the sense strand comprises, or consists of, SEQ ID NO: 1688.

In some embodiments, any one of the RNAi agents disclosed herein comprises a modification pattern wherein the antisense strand comprises the motif F(SF)nSnn, wherein n is from 2 to about 20, nn is 0 or 1, one of F and S is a 2′-deoxy-2′-fluoro modified nucleoside and the other of F and S is a 2′-O-methyl modified nucleoside. In some embodiments, each of the antisense and the sense strand is 17-23 nucleotides in length. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand comprises the chemical modification pattern of nNfnnnNfnNfNfnnnnNfnNfnnnnn and the sense strand comprises the chemical modification nnnnnnNfnNfNfNfnnnnnnnn, wherein n is a 2′-O-methyl-nucleoside and Nf is a 2′-deoxy-2′-fluoro-nucleoside. In some embodiments, the antisense strand corresponds to the unmodified nucleobase sequence according to any one of SEQ ID NOs: 629-1256. In some embodiments, the sense strand corresponds to the unmodified nucleobase sequence of any one according to SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand comprises a region having the formula X1-Y—X2, wherein Y is a subregion of from about 5 to about 12 linked nucleosides and each of X1 subregion and X2 subregion is, independently, a plurality of linked nucleosides having the formula FSFS, where one of F and S is a 2′-deoxy-2′-fluoro modified nucleoside and the other of F and S is a 2′-O-methyl modified nucleoside; and each internucleoside linkage of said X1 subregion and X2 subregion is, independently, a phosphodiester or a phosphorothioate internucleoside linkage. In some embodiments, each of the antisense and the sense strand is 20-23 nucleotides in length. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628.

In some embodiments, any one of the RNAi agents disclosed herein comprises a modification pattern wherein the antisense strand comprises a contiguous sequence of linked nucleosides that define an alternating motif of the formula: 5′-Q(-L-Z-L-Q)n(-L-Z)nn-3′, wherein: each L is an internucleoside linking group; either each Q is a 2′-deoxy-2′-fluoro nucleoside and each Z is a 2′-O-methyl nucleoside; or each Q is a 2′-O-methyl nucleoside and each Z is a 2′-deoxy-2′-fluoro nucleoside; and n is from 8 to 14 and nn is 0 or 1. In some embodiments, the antisense strand corresponds to the unmodified nucleobase sequence according to any one of SEQ ID NOs: 629-1256. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand is represented by the formula:

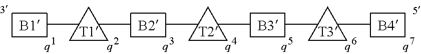

wherein, B1′, B2′, B3′, and B4′ each independently represent a nucleotide containing a modification selected from the group consisting of 2′-O-alkyl, 2′-substituted alkoxy, 2′-substituted alkyl, 2′-halo, ENA, and BNA/LNA; T1′, T2′, and T3′ each independently represent a nucleotide comprising a chemical modification selected from the group consisting of DNA, RNA, LNA, 2′-deoxy-2′-fluoronucleotide, and 5′-(S)-methyl2′-deoxy-2′-fluoronucleotide; q1 is 4 to 15 nucleotides in length; q3 or q7 is independently 1-6 nucleotide(s) in length; q2 or q6 is independently 1-3 nucleotide(s) in length; q4 is 0-3 nucleotide(s) in length; and q5 is 0-10 nucleotide(s) in length; and wherein: the antisense strand has 2′-deoxy-2′-fluoro modifications, and wherein the 2′-deoxy-2′-fluoro modifications on the antisense strand consist of four, and only four, 2′-deoxy-2′-fluoro modifications or six, and only six, 2′-deoxy-2′-fluoro modifications. In some embodiments, the antisense strand is 19-25 nucleotides in length. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand and the sense strand are represented by the formula:

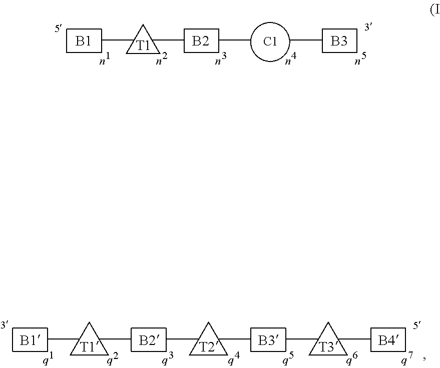

wherein B1, B1′, B2′, B3′, and B4′ each independently represent a nucleotide containing a modification selected from the group consisting of 2′-O-alkyl, 2′-substituted alkoxy, 2′-substituted alkyl, 2′-halo, ENA, and BNA/LNA; C1 is a thermally destabilizing nucleotide, selected from the group consisting of i) a nucleotide that forms a mismatch pair with the opposing nucleotide in the antisense strand, ii) a nucleotide having an abasic modification, and iii) a nucleotide having a sugar modification, and placed at a site opposite to the seed region (positions 2-8) of the antisense strand; T1′, T2′, and T3′ each independently represent a nucleotide comprising a modification providing the nucleotide a steric bulk that is less than or equal to the steric bulk of a 2′-OMe modification, wherein the modification is at the 2′-position of a ribose sugar of the nucleotide or at a position of a non-ribose nucleotide similar to the 2′-position of a ribose sugar; each n1, and q1 is independently 4 to 15 nucleotides in length; each q3, and q7 is independently 1-6 nucleotide(s) in length; each q2 and q6 is independently 1-3 nucleotide(s) in length; q5 is 0-10 nucleotide(s) in length; each n4, and q4 is independently 0-3 nucleotide(s) in length; n2 is 3 nucleotides in length, and T1 each are 2′-deoxy-2′-fluoronucleotides; n3 is 7 nucleotides in length, and B2 each are 2′-OMe nucleotides; and n5 is 3 nucleotides in length, and B3 each are 2′-OMe nucleotides. In some embodiments, wherein the antisense strand and sense strand are each 14 to 40 nucleotides. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand is represented by the formula:

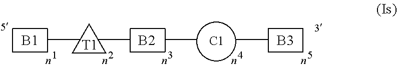

wherein: B1, B2, and B3 each independently represent a nucleotide containing a modification selected from the group consisting of 2′-O-alkyl, 2′-substituted alkoxy, 2′-substituted alkyl, 2′-halo, ENA, and BNA/LNA; C1 is a thermally destabilizing nucleotide, selected from the group consisting of i) a nucleotide that forms a mismatch pair with the opposing nucleotide in the antisense strand, ii) a nucleotide having an abasic modification, and iii) a nucleotide having a sugar modification, and placed at a site opposite to the seed region (positions 2-8) of the antisense strand; T1 represents a nucleotide comprising a 2′-deoxy-2′-fluoro modification; n1 or n3 is independently 4 to 15 nucleotides in length; n5 is 1-6 nucleotide(s) in length; n2 is 3; n4 is 0-3 nucleotide(s) in length; and wherein the sense strand has 2′-deoxy-2′-fluoro modifications, and wherein the 2′-deoxy-2′-fluoro modifications on the sense strand consist of four, and only four, 2′-deoxy-2′-fluoro modifications, wherein the four 2′-deoxy-2′-fluoro modifications are at positions 7 and 9-11 from the 5′-end of the sense strand. In some embodiments, wherein the antisense strand and sense strand are each 19-25 nucleotides. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand is complementary to at least one portion of a mRNA of the target gene (e.g., PLN), wherein the oligonucleotide is represented by the formula:

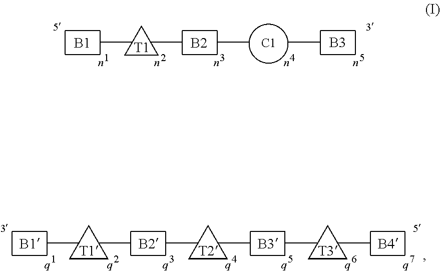

wherein: B1′, B2′, B3′, and B4′ each independently represent a nucleotide containing a modification selected from the group consisting of 2′-O-methyl and 2′-deoxy-2′-fluoro; each B1, B2, and B3 is 2′-OMe; C1 is glycerol nucleic acid (GNA) placed at a site opposite to the seed region (positions 2-8) of the antisense strand; T1′, T2′, and T3′ are each 2′-deoxy-2′-fluoronucleotides, wherein: T1′ is at position 14 from the 5′ end of the antisense strand, and q2 is 1; and T3′ is at position 2 from the 5′ end of the antisense strand, and q6 and q7 are 1; each n1, n3, and q1 is independently 4 to 15 nucleotides in length; each n5 and q3 is independently 1-6 nucleotide(s) in length; q5 is 0-10 nucleotide(s) in length; each n4 and q4 is independently 0-3 nucleotide(s) in length; n2 is 3 nucleotides in length, and T1 each are 2′-deoxy-2′-fluoro nucleotides, and wherein (a) the oligonucleotide is covalently conjugated to at least one ligand; and (b) one of the T1 nucleotides is at position 11 from the 5′ end of the sense strand. In some embodiments, the sense strand comprises 19-22 nucleotides and the antisense strand comprises 19-25 nucleotides. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the oligonucleotide is represented by the formula:

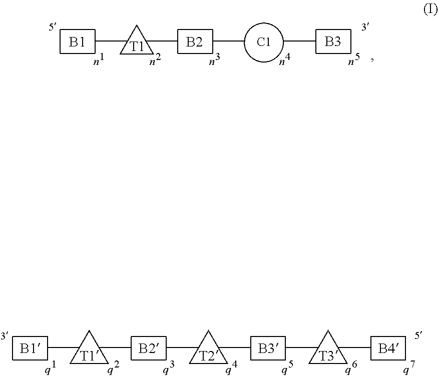

wherein B1′, B2′, B3′, and B4′ each independently represent a nucleotide containing a modification selected from the group consisting of 2′-O-methyl and 2′-deoxy-2′-fluoro; each B1, B2, and B3 is 2′-Ome nucleotides; C1 is glycerol nucleic acid (GNA) placed at a site opposite to the seed region (positions 2-8) of the antisense strand; T1′, T2′, and T3′ are each 2′-deoxy-2′-fluoro nucleotides, wherein: T1′ is at position 14 from the 5′ end of the antisense strand, and q2 is 1; and T3′ is at position 2 from the 5′ end of the antisense strand, and q6 and q7 are 1; each n1, n3, and q1 is independently 4 to 15 nucleotides in length; each n5 and q3 is independently 1-6 nucleotide(s) in length; q5 is 0-10 nucleotide(s) in length; each n4 and q4 is independently 0-3 nucleotide(s) in length; n2 is 3 nucleotides in length, and T1 each are 2′-deoxy-2′-fluoro nucleotides, and wherein (a) the oligonucleotide is covalently conjugated to at least one ligand; and (b) one of the T1 nucleotides is at a position in the sense strand that is opposite to position 11 from the 5′ end of the antisense strand; and (c) the oligonucleotide comprises at least one phosphorothioate internucleoside linkage. In some embodiments, the sense strand comprises 19-22 nucleotides and the antisense strand comprises 19-25 nucleotides. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

In some embodiments, any one of the RNAi agents disclosed herein comprises a modification pattern wherein the antisense strand has sufficient complementarity to a target sequence to mediate RNA interference, wherein said sense strand comprises at least one thermally destabilizing modification of the duplex within the first 9 nucleotide positions of the 5′ region of the antisense strand or a precursor thereof, wherein the antisense strand further comprises one or both of the following characteristics: (i) 2, 3, 4, 5 or 6 2′-deoxy-2′-fluoro modifications; and (ii) 1, 2, 3, 4 or 5 phosphorothioate internucleoside linkages; and said sense strand comprises one or both of the following characteristics: (iii) 2, 3, 4, or 5 2′-deoxy-2′-fluoro modifications; and (iv) 1, 2, 3, 4 or 5 phosphorothioate internucleoside linkages. In some embodiments, the antisense strand and sense strand are each 14 to 40 nucleotides. In some embodiments, the antisense strand corresponds to the unmodified nucleobase sequence according to any one of SEQ ID NOs: 629-1256. In some embodiments, the sense strand corresponds to the unmodified nucleobase sequence according to any one of SEQ ID NOs: 1-628. In some embodiments, the antisense strand corresponds to the unmodified antisense strand in Table 1C. In some embodiments, the sense strand corresponds to the unmodified sense strand in Table 1C.

### Synthesis, Purification, and Analysis of the Oligonucleotides Described Herein

Oligomer synthesis of modified and unmodified nucleosides and nucleotides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713). The oligonucleotides provided herein can be conveniently and routinely made through the well-known technique of solid phase synthesis. Equipment for such synthesis is sold by several vendors including, for example, LGC (Alexandria, MN). Any other means for such synthesis known in the art may additionally or alternatively be employed. It is well known to use similar techniques to prepare oligonucleotides such as the phosphorothioates and alkylated derivatives. Methods of purification and analysis of oligomeric compounds are known to those skilled in the art. Analysis methods include capillary electrophoresis (CE), reverse-phase high performance liquid chromatography (RP-HPLC), and electrospray-mass spectroscopy (ESI-MS). Such synthesis and analysis methods can be performed in multi-well plates. The oligonucleotides of the invention can be prepared using solution-phase or solid-phase organic synthesis, or enzymatically by methods known in the art. Organic synthesis offers the advantage that the oligomeric strands comprising non-natural or modified nucleotides can be easily prepared. Any other means for such synthesis known in the art can additionally or alternatively be employed. It is also known to use similar techniques to prepare other oligonucleotides, such as those comprising phosphorothioates, phosphorodithioates and alkylated derivatives of internucleoside linkages. The double-stranded oligonucleotides of the invention can be prepared using a two-step procedure. First, the individual strands of the double-stranded oligonucleotides are prepared separately. Then, the component strands are annealed. Regardless of the method of synthesis, the oligonucleotides can be prepared in a solution (e.g., an aqueous and/or organic solution) that is appropriate for formulation. For example, the oligonucleotides preparation can be precipitated and redissolved in pure double-distilled water, and lyophilized. The dried oligonucleotides can then be resuspended in a solution appropriate for the intended formulation process. Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof, U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having beta-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. Nos. 5,223,168, and 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-deoxy-2′-fluoro-oligonucleotides. In some embodiments, the oligonucleotides described herein are chemically synthesized using phosphoramidite approach, e.g., the method by Roy et al. Molecules. 2013; 18(11):14268-14284; Hayakawa et al. Journal of the American Chemical Society. 1998; 120(48):12395-401; Beaucage, S. L. (1993). Oligodeoxyribonucleotides synthesis: Phosphoramidite approach. In Protocols for Oligonucleotides and Analogs: Synthesis and Properties (pp. 33-61). Humana Press, each of which is hereby incorporated by reference.

### Pharmaceutical Compositions

The present disclosure also includes pharmaceutical compositions and formulations comprising the oligonucleotides (e.g., an RNAi agent or ASO) described herein, or a pharmaceutically acceptable salt thereof. In one embodiment, provided herein are pharmaceutical compositions comprising an oligonucleotide (e.g., an RNAi agent or ASO), as described herein, or a pharmaceutically acceptable salt thereof, and a pharmaceutically acceptable carrier or excipient. The pharmaceutical compositions comprising the oligonucleotides described herein are useful for treating or preventing a condition or symptoms associated with PLN expression. Such pharmaceutical compositions are formulated based on the mode of delivery. One example is compositions that are formulated for systemic administration via parenteral delivery, e.g., by subcutaneous (SC), intramuscular (IM), or intravenous (IV) delivery. In some embodiments, an oligonucleotide is formulated in buffer solutions such as phosphate-buffered saline solutions, liposomes, micellar structures, and capsids. In some embodiments, naked oligonucleotides or conjugates thereof are formulated in water or in an aqueous solution (e.g., water with pH adjustments). In some embodiments, naked oligonucleotides or conjugates thereof are formulated in basic buffered aqueous solutions (e.g., PBS). The pharmaceutical compositions of the invention may be administered in dosages sufficient to inhibit expression of the target gene.

A “pharmaceutically acceptable carrier or excipient” is a pharmaceutically acceptable solvent, suspending agent, or any other pharmacologically inert vehicle for delivering one or more oligonucleotides to a subject. The excipient can be liquid or solid and is selected, with the planned manner of administration in mind, so as to provide for the desired bulk, consistency, etc., when combined with the oligonucleotide and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, com starch, polyethylene glycols, sodium benzoate, sodium acetate, etc.); disintegrants (e.g., starch, sodium starch glycolate, etc.); and wetting agents (e.g., sodium lauryl sulphate, etc.).

In some embodiments, the present disclosure also includes a pharmaceutical composition suitable for injectable use, which comprises sterile aqueous solutions (where water soluble) or dispersions and sterile powders for the extemporaneous preparation of sterile injectable solutions or dispersions. For intravenous or subcutaneous administration, suitable carriers include physiological saline, bacteriostatic water, Cremophor EL® (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

Formulations for topical administration of oligonucleotides can include sterile and non-sterile aqueous solutions, non-aqueous solutions in common solvents such as alcohols, or solutions of the oligonucleotides in liquid or solid oil bases. The solutions can also contain buffers, diluents and other suitable additives. Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

In some embodiments, the oligonucleotides herein are conjugated to one or more non-nucleotide groups including, but not limited to, a targeting group/targeting moiety, linking group, delivery polymer, or a delivery vehicle. The non-nucleotide group can enhance targeting, delivery, or attachment of the oligonucleotide.

In some embodiments, at least one nucleotide of the oligonucleotide herein is conjugated to one or more targeting ligands, such as a carbohydrate, amino sugar, cholesterol, polypeptide, or lipid. Exemplary targeting ligands include, but are not limited to, thyrotropin, melanotropin, lectin, a glycoprotein, surfactant protein A, mucin carbohydrate, a multivalent lactose moiety, a multivalent galactose moiety, a N-acetylgalactosamine (GalNAc) moiety, a N-acetyl-glucosamine moiety, a multivalent mannose moiety, a multivalent fucose moiety, a glycosylated polypeptide, transferrin or an antibody, antibody fragment, protein, peptide, or aptamer capable of binding the transferrin receptor, bisphosphonate, polyglutamate, polyaspartate, cholesterol, bile acid, folate, vitamin B12, biotin, an RGD peptide, or an RGD peptide mimetic. In some embodiments, the targeting ligand comprises a N-acetylgalactosamine (GalNAc) moiety. In some embodiments, the GalNAc moiety comprises a monovalent GalNAc moiety, a bivalent GalNAc moiety, a trivalent GalNAc moiety, or a tetravalent GalNAc moiety.

The non-nucleotide group can be covalently linked to the 3′ and/or 5′ end of either the antisense strand and/or the sense strand, when present. In some embodiments, when the oligonucleotide is an RNAi agent, the RNAi agent contains a non-nucleotide group linked to the 3′ and/or 5′ end of the sense strand. In some embodiments, a non-nucleotide group is linked to the 5′ end of a PLN RNAi agent sense strand. A non-nucleotide group may be linked directly or indirectly to the oligonucleotide via a linker/linking group. In some embodiments, a non-nucleotide group is linked to the oligonucleotide via a labile, cleavable, or reversible bond or linker.

In some embodiments, the targeting moiety is a targeting ligand. In some embodiments, the targeting ligand is small molecule-based, sugar-based (e.g., saccharide-based), fatty acid-based, protein-based, or nucleic acid-based targeting ligand. In some embodiments, the targeting moiety is a protein-based targeting ligand. In some embodiments, the protein-based targeting ligand is an antibody, nanobody, affibody, a peptibody, or a peptide. In some embodiments, the protein-based targeting ligand is an antibody, a functional fragment thereof, or an antigen-binding fragment thereof.

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, an oligonucleotide herein is formulated in buffer solutions such as phosphate buffered saline solutions, liposomes, micellar structures and capsids. Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine), can be used. Suitable lipids include Oligofectamine, Lipofectamine (Life Technologies), NC388 (Ribozyme Pharmaceuticals, Inc., Boulder, Colo.), or FuGene 6 (Roche), all of which can be used according to the manufacturer's instructions. Accordingly, in some embodiments, a formulation herein comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013). In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethylsulfoxide or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll® or gelatin).

### Methods of Treatment

The present disclosure also provides methods of using any one of the oligonucleotides disclosed herein, or a pharmaceutically acceptable salt thereof, or any one of the pharmaceutical compositions as described herein to reduce or inhibit PLN expression in a subject. The methods include contacting one or more cells in a subject with an oligonucleotide of the disclosure, or a pharmaceutically acceptable salt thereof, or any one of the pharmaceutical compositions as described herein, thereby inhibiting expression of PLN in the cells. The present disclosure also provides use of any one of the oligonucleotides disclosed herein, or a pharmaceutically acceptable salt thereof, or any one of the pharmaceutical compositions as described herein, in the manufacture of a medicament for reducing or inhibiting PLN expression in a subject, or for preventing or treating a PLN related disorder in a subject. In some embodiments, the terms “inhibiting PLN expression” and “reducing PLN expression” are interchangeable.

Reduction in PLN expression can be assessed by any methods known in the art. For example, a reduction in the expression of PLN may be determined by determining the mRNA expression level of PLN using methods routine to one of ordinary skill in the art, e.g., northern blotting, qRT-PCR; by determining the protein level of PLN using methods routine to one of ordinary skill in the art, such as western blotting, immunological techniques. In some embodiments, the reduction of PLN expression is determined by measuring PLN expression in a population of cardiomyocytes or muscle cells derived from the subject. In some embodiments, the reduction of PLN expression is determined by measuring PLN expression in serum derived from the subject.

The present disclosure further provides methods of treatment of a subject in need thereof. The methods of treatment include administering an oligonucleotide of the disclosure to a subject, e.g., a subject that would benefit from inhibition of PLN expression, in a therapeutically effective amount of an oligonucleotide targeting a PLN gene or a pharmaceutical composition comprising an oligonucleotide targeting a PLN gene. In some embodiments, a method of the present disclosure comprises administering an oligonucleotide of the disclosure to a subject that would benefit from or that suffers from a disease or disorder related to abnormal SERCA2A expression or activity (for example, underexpression or hypoactivity relative to a healthy subject).

The in vivo methods of the disclosure may include administering to a subject a composition containing an oligonucleotide disclosed herein, or a pharmaceutically acceptable salt, wherein the oligonucleotide includes a nucleotide sequence that is complementary to at least a part of a sequence of nucleotides encoding PLN of the subject to be treated.

The oligonucleotide can be administered by any means known in the art including, but not limited to oral, intraperitoneal, or parenteral routes, including intracranial (e.g., intraventricular, intraparenchymal, and intrathecal), intravenous, intramuscular, intravitreal, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intrathecal injection. The administration of the oligonucleotide may be repeated over a period of time. The administration may be repeated on a regular basis. In some embodiments, after an initial treatment regimen, the treatments can be administered on a less frequent basis. A repeat-dose regimen may include administration of a therapeutic amount of an oligonucleotide on a regular basis.

The present disclosure also provides methods of using any one of the oligonucleotides disclosed herein, or a pharmaceutically acceptable salt thereof, to treat or prevent a PLN-related disorder in a subject.

The present disclosure also provides methods of using any one of the oligonucleotides disclosed herein, or a pharmaceutically acceptable salt thereof, or a pharmaceutical composition comprising the oligonucleotides described herein, to treat or prevent a PLN-related disorder in a subject. The present disclosure also provides a method for inhibiting PLN expression or treating a PLN related disorder in a subject, the method comprising administering to the subject an effective amount of an oligonucleotide for inhibiting expression of PLN, or a pharmaceutically acceptable salt thereof, or a pharmaceutical composition comprising the oligonucleotide for inhibiting expression of PLN. Non-limiting examples of PLN related disorders include inherited human dilated cardiomyopathy, familial hypertrophic cardiomyopathy, heart failure, PLN-R14Del heart disease, heart disease associated with a gain-of-function mutation in PLN, inherited dilated cardiomyopathy associated with a PLN mutation (e.g., a mutation that results in protein aggregation and/or unfolded protein response), heart failure with reduced ejection fraction (e.g., non-familial form of heart failure with reduced ejection fraction), arrhythmia, cardiomyopathy, and cardiomyopathy with heart failure.

In some embodiments, the present disclosure provides a method of treating inherited human dilated cardiomyopathy, comprising administering an oligonucleotide disclosed herein, or a pharmaceutically acceptable salt thereof, to a subject in need thereof.

In some embodiments, the present disclosure provides a method of treating familial hypertrophic cardiomyopathy, comprising administering an oligonucleotide disclosed herein, or a pharmaceutically acceptable salt thereof, to a subject in need thereof.

In some embodiments, the present disclosure provides a method of treating heart failure, comprising administering an oligonucleotide disclosed herein, or a pharmaceutically acceptable salt thereof, to a subject in need thereof.

In some embodiments, the present disclosure provides a method of treating heart failure with reduced ejection fraction (e.g., non-familial form of heart failure with reduced ejection fraction), comprising administering an oligonucleotide disclosed herein, or a pharmaceutically acceptable salt thereof, to a subject in need thereof.

In some embodiments, the present disclosure provides a method of treating arrhythmia, comprising administering an oligonucleotide disclosed herein, or a pharmaceutically acceptable salt thereof, to a subject in need thereof.

In some embodiments, the present disclosure provides a method of treating cardiomyopathy, comprising administering an oligonucleotide disclosed herein, or a pharmaceutically acceptable salt thereof, to a subject in need thereof.

In some embodiments, the present disclosure provides a method of treating cardiomyopathy with heart failure, comprising administering an oligonucleotide disclosed herein, or a pharmaceutically acceptable salt thereof, to a subject in need thereof.

In some embodiments, the present disclosure provides a method of treating PLN-R14Del heart disease, comprising administering an oligonucleotide disclosed herein, or a pharmaceutically acceptable salt thereof, to a subject in need thereof.

In some embodiments, the present disclosure provides a method of treating heart disease associated with a gain-of-function mutation in PLN, comprising administering an oligonucleotide disclosed herein, or a pharmaceutically acceptable salt thereof, to a subject in need thereof.

In some embodiments, the present disclosure provides a method of treating inherited dilated cardiomyopathy associated with a PLN mutation (e.g., a mutation that results in protein aggregation and/or unfolded protein response), comprising administering an oligonucleotide disclosed herein, or a pharmaceutically acceptable salt thereof, to a subject in need thereof.

An oligonucleotide of the disclosure may be administered as a “free oligonucleotide.” A free oligonucleotide is administered in the absence of a pharmaceutical composition. In some embodiments, a free oligonucleotide is administered in the absence of a transfection agent. The naked oligonucleotide may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the oligonucleotide can be adjusted such that it is suitable for administering to a subject. Alternatively, an RNAi agent of the disclosure may be administered as a pharmaceutical composition, such as a dsRNA liposomal formulation.

In some embodiments, the methods herein may further comprise administering to the subject an additional agent or therapy suitable for treatment or prevention of a PLN related disorder. Non-limiting examples of an additional agent or therapy include anti-PLN antibodies.

### EXEMPLARY EMBODIMENTS

Embodiment 1: An oligonucleotide for inhibiting expression of phospholamban (PLN), wherein the oligonucleotide comprises an antisense strand comprising at least 14 contiguous nucleotides substantially complementary to a sequence of nucleotides encoding PLN, with no more than 4 mismatched nucleotides.

Embodiment 2: The oligonucleotide of Embodiment 1, wherein the sequence of nucleotides encoding PLN comprises a nucleotide sequence having at least 90% nucleotide sequence identity to SEQ ID NO: 2523.

Embodiment 3: The oligonucleotide of Embodiment 1 or 2, wherein the oligonucleotide targets a sequence of nucleotides corresponding to positions of SEQ ID NO: 2523 (NM_002667.5) selected from Table 4.

Embodiment 4: The oligonucleotide of any one of Embodiments 1-3, wherein the antisense strand is substantially or completely complementary to a sequence of nucleotides corresponding to an untranslated region of the PLN transcript.

Embodiment 5: The oligonucleotide of any one of Embodiments 1-4, wherein the oligonucleotide is an RNAi agent.

Embodiment 6: The oligonucleotide of Embodiment 5, wherein the oligonucleotide is a double stranded small interfering RNA, a short hairpin RNA, or a Dicer-substrate siRNA (DsiRNA).

Embodiment 7: The oligonucleotide of Embodiment 6, wherein the RNAi agent is a double-stranded small interfering RNA (siRNA) further comprising a sense strand, wherein the sense strand and antisense strand form a double stranded region.

Embodiment 8: The oligonucleotide of any one of Embodiments 1-7, wherein the antisense strand comprises a nucleotide sequence comprising at least 15 contiguous nucleotides (for example, 15, 16, 17, 18, 19, 20, or 21 contiguous nucleotides) differing by no more than 4, 3, 2, or 1 nucleotides from the nucleotide sequence of any one of SEQ ID NOs: 629-1256, 1885-2522, or 2644-2763, or a nucleotide sequence having at least 90% nucleotide sequence identity to a portion of the nucleotide sequence of any one of SEQ ID NOs: 629-1256, 1885-2522, or 2644-2763.

Embodiment 9: The oligonucleotide of any one of Embodiments 1-8, wherein the antisense strand comprises the nucleotide sequence of any one of SEQ ID NOs: 629-1256, 1885-2522, or 2644-2763.

Embodiment 10: The oligonucleotide of any one of Embodiments 7-9, wherein the sense strand comprises a nucleotide sequence differing by no more than 4, 3, 3, or 1 nucleotides from the nucleotide sequence of any one of SEQ ID NOs: 1-628, 1257-1884, or 2524-2643, or a nucleotide sequence having at least 90% nucleotide sequence identity to a portion of the nucleotide sequence of any one of SEQ ID NOs: 1-628, 1257-1884, or 2524-2643.

Embodiment 11: The oligonucleotide of any one of Embodiments 7-10, wherein the sense strand comprises the nucleotide sequence of any one of SEQ ID NOs: 1-628, 1257-1884, or 2524-2643.

Embodiment 12: The oligonucleotide of any one of Embodiments 7-11, wherein the antisense strand comprises a nucleotide sequence of any one of SEQ ID NOs: 629-1256, 1885-2522, or 2644-2763, and the sense strand comprises a nucleotide sequence of any one of SEQ ID NOs: 1-628, 1257-1884, or 2524-2643.

Embodiment 13: The oligonucleotide of any one of Embodiments 7-12, wherein each strand is no more than 30 nucleotides in length, or 19-30 nucleotides in length, or 19-23 nucleotides in length, or 19-21 nucleotides in length.

Embodiment 14: The oligonucleotide of any one of Embodiments 7-13, wherein at least one strand comprises a 3′ overhang of at least 1 nucleotide or at least 2 nucleotides.

Embodiment 15: The oligonucleotide of any one of Embodiments 7-14, wherein at least one strand comprises a 5′ overhang of at least 1 nucleotide, or at least 2 nucleotides.

Embodiment 16: The oligonucleotide of any one of Embodiments 7-15, wherein the double stranded region is 15-30 nucleotide pairs in length, or 15-23 nucleotide pairs in length, or 17-25 nucleotide pairs in length, or 19-23 nucleotide pairs in length, or 19-21 nucleotide pairs in length.

Embodiment 17: The oligonucleotide of any one of Embodiments 7-16, wherein the antisense strand is 21 nucleotides in length and the sense strand is 19 nucleotides in length, or the antisense strand is 19 nucleotides in length and the sense strand is 19 nucleotides in length.

Embodiment 18: The oligonucleotide of any one of Embodiments 1-4, wherein the oligonucleotide is a single-stranded antisense oligonucleotide (ASO).

Embodiment 19: The oligonucleotide of Embodiment 18, wherein the ASO comprises a nucleic acid sequence comprising at least 14 contiguous nucleotides (for example, 14, 15, 16, 17, 18, 19, 20, or 21 contiguous nucleotides) that differ by no more than 1, 2, 3, or 4 nucleotides from any one of SEQ ID NOs: 629-1256 or 2644-2763.

Embodiment 20: The oligonucleotide of Embodiment 18 or 19, wherein the ASO comprises a nucleic acid sequence comprising at least 14 contiguous nucleotides (for example, 14, 15, 16, 17, 18, 19, 20, or 21 contiguous nucleotides) that differ by no more than 1, 2, 3 or 4 nucleotides from any one of SEQ ID NOs: 1885-2522.

Embodiment 21: The oligonucleotide of any one of Embodiments 1-20, wherein the oligonucleotide comprises at least one or more modifications.

Embodiment 22: The oligonucleotide of Embodiment 21, wherein the one or more modifications is selected from a ribose modification, a backbone modification, a nucleobase modification, or a combination thereof.

Embodiment 23: The oligonucleotide of Embodiment 22, wherein the ribose modification comprises a locked nucleic acid (LNA), a tricyclo-DNA (tcDNA), 2′-deoxy-2′-fluoro, 2′-O-methyl, 2′-methoxyethyl (2′-MOE), 2′-deoxy-2′-arabino-fluoro, 2′-O-benzyl, 2′-O-methyl-4-pyridine, 2′ cyclic ethyl (cET), phosphorodiamidate morpholino (PMO), glycol nucleic acid (GNA), unlocked nucleic acid (UNA), a threose nucleic acid (TNA), or a combination thereof.

Embodiment 24: The oligonucleotide of Embodiment 23, wherein the ribose modification comprises a 2′-deoxy-2′-fluoro, 2′-O-methyl, glycol nucleic acid (GNA), unlocked nucleic acid (UNA), a threose nucleic acid (TNA), or a combination thereof.

Embodiment 25: The oligonucleotide of Embodiment 23 or 24, wherein the ribose modification is a 2′-deoxy-2′-fluoro modification, a 2′-O-methyl modification, or a combination thereof.

Embodiment 26: The oligonucleotide of any one of Embodiments 22-25, wherein the backbone modification comprises phosphorothioate, phosphorodithioate, methylphosphonate, methyoxypropyl-phosphonate, 5′-(E)-vinylphosphonate, 5′-methyl phosphonate, 5′-methyl phosphate, 5′-phosphorothioate, peptide nucleic acid (PNA), or a combination thereof.

Embodiment 27: The oligonucleotide of Embodiment 26, wherein the backbone modification comprises a phosphorothioate.

Embodiment 28: The oligonucleotide of any one of Embodiments 22-27, wherein the backbone modification comprises a phosphorothioate modification.

Embodiment 29: The oligonucleotide of any one of Embodiments 22-28, wherein the nucleobase modification comprises 5-substituted pyrimidines, 6-azapyrimidines and N-2, N-6 and 0-6 substituted purines, 5-methylcytosine (5-Me-C), 5-hydroxymethyl cytosine, xanthine, hypoxanthine, 2-aminoadenine, N6-alkyl derivatives, N2-alkyl, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxy, 5-halo, 5-trifluoromethyl, N7-methylguanine, N7-methyladenine, 8-azaguanine, 8-azaadenine, 7-deazaguanine, 7-deazaadenine, 3-deazaguanine, 3-deazaadenine, or any combination thereof.

Embodiment 30: The oligonucleotide of Embodiment 29, wherein the oligonucleotide comprises at least one modified nucleotide selected from the group consisting of a deoxy nucleotide, a 3′-terminal deoxythymidine (dT) nucleotide, a 2′-O-methyl modified nucleotide, a 2′-deoxy-2′-fluoro modified nucleotide, a 2′-deoxy-modified nucleotide, a 2′-5′-linked ribonucleotide (3′-RNA), a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-O-(methoxy ethyl) modified nucleotide, a 2′-O-alkyl-modified nucleotide, a morpholino nucleotide, a phosphoramidate morpholino, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′-methylphosphate group, a nucleotide comprising a 5′ phosphate or 5′ phosphate mimic, a nucleotide comprising vinyl phosphonate, a glycol nucleic acid (GNA), a glycol nucleic acid S-Isomer (S-GNA), a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate; a cytidine-2′-phosphate, a guanosine-2′-phosphate, a uridine-2′-phosphate, an adenosine-2′-phosphate, a 2′-O-hexadecyl-adenosine-3′-phosphate, a 2′-O-hexadecyl-cytidine-3′-phosphate, a 2′-O-hexadecyl-guanosine-3′-phosphate, and a 2′-O-hexadecyl-uridine-3′-phosphate, a 3′-3′ inverted nucleotide linkage, a 5′-5′ inverted nucleotide linkage, TNA, and combinations thereof.

Embodiment 31: The oligonucleotide of Embodiment 30, wherein oligonucleotide comprises a modified nucleotide selected from the group consisting of a deoxy nucleotide, a 3′-terminal deoxythymidine (dT) nucleotide, a 3′-3′ inverted nucleotide linkage, a 5′-5′ inverted nucleotide linkage, or a 5′-(E)-vinylphosphonate-2′-O-methyl-uridine-3′-phosphate, and a combination thereof.

Embodiment 32: The oligonucleotide of Embodiment 30 or 31, wherein the oligonucleotide comprises 5′-(E)-vinylphosphonate-2′-O-methyl-uridine-3′-phosphate.

Embodiment 33: The oligonucleotide of Embodiment 21, wherein at least one of the modifications is a thermally destabilizing nucleotide modification.

Embodiment 34: The oligonucleotide of Embodiment 33, wherein the thermally destabilizing nucleotide modification is selected from the group consisting of an abasic modification; a mismatch with the opposing nucleotide in the duplex; destabilizing sugar modification, a 2′-deoxy modification, an acyclic nucleotide, an unlocked nucleic acids (UNA); a glycerol nucleic acid (GNA), and a combination thereof.

Embodiment 35: The oligonucleotide of Embodiment 31, wherein the modification comprises a short sequence of 3′-terminal deoxythymidine nucleotide (dT).

Embodiment 36: The oligonucleotide of any one of Embodiments 21-35, wherein the modifications on the nucleotides are 2′-O-methyl and 2′deoxy-2′-fluoro modifications.

Embodiment 37: The oligonucleotide of any one of Embodiments 21-36, wherein the oligonucleotide comprises at least one phosphorothioate internucleoside or phosphorodithioate internucleoside linkage.

Embodiment 38: The oligonucleotide of Embodiment 37, wherein the oligonucleotide comprises 6-8 phosphorothioate internucleoside linkages.

Embodiment 39: The oligonucleotide of Embodiments 37 or 38, wherein the oligonucleotide comprises at least 1, or at least 2, phosphorothioate internucleoside linkage at a 5′ end of the sense strand.

Embodiment 40: The oligonucleotide of any one of Embodiments 37-39, wherein the oligonucleotide comprises at least 1 phosphorothioate internucleoside linkage at a 3′ end of the sense strand.

Embodiment 41: The oligonucleotide of any one of Embodiments 37-40, wherein the oligonucleotide comprises at least 1, or at least 2, phosphorothioate internucleoside linkage at a 5′ end of the antisense strand.

Embodiment 42: The oligonucleotide of any one of Embodiments 37-41, wherein the oligonucleotide comprises at least 1 phosphorothioate internucleoside linkage at a 3′ end of the antisense strand.

Embodiment 43: The oligonucleotide of any one of Embodiments 37-42, wherein no more than five of the nucleotides of the antisense strand are unmodified nucleotides.

Embodiment 44: The oligonucleotide of any one of Embodiments 37-43, wherein all the nucleotides of the antisense strand are modified nucleotides.

Embodiment 45: The oligonucleotide of any one of Embodiments 37-44, wherein no more than five of the nucleotides of the sense strand are unmodified nucleotides.

Embodiment 46: The oligonucleotide of any one of Embodiments 37-45, wherein all the nucleotides of the sense strand are modified nucleotides.

Embodiment 47: The oligonucleotide of any one of Embodiments Error! Reference source not found.-Error! Reference source not found., wherein the antisense strand comprises a chemical modification pattern according to (Nfs)ₐ(nNf)_{b}(ns)_{c}n, wherein:
n is a 2′-O-methyl-nucleoside-3′-phosphate;
Nfs is a 2′-deoxy-2′-fluoro-nucleoside-3′-phosphorothioate;
Nf is a 2′-deoxy-2′-fluoro-nucleoside-3′-phosphate;
ns is a 2′-O-methyl-nucleoside-3′-phosphorothioate;
a is at least 1;
b is at least 5-10; and
c is at least 1.

Embodiment 48: The oligonucleotide of Embodiment Error! Reference source not found., wherein the antisense strand comprises a chemical modification pattern NfsNfsnNfnNfnNfnNfnNfnNfnNfnNfnsnsn.

Embodiment 49: The oligonucleotide of any one of Embodiments Error! Reference source not found.-Error! Reference source not found., wherein the sense strand comprises a chemical modification pattern according to (ns)_{d}(Nfn)ₑNf, wherein:
n is a 2′-O-methyl-nucleoside-3′-phosphate;
Nf is a 2′-deoxy-2′-fluoro-nucleoside-3′-phosphate;
ns is a 2′-O-methyl-nucleoside-3′-phosphorothioate;
d is at least 1; and
e is 5-10.

Embodiment 50: The oligonucleotide of Embodiment Error! Reference source not found., wherein the sense strand comprises a chemical modification pattern nsnsNfnNfnNfnNfnNfnNfnNfnNfnNf.

Embodiment 51: The oligonucleotide of any one of Embodiments Error! Reference source not found.-Error! Reference source not found., wherein each of the antisense and the sense strand is independently 17-23 nucleotides in length, wherein the antisense strand comprises the motif F(SF)nSnn, wherein n is from 2 to about 20, nn is 0 or 1, one of F and S is a 2′-deoxy-2′-fluoro modified nucleoside and the other of F and S is a 2′-O-methyl modified nucleoside.

Embodiment 52: The oligonucleotide of any one of Embodiments Error! Reference source not found.-Error! Reference source not found., wherein the antisense strand comprises the chemical modification pattern of nNfnnnNfnNfNfnnnnNfnNfnnnnn and the sense strand comprises the chemical modification nnnnnnNfnNfNfNfnnnnnnnn, wherein n is a 2′-O-methyl-nucleoside and Nf is a 2′-deoxy-2′-fluoro-nucleoside.

Embodiment 53: The oligonucleotide of any one of Embodiments Error! Reference source not found.-Error! Reference source not found., wherein each of the antisense and the sense strand is independently 20-23 nucleotides in length, wherein the antisense strand comprises a region having the formula X1-Y—X2, wherein Y is a subregion of from about 5 to about 12 linked nucleosides and each of X1 subregion and X2 subregion is, independently, a plurality of linked nucleosides having the formula FSFS, where one of F and S is a 2′-deoxy-2′-fluoro modified nucleoside and the other of F and S is a 2′-O-methyl modified nucleoside; and each internucleoside linkage of X1 subregion and X2 subregion is, independently, a phosphodiester or a phosphorothioate internucleoside linkage.

Embodiment 54: The oligonucleotide of any one of Embodiments Error! Reference source not found.-Error! Reference source not found., wherein each of the antisense and the sense strand is independently 17-23 nucleotides in length, wherein the antisense strand comprises a contiguous sequence of linked nucleosides that define an alternating motif of the formula:

5′-Q(-L-Z-L-Q)n(-L-Z)nn-3′
wherein:
each L is an internucleoside linking group;
either each Q is a 2′-deoxy-2′-fluoro-nucleoside and each Z is a 2′-O-methyl nucleoside;
or each Q is a 2′-O-methyl nucleoside and each Z is a 2′-deoxy-2′-fluoro nucleoside;
n is from 8 to 14; and
nn is 0 or 1.

Embodiment 55: The oligonucleotide of any one of Embodiments 7-Error! Reference source not found., wherein the antisense strand is 19-25 nucleotides in length and is represented by the formula:

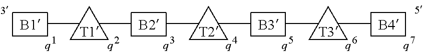

wherein:
B1′, B2′, B3′, and B4′ each independently represent a nucleotide containing a modification selected from the group consisting of 2′-O-alkyl, 2′-substituted alkoxy, 2′-substituted alkyl, 2′-halo, ENA, and BNA/LNA;
T1′, T2′, and T3′ each independently represent a nucleotide comprising a chemical modification selected from the group consisting of DNA, RNA, LNA, 2′-deoxy-2′-fluoro, and 5′-methyl-2′-deoxy-2′-fluoro nucleotides;
q1 is 4 to 15 nucleotides in length;
q3 or q7 is independently 1-6 nucleotide(s) in length;
q2 or q6 is independently 1-3 nucleotide(s) in length;
q4 is 0-3 nucleotide(s) in length; and
q5 is 0-10 nucleotide(s) in length;

wherein:

the antisense strand has 2′-deoxy-2′-fluoro modifications, and wherein the 2′-deoxy-2′-fluoro modifications on the antisense strand consist of four, and only four, 2′-deoxy-2′-fluoro modifications or six, and only six, 2′-deoxy-2′-fluoro modifications.

Embodiment 56: The oligonucleotide of any one of Embodiments Error! Reference source not found.-Error! Reference source not found., wherein the antisense strand and sense strand are each 14 to 40 nucleotides, and are represented by the formula:

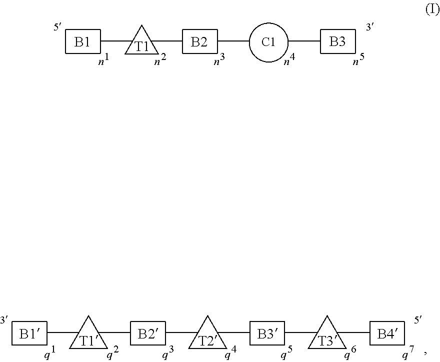

wherein:
B1, B1′, B2′, B3′, and B4′ each independently represent a nucleotide containing a modification selected from the group consisting of 2′-O-alkyl, 2′-substituted alkoxy, 2′-substituted alkyl, 2′-halo, ENA, and BNA/LNA;
C1 is a thermally destabilizing nucleotide, selected from the group consisting of i) a nucleotide that forms a mismatch pair with the opposing nucleotide in the antisense strand, ii) a nucleotide having an abasic modification, and iii) a nucleotide having a sugar modification, and placed at a site opposite to the seed region (positions 2-8) of the antisense strand;
T1′, T2′, and T3′ each independently represent a nucleotide comprising a modification providing the nucleotide a steric bulk that is less than or equal to the steric bulk of a 2′-OMe modification, wherein the modification is at the 2′-position of a ribose sugar of the nucleotide or at a position of a non-ribose nucleotide similar to the 2′-position of a ribose sugar;
each n1, and q1 is independently 4 to 15 nucleotides in length;
each q3, and q7 is independently 1-6 nucleotide(s) in length;
each q2 and q6 is independently 1-3 nucleotide(s) in length;
q5 is 0-10 nucleotide(s) in length;
each n4, and q4 is independently 0-3 nucleotide(s) in length;
n2 is 3 nucleotides in length, and T1 each are 2′-deoxy-2′-fluoro nucleotides;
n3 is 7 nucleotides in length, and B2 each are 2′-OMe nucleotides; and
n5 is 3 nucleotides in length, and B3 each are 2′-OMe nucleotides.

Embodiment 57: The oligonucleotide of any one of Embodiments Error! Reference source not found.-Error! Reference source not found., wherein the antisense strand and sense strand are each 19-25 nucleotides in length, wherein the antisense strand is represented by the formula:

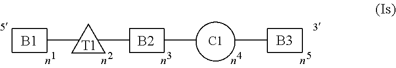

wherein:
B1, B2, and B3 each independently represent a nucleotide containing a modification selected from the group consisting of 2′-O-alkyl, 2′-substituted alkoxy, 2′-substituted alkyl, 2′-halo, ENA, and BNA/LNA;
C1 is a thermally destabilizing nucleotide, selected from the group consisting of i) a nucleotide that forms a mismatch pair with the opposing nucleotide in the antisense strand, ii) a nucleotide having an abasic modification, and iii) a nucleotide having a sugar modification, and placed at a site opposite to the seed region (positions 2-8) of the antisense strand;
T1 represents a nucleotide comprising a 2′-deoxy-2′-fluoro modification;
n1 or n3 is independently 4 to 15 nucleotides in length;
n5 is 1-6 nucleotide(s) in length;
n2 is 3;
n4 is 0-3 nucleotide(s) in length; and
wherein the sense strand has 2′-deoxy-2′-fluoro modifications, and wherein the 2′-deoxy-2′-fluoro modifications on the sense strand consist of four, and only four, 2′-deoxy-2′-fluoro modifications, wherein the four 2′-deoxy-2′-fluoro modifications are at positions 7 and 9-11 from the 5′-end of the sense strand.

Embodiment 58: The oligonucleotide of any one of Embodiments Error! Reference source not found.-Error! Reference source not found., wherein the antisense strand is complementary to at least one portion of a mRNA of the target gene (e.g., PLN), wherein:
the sense strand comprises 19-22 nucleotides,
the antisense strand comprises 19-25 nucleotides; and
the oligonucleotide is represented by the formula:

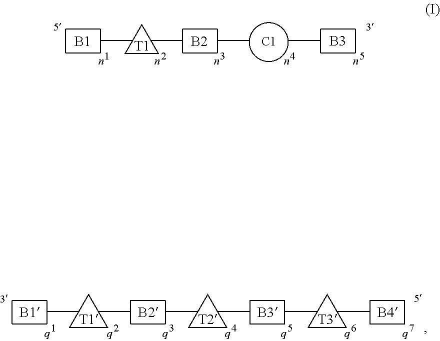

wherein:
   B1′, B2′, B3′, and B4′ each independently represent a nucleotide containing a modification selected from the group consisting of 2′-O-methyl and 2′-deoxy-2′-fluoro;
   each B1, B2, and B3 is 2′-O-methyl nucleotide;
   C1 is glycerol nucleic acid (GNA) placed at a site opposite to the seed region (positions 2-8) of the antisense strand;
   T1′, T2′, and T3′ are each 2′-F, wherein:
      T1′ is at position 14 from the 5′ end of the antisense strand, and q2 is 1; and
      T3′ is at position 2 from the 5′ end of the antisense strand, and q6 and q7 are 1;
each n1, n3, and q1 is independently 4 to 15 nucleotides in length;
each n5 and q3 is independently 1-6 nucleotide(s) in length;
q5 is 0-10 nucleotide(s) in length;
each n4 and q4 is independently 0-3 nucleotide(s) in length;
n2 is 3 nucleotides in length;
T1 each are 2′-deoxy-2′-fluoro nucleotides;
wherein
(a) the oligonucleotide is covalently conjugated to at least one ligand; and
(b) one of the T1 nucleotides is at position 11 from the 5′ end of the sense strand.

Embodiment 59: The oligonucleotide of any one of Embodiments Error! Reference source not found.-Error! Reference source not found., wherein the sense strand comprises 19-22 nucleotides, the antisense strand comprises 19-25 nucleotides; and the oligonucleotide is represented by the formula:

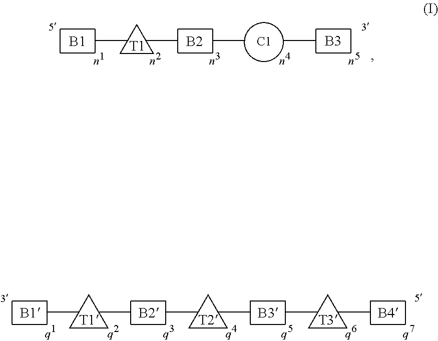

wherein:
B1′, B2′, B3′, and B4′ each independently represent a nucleotide containing a modification selected from the group consisting of 2′-O-methyl and 2′-deoxy-2′-fluoro;
each B1, B2, and B3 is 2′-OMe;
C1 is glycerol nucleic acid (GNA) placed at a site opposite to the seed region (positions 2-8) of the antisense strand;
   T1′, T2′, and T3′ are each 2′-deoxy-2′-fluoro, wherein: T1′ is at position 14 from the 5′ end of the antisense strand, and q2 is 1; and
   T3′ is at position 2 from the 5′ end of the antisense strand, and q6 and q7 are 1;
each n1, n3, and q1 is independently 4 to 15 nucleotides in length;
each n5 and q3 is independently 1-6 nucleotide(s) in length;
q5 is 0-10 nucleotide(s) in length;
each n4 and q4 is independently 0-3 nucleotide(s) in length;
n2 is 3 nucleotides in length; and
T1 each are 2′-deoxy-2′-fluoro, and
wherein
   (a) the oligonucleotide is covalently conjugated to at least one ligand;
   (b) one of the T1 nucleotides is at a position in the sense strand that is opposite to position 11 from the 5′ end of the antisense strand; and
   (c) the oligonucleotide comprises at least one phosphorothioate internucleoside linkage.

Embodiment 60: The oligonucleotide of any one of Embodiments 7-59, wherein the antisense strand and sense strand are each 14 to 40 nucleotides, wherein the antisense strand has sufficient complementarity to a target sequence to mediate RNA interference, wherein said sense strand comprises at least one thermally destabilizing modification of the duplex within the first 9 nucleotide positions of the 5′ region of the antisense strand or a precursor thereof, wherein the antisense strand further comprises one or both of the following characteristics:
(i) 2, 3, 4, 5 or 6 2′-deoxy-2′-fluoro modifications; and
(ii) 1, 2, 3, 4 or 5 phosphorothioate internucleoside linkages; and
said sense strand comprises one, or both of the following characteristics:
(iii) 2, 3, 4, or 5 2′-deoxy-2′-fluoro modifications; and
(iv) 1, 2, 3, 4 or 5 phosphorothioate internucleoside linkages.

Embodiment 61: The oligonucleotide of any one of Embodiments 7-46, wherein the antisense strand comprises a modification pattern nsNfsnnnNfnNfNfnnnnNfnNfnnnsnsn, wherein ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

Embodiment 62: The oligonucleotide of any one of Embodiments 7-46, wherein the antisense strand disclosed herein comprises a modification pattern vpUsNfsnnnNfnNfNfnnnnNfnNfnnnsnsn, wherein vpUs is a 5′-vinylphosphonate-2′-O-methyl-uridine-3′-phosphorothioate; ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

Embodiment 63: The oligonucleotide of any one of Embodiments 7-46, wherein the sense strand disclosed herein comprises a modification pattern nsnsnnNfnNfNfNfnnnnnnnnnn, wherein ns is a 2′-O-methyl-nucleoside-3′-phosphorothioate; n is a 2′-O-methyl-nucleoside-3′-phosphate; and Nf is a 2′-deoxy-2′-fluoro-nucleoside-3′-phosphate.

Embodiment 64: The oligonucleotide of any one of Embodiments 7-46, wherein the sense strand disclosed herein comprises a modification pattern NfsnsNfnNfnNfnNfnNfnNfnNfnNfnNf, wherein ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

Embodiment 65: The oligonucleotide of any one of Embodiments 7-46, wherein the sense strand comprises a modification pattern nsnsnnNfnNfNfNfnnnnnnnnnn, and the antisense strand comprises a modification pattern nsNfsnnnNfnNfNfnnnnNfnNfnnnsnsn, wherein ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

Embodiment 66: The oligonucleotide of any one of Embodiments 7-46, wherein the sense strand comprises a modification pattern nsnsnnNfnNfNfNfnnnnnnnnnn, and the antisense strand comprises a modification pattern vpUsNfsnnnNfnNfNfnnnnNfnNfnnnsnsn, wherein vpUs is a 5′-vinylphosphonate-2′-O-methyl-uridine-3′-phosphorothioate; ns is a 2′-O-methyl-nucleoside-3′-phosphorothioate; Nfs is a 2′-deoxy-2′-fluoro-nucleoside-3′-phosphorothioate; n is a 2′-O-methyl-nucleoside-3′-phosphate; and Nf is a 2′-deoxy-2′-fluoro-nucleoside-3′-phosphate.

Embodiment 67: The oligonucleotide of any one of Embodiments 7-66, wherein the antisense strand comprises a nucleotide sequence according to any one of SEQ ID NOs: 1885-2522.

Embodiment 68: The oligonucleotide of Embodiment 67, wherein the sense strand comprises a nucleotide sequence according to any one of SEQ ID NOs: 1257-1884.

Embodiment 69: The oligonucleotide of any one of Embodiments 7-68, further comprising a terminal, chiral modification occurring at the first internucleoside linkage at the 3′-end of the antisense strand, having the linkage phosphorus atom in Sp configuration, a terminal, chiral modification occurring at the first internucleoside linkage at the 5′-end of the antisense strand, having the linkage phosphorus atom in Rp configuration, and a terminal, chiral modification occurring at the first internucleoside linkage at the 5′-end of the sense strand, having the linkage phosphorus atom in either Rp configuration or Sp configuration.

Embodiment 70: The oligonucleotide of any one of Embodiments 7-69, further comprising a terminal, chiral modification occurring at the first and second internucleoside linkages at the 3′-end of the antisense strand, having the linkage phosphorus atom in Sp configuration, a terminal, chiral modification occurring at the first internucleoside linkage at the 5′-end of the antisense strand, having the linkage phosphorus atom in Rp configuration, and a terminal, chiral modification occurring at the first internucleoside linkage at the 5′-end of the sense strand, having the linkage phosphorus atom in either Rp or Sp configuration.

Embodiment 71: The oligonucleotide of any one of Embodiments 7-70, further comprising a terminal, chiral modification occurring at the first, second and third internucleoside linkages at the 3′ end of the antisense strand, having the linkage phosphorus atom in Sp configuration, a terminal, chiral modification occurring at the first internucleoside linkage at the 5′-end of the antisense strand, having the linkage phosphorus atom in Rp configuration, and a terminal, chiral modification occurring at the first internucleoside linkage at the 5′-end of the sense strand, having the linkage phosphorus atom in either Rp or Sp configuration.

Embodiment 72: The oligonucleotide of any one of Embodiments 7-71, further comprising a terminal, chiral modification occurring at the first, and second internucleoside linkages at the 3′ end of the antisense strand, having the linkage phosphorus atom in Sp configuration, a terminal, chiral modification occurring at the third internucleoside linkages at the 3′-end of the antisense strand, having the linkage phosphorus atom in Rp configuration, a terminal, chiral modification occurring at the first internucleoside linkage at the 5′-end of the antisense strand, having the linkage phosphorus atom in Rp configuration, and a terminal, chiral modification occurring at the first internucleoside linkage at the 5′-end of the sense strand, having the linkage phosphorus atom in either Rp or Sp configuration.

Embodiment 73: The oligonucleotide of any one of Embodiments 7-72, further comprising a terminal, chiral modification occurring at the first, and second internucleoside linkages at the 3′-end of the antisense strand, having the linkage phosphorus atom in Sp configuration, a terminal, chiral modification occurring at the first, and second internucleoside linkages at the 5′-end of the antisense strand, having the linkage phosphorus atom in Rp configuration, and a terminal, chiral modification occurring at the first internucleoside linkage at the 5′-end of the sense strand, having the linkage phosphorus atom in either Rp or Sp configuration.

Embodiment 74: The oligonucleotide of any one of Embodiments 7-73, further comprising a phosphate or phosphate mimic at the 5′-end of the antisense strand.

Embodiment 75: The oligonucleotide of Embodiment 74, wherein the phosphate mimic is a 5′-vinyl phosphonate (VP).

Embodiment 76: The oligonucleotide of any one of Embodiments 1-75, further comprising a targeting moiety.

Embodiment 77: The oligonucleotide of Embodiment 76, wherein the targeting ligand is a small molecule-based, sugar-based (e.g., saccharide-based), fatty acid-based, protein-based, or nucleic acid-based targeting ligand.

Embodiment 78: The oligonucleotide of Embodiment 77, wherein the protein-based targeting ligand is an antibody, nanobody, affibody, peptibody, or a peptide.

Embodiment 79: An oligonucleotide selected from the sense strands, antisense strands, and RNAi agents as listed in Tables 1A, 1B, and 1C, or a pharmaceutically acceptable salt thereof.

Embodiment 80: A pharmaceutical composition comprising the oligonucleotide of any one of Embodiments 1-79, or a pharmaceutically acceptable salt thereof, and a pharmaceutically acceptable carrier or excipient.

Embodiment 81: A method for inhibiting PLN expression or treating a PLN related disorder in a subject, the method comprising administering an effective amount of the oligonucleotide of any one of Embodiments 1-79, or a pharmaceutically acceptable salt thereof, or the pharmaceutical composition of Embodiment 80 to the subject.

Embodiment 82: The method of Embodiment 81, wherein the subject is a human.

Embodiment 83: The method of Embodiment 81 or 82, wherein the reduction of PLN mRNA or protein expression levels is measured in a population of myocytes or cardiomyocytes derived from the subject.

Embodiment 84: The method of any one of Embodiments 81-83, wherein the reduction of PLN mRNA or protein expression levels is measured in tissues derived from the subject.

Embodiment 85: The method of any one of Embodiments 81-84, wherein the PLN-related disorder is inherited human dilated cardiomyopathy, familial hypertrophic cardiomyopathy, heart failure, PLN-R14Del heart disease, heart disease associated with a gain-of-function mutation in PLN, inherited dilated cardiomyopathy associated with a PLN mutation (e.g., a mutation that results in protein aggregation and/or unfolded protein response), non-familial form of heart failure with reduced ejection fraction, arrhythmia, or cardiomyopathy.

Embodiment 86: The method of any one of Embodiments 81-85, further comprising administering to the subject an additional agent or a therapy suitable for treatment or prevention of a PLN related disorder.

Embodiment 87: An RNAi oligonucleotide comprising a sense strand and an antisense strand, wherein the sense strand and the antisense strand form a duplex, wherein the sense strand and antisense strands are selected from the group consisting of:

| SEQ | | SEQ | |
|---|---|---|---|
| ID NO: | Sense strand | ID NO: | Antisense strand |
| 1402 | ususguGfaAfAfAfggucaagaua | 2030 | usAfsucuUfgAfCfcuuuUfcAfcaasusu |
| 1403 | usgsugAfaAfAfGfgucaagauua | 2031 | usAfsaucUfuGfAfccuuUfuCfacasusu |
| 1404 | asasaaGfgUfCfAfagauuaagaa | 2032 | usUfscuuAfaUfCfuugaCfcUfuuususu |
| 1471 | uscscuGfaGfUfAfgaagaguuua | 2099 | usAfsaacUfcUfUfcuacUfcAfggasusu |
| 1472 | csusgaGfuAfGfAfagaguuucua | 2100 | usAfsgaaAfcUfCfuucuAfcUfcagsusu |
| 1474 | gsgsauCfuUfGfUfaaacaugaaa | 2102 | usUfsucaUfgUfUfuacaAfgAfuccsusu |
| 1670 | gscsuuGfcCfAfCfaucagcuuaa | 2298 | usUfsaagCfuGfAfugugGfcAfagcsusu |
| 1671 | csusugCfcAfCfAfucagcuuaaa | 2299 | usUfsuaaGfcUfGfauguGfgCfaagsusu |
| 1672 | ususgcCfaCfAfUfcagcuuaaaa | 2300 | usUfsuuaAfgCfUfgaugUfgGfcaasusu |
| 1688 | gsusgaAfaAfGfGfucaagauuaa | 2316 | usUfsaauCfuUfGfaccuUfuUfcacsusu |
| 1402 | ususguGfaAfAfAfggucaagaua | 2513 | vpUsAfsucuUfgAfCfcuuuUfcAfcaasusu |
| 1403 | usgsugAfaAfAfGfgucaagauua | 2514 | vpUsAfsaucUfuGfAfccuuUfuCfacasusu |
| 1404 | asasaaGfgUfCfAfagauuaagaa | 2515 | vpUsUfscuuAfaUfCfuugaCfcUfuuususu |
| 1471 | uscscuGfaGfUfAfgaagaguuua | 2516 | vpUsAfsaacUfcUfUfcuacUfcAfggasusu |
| 1472 | csusgaGfuAfGfAfagaguuucua | 2517 | vpUsAfsgaaAfcUfCfuucuAfcUfcagsusu |
| 1474 | gsgsauCfuUfGfUfaaacaugaaa | 2518 | vpUsUfsucaUfgUfUfuacaAfgAfuccsusu |
| 1670 | gscsuuGfcCfAfCfaucagcuuaa | 2519 | vpUsUfsaagCfuGfAfugugGfcAfagcsusu |
| 1671 | csusugCfcAfCfAfucagcuuaaa | 2520 | vpUsUfsuaaGfcUfGfauguGfgCfaagsusu |
| 1672 | ususgcCfaCfAfUfcagcuuaaaa | 2521 | vpUsUfsuuaAfgCfUfgaugUfgGfcaasusu |
| 1688 | gsusgaAfaAfGfGfucaagauuaa | 2522 | vpUsUfsaauCfuUfGfaccuUfuUfcacsusu |

wherein:
Af represents 2′-deoxy-2′-fluoro-adenosine-3′-phosphate;
Afs represents 2′-deoxy-2′-fluoro-adenosine-3′-phosphorothioate;
Cf represents 2′-deoxy-2′-fluoro-cytidine-3′-phosphate;
Gf represents 2′-deoxy-2′-fluoro-guanosine-3′-phosphate;
Uf represents 2′-deoxy-2′-fluoro-uridine-3′-phosphate;
Ufs represents 2′-deoxy-2′-fluoro-uridine-3′-phosphorothioate;
a represents 2′-O-methyl-adenosine-3′-phosphate;
as represents 2′-O-methyl-adenosine-3′-phosphorothioate;
c represents 2′-O-methyl-cytidine-3′-phosphate;
cs represents 2′-O-methyl-cytidine-3′-phosphorothioate;
g represents 2′-O-methyl-guanosine-3′-phosphate;
gs represents 2′-O-methyl-guanosine-3′-phosphorothioate;
u represents 2′-O-methyl-uridine-3′-phosphate;
us represents 2′-O-methyl-uridine-3′-phosphorothioate; and
vpUs represents 5′-vinylphosphonate-2′-O-methyl-uridine-3′-phosphorothioate.

Embodiment 88. The RNAi oligonucleotide of Embodiment 87, wherein the sense strand is ususguGfaAfAfAfggucaagaua (SEQ ID NO: 1402), and the antisense strand is usAfsucuUfgAfCfcuuuUfcAfcaasusu (SEQ ID NO: 2030).

Embodiment 89: The RNAi oligonucleotide of Embodiment 87, wherein the sense strand is usgsugAfaAfAfGfgucaagauua (SEQ ID NO: 1403), and the antisense strand is usAfsaucUfuGfAfccuuUfuCfacasusu (SEQ ID NO: 2031).

4. The RNAi oligonucleotide of Embodiment 87, wherein the sense strand is asasaaGfgUfCfAfagauuaagaa(SEQ ID NO: 1404), and the antisense strand is usUfscuuAfaUfCfuugaCfcUfuuususu (SEQ ID NO: 2032).

5. The RNAi oligonucleotide of Embodiment 87, wherein the sense strand is uscscuGfaGfUfAfgaagaguuua (SEQ ID NO: 1471), and the antisense strand is usAfsaacUfcUfUfcuacUfcAfggasusu (SEQ ID NO: 2099).

6. The RNAi oligonucleotide of Embodiment 87, wherein the sense strand is csusgaGfuAfGfAfagaguuucua (SEQ ID NO: 1472), and the antisense strand is usAfsgaaAfcUfCfuucuAfcUfcagsusu (SEQ ID NO: 2100).

7. The RNAi oligonucleotide of Embodiment 87, wherein the sense strand is gsgsauCfuUfGfUfaaacaugaaa(SEQ ID NO: 1474), and the antisense strand is usUfsucaUfgUfUfuacaAfgAfuccsusu (SEQ ID NO: 2102).

8. The RNAi oligonucleotide of Embodiment 87, wherein the sense strand is gscsuuGfcCfAfCfaucagcuuaa(SEQ ID NO: 1670), and the antisense strand is usUfsaagCfuGfAfugugGfcAfagcsusu (SEQ ID NO: 2298).

9. The RNAi oligonucleotide of Embodiment 87, wherein the sense strand is csusugCfcAfCfAfucagcuuaaa (SEQ ID NO: 1671), and the antisense strand is usUfsuaaGfcUfGfauguGfgCfaagsusu (SEQ ID NO: 2299).

10. The RNAi oligonucleotide of Embodiment 87, wherein the sense strand is ususgcCfaCfAfUfcagcuuaaaa(SEQ ID NO: 1672), and the antisense strand is usUfsuuaAfgCfUfgaugUfgGfcaasusu (SEQ ID NO: 2300).

11. The RNAi oligonucleotide of Embodiment 87, wherein the sense strand is gsusgaAfaAfGfGfucaagauuaa (SEQ ID NO: 1688), and the antisense strand is usUfsaauCfuUfGfaccuUfuUfcacsusu (SEQ ID NO: 2316).

12. The RNAi oligonucleotide of Embodiment 87, wherein the sense strand is ususguGfaAfAfAfggucaagaua (SEQ ID NO: 1402), and the antisense strand is vpUsAfsucuUfgAfCfcuuuUfcAfcaasusu (SEQ ID NO: 2513).

13. The RNAi oligonucleotide of Embodiment 87, wherein
the sense strand is usgsugAfaAfAfGfgucaagauua (SEQ ID NO: 1403), and the antisense strand is vpUsAfsaucUfuGfAfccuuUfuCfacasusu (SEQ ID NO: 2514).

14. The RNAi oligonucleotide of Embodiment 87, wherein the sense strand is asasaaGfgUfCfAfagauuaagaa(SEQ ID NO: 1404), and the antisense strand is vpUsUfscuuAfaUfCfuugaCfcUfuuususu (SEQ ID NO: 2515).

15. The RNAi oligonucleotide of Embodiment 87, wherein the sense strand is uscscuGfaGfUfAfgaagaguuua (SEQ ID NO: 1471), and the antisense strand is vpUsAfsaacUfcUfUfcuacUfcAfggasusu (SEQ ID NO: 2516).

16. The RNAi oligonucleotide of Embodiment 87, wherein the sense strand is csusgaGfuAfGfAfagaguuucua (SEQ ID NO: 1472), and the antisense strand is vpUsAfsgaaAfcUfCfuucuAfcUfcagsusu (SEQ ID NO: 2517).

17. The RNAi oligonucleotide of Embodiment 87, wherein the sense strand is gsgsauCfuUfGfUfaaacaugaaa (SEQ ID NO: 1474), and the antisense strand is vpUsUfsucaUfgUfUfuacaAfgAfuccsusu (SEQ ID NO: 2518).

18. The RNAi oligonucleotide of Embodiment 87, wherein the sense strand is gscsuuGfcCfAfCfaucagcuuaa (SEQ ID NO: 1670), and the antisense strand is vpUsUfsaagCfuGfAfugugGfcAfagcsusu (SEQ ID NO: 2519).

19. The RNAi oligonucleotide of Embodiment 87, wherein the sense strand is csusugCfcAfCfAfucagcuuaaa (SEQ ID NO: 1671), and the antisense strand is vpUsUfsuaaGfcUfGfauguGfgCfaagsusu (SEQ ID NO: 2520).

20. The RNAi oligonucleotide of Embodiment 87, wherein the sense strand is ususgcCfaCfAfUfcagcuuaaaa(SEQ ID NO: 1672), and the antisense strand is vpUsUfsuuaAfgCfUfgaugUfgGfcaasusu (SEQ ID NO: 2521).

21. The RNAi oligonucleotide of Embodiment 87, wherein the sense strand is gsusgaAfaAfGfGfucaagauuaa (SEQ ID NO: 1688), and the antisense strand is vpUsUfsaauCfuUfGfaccuUfuUfcacsusu (SEQ ID NO: 2522).

22. A pharmaceutical composition comprising the RNAi oligonucleotide of Embodiment 87, or a pharmaceutically acceptable salt thereof, and a pharmaceutically acceptable carrier or excipient.

23. A method for inhibiting Phospholamban (PLN) expression or treating a PLN related disorder in a subject, the method comprising administering to the subject an effective amount of the RNAi oligonucleotide of Embodiment 87, or a pharmaceutically acceptable salt thereof.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: PLN siRNA Design and Evaluation

A set of siRNAs were designed to target sequences encoding human phospholamban (PLN) (shown here as SEQ ID NO: 2523). A detailed list of the unmodified siRNA sense and antisense strand sequences is shown in Table 1A and Table 1C below. A detailed list of modified siRNAs is shown in Table 1B below. Table 2 provides names for the nucleotide abbreviations used in the nucleic acid sequences herein. Oligonucleotides were chemically synthesized using phosphoramidite approach and after quality control using UV- and mass-spectroscopy sense and antisense strands were annealed to perform screening. Table 3 provides exemplary sequences of PLN mRNA transcripts.

Human iCell iPSC derived cardiomyocytes were purchased directly from (FujiFilm Cellular Dynamics, R1017) along with associated supplemental thawing and culturing medias. Four days prior to transfection, 96-well tissue culture plates (USA Scientific, CC7682-7596) were coated with 0.1% gelatin for 1 hour at 37° C. iCell cardiomyocytes were thawed according to manufacturer recommendations. Briefly, each vial of cells was thawed in 37° C. water bath and transferred to a 50 mL centrifuge tube. The interior of the cryovial was then rinsed with 1 mL of room temperature Plating Media (FujiFilm) and transferred to the 50 mL tube by adding dropwise to the cells over 90 seconds. An additional 8 mL of Plating Media (per vial of iCell2s) was then added slowly over 60 seconds and mixed through gentle inversion. Cells were then plated on gelatin plates (after aspiration of gelatin) at a density of 50,000 cells/well at 100 μL per well of Plating Media and placed in a tissue culture incubator at 37° C., 5% CO₂. Four hours post plating, the media was carefully aspirated and replaced by 100 μL per well of 37° C. Maintenance Media (FujiFilm) and allowed to incubate as before. Media was carefully aspirated and replaced with fresh Maintenance Media every 48 hours and prior to transfection.

siRNAs were used as stock solutions in PBS at a concentration of 5 μM each. A layout for transfection was designed and siRNA were transferred to a secondary plate of the same layout. On the day of transfection, iCell media was changed to 80 μL fresh Maintenance Media. Transfection mixes were prepared by adding 5 μL of 5 μM siRNA to 45 μL of fresh OptiMEM® media (ThermoFisher, 31985062) in a v-bottom polypropylene 96 well plate (USA Scientific, 1833-9610). A master mix of Lipofectamine® RNAiMax (ThermoFisher, 13778150) and OptiMEM® media was created at a ratio of 0.3 μL RNAiMax and 10 μL of OptiMEM® per reaction. 50 μL of RNAiMax master mix was added to each 50 μL of siRNA/OptiMEM® and the plate was sealed with sterile plate tape (USA Scientific, 2920-0010). Each transfection mix plate was then rotated at 400 rpm for 5 minutes, then removed and allowed to sit at room temperature in the tissue culture hood for 15 minutes. Following incubation, 20 μL of each siRNA/RNAiMax was added to respective wells of the pre-plated iCell cardiomyocytes, in triplicate. This created a final concentration of 50 nM siRNA per well. At 48 hours post transfection, 50 μL of fresh Maintenance Media was added to the cells for feeding. Transfections were allowed to incubate for 72 hours prior to harvest and RNA isolation. Multiple dose validation screens were performed just as the initial screens with small changes to preparation and final siRNA dosing. Individual siRNA at 5 μM were diluted appropriately into OptiMEM® media to make a highest dose. Serial dilutions were prepared in separate plates horizontally across (60, 20, 6, 2, 0.6, 0.2, 0.06, 0.02, 0.006, 0.002, 0.0006, 0.0002 nM or 3, 1, 0.3, 0.1, 0.03, 0.01, 0.003, 0.001, 0.0003, 0.0001, 0.00003, 0.00001 nM) using fresh OptiMEM®. Just as with the single dose screens, 50 μL of RNAiMax master mix (0.3 μL RNAiMax to 10 μL OptiMEM®) was added to each diluted siRNA. Each plate was then taped, rotated at 400 rpm for 5 minutes, and allowed to incubate at room temperature for 15 minutes. 20 μL of each siRNA reaction was then added to pre-plated iCell2 cardiomyocytes. Transfections were allowed to incubate for 72 hours with a 48-hour feeding of Maintenance Media prior to harvest and RNA isolation.

72 hours post transfection, cells were harvested, and RNA was isolated using the Quick-RNA 96 Kit (Zymo Research, R1053) according to the manufacturer protocol using vacuum manifolds for washing and eluting via swing bucket centrifuging at 1500 g. Harvested RNA was assayed for PLN expression via Taqman® qPCR using the Luna Universal Probe One-Step RT-qPCR Kit (NEB, E3006). Untransfected controls from each transfection plate were pooled together and a 1:5 dilution series is produced for each qPCR assay plate for direct normalization. A single qPCR assay was performed for each sample using a PLN Taqman® probe set (Hs00160179_ml-FAM) multiplexed with a common GAPDH VIC probe (ThermoFisher, 4326317E) according to the manufacturer instructions for a combined primer/probe set. Thermocycling and data acquisition was performed with an Applied Biosystems QuantStudio 3/5 Real-Time PCR System.

TABLE 1A
provides exemplary siRNAs with the sequences of the sense strand and the
antisense strand.

| Position in | SEQ ID | | SEQ ID | |
|---|---|---|---|---|
| transcript | NO | Sense strand | NO | Antisense strand |
| 202 | 1 | AAGTCCAATACCTCACTCG | 629 | CGAGTGAGGTATTGGACTT |
| 1209 | 2 | TAGCTTACCATACTATATC | 630 | GATATAGTATGGTAAGCTA |
| 1211 | 3 | GCTTACCATACTATATCTT | 631 | AAGATATAGTATGGTAAGC |
| 1481 | 4 | GACAATTCGTGGGTCCGCA | 632 | TGCGGACCCACGAATTGTC |
| 2424 | 5 | AGATTTCTTAAGTGACGCC | 633 | GGCGTCACTTAAGAAATCT |
| 2430 | 6 | CTTAAGTGACGCCTCATCT | 634 | AGATGAGGCGTCACTTAAG |
| 42 | 7 | CCCGTAAGACTTCATACAA | 635 | TTGTATGAAGTCTTACGGG |
| 219 | 8 | CGCTCAGCTATAAGAAGAG | 636 | CTCTTCTTATAGCTGAGCG |
| 329 | 9 | CTGTATCATCGTGATGCTT | 637 | AAGCATCACGATGATACAG |
| 330 | 10 | TGTATCATCGTGATGCTTC | 638 | GAAGCATCACGATGATACA |
| 1130 | 11 | GTCATATCACTAATATACT | 639 | AGTATATTAGTGATATGAC |
| 1202 | 12 | AGTTACCTAGCTTACCATA | 640 | TATGGTAAGCTAGGTAACT |
| 1503 | 13 | TCTTAACTACCTAATAGCC | 641 | GGCTATTAGGTAGTTAAGA |
| 1504 | 14 | CTTAACTACCTAATAGCCT | 642 | AGGCTATTAGGTAGTTAAG |
| 1900 | 15 | CTCTATCAACCAAATGGTA | 643 | TACCATTTGGTTGATAGAG |
| 1906 | 16 | CAACCAAATGGTAAGCATC | 644 | GATGCTTACCATTTGGTTG |
| 1939 | 17 | CTCTACTGAGCTAAATTAT | 645 | ATAATTTAGCTCAGTAGAG |
| 1940 | 18 | TCTACTGAGCTAAATTATA | 646 | TATAATTTAGCTCAGTAGA |
| 1948 | 19 | GCTAAATTATAGATCCAGC | 647 | GCTGGATCTATAATTTAGC |
| 2301 | 20 | ACACAAGTGTTGCTAACTC | 648 | GAGTTAGCAACACTTGTGT |
| 2309 | 21 | GTTGCTAACTCAATAGTGA | 649 | TCACTATTGAGTTAGCAAC |
| 2425 | 22 | GATTTCTTAAGTGACGCCT | 650 | AGGCGTCACTTAAGAAATC |
| 2429 | 23 | TCTTAAGTGACGCCTCATC | 651 | GATGAGGCGTCACTTAAGA |
| 2431 | 24 | TTAAGTGACGCCTCATCTA | 652 | TAGATGAGGCGTCACTTAA |
| 226 | 25 | CTATAAGAAGAGCCTCAAC | 653 | GTTGAGGCTCTTCTTATAG |
| 227 | 26 | TATAAGAAGAGCCTCAACC | 654 | GGTTGAGGCTCTTCTTATA |
| 228 | 27 | ATAAGAAGAGCCTCAACCA | 655 | TGGTTGAGGCTCTTCTTAT |
| 260 | 28 | ACAAGCACGTCAAAAGCTA | 656 | TAGCTTTTGACGTGCTTGT |
| 276 | 29 | CTACAGAATCTATTTATCA | 657 | TGATAAATAGATTCTGTAG |
| 667 | 30 | AAAACTGCACTGCCAACAA | 658 | TTGTTGGCAGTGCAGTTTT |
| 668 | 31 | AAACTGCACTGCCAACAAG | 659 | CTTGTTGGCAGTGCAGTTT |
| 959 | 32 | TGAGAATCAAGTATGGAAA | 660 | TTTCCATACTTGATTCTCA |
| 1099 | 33 | AACTGGTGGTTAATATGTG | 661 | CACATATTAACCACCAGTT |
| 1100 | 34 | ACTGGTGGTTAATATGTGA | 662 | TCACATATTAACCACCAGT |
| 1590 | 35 | ATTATACACTATATTCCTA | 663 | TAGGAATATAGTGTATAAT |
| 2522 | 36 | GTAAATTACCAATACAGAA | 664 | TTCTGTATTGGTAATTTAC |
| 2576 | 37 | AACTGATTAGTCATATTCC | 665 | GGAATATGACTAATCAGTT |
| 2837 | 38 | ACTATAGAATAAGTTCTTA | 666 | TAAGAACTTATTCTATAGT |
| 225 | 39 | GCTATAAGAAGAGCCTCAA | 667 | TTGAGGCTCTTCTTATAGC |
| 476 | 40 | AAAGGTCAAGATTAAGACT | 668 | AGTCTTAATCTTGACCTTT |
| 477 | 41 | AAGGTCAAGATTAAGACTA | 669 | TAGTCTTAATCTTGACCTT |
| 484 | 42 | AGATTAAGACTAAAACTTA | 670 | TAAGTTTTAGTCTTAATCT |
| 809 | 43 | ACAAATGAAGTGTCATTAT | 671 | ATAATGACACTTCATTTGT |
| 617 | 44 | CTCACAAATTTCTATCCCA | 672 | TGGGATAGAAATTTGTGAG |
| 666 | 45 | TAAAACTGCACTGCCAACA | 673 | TGTTGGCAGTGCAGTTTTA |
| 669 | 46 | AACTGCACTGCCAACAAGT | 674 | ACTTGTTGGCAGTGCAGTT |
| 274 | 47 | AGCTACAGAATCTATTTAT | 675 | ATAAATAGATTCTGTAGCT |
| 275 | 48 | GCTACAGAATCTATTTATC | 676 | GATAAATAGATTCTGTAGC |
| 1098 | 49 | AAAACTTATTGTTACCATA | 677 | TATGGTAACAATAAGTTTT |
| 1101 | 50 | GAACTGGTGGTTAATATGT | 678 | ACATATTAACCACCAGTTC |
| 1413 | 51 | AACTGGTGGTTAATATGTG | 679 | CACATATTAACCACCAGTT |
| 1483 | 52 | ACTGGTGGTTAATATGTGA | 680 | TCACATATTAACCACCAGT |
| 1484 | 53 | CTGGTGGTTAATATGTGAC | 681 | GTCACATATTAACCACCAG |
| 1485 | 54 | ATTCGTGGGTCCGCAAAAT | 682 | ATTTTGCGGACCCACGAAT |
| 1600 | 55 | ATATTCCTACAATAAAGTA | 683 | TACTTTATTGTAGGAATAT |
| 1876 | 56 | AATAGGATATAGCTATTTT | 684 | AAAATAGCTATATCCTATT |
| 1933 | 57 | AGTCCACTCTACTGAGCTA | 685 | TAGCTCAGTAGAGTGGACT |
| 2158 | 58 | ATAATCCAACAATTTTAAT | 686 | ATTAAAATTGTTGGATTAT |
| 2269 | 59 | ATATGTAACATCAATATTG | 687 | CAATATTGATGTTACATAT |
| 2349 | 60 | TGAACCCATGAGAGATACT | 688 | AGTATCTCTCATGGGTTCA |
| 2856 | 61 | TCTTAATTTACAGGGCACT | 689 | AGTGCCCTGTAAATTAAGA |
| 2857 | 62 | CTTAATTTACAGGGCACTA | 690 | TAGTGCCCTGTAAATTAAG |
| 278 | 63 | ACAGAATCTATTTATCAAT | 691 | ATTGATAAATAGATTCTGT |
| 2692 | 64 | TGAGGATTACAGAATACTA | 692 | TAGTATTCTGTAATCCTCA |
| 757 | 65 | TTCTTTAATACTAAGTATT | 693 | AATACTTAGTATTAAAGAA |
| 1379 | 66 | TCTCCAAATATTAACTAAT | 694 | ATTAGTTAATATTTGGAGA |
| 2220 | 67 | TAGAGGATGTGTAATTAAC | 695 | GTTAATTACACATCCTCTA |
| 2102 | 68 | AATTTAAGTTGACTAAAGT | 696 | ACTTTAGTCAACTTAAATT |
| 2690 | 69 | TGTGAGGATTACAGAATAC | 697 | GTATTCTGTAATCCTCACA |
| 52 | 70 | TTCATACAACACAATACTC | 698 | GAGTATTGTGTTGTATGAA |
| 1380 | 71 | CTCCAAATATTAACTAATT | 699 | AATTAGTTAATATTTGGAG |
| 1132 | 72 | CATATCACTAATATACTAA | 700 | TTAGTATATTAGTGATATG |
| 1126 | 73 | ATTAGTCATATCACTAATA | 701 | TATTAGTGATATGACTAAT |
| 281 | 74 | GAATCTATTTATCAATTTC | 702 | GAAATTGATAAATAGATTC |
| 2232 | 75 | AATTAACCATATCTTCTAA | 703 | TTAGAAGATATGGTTAATT |
| 2219 | 76 | GTAGAGGATGTGTAATTAA | 704 | TTAATTACACATCCTCTAC |
| 199 | 77 | AGAAAGTCCAATACCTCAC | 705 | GTGAGGTATTGGACTTTCT |
| 2093 | 78 | AACTTTGGTAATTTAAGTT | 706 | AACTTAAATTACCAAAGTT |
| 1378 | 79 | CTCTCCAAATATTAACTAA | 707 | TTAGTTAATATTTGGAGAG |
| 2840 | 80 | ATAGAATAAGTTCTTATCT | 708 | AGATAAGAACTTATTCTAT |
| 1134 | 81 | TATCACTAATATACTAACA | 709 | TGTTAGTATATTAGTGATA |
| 2222 | 82 | GAGGATGTGTAATTAACCA | 710 | TGGTTAATTACACATCCTC |
| 2703 | 83 | GAATACTATAACTCAAATT | 711 | AATTTGAGTTATAGTATTC |
| 358 | 84 | CTGCTACAACCTCTAGATC | 712 | GATCTAGAGGTTGTAGCAG |
| 285 | 85 | CTATTTATCAATTTCTGTC | 713 | GACAGAAATTGATAAATAG |
| 2184 | 86 | GAAGACATGTTACTAATAT | 714 | ATATTAGTAACATGTCTTC |
| 1377 | 87 | TCTCTCCAAATATTAACTA | 715 | TAGTTAATATTTGGAGAGA |
| 359 | 88 | TGCTACAACCTCTAGATCT | 716 | AGATCTAGAGGTTGTAGCA |
| 194 | 89 | CATGGAGAAAGTCCAATAC | 717 | GTATTGGACTTTCTCCATG |
| 888 | 90 | ACTATCAGAATCTACATTC | 718 | GAATGTAGATTCTGATAGT |
| 193 | 91 | TCATGGAGAAAGTCCAATA | 719 | TATTGGACTTTCTCCATGA |
| 209 | 92 | AUACCUCACUCGCUCAGCA | 720 | UGCUGAGCGAGUGAGGUAU |
| 210 | 93 | UACCUCACUCGCUCAGCUA | 721 | UAGCUGAGCGAGUGAGGUA |
| 211 | 94 | ACCUCACUCGCUCAGCUAA | 722 | UUAGCUGAGCGAGUGAGGU |
| 248 | 95 | UGAAAUGCCUCAACAAGCA | 723 | UGCUUGUUGAGGCAUUUCA |
| 249 | 96 | GAAAUGCCUCAACAAGCAA | 724 | UUGCUUGUUGAGGCAUUUC |
| 250 | 97 | AAAUGCCUCAACAAGCACA | 725 | UGUGCUUGUUGAGGCAUUU |
| 251 | 98 | AAUGCCUCAACAAGCACGA | 726 | UCGUGCUUGUUGAGGCAUU |
| 252 | 99 | AUGCCUCAACAAGCACGUA | 727 | UACGUGCUUGUUGAGGCAU |
| 253 | 100 | UGCCUCAACAAGCACGUCA | 728 | UGACGUGCUUGUUGAGGCA |
| 606 | 101 | CCUCAACAUGGCUCACAAA | 729 | UUUGUGAGCCAUGUUGAGG |
| 607 | 102 | CUCAACAUGGCUCACAAAA | 730 | UUUUGUGAGCCAUGUUGAG |
| 608 | 103 | UCAACAUGGCUCACAAAUA | 731 | UAUUUGUGAGCCAUGUUGA |
| 609 | 104 | CAACAUGGCUCACAAAUUA | 732 | UAAUUUGUGAGCCAUGUUG |
| 203 | 105 | AGUCCAAUACCUCACUCGA | 733 | UCGAGUGAGGUAUUGGACU |
| 204 | 106 | GUCCAAUACCUCACUCGCA | 734 | UGCGAGUGAGGUAUUGGAC |
| 206 | 107 | CCAAUACCUCACUCGCUCA | 735 | UGAGCGAGUGAGGUAUUGG |
| 207 | 108 | CAAUACCUCACUCGCUCAA | 736 | UUGAGCGAGUGAGGUAUUG |
| 223 | 109 | CAGCUAUAAGAAGAGCCUA | 737 | UAGGCUCUUCUUAUAGCUG |
| 230 | 110 | AAGAAGAGCCUCAACCAUA | 738 | UAUGGUUGAGGCUCUUCUU |
| 300 | 111 | UGUCUCAUCUUAAUAUGUA | 739 | UACAUAUUAAGAUGAGACA |
| 301 | 112 | GUCUCAUCUUAAUAUGUCA | 740 | UGACAUAUUAAGAUGAGAC |
| 302 | 113 | UCUCAUCUUAAUAUGUCUA | 741 | UAGACAUAUUAAGAUGAGA |
| 303 | 114 | CUCAUCUUAAUAUGUCUCA | 742 | UGAGACAUAUUAAGAUGAG |
| 306 | 115 | AUCUUAAUAUGUCUCUUGA | 743 | UCAAGAGACAUAUUAAGAU |
| 307 | 116 | UCUUAAUAUGUCUCUUGCA | 744 | UGCAAGAGACAUAUUAAGA |
| 308 | 117 | CUUAAUAUGUCUCUUGCUA | 745 | UAGCAAGAGACAUAUUAAG |
| 309 | 118 | UUAAUAUGUCUCUUGCUGA | 746 | UCAGCAAGAGACAUAUUAA |
| 310 | 119 | UAAUAUGUCUCUUGCUGAA | 747 | UUCAGCAAGAGACAUAUUA |
| 311 | 120 | AAUAUGUCUCUUGCUGAUA | 748 | UAUCAGCAAGAGACAUAUU |
| 312 | 121 | AUAUGUCUCUUGCUGAUCA | 749 | UGAUCAGCAAGAGACAUAU |
| 314 | 122 | AUGUCUCUUGCUGAUCUGA | 750 | UCAGAUCAGCAAGAGACAU |
| 315 | 123 | UGUCUCUUGCUGAUCUGUA | 751 | UACAGAUCAGCAAGAGACA |
| 321 | 124 | UUGCUGAUCUGUAUCAUCA | 752 | UGAUGAUACAGAUCAGCAA |
| 322 | 125 | UGCUGAUCUGUAUCAUCGA | 753 | UCGAUGAUACAGAUCAGCA |
| 323 | 126 | GCUGAUCUGUAUCAUCGUA | 754 | UACGAUGAUACAGAUCAGC |
| 324 | 127 | CUGAUCUGUAUCAUCGUGA | 755 | UCACGAUGAUACAGAUCAG |
| 325 | 128 | UGAUCUGUAUCAUCGUGAA | 756 | UUCACGAUGAUACAGAUCA |
| 326 | 129 | GAUCUGUAUCAUCGUGAUA | 757 | UAUCACGAUGAUACAGAUC |
| 327 | 130 | AUCUGUAUCAUCGUGAUGA | 758 | UCAUCACGAUGAUACAGAU |
| 328 | 131 | UCUGUAUCAUCGUGAUGCA | 759 | UGCAUCACGAUGAUACAGA |
| 331 | 132 | GUAUCAUCGUGAUGCUUCA | 760 | UGAAGCAUCACGAUGAUAC |
| 334 | 133 | UCAUCGUGAUGCUUCUCUA | 761 | UAGAGAAGCAUCACGAUGA |
| 335 | 134 | CAUCGUGAUGCUUCUCUGA | 762 | UCAGAGAAGCAUCACGAUG |
| 336 | 135 | AUCGUGAUGCUUCUCUGAA | 763 | UUCAGAGAAGCAUCACGAU |
| 337 | 136 | UCGUGAUGCUUCUCUGAAA | 764 | UUUCAGAGAAGCAUCACGA |
| 338 | 137 | CGUGAUGCUUCUCUGAAGA | 765 | UCUUCAGAGAAGCAUCACG |
| 349 | 138 | UCUGAAGUUCUGCUACAAA | 766 | UUUGUAGCAGAACUUCAGA |
| 350 | 139 | CUGAAGUUCUGCUACAACA | 767 | UGUUGUAGCAGAACUUCAG |
| 351 | 140 | UGAAGUUCUGCUACAACCA | 768 | UGGUUGUAGCAGAACUUCA |
| 356 | 141 | UUCUGCUACAACCUCUAGA | 769 | UCUAGAGGUUGUAGCAGAA |
| 360 | 142 | GCUACAACCUCUAGAUCUA | 770 | UAGAUCUAGAGGUUGUAGC |
| 399 | 143 | AAAUCUGUCAUCCCAUGCA | 771 | UGCAUGGGAUGACAGAUUU |
| 400 | 144 | AAUCUGUCAUCCCAUGCAA | 772 | UUGCAUGGGAUGACAGAUU |
| 451 | 145 | CCUGAGUAGAAGAGUUUCA | 773 | UGAAACUCUUCUACUCAGG |
| 470 | 146 | UUGUGAAAAGGUCAAGAUA | 774 | UAUCUUGACCUUUUCACAA |
| 471 | 147 | UGUGAAAAGGUCAAGAUUA | 775 | UAAUCUUGACCUUUUCACA |
| 475 | 148 | AAAAGGUCAAGAUUAAGAA | 776 | UUCUUAAUCUUGACCUUUU |
| 478 | 149 | AGGUCAAGAUUAAGACUAA | 777 | UUAGUCUUAAUCUUGACCU |
| 480 | 150 | GUCAAGAUUAAGACUAAAA | 778 | UUUUAGUCUUAAUCUUGAC |
| 481 | 151 | UCAAGAUUAAGACUAAAAA | 779 | UUUUUAGUCUUAAUCUUGA |
| 482 | 152 | CAAGAUUAAGACUAAAACA | 780 | UGUUUUAGUCUUAAUCUUG |
| 483 | 153 | AAGAUUAAGACUAAAACUA | 781 | UAGUUUUAGUCUUAAUCUU |
| 683 | 154 | CAAGUUCACUUCAUAUAUA | 782 | UAUAUAUGAAGUGAACUUG |
| 684 | 155 | AAGUUCACUUCAUAUAUAA | 783 | UUAUAUAUGAAGUGAACUU |
| 715 | 156 | ACUCUUUUGAGGUGAAUAA | 784 | UUAUUCACCUCAAAAGAGU |
| 716 | 157 | CUCUUUUGAGGUGAAUAUA | 785 | UAUAUUCACCUCAAAAGAG |
| 717 | 158 | UCUUUUGAGGUGAAUAUAA | 786 | UUAUAUUCACCUCAAAAGA |
| 786 | 159 | UCACCAAGUAUCAAAGUAA | 787 | UUACUUUGAUACUUGGUGA |
| 788 | 160 | ACCAAGUAUCAAAGUAAUA | 788 | UAUUACUUUGAUACUUGGU |
| 789 | 161 | CCAAGUAUCAAAGUAAUAA | 789 | UUAUUACUUUGAUACUUGG |
| 810 | 162 | CAAAUGAAGUGUCAUUAUA | 790 | UAUAAUGACACUUCAUUUG |
| 811 | 163 | AAAUGAAGUGUCAUUAUUA | 791 | UAAUAAUGACACUUCAUUU |
| 812 | 164 | AAUGAAGUGUCAUUAUUCA | 792 | UGAAUAAUGACACUUCAUU |
| 264 | 165 | GCACGUCAAAAGCUACAGA | 793 | UCUGUAGCUUUUGACGUGC |
| 521 | 166 | AUCUGUUGGAUCUUGUAAA | 794 | UUUACAAGAUCCAACAGAU |
| 522 | 167 | UCUGUUGGAUCUUGUAAAA | 795 | UUUUACAAGAUCCAACAGA |
| 523 | 168 | CUGUUGGAUCUUGUAAACA | 796 | UGUUUACAAGAUCCAACAG |
| 524 | 169 | UGUUGGAUCUUGUAAACAA | 797 | UUGUUUACAAGAUCCAACA |
| 525 | 170 | GUUGGAUCUUGUAAACAUA | 798 | UAUGUUUACAAGAUCCAAC |
| 593 | 171 | CAACUGUUGAUUUCCUCAA | 799 | UUGAGGAAAUCAACAGUUG |
| 610 | 172 | AACAUGGCUCACAAAUUUA | 800 | UAAAUUUGUGAGCCAUGUU |
| 618 | 173 | UCACAAAUUUCUAUCCCAA | 801 | UUGGGAUAGAAAUUUGUGA |
| 623 | 174 | AAUUUCUAUCCCAAAUCUA | 802 | UAGAUUUGGGAUAGAAAUU |
| 624 | 175 | AUUUCUAUCCCAAAUCUUA | 803 | UAAGAUUUGGGAUAGAAAU |
| 718 | 176 | CUUUUGAGGUGAAUAUAAA | 804 | UUUAUAUUCACCUCAAAAG |
| 776 | 177 | UUUCAGGUCUUCACCAAGA | 805 | UCUUGGUGAAGACCUGAAA |
| 777 | 178 | UUCAGGUCUUCACCAAGUA | 806 | UACUUGGUGAAGACCUGAA |
| 778 | 179 | UCAGGUCUUCACCAAGUAA | 807 | UUACUUGGUGAAGACCUGA |
| 779 | 180 | CAGGUCUUCACCAAGUAUA | 808 | UAUACUUGGUGAAGACCUG |
| 780 | 181 | AGGUCUUCACCAAGUAUCA | 809 | UGAUACUUGGUGAAGACCU |
| 813 | 182 | AUGAAGUGUCAUUAUUCAA | 810 | UUGAAUAAUGACACUUCAU |
| 839 | 183 | CACUGACUCCUCACAUCUA | 811 | UAGAUGUGAGGAGUCAGUG |
| 926 | 184 | UUUCUAUGCCACAUUAACA | 812 | UGUUAAUGUGGCAUAGAAA |
| 49 | 185 | GACUUCAUACAACACAAUA | 813 | UAUUGUGUUGUAUGAAGUC |
| 54 | 186 | CAUACAACACAAUACUCUA | 814 | UAGAGUAUUGUGUUGUAUG |
| 55 | 187 | AUACAACACAAUACUCUAA | 815 | UUAGAGUAUUGUGUUGUAU |
| 109 | 188 | GAAGACAGUUAUCUCAUAA | 816 | UUAUGAGAUAACUGUCUUC |
| 110 | 189 | AAGACAGUUAUCUCAUAUA | 817 | UAUAUGAGAUAACUGUCUU |
| 113 | 190 | ACAGUUAUCUCAUAUUUGA | 818 | UCAAAUAUGAGAUAACUGU |
| 115 | 191 | AGUUAUCUCAUAUUUGGCA | 819 | UGCCAAAUAUGAGAUAACU |
| 195 | 192 | AUGGAGAAAGUCCAAUACA | 820 | UGUAUUGGACUUUCUCCAU |
| 231 | 193 | AGAAGAGCCUCAACCAUUA | 821 | UAAUGGUUGAGGCUCUUCU |
| 232 | 194 | GAAGAGCCUCAACCAUUGA | 822 | UCAAUGGUUGAGGCUCUUC |
| 233 | 195 | AAGAGCCUCAACCAUUGAA | 823 | UUCAAUGGUUGAGGCUCUU |
| 234 | 196 | AGAGCCUCAACCAUUGAAA | 824 | UUUCAAUGGUUGAGGCUCU |
| 237 | 197 | GCCUCAACCAUUGAAAUGA | 825 | UCAUUUCAAUGGUUGAGGC |
| 240 | 198 | UCAACCAUUGAAAUGCCUA | 826 | UAGGCAUUUCAAUGGUUGA |
| 244 | 199 | CCAUUGAAAUGCCUCAACA | 827 | UGUUGAGGCAUUUCAAUGG |
| 296 | 200 | UUUCUGUCUCAUCUUAAUA | 828 | UAUUAAGAUGAGACAGAAA |
| 304 | 201 | UCAUCUUAAUAUGUCUCUA | 829 | UAGAGACAUAUUAAGAUGA |
| 305 | 202 | CAUCUUAAUAUGUCUCUUA | 830 | UAAGAGACAUAUUAAGAUG |
| 316 | 203 | GUCUCUUGCUGAUCUGUAA | 831 | UUACAGAUCAGCAAGAGAC |
| 317 | 204 | UCUCUUGCUGAUCUGUAUA | 832 | UAUACAGAUCAGCAAGAGA |
| 318 | 205 | CUCUUGCUGAUCUGUAUCA | 833 | UGAUACAGAUCAGCAAGAG |
| 319 | 206 | UCUUGCUGAUCUGUAUCAA | 834 | UUGAUACAGAUCAGCAAGA |
| 320 | 207 | CUUGCUGAUCUGUAUCAUA | 835 | UAUGAUACAGAUCAGCAAG |
| 341 | 208 | GAUGCUUCUCUGAAGUUCA | 836 | UGAACUUCAGAGAAGCAUC |
| 342 | 209 | AUGCUUCUCUGAAGUUCUA | 837 | UAGAACUUCAGAGAAGCAU |
| 347 | 210 | UCUCUGAAGUUCUGCUACA | 838 | UGUAGCAGAACUUCAGAGA |
| 352 | 211 | GAAGUUCUGCUACAACCUA | 839 | UAGGUUGUAGCAGAACUUC |
| 362 | 212 | UACAACCUCUAGAUCUGCA | 840 | UGCAGAUCUAGAGGUUGUA |
| 402 | 213 | UCUGUCAUCCCAUGCAGAA | 841 | UUCUGCAUGGGAUGACAGA |
| 446 | 214 | CACUUCCUGAGUAGAAGAA | 842 | UUCUUCUACUCAGGAAGUG |
| 450 | 215 | UCCUGAGUAGAAGAGUUUA | 843 | UAAACUCUUCUACUCAGGA |
| 452 | 216 | CUGAGUAGAAGAGUUUCUA | 844 | UAGAAACUCUUCUACUCAG |
| 479 | 217 | GGUCAAGAUUAAGACUAAA | 845 | UUUAGUCUUAAUCUUGACC |
| 528 | 218 | GGAUCUUGUAAACAUGAAA | 846 | UUUCAUGUUUACAAGAUCC |
| 595 | 219 | ACUGUUGAUUUCCUCAACA | 847 | UGUUGAGGAAAUCAACAGU |
| 598 | 220 | GUUGAUUUCCUCAACAUGA | 848 | UCAUGUUGAGGAAAUCAAC |
| 600 | 221 | UGAUUUCCUCAACAUGGCA | 849 | UGCCAUGUUGAGGAAAUCA |
| 601 | 222 | GAUUUCCUCAACAUGGCUA | 850 | UAGCCAUGUUGAGGAAAUC |
| 602 | 223 | AUUUCCUCAACAUGGCUCA | 851 | UGAGCCAUGUUGAGGAAAU |
| 604 | 224 | UUCCUCAACAUGGCUCACA | 852 | UGUGAGCCAUGUUGAGGAA |
| 605 | 225 | UCCUCAACAUGGCUCACAA | 853 | UUGUGAGCCAUGUUGAGGA |
| 671 | 226 | CUGCACUGCCAACAAGUUA | 854 | UAACUUGUUGGCAGUGCAG |
| 672 | 227 | UGCACUGCCAACAAGUUCA | 855 | UGAACUUGUUGGCAGUGCA |
| 674 | 228 | CACUGCCAACAAGUUCACA | 856 | UGUGAACUUGUUGGCAGUG |
| 675 | 229 | ACUGCCAACAAGUUCACUA | 857 | UAGUGAACUUGUUGGCAGU |
| 676 | 230 | CUGCCAACAAGUUCACUUA | 858 | UAAGUGAACUUGUUGGCAG |
| 679 | 231 | CCAACAAGUUCACUUCAUA | 859 | UAUGAAGUGAACUUGUUGG |
| 680 | 232 | CAACAAGUUCACUUCAUAA | 860 | UUAUGAAGUGAACUUGUUG |
| 681 | 233 | AACAAGUUCACUUCAUAUA | 861 | UAUAUGAAGUGAACUUGUU |
| 682 | 234 | ACAAGUUCACUUCAUAUAA | 862 | UUAUAUGAAGUGAACUUGU |
| 690 | 235 | ACUUCAUAUAUAAAGCAUA | 863 | UAUGCUUUAUAUAUGAAGU |
| 782 | 236 | GUCUUCACCAAGUAUCAAA | 864 | UUUGAUACUUGGUGAAGAC |
| 783 | 237 | UCUUCACCAAGUAUCAAAA | 865 | UUUUGAUACUUGGUGAAGA |
| 784 | 238 | CUUCACCAAGUAUCAAAGA | 866 | UCUUUGAUACUUGGUGAAG |
| 785 | 239 | UUCACCAAGUAUCAAAGUA | 867 | UACUUUGAUACUUGGUGAA |
| 787 | 240 | CACCAAGUAUCAAAGUAAA | 868 | UUUACUUUGAUACUUGGUG |
| 790 | 241 | CAAGUAUCAAAGUAAUAAA | 869 | UUUAUUACUUUGAUACUUG |
| 791 | 242 | AAGUAUCAAAGUAAUAACA | 870 | UGUUAUUACUUUGAUACUU |
| 792 | 243 | AGUAUCAAAGUAAUAACAA | 871 | UUGUUAUUACUUUGAUACU |
| 803 | 244 | AAUAACACAAAUGAAGUGA | 872 | UCACUUCAUUUGUGUUAUU |
| 838 | 245 | CCACUGACUCCUCACAUCA | 873 | UGAUGUGAGGAGUCAGUGG |
| 848 | 246 | CUCACAUCUGUUAUCUUAA | 874 | UUAAGAUAACAGAUGUGAG |
| 849 | 247 | UCACAUCUGUUAUCUUAUA | 875 | UAUAAGAUAACAGAUGUGA |
| 850 | 248 | CACAUCUGUUAUCUUAUUA | 876 | UAAUAAGAUAACAGAUGUG |
| 872 | 249 | AGAACUAUUUGUAGUAACA | 877 | UGUUACUACAAAUAGUUCU |
| 873 | 250 | GAACUAUUUGUAGUAACUA | 878 | UAGUUACUACAAAUAGUUC |
| 876 | 251 | CUAUUUGUAGUAACUAUCA | 879 | UGAUAGUUACUACAAAUAG |
| 877 | 252 | UAUUUGUAGUAACUAUCAA | 880 | UUGAUAGUUACUACAAAUA |
| 878 | 253 | AUUUGUAGUAACUAUCAGA | 881 | UCUGAUAGUUACUACAAAU |
| 882 | 254 | GUAGUAACUAUCAGAAUCA | 882 | UGAUUCUGAUAGUUACUAC |
| 883 | 255 | UAGUAACUAUCAGAAUCUA | 883 | UAGAUUCUGAUAGUUACUA |
| 886 | 256 | UAACUAUCAGAAUCUACAA | 884 | UUGUAGAUUCUGAUAGUUA |
| 889 | 257 | CUAUCAGAAUCUACAUUCA | 885 | UGAAUGUAGAUUCUGAUAG |
| 950 | 258 | UAAAGUUGAUGAGAAUCAA | 886 | UUGAUUCUCAUCAACUUUA |
| 951 | 259 | AAAGUUGAUGAGAAUCAAA | 887 | UUUGAUUCUCAUCAACUUU |
| 952 | 260 | AAGUUGAUGAGAAUCAAGA | 888 | UCUUGAUUCUCAUCAACUU |
| 953 | 261 | AGUUGAUGAGAAUCAAGUA | 889 | UACUUGAUUCUCAUCAACU |
| 978 | 262 | AGUAAGGCCAUACUCUUAA | 890 | UUAAGAGUAUGGCCUUACU |
| 979 | 263 | GUAAGGCCAUACUCUUACA | 891 | UGUAAGAGUAUGGCCUUAC |
| 980 | 264 | UAAGGCCAUACUCUUACAA | 892 | UUGUAAGAGUAUGGCCUUA |
| 983 | 265 | GGCCAUACUCUUACAUAAA | 893 | UUUAUGUAAGAGUAUGGCC |
| 984 | 266 | GCCAUACUCUUACAUAAUA | 894 | UAUUAUGUAAGAGUAUGGC |
| 1066 | 267 | AAUCUGUUCUAAGACAUAA | 895 | UUAUGUCUUAGAACAGAUU |
| 1068 | 268 | UCUGUUCUAAGACAUAUGA | 896 | UCAUAUGUCUUAGAACAGA |
| 1071 | 269 | GUUCUAAGACAUAUGAUCA | 897 | UGAUCAUAUGUCUUAGAAC |
| 1072 | 270 | UUCUAAGACAUAUGAUCAA | 898 | UUGAUCAUAUGUCUUAGAA |
| 1073 | 271 | UCUAAGACAUAUGAUCAAA | 899 | UUUGAUCAUAUGUCUUAGA |
| 1077 | 272 | AGACAUAUGAUCAACAGAA | 900 | UUCUGUUGAUCAUAUGUCU |
| 1078 | 273 | GACAUAUGAUCAACAGAUA | 901 | UAUCUGUUGAUCAUAUGUC |
| 1079 | 274 | ACAUAUGAUCAACAGAUGA | 902 | UCAUCUGUUGAUCAUAUGU |
| 1082 | 275 | UAUGAUCAACAGAUGAGAA | 903 | UUCUCAUCUGUUGAUCAUA |
| 1083 | 276 | AUGAUCAACAGAUGAGAAA | 904 | UUUCUCAUCUGUUGAUCAU |
| 1118 | 277 | ACAGUGAGAUUAGUCAUAA | 905 | UUAUGACUAAUCUCACUGU |
| 1119 | 278 | CAGUGAGAUUAGUCAUAUA | 906 | UAUAUGACUAAUCUCACUG |
| 1121 | 279 | GUGAGAUUAGUCAUAUCAA | 907 | UUGAUAUGACUAAUCUCAC |
| 1205 | 280 | UACCUAGCUUACCAUACUA | 908 | UAGUAUGGUAAGCUAGGUA |
| 1206 | 281 | ACCUAGCUUACCAUACUAA | 909 | UUAGUAUGGUAAGCUAGGU |
| 1207 | 282 | CCUAGCUUACCAUACUAUA | 910 | UAUAGUAUGGUAAGCUAGG |
| 1208 | 283 | CUAGCUUACCAUACUAUAA | 911 | UUAUAGUAUGGUAAGCUAG |
| 1210 | 284 | AGCUUACCAUACUAUAUCA | 912 | UGAUAUAGUAUGGUAAGCU |
| 1375 | 285 | UCUCUCUCCAAAUAUUAAA | 913 | UUUAAUAUUUGGAGAGAGA |
| 1417 | 286 | AUGAACUUGUUGGCCCAUA | 914 | UAUGGGCCAACAAGUUCAU |
| 1418 | 287 | UGAACUUGUUGGCCCAUCA | 915 | UGAUGGGCCAACAAGUUCA |
| 1419 | 288 | GAACUUGUUGGCCCAUCUA | 916 | UAGAUGGGCCAACAAGUUC |
| 1420 | 289 | AACUUGUUGGCCCAUCUAA | 917 | UUAGAUGGGCCAACAAGUU |
| 1422 | 290 | CUUGUUGGCCCAUCUAUUA | 918 | UAAUAGAUGGGCCAACAAG |
| 1424 | 291 | UGUUGGCCCAUCUAUUACA | 919 | UGUAAUAGAUGGGCCAACA |
| 1956 | 292 | AUAGAUCCAGCUAUGCUAA | 920 | UUAGCAUAGCUGGAUCUAU |
| 1957 | 293 | UAGAUCCAGCUAUGCUAUA | 921 | UAUAGCAUAGCUGGAUCUA |
| 1958 | 294 | AGAUCCAGCUAUGCUAUUA | 922 | UAAUAGCAUAGCUGGAUCU |
| 2025 | 295 | UCAGAACAUCUUCCAAUAA | 923 | UUAUUGGAAGAUGUUCUGA |
| 2030 | 296 | ACAUCUUCCAAUAACUCAA | 924 | UUGAGUUAUUGGAAGAUGU |
| 2087 | 297 | UCACCAAACUUUGGUAAUA | 925 | UAUUACCAAAGUUUGGUGA |
| 2088 | 298 | CACCAAACUUUGGUAAUUA | 926 | UAAUUACCAAAGUUUGGUG |
| 2094 | 299 | ACUUUGGUAAUUUAAGUUA | 927 | UAACUUAAAUUACCAAAGU |
| 2099 | 300 | GGUAAUUUAAGUUGACUAA | 928 | UUAGUCAACUUAAAUUACC |
| 2145 | 301 | ACACCUAUACUGCAUAAUA | 929 | UAUUAUGCAGUAUAGGUGU |
| 2146 | 302 | CACCUAUACUGCAUAAUCA | 930 | UGAUUAUGCAGUAUAGGUG |
| 2147 | 303 | ACCUAUACUGCAUAAUCCA | 931 | UGGAUUAUGCAGUAUAGGU |
| 2148 | 304 | CCUAUACUGCAUAAUCCAA | 932 | UUGGAUUAUGCAGUAUAGG |
| 2150 | 305 | UAUACUGCAUAAUCCAACA | 933 | UGUUGGAUUAUGCAGUAUA |
| 2151 | 306 | AUACUGCAUAAUCCAACAA | 934 | UUGUUGGAUUAUGCAGUAU |
| 2152 | 307 | UACUGCAUAAUCCAACAAA | 935 | UUUGUUGGAUUAUGCAGUA |
| 2153 | 308 | ACUGCAUAAUCCAACAAUA | 936 | UAUUGUUGGAUUAUGCAGU |
| 2182 | 309 | UUGAAGACAUGUUACUAAA | 937 | UUUAGUAACAUGUCUUCAA |
| 2183 | 310 | UGAAGACAUGUUACUAAUA | 938 | UAUUAGUAACAUGUCUUCA |
| 2188 | 311 | ACAUGUUACUAAUAUAACA | 939 | UGUUAUAUUAGUAACAUGU |
| 2216 | 312 | AGAGUAGAGGAUGUGUAAA | 940 | UUUACACAUCCUCUACUCU |
| 2228 | 313 | GUGUAAUUAACCAUAUCUA | 941 | UAGAUAUGGUUAAUUACAC |
| 2281 | 314 | AAUAUUGACCUUGGUUUCA | 942 | UGAAACCAAGGUCAAUAUU |
| 2282 | 315 | AUAUUGACCUUGGUUUCUA | 943 | UAGAAACCAAGGUCAAUAU |
| 2284 | 316 | AUUGACCUUGGUUUCUUAA | 944 | UUAAGAAACCAAGGUCAAU |
| 2288 | 317 | ACCUUGGUUUCUUACACAA | 945 | UUGUGUAAGAAACCAAGGU |
| 2293 | 318 | GGUUUCUUACACAAGUGUA | 946 | UACACUUGUGUAAGAAACC |
| 2295 | 319 | UUUCUUACACAAGUGUUGA | 947 | UCAACACUUGUGUAAGAAA |
| 2297 | 320 | UCUUACACAAGUGUUGCUA | 948 | UAGCAACACUUGUGUAAGA |
| 2303 | 321 | ACAAGUGUUGCUAACUCAA | 949 | UUGAGUUAGCAACACUUGU |
| 2306 | 322 | AGUGUUGCUAACUCAAUAA | 950 | UUAUUGAGUUAGCAACACU |
| 2307 | 323 | GUGUUGCUAACUCAAUAGA | 951 | UCUAUUGAGUUAGCAACAC |
| 2308 | 324 | UGUUGCUAACUCAAUAGUA | 952 | UACUAUUGAGUUAGCAACA |
| 2310 | 325 | UUGCUAACUCAAUAGUGAA | 953 | UUCACUAUUGAGUUAGCAA |
| 2311 | 326 | UGCUAACUCAAUAGUGAAA | 954 | UUUCACUAUUGAGUUAGCA |
| 2315 | 327 | AACUCAAUAGUGAAGGAGA | 955 | UCUCCUUCACUAUUGAGUU |
| 2317 | 328 | CUCAAUAGUGAAGGAGACA | 956 | UGUCUCCUUCACUAUUGAG |
| 2380 | 329 | UGGAAAGUGUUUGGUUCAA | 957 | UUGAACCAAACACUUUCCA |
| 2389 | 330 | UUUGGUUCAGGGAUAUCUA | 958 | UAGAUAUCCCUGAACCAAA |
| 2390 | 331 | UUGGUUCAGGGAUAUCUGA | 959 | UCAGAUAUCCCUGAACCAA |
| 2420 | 332 | GCAGAGAUUUCUUAAGUGA | 960 | UCACUUAAGAAAUCUCUGC |
| 2432 | 333 | UAAGUGACGCCUCAUCUAA | 961 | UUAGAUGAGGCGUCACUUA |
| 2434 | 334 | AGUGACGCCUCAUCUACAA | 962 | UUGUAGAUGAGGCGUCACU |
| 2437 | 335 | GACGCCUCAUCUACAAGCA | 963 | UGCUUGUAGAUGAGGCGUC |
| 2443 | 336 | UCAUCUACAAGCUGGAAAA | 964 | UUUUCCAGCUUGUAGAUGA |
| 2601 | 337 | UACACUGUUUGUUACAAUA | 965 | UAUUGUAACAAACAGUGUA |
| 2681 | 338 | AUCUUAUUCUGUGAGGAUA | 966 | UAUCCUCACAGAAUAAGAU |
| 2697 | 339 | AUUACAGAAUACUAUAACA | 967 | UGUUAUAGUAUUCUGUAAU |
| 2700 | 340 | ACAGAAUACUAUAACUCAA | 968 | UUGAGUUAUAGUAUUCUGU |
| 2947 | 341 | AAAUCAGAAUCACUAUAUA | 969 | UAUAUAGUGAUUCUGAUUU |
| 17 | 342 | AGUCAGAAAACUCCCCAGA | 970 | UCUGGGGAGUUUUCUGACU |
| 18 | 343 | GUCAGAAAACUCCCCAGCA | 971 | UGCUGGGGAGUUUUCUGAC |
| 19 | 344 | UCAGAAAACUCCCCAGCUA | 972 | UAGCUGGGGAGUUUUCUGA |
| 20 | 345 | CAGAAAACUCCCCAGCUAA | 973 | UUAGCUGGGGAGUUUUCUG |
| 21 | 346 | AGAAAACUCCCCAGCUAAA | 974 | UUUAGCUGGGGAGUUUUCU |
| 45 | 347 | GUAAGACUUCAUACAACAA | 975 | UUGUUGUAUGAAGUCUUAC |
| 47 | 348 | AAGACUUCAUACAACACAA | 976 | UUGUGUUGUAUGAAGUCUU |
| 48 | 349 | AGACUUCAUACAACACAAA | 977 | UUUGUGUUGUAUGAAGUCU |
| 91 | 350 | UGCCAAGGCUACCUAAAAA | 978 | UUUUUAGGUAGCCUUGGCA |
| 94 | 351 | CAAGGCUACCUAAAAGAAA | 979 | UUUCUUUUAGGUAGCCUUG |
| 95 | 352 | AAGGCUACCUAAAAGAAGA | 980 | UCUUCUUUUAGGUAGCCUU |
| 96 | 353 | AGGCUACCUAAAAGAAGAA | 981 | UUCUUCUUUUAGGUAGCCU |
| 97 | 354 | GGCUACCUAAAAGAAGACA | 982 | UGUCUUCUUUUAGGUAGCC |
| 98 | 355 | GCUACCUAAAAGAAGACAA | 983 | UUGUCUUCUUUUAGGUAGC |
| 99 | 356 | CUACCUAAAAGAAGACAGA | 984 | UCUGUCUUCUUUUAGGUAG |
| 100 | 357 | UACCUAAAAGAAGACAGUA | 985 | UACUGUCUUCUUUUAGGUA |
| 101 | 358 | ACCUAAAAGAAGACAGUUA | 986 | UAACUGUCUUCUUUUAGGU |
| 102 | 359 | CCUAAAAGAAGACAGUUAA | 987 | UUAACUGUCUUCUUUUAGG |
| 103 | 360 | CUAAAAGAAGACAGUUAUA | 988 | UAUAACUGUCUUCUUUUAG |
| 104 | 361 | UAAAAGAAGACAGUUAUCA | 989 | UGAUAACUGUCUUCUUUUA |
| 105 | 362 | AAAAGAAGACAGUUAUCUA | 990 | UAGAUAACUGUCUUCUUUU |
| 106 | 363 | AAAGAAGACAGUUAUCUCA | 991 | UGAGAUAACUGUCUUCUUU |
| 107 | 364 | AAGAAGACAGUUAUCUCAA | 992 | UUGAGAUAACUGUCUUCUU |
| 108 | 365 | AGAAGACAGUUAUCUCAUA | 993 | UAUGAGAUAACUGUCUUCU |
| 111 | 366 | AGACAGUUAUCUCAUAUUA | 994 | UAAUAUGAGAUAACUGUCU |
| 112 | 367 | GACAGUUAUCUCAUAUUUA | 995 | UAAAUAUGAGAUAACUGUC |
| 114 | 368 | CAGUUAUCUCAUAUUUGGA | 996 | UCCAAAUAUGAGAUAACUG |
| 161 | 369 | CUUAAAACUUCAGACUUCA | 997 | UGAAGUCUGAAGUUUUAAG |
| 163 | 370 | UAAAACUUCAGACUUCCUA | 998 | UAGGAAGUCUGAAGUUUUA |
| 181 | 371 | GUCCUGCUGGUAUCAUGGA | 999 | UCCAUGAUACCAGCAGGAC |
| 182 | 372 | UCCUGCUGGUAUCAUGGAA | 1000 | UUCCAUGAUACCAGCAGGA |
| 183 | 373 | CCUGCUGGUAUCAUGGAGA | 1001 | UCUCCAUGAUACCAGCAGG |
| 184 | 374 | CUGCUGGUAUCAUGGAGAA | 1002 | UUCUCCAUGAUACCAGCAG |
| 185 | 375 | UGCUGGUAUCAUGGAGAAA | 1003 | UUUCUCCAUGAUACCAGCA |
| 186 | 376 | GCUGGUAUCAUGGAGAAAA | 1004 | UUUUCUCCAUGAUACCAGC |
| 187 | 377 | CUGGUAUCAUGGAGAAAGA | 1005 | UCUUUCUCCAUGAUACCAG |
| 188 | 378 | UGGUAUCAUGGAGAAAGUA | 1006 | UACUUUCUCCAUGAUACCA |
| 189 | 379 | GGUAUCAUGGAGAAAGUCA | 1007 | UGACUUUCUCCAUGAUACC |
| 197 | 380 | GGAGAAAGUCCAAUACCUA | 1008 | UAGGUAUUGGACUUUCUCC |
| 198 | 381 | GAGAAAGUCCAAUACCUCA | 1009 | UGAGGUAUUGGACUUUCUC |
| 200 | 382 | GAAAGUCCAAUACCUCACA | 1010 | UGUGAGGUAUUGGACUUUC |
| 201 | 383 | AAAGUCCAAUACCUCACUA | 1011 | UAGUGAGGUAUUGGACUUU |
| 205 | 384 | UCCAAUACCUCACUCGCUA | 1012 | UAGCGAGUGAGGUAUUGGA |
| 212 | 385 | CCUCACUCGCUCAGCUAUA | 1013 | UAUAGCUGAGCGAGUGAGG |
| 213 | 386 | CUCACUCGCUCAGCUAUAA | 1014 | UUAUAGCUGAGCGAGUGAG |
| 214 | 387 | UCACUCGCUCAGCUAUAAA | 1015 | UUUAUAGCUGAGCGAGUGA |
| 215 | 388 | CACUCGCUCAGCUAUAAGA | 1016 | UCUUAUAGCUGAGCGAGUG |
| 217 | 389 | CUCGCUCAGCUAUAAGAAA | 1017 | UUUCUUAUAGCUGAGCGAG |
| 221 | 390 | CUCAGCUAUAAGAAGAGCA | 1018 | UGCUCUUCUUAUAGCUGAG |
| 222 | 391 | UCAGCUAUAAGAAGAGCCA | 1019 | UGGCUCUUCUUAUAGCUGA |
| 224 | 392 | AGCUAUAAGAAGAGCCUCA | 1020 | UGAGGCUCUUCUUAUAGCU |
| 229 | 393 | UAAGAAGAGCCUCAACCAA | 1021 | UUGGUUGAGGCUCUUCUUA |
| 254 | 394 | GCCUCAACAAGCACGUCAA | 1022 | UUGACGUGCUUGUUGAGGC |
| 255 | 395 | CCUCAACAAGCACGUCAAA | 1023 | UUUGACGUGCUUGUUGAGG |
| 256 | 396 | CUCAACAAGCACGUCAAAA | 1024 | UUUUGACGUGCUUGUUGAG |
| 257 | 397 | UCAACAAGCACGUCAAAAA | 1025 | UUUUUGACGUGCUUGUUGA |
| 290 | 398 | UAUCAAUUUCUGUCUCAUA | 1026 | UAUGAGACAGAAAUUGAUA |
| 291 | 399 | AUCAAUUUCUGUCUCAUCA | 1027 | UGAUGAGACAGAAAUUGAU |
| 292 | 400 | UCAAUUUCUGUCUCAUCUA | 1028 | UAGAUGAGACAGAAAUUGA |
| 293 | 401 | CAAUUUCUGUCUCAUCUUA | 1029 | UAAGAUGAGACAGAAAUUG |
| 297 | 402 | UUCUGUCUCAUCUUAAUAA | 1030 | UUAUUAAGAUGAGACAGAA |
| 298 | 403 | UCUGUCUCAUCUUAAUAUA | 1031 | UAUAUUAAGAUGAGACAGA |
| 299 | 404 | CUGUCUCAUCUUAAUAUGA | 1032 | UCAUAUUAAGAUGAGACAG |
| 313 | 405 | UAUGUCUCUUGCUGAUCUA | 1033 | UAGAUCAGCAAGAGACAUA |
| 346 | 406 | UUCUCUGAAGUUCUGCUAA | 1034 | UUAGCAGAACUUCAGAGAA |
| 348 | 407 | CUCUGAAGUUCUGCUACAA | 1035 | UUGUAGCAGAACUUCAGAG |
| 353 | 408 | AAGUUCUGCUACAACCUCA | 1036 | UGAGGUUGUAGCAGAACUU |
| 354 | 409 | AGUUCUGCUACAACCUCUA | 1037 | UAGAGGUUGUAGCAGAACU |
| 355 | 410 | GUUCUGCUACAACCUCUAA | 1038 | UUAGAGGUUGUAGCAGAAC |
| 357 | 411 | UCUGCUACAACCUCUAGAA | 1039 | UUCUAGAGGUUGUAGCAGA |
| 361 | 412 | CUACAACCUCUAGAUCUGA | 1040 | UCAGAUCUAGAGGUUGUAG |
| 363 | 413 | ACAACCUCUAGAUCUGCAA | 1041 | UUGCAGAUCUAGAGGUUGU |
| 381 | 414 | GCUUGCCACAUCAGCUUAA | 1042 | UUAAGCUGAUGUGGCAAGC |
| 382 | 415 | CUUGCCACAUCAGCUUAAA | 1043 | UUUAAGCUGAUGUGGCAAG |
| 383 | 416 | UUGCCACAUCAGCUUAAAA | 1044 | UUUUAAGCUGAUGUGGCAA |
| 387 | 417 | CACAUCAGCUUAAAAUCUA | 1045 | UAGAUUUUAAGCUGAUGUG |
| 388 | 418 | ACAUCAGCUUAAAAUCUGA | 1046 | UCAGAUUUUAAGCUGAUGU |
| 389 | 419 | CAUCAGCUUAAAAUCUGUA | 1047 | UACAGAUUUUAAGCUGAUG |
| 390 | 420 | AUCAGCUUAAAAUCUGUCA | 1048 | UGACAGAUUUUAAGCUGAU |
| 391 | 421 | UCAGCUUAAAAUCUGUCAA | 1049 | UUGACAGAUUUUAAGCUGA |
| 392 | 422 | CAGCUUAAAAUCUGUCAUA | 1050 | UAUGACAGAUUUUAAGCUG |
| 393 | 423 | AGCUUAAAAUCUGUCAUCA | 1051 | UGAUGACAGAUUUUAAGCU |
| 394 | 424 | GCUUAAAAUCUGUCAUCCA | 1052 | UGGAUGACAGAUUUUAAGC |
| 438 | 425 | UAACAGACCACUUCCUGAA | 1053 | UUCAGGAAGUGGUCUGUUA |
| 440 | 426 | ACAGACCACUUCCUGAGUA | 1054 | UACUCAGGAAGUGGUCUGU |
| 441 | 427 | CAGACCACUUCCUGAGUAA | 1055 | UUACUCAGGAAGUGGUCUG |
| 442 | 428 | AGACCACUUCCUGAGUAGA | 1056 | UCUACUCAGGAAGUGGUCU |
| 443 | 429 | GACCACUUCCUGAGUAGAA | 1057 | UUCUACUCAGGAAGUGGUC |
| 449 | 430 | UUCCUGAGUAGAAGAGUUA | 1058 | UAACUCUUCUACUCAGGAA |
| 469 | 431 | UUUGUGAAAAGGUCAAGAA | 1059 | UUCUUGACCUUUUCACAAA |
| 472 | 432 | GUGAAAAGGUCAAGAUUAA | 1060 | UUAAUCUUGACCUUUUCAC |
| 473 | 433 | UGAAAAGGUCAAGAUUAAA | 1061 | UUUAAUCUUGACCUUUUCA |
| 474 | 434 | GAAAAGGUCAAGAUUAAGA | 1062 | UCUUAAUCUUGACCUUUUC |
| 526 | 435 | UUGGAUCUUGUAAACAUGA | 1063 | UCAUGUUUACAAGAUCCAA |
| 527 | 436 | UGGAUCUUGUAAACAUGAA | 1064 | UUCAUGUUUACAAGAUCCA |
| 531 | 437 | UCUUGUAAACAUGAAAAGA | 1065 | UCUUUUCAUGUUUACAAGA |
| 532 | 438 | CUUGUAAACAUGAAAAGGA | 1066 | UCCUUUUCAUGUUUACAAG |
| 533 | 439 | UUGUAAACAUGAAAAGGGA | 1067 | UCCCUUUUCAUGUUUACAA |
| 537 | 440 | AAACAUGAAAAGGGCUUUA | 1068 | UAAAGCCCUUUUCAUGUUU |
| 538 | 441 | AACAUGAAAAGGGCUUUAA | 1069 | UUAAAGCCCUUUUCAUGUU |
| 628 | 442 | CUAUCCCAAAUCUUUUCUA | 1070 | UAGAAAAGAUUUGGGAUAG |
| 630 | 443 | AUCCCAAAUCUUUUCUGAA | 1071 | UUCAGAAAAGAUUUGGGAU |
| 631 | 444 | UCCCAAAUCUUUUCUGAAA | 1072 | UUUCAGAAAAGAUUUGGGA |
| 639 | 445 | CUUUUCUGAAGAUGAAGAA | 1073 | UUCUUCAUCUUCAGAAAAG |
| 640 | 446 | UUUUCUGAAGAUGAAGAGA | 1074 | UCUCUUCAUCUUCAGAAAA |
| 641 | 447 | UUUCUGAAGAUGAAGAGUA | 1075 | UACUCUUCAUCUUCAGAAA |
| 642 | 448 | UUCUGAAGAUGAAGAGUUA | 1076 | UAACUCUUCAUCUUCAGAA |
| 643 | 449 | UCUGAAGAUGAAGAGUUUA | 1077 | UAAACUCUUCAUCUUCAGA |
| 644 | 450 | CUGAAGAUGAAGAGUUUAA | 1078 | UUAAACUCUUCAUCUUCAG |
| 645 | 451 | UGAAGAUGAAGAGUUUAGA | 1079 | UCUAAACUCUUCAUCUUCA |
| 646 | 452 | GAAGAUGAAGAGUUUAGUA | 1080 | UACUAAACUCUUCAUCUUC |
| 670 | 453 | ACUGCACUGCCAACAAGUA | 1081 | UACUUGUUGGCAGUGCAGU |
| 673 | 454 | GCACUGCCAACAAGUUCAA | 1082 | UUGAACUUGUUGGCAGUGC |
| 677 | 455 | UGCCAACAAGUUCACUUCA | 1083 | UGAAGUGAACUUGUUGGCA |
| 687 | 456 | UUCACUUCAUAUAUAAAGA | 1084 | UCUUUAUAUAUGAAGUGAA |
| 688 | 457 | UCACUUCAUAUAUAAAGCA | 1085 | UGCUUUAUAUAUGAAGUGA |
| 689 | 458 | CACUUCAUAUAUAAAGCAA | 1086 | UUGCUUUAUAUAUGAAGUG |
| 711 | 459 | UUUUACUCUUUUGAGGUGA | 1087 | UCACCUCAAAAGAGUAAAA |
| 712 | 460 | UUUACUCUUUUGAGGUGAA | 1088 | UUCACCUCAAAAGAGUAAA |
| 713 | 461 | UUACUCUUUUGAGGUGAAA | 1089 | UUUCACCUCAAAAGAGUAA |
| 714 | 462 | UACUCUUUUGAGGUGAAUA | 1090 | UAUUCACCUCAAAAGAGUA |
| 781 | 463 | GGUCUUCACCAAGUAUCAA | 1091 | UUGAUACUUGGUGAAGACC |
| 793 | 464 | GUAUCAAAGUAAUAACACA | 1092 | UGUGUUAUUACUUUGAUAC |
| 797 | 465 | CAAAGUAAUAACACAAAUA | 1093 | UAUUUGUGUUAUUACUUUG |
| 800 | 466 | AGUAAUAACACAAAUGAAA | 1094 | UUUCAUUUGUGUUAUUACU |
| 842 | 467 | UGACUCCUCACAUCUGUUA | 1095 | UAACAGAUGUGAGGAGUCA |
| 844 | 468 | ACUCCUCACAUCUGUUAUA | 1096 | UAUAACAGAUGUGAGGAGU |
| 845 | 469 | CUCCUCACAUCUGUUAUCA | 1097 | UGAUAACAGAUGUGAGGAG |
| 846 | 470 | UCCUCACAUCUGUUAUCUA | 1098 | UAGAUAACAGAUGUGAGGA |
| 928 | 471 | UCUAUGCCACAUUAACAUA | 1099 | UAUGUUAAUGUGGCAUAGA |
| 949 | 472 | UUAAAGUUGAUGAGAAUCA | 1100 | UGAUUCUCAUCAACUUUAA |
| 957 | 473 | GAUGAGAAUCAAGUAUGGA | 1101 | UCCAUACUUGAUUCUCAUC |
| 958 | 474 | AUGAGAAUCAAGUAUGGAA | 1102 | UUCCAUACUUGAUUCUCAU |
| 960 | 475 | GAGAAUCAAGUAUGGAAAA | 1103 | UUUUCCAUACUUGAUUCUC |
| 961 | 476 | AGAAUCAAGUAUGGAAAAA | 1104 | UUUUUCCAUACUUGAUUCU |
| 962 | 477 | GAAUCAAGUAUGGAAAAGA | 1105 | UCUUUUCCAUACUUGAUUC |
| 963 | 478 | AAUCAAGUAUGGAAAAGUA | 1106 | UACUUUUCCAUACUUGAUU |
| 964 | 479 | AUCAAGUAUGGAAAAGUAA | 1107 | UUACUUUUCCAUACUUGAU |
| 965 | 480 | UCAAGUAUGGAAAAGUAAA | 1108 | UUUACUUUUCCAUACUUGA |
| 966 | 481 | CAAGUAUGGAAAAGUAAGA | 1109 | UCUUACUUUUCCAUACUUG |
| 967 | 482 | AAGUAUGGAAAAGUAAGGA | 1110 | UCCUUACUUUUCCAUACUU |
| 968 | 483 | AGUAUGGAAAAGUAAGGCA | 1111 | UGCCUUACUUUUCCAUACU |
| 969 | 484 | GUAUGGAAAAGUAAGGCCA | 1112 | UGGCCUUACUUUUCCAUAC |
| 972 | 485 | UGGAAAAGUAAGGCCAUAA | 1113 | UAUGGCCUUACUUUUCCA |
| 981 | 486 | AAGGCCAUACUCUUACAUA | 1114 | UAUGUAAGAGUAUGGCCUU |
| 985 | 487 | CCAUACUCUUACAUAAUAA | 1115 | UUAUUAUGUAAGAGUAUGG |
| 1022 | 488 | UUUUCAAAGAAUCACAGAA | 1116 | UUCUGUGAUUCUUUGAAAA |
| 1023 | 489 | UUUCAAAGAAUCACAGAAA | 1117 | UUUCUGUGAUUCUUUGAAA |
| 1024 | 490 | UUCAAAGAAUCACAGAAUA | 1118 | UAUUCUGUGAUUCUUUGAA |
| 1031 | 491 | AAUCACAGAAUUCUAGUAA | 1119 | UUACUAGAAUUCUGUGAUU |
| 1032 | 492 | AUCACAGAAUUCUAGUACA | 1120 | UGUACUAGAAUUCUGUGAU |
| 1061 | 493 | UCAUAAAUCUGUUCUAAGA | 1121 | UCUUAGAACAGAUUUAUGA |
| 1062 | 494 | CAUAAAUCUGUUCUAAGAA | 1122 | UUCUUAGAACAGAUUUAUG |
| 1063 | 495 | AUAAAUCUGUUCUAAGACA | 1123 | UGUCUUAGAACAGAUUUAU |
| 1064 | 496 | UAAAUCUGUUCUAAGACAA | 1124 | UUGUCUUAGAACAGAUUUA |
| 1067 | 497 | AUCUGUUCUAAGACAUAUA | 1125 | UAUAUGUCUUAGAACAGAU |
| 1069 | 498 | CUGUUCUAAGACAUAUGAA | 1126 | UUCAUAUGUCUUAGAACAG |
| 1070 | 499 | UGUUCUAAGACAUAUGAUA | 1127 | UAUCAUAUGUCUUAGAACA |
| 1074 | 500 | CUAAGACAUAUGAUCAACA | 1128 | UGUUGAUCAUAUGUCUUAG |
| 1075 | 501 | UAAGACAUAUGAUCAACAA | 1129 | UUGUUGAUCAUAUGUCUUA |
| 1080 | 502 | CAUAUGAUCAACAGAUGAA | 1130 | UUCAUCUGUUGAUCAUAUG |
| 1081 | 503 | AUAUGAUCAACAGAUGAGA | 1131 | UCUCAUCUGUUGAUCAUAU |
| 1084 | 504 | UGAUCAACAGAUGAGAACA | 1132 | UGUUCUCAUCUGUUGAUCA |
| 1086 | 505 | AUCAACAGAUGAGAACUGA | 1133 | UCAGUUCUCAUCUGUUGAU |
| 1087 | 506 | UCAACAGAUGAGAACUGGA | 1134 | UCCAGUUCUCAUCUGUUGA |
| 1120 | 507 | AGUGAGAUUAGUCAUAUCA | 1135 | UGAUAUGACUAAUCUCACU |
| 1122 | 508 | UGAGAUUAGUCAUAUCACA | 1136 | UGUGAUAUGACUAAUCUCA |
| 1136 | 509 | UCACUAAUAUACUAACAAA | 1137 | UUUGUUAGUAUAUUAGUGA |
| 1178 | 510 | ACUGUAGUGAAUUAUCUGA | 1138 | UCAGAUAAUUCACUACAGU |
| 1179 | 511 | CUGUAGUGAAUUAUCUGAA | 1139 | UUCAGAUAAUUCACUACAG |
| 1231 | 512 | GGAAUCAUGAAACCUUAAA | 1140 | UUUAAGGUUUCAUGAUUCC |
| 1232 | 513 | GAAUCAUGAAACCUUAAGA | 1141 | UCUUAAGGUUUCAUGAUUC |
| 1233 | 514 | AAUCAUGAAACCUUAAGAA | 1142 | UUCUUAAGGUUUCAUGAUU |
| 1234 | 515 | AUCAUGAAACCUUAAGACA | 1143 | UGUCUUAAGGUUUCAUGAU |
| 1235 | 516 | UCAUGAAACCUUAAGACUA | 1144 | UAGUCUUAAGGUUUCAUGA |
| 1236 | 517 | CAUGAAACCUUAAGACUUA | 1145 | UAAGUCUUAAGGUUUCAUG |
| 1239 | 518 | GAAACCUUAAGACUUCAGA | 1146 | UCUGAAGUCUUAAGGUUUC |
| 1240 | 519 | AAACCUUAAGACUUCAGAA | 1147 | UUCUGAAGUCUUAAGGUUU |
| 1266 | 520 | GCAGGUUGUCUUCCAUUCA | 1148 | UGAAUGGAAGACAACCUGC |
| 1267 | 521 | CAGGUUGUCUUCCAUUCCA | 1149 | UGGAAUGGAAGACAACCUG |
| 1268 | 522 | AGGUUGUCUUCCAUUCCAA | 1150 | UUGGAAUGGAAGACAACCU |
| 1286 | 523 | GCCUAACAUCCAAUGCAGA | 1151 | UCUGCAUUGGAUGUUAGGC |
| 1287 | 524 | CCUAACAUCCAAUGCAGGA | 1152 | UCCUGCAUUGGAUGUUAGG |
| 1288 | 525 | CUAACAUCCAAUGCAGGCA | 1153 | UGCCUGCAUUGGAUGUUAG |
| 1289 | 526 | UAACAUCCAAUGCAGGCAA | 1154 | UUGCCUGCAUUGGAUGUUA |
| 1290 | 527 | AACAUCCAAUGCAGGCAAA | 1155 | UUUGCCUGCAUUGGAUGUU |
| 1291 | 528 | ACAUCCAAUGCAGGCAAGA | 1156 | UCUUGCCUGCAUUGGAUGU |
| 1292 | 529 | CAUCCAAUGCAGGCAAGGA | 1157 | UCCUUGCCUGCAUUGGAUG |
| 1293 | 530 | AUCCAAUGCAGGCAAGGAA | 1158 | UUCCUUGCCUGCAUUGGAU |
| 1294 | 531 | UCCAAUGCAGGCAAGGAAA | 1159 | UUUCCUUGCCUGCAUUGGA |
| 1295 | 532 | CCAAUGCAGGCAAGGAAAA | 1160 | UUUUCCUUGCCUGCAUUGG |
| 1296 | 533 | CAAUGCAGGCAAGGAAAAA | 1161 | UUUUUCCUUGCCUGCAUUG |
| 1297 | 534 | AAUGCAGGCAAGGAAAAUA | 1162 | UAUUUUCCUUGCCUGCAUU |
| 1304 | 535 | GCAAGGAAAAUAAAAGAUA | 1163 | UAUCUUUUAUUUUCCUUGC |
| 1312 | 536 | AAUAAAAGAUUUCCAGUGA | 1164 | UCACUGGAAAUCUUUUAUU |
| 1313 | 537 | AUAAAAGAUUUCCAGUGAA | 1165 | UUCACUGGAAAUCUUUUAU |
| 1314 | 538 | UAAAAGAUUUCCAGUGACA | 1166 | UGUCACUGGAAAUCUUUUA |
| 1315 | 539 | AAAAGAUUUCCAGUGACAA | 1167 | UUGUCACUGGAAAUCUUUU |
| 1366 | 540 | UAUAUGAAUUCUCUCUCCA | 1168 | UGGAGAGAGAAUUCAUAUA |
| 1367 | 541 | AUAUGAAUUCUCUCUCCAA | 1169 | UUGGAGAGAGAAUUCAUAU |
| 1368 | 542 | UAUGAAUUCUCUCUCCAAA | 1170 | UUUGGAGAGAGAAUUCAUA |
| 1369 | 543 | AUGAAUUCUCUCUCCAAAA | 1171 | UUUUGGAGAGAGAAUUCAU |
| 1370 | 544 | UGAAUUCUCUCUCCAAAUA | 1172 | UAUUUGGAGAGAGAAUUCA |
| 1371 | 545 | GAAUUCUCUCUCCAAAUAA | 1173 | UUAUUUGGAGAGAGAAUUC |
| 1372 | 546 | AAUUCUCUCUCCAAAUAUA | 1174 | UAUAUUUGGAGAGAGAAUU |
| 1373 | 547 | AUUCUCUCUCCAAAUAUUA | 1175 | UAAUAUUUGGAGAGAGAAU |
| 1412 | 548 | UUGAAAUGAACUUGUUGGA | 1176 | UCCAACAAGUUCAUUUCAA |
| 1414 | 549 | GAAAUGAACUUGUUGGCCA | 1177 | UGGCCAACAAGUUCAUUUC |
| 1415 | 550 | AAAUGAACUUGUUGGCCCA | 1178 | UGGGCCAACAAGUUCAUUU |
| 1416 | 551 | AAUGAACUUGUUGGCCCAA | 1179 | UUGGGCCAACAAGUUCAUU |
| 1421 | 552 | ACUUGUUGGCCCAUCUAUA | 1180 | UAUAGAUGGGCCAACAAGU |
| 1423 | 553 | UUGUUGGCCCAUCUAUUAA | 1181 | UUAAUAGAUGGGCCAACAA |
| 1426 | 554 | UUGGCCCAUCUAUUACAUA | 1182 | UAUGUAAUAGAUGGGCCAA |
| 1430 | 555 | CCCAUCUAUUACAUCUACA | 1183 | UGUAGAUGUAAUAGAUGGG |
| 1431 | 556 | CCAUCUAUUACAUCUACAA | 1184 | UUGUAGAUGUAAUAGAUGG |
| 1432 | 557 | CAUCUAUUACAUCUACAGA | 1185 | UCUGUAGAUGUAAUAGAUG |
| 1433 | 558 | AUCUAUUACAUCUACAGCA | 1186 | UGCUGUAGAUGUAAUAGAU |
| 1434 | 559 | UCUAUUACAUCUACAGCUA | 1187 | UAGCUGUAGAUGUAAUAGA |
| 1435 | 560 | CUAUUACAUCUACAGCUGA | 1188 | UCAGCUGUAGAUGUAAUAG |
| 1436 | 561 | UAUUACAUCUACAGCUGAA | 1189 | UUCAGCUGUAGAUGUAAUA |
| 1437 | 562 | AUUACAUCUACAGCUGACA | 1190 | UGUCAGCUGUAGAUGUAAU |
| 1438 | 563 | UUACAUCUACAGCUGACCA | 1191 | UGGUCAGCUGUAGAUGUAA |
| 1439 | 564 | UACAUCUACAGCUGACCCA | 1192 | UGGGUCAGCUGUAGAUGUA |
| 1440 | 565 | ACAUCUACAGCUGACCCUA | 1193 | UAGGGUCAGCUGUAGAUGU |
| 1441 | 566 | CAUCUACAGCUGACCCUUA | 1194 | UAAGGGUCAGCUGUAGAUG |
| 1442 | 567 | AUCUACAGCUGACCCUUGA | 1195 | UCAAGGGUCAGCUGUAGAU |
| 1466 | 568 | GGGGUUAGGGGAGCUGACA | 1196 | UGUCAGCUCCCCUAACCCC |
| 1467 | 569 | GGGUUAGGGGAGCUGACAA | 1197 | UUGUCAGCUCCCCUAACCC |
| 1468 | 570 | GGUUAGGGGAGCUGACAAA | 1198 | UUUGUCAGCUCCCCUAACC |
| 1469 | 571 | GUUAGGGGAGCUGACAAUA | 1199 | UAUUGUCAGCUCCCCUAAC |
| 1498 | 572 | CAAAAUCUUAACUACCUAA | 1200 | UUAGGUAGUUAAGAUUUUG |
| 1501 | 573 | AAUCUUAACUACCUAAUAA | 1201 | UUAUUAGGUAGUUAAGAUU |
| 1502 | 574 | AUCUUAACUACCUAAUAGA | 1202 | UCUAUUAGGUAGUUAAGAU |
| 1505 | 575 | UUAACUACCUAAUAGCCUA | 1203 | UAGGCUAUUAGGUAGUUAA |
| 1506 | 576 | UAACUACCUAAUAGCCUAA | 1204 | UUAGGCUAUUAGGUAGUUA |
| 1508 | 577 | ACUACCUAAUAGCCUACUA | 1205 | UAGUAGGCUAUUAGGUAGU |
| 1526 | 578 | AUUGACCAUAAACCUUACA | 1206 | UGUAAGGUUUAUGGUCAAU |
| 1527 | 579 | UUGACCAUAAACCUUACUA | 1207 | UAGUAAGGUUUAUGGUCAA |
| 1528 | 580 | UGACCAUAAACCUUACUGA | 1208 | UCAGUAAGGUUUAUGGUCA |
| 1529 | 581 | GACCAUAAACCUUACUGAA | 1209 | UUCAGUAAGGUUUAUGGUC |
| 1530 | 582 | ACCAUAAACCUUACUGAUA | 1210 | UAUCAGUAAGGUUUAUGGU |
| 1531 | 583 | CCAUAAACCUUACUGAUAA | 1211 | UUAUCAGUAAGGUUUAUGG |
| 1532 | 584 | CAUAAACCUUACUGAUAAA | 1212 | UUUAUCAGUAAGGUUUAUG |
| 1533 | 585 | AUAAACCUUACUGAUAACA | 1213 | UGUUAUCAGUAAGGUUUAU |
| 1543 | 586 | CUGAUAACAUAAACAGUAA | 1214 | UUACUGUUUAUGUUAUCAG |
| 1674 | 587 | UCAUUAAAUGGAAGUGGGA | 1215 | UCCCACUUCCAUUUAAUGA |
| 1675 | 588 | CAUUAAAUGGAAGUGGGUA | 1216 | UACCCACUUCCAUUUAAUG |
| 1706 | 589 | UUCAUUCUCAUUGUCUUCA | 1217 | UGAAGACAAUGAGAAUGAA |
| 1707 | 590 | UCAUUCUCAUUGUCUUCAA | 1218 | UUGAAGACAAUGAGAAUGA |
| 1708 | 591 | CAUUCUCAUUGUCUUCACA | 1219 | UGUGAAGACAAUGAGAAUG |
| 1711 | 592 | UCUCAUUGUCUUCACAUUA | 1220 | UAAUGUGAAGACAAUGAGA |
| 1782 | 593 | GUCUUGUCUUAGGGGUGUA | 1221 | UACACCCCUAAGACAAGAC |
| 1783 | 594 | UCUUGUCUUAGGGGUGUGA | 1222 | UCACACCCCUAAGACAAGA |
| 1817 | 595 | AUAUUCAUGUAUAAGUGGA | 1223 | UCCACUUAUACAUGAAUAU |
| 1822 | 596 | CAUGUAUAAGUGGACCCUA | 1224 | UAGGGUCCACUUAUACAUG |
| 2000 | 597 | AUUUUCAAUUUCUCCUCUA | 1225 | UAGAGGAGAAAUUGAAAAU |
| 2001 | 598 | UUUUCAAUUUCUCCUCUGA | 1226 | UCAGAGGAGAAAUUGAAAA |
| 2002 | 599 | UUUCAAUUUCUCCUCUGAA | 1227 | UUCAGAGGAGAAAUUGAAA |
| 116 | 600 | GTTATCTCATATTTGGCTA | 1228 | UAGCCAAATATGAGATAAC |
| 261 | 601 | CAAGCACGTCAAAAGCTAA | 1229 | UTAGCTTTTGACGTGCTTG |
| 263 | 602 | AGCACGTCAAAAGCTACAA | 1230 | UTGTAGCTTTTGACGTGCT |
| 267 | 603 | CGTCAAAAGCTACAGAATA | 1231 | UATTCTGTAGCTTTTGACG |
| 268 | 604 | GTCAAAAGCTACAGAATCA | 1232 | UGATTCTGTAGCTTTTGAC |
| 534 | 605 | TGTAAACATGAAAAGGGCA | 1233 | UGCCCTTTTCATGTTTACA |
| 535 | 606 | GTAAACATGAAAAGGGCTA | 1234 | UAGCCCTTTTCATGTTTAC |
| 1089 | 607 | AACAGATGAGAACTGGTGA | 1235 | UCACCAGTTCTCATCTGTT |
| 1090 | 608 | ACAGATGAGAACTGGTGGA | 1236 | UCCACCAGTTCTCATCTGT |
| 1091 | 609 | CAGATGAGAACTGGTGGTA | 1237 | UACCACCAGTTCTCATCTG |
| 1092 | 610 | AGATGAGAACTGGTGGTTA | 1238 | UAACCACCAGTTCTCATCT |
| 1093 | 611 | GATGAGAACTGGTGGTTAA | 1239 | UTAACCACCAGTTCTCATC |
| 1094 | 612 | ATGAGAACTGGTGGTTAAA | 1240 | UTTAACCACCAGTTCTCAT |
| 1095 | 613 | TGAGAACTGGTGGTTAATA | 1241 | UATTAACCACCAGTTCTCA |
| 1096 | 614 | GAGAACTGGTGGTTAATAA | 1242 | UTATTAACCACCAGTTCTC |
| 1097 | 615 | AGAACTGGTGGTTAATATA | 1243 | UATATTAACCACCAGTTCT |
| 1228 | 616 | TTTGGAATCATGAAACCTA | 1244 | UAGGTTTCATGATTCCAAA |
| 1229 | 617 | TTGGAATCATGAAACCTTA | 1245 | UAAGGTTTCATGATTCCAA |
| 1614 | 618 | AAGTAAGCTAGAGAAAATA | 1246 | UATTTTCTCTAGCTTACTT |
| 1615 | 619 | AGTAAGCTAGAGAAAATGA | 1247 | UCATTTTCTCTAGCTTACT |
| 1616 | 620 | GTAAGCTAGAGAAAATGTA | 1248 | UACATTTTCTCTAGCTTAC |
| 1673 | 621 | TTCATTAAATGGAAGTGGA | 1249 | UCCACTTCCATTTAATGAA |
| 1701 | 622 | AAGTCTTCATTCTCATTGA | 1250 | UCAATGAGAATGAAGACTT |
| 1702 | 623 | AGTCTTCATTCTCATTGTA | 1251 | UACAATGAGAATGAAGACT |
| 1703 | 624 | GTCTTCATTCTCATTGTCA | 1252 | UGACAATGAGAATGAAGAC |
| 1704 | 625 | TCTTCATTCTCATTGTCTA | 1253 | UAGACAATGAGAATGAAGA |
| 1784 | 626 | CTTGTCTTAGGGGTGTGGA | 1254 | UCCACACCCCTAAGACAAG |
| 1818 | 627 | TATTCATGTATAAGTGGAA | 1255 | UTCCACTTATACATGAATA |
| 1821 | 628 | TCATGTATAAGTGGACCCA | 1256 | UGGGTCCACTTATACATGA |

Column 1 shows the beginning position of the target sequence in PLN human # NM_002667.5 (SEQ ID NO: 2523). The target sequence is 19 nucleotide long starting from the beginning position. For example, row 1 shows that the beginning position of the target sequence is 202, which means that the target sequence is the sequence corresponding to positions 202-220 of PLN_human # NM_002667.5 (SEQ ID NO: 2523). The sequences of the sense strands and antisense strands are shown from the 5′ end to the 3′ end.

TABLE 1B
provides exemplary IC50, Knockdown, and Species Selectivity datafor certain
siRNAs comprising modified sense strand and modified antisense strand. Column 1 shows
the beginning position of the target sequence in PLN_human # NM_002667.5 (SEQ ID NO:
2523). The target sequence is 19 nucleotide long starting from the beginning position. For
example, row 1 shows that the beginning position of the target sequence is 202, which means
that the target sequence is the sequence corresponding to positions 202-220 of PLN_human_#
NM_002667.5 (SEQ ID NO: 2523). The sequences of the sense strands and antisense strands are
shown from the 5′ end to the 3′ end.

| Position in | SEQ | | SEQ | | mRNA level | IC50 |
|---|---|---|---|---|---|---|
| transcript | ID NO | Sense strand | ID NO | Antisense strand | % at 50 nM | (nM) |
| 202 | 1257 | asasguCfcAfAfUf | 1885 | csGfsaguGfaGfGfuauu | 65.23 | |
| | | accucacucg | | GfgAfcuususu | | |
| 1209 | 1258 | usasgcUfuAfCfCf | 1886 | gsAfsuauAfgUfAfugguA | 31.97 | |
| | | auacuauauc | | faGfcuasusu | | |
| 1211 | 1259 | gscsuuAfcCfAfUf | 1887 | asAfsgauAfuAfGfuaug | 36.91 | |
| | | acuauaucuu | | GfuAfagcsusu | | |
| 1481 | 1260 | gsascaAfuUfCfGf | 1888 | usGfscggAfcCfCfacgaA | 43.96 | |
| | | uggguccgca | | fuUfgucsusu | | |
| 2424 | 1261 | asgsauUfuCfUfUf | 1889 | gsGfscguCfaCfUfuaag | 78.95 | |
| | | aagugacgcc | | AfaAfucususu | | |
| 2430 | 1262 | csusuaAfgUfGfAf | 1890 | asGfsaugAfgGfCfguca | 83.94 | |
| | | cgccucaucu | | CfuUfaagsusu | | |
| 42 | 1263 | cscscgUfaAfGfAf | 1891 | usUfsguaUfgAfAfgucu | 6.70 | |
| | | cuucauacaa | | UfaCfgggsusu | | |
| 219 | 1264 | csgscuCfaGfCfUf | 1892 | csUfscuuCfuUfAfuagc | 20.86 | |
| | | auaagaagag | | UfgAfgcgsusu | | |
| 329 | 1265 | csusguAfuCfAfUf | 1893 | asAfsgcaUfcAfCfgaugA | 46.65 | |
| | | cgugaugcuu | | fuAfcagsusu | | |
| 330 | 1266 | usgsuaUfcAfUfCf | 1894 | gsAfsagcAfuCfAfcgauG | 98.95 | |
| | | gugaugcuuc | | faUfacasusu | | |
| 1130 | 1267 | gsuscaUfaUfCfAf | 1895 | asGfsuauAfuUfAfguga | 112.98 | |
| | | cuaauauacu | | UfaUfgacsusu | | |
| 1202 | 1268 | asgsuuAfcCfUfAf | 1896 | usAfsuggUfaAfGfcuag | 78.61 | |
| | | gcuuaccaua | | GfuAfacususu | | |
| 1503 | 1269 | uscsuuAfaCfUfAf | 1897 | gsGfscuaUfuAfGfguag | 39.33 | |
| | | ccuaauagcc | | UfuAfagasusu | | |
| 1504 | 1270 | csusuaAfcUfAfCf | 1898 | asGfsgcuAfuUfAfggua | 37.17 | |
| | | cuaauagccu | | GfuUfaagsusu | | |
| 1900 | 1271 | csuscuAfuCfAfAf | 1899 | usAfsccaUfuUfGfguug | 69.16 | |
| | | ccaaauggua | | AfuAfgagsusu | | |
| 1906 | 1272 | csasacCfaAfAfUf | 1900 | gsAfsugcUfuAfCfcauu | 63.34 | |
| | | gguaagcauc | | UfgGfuugsusu | | |
| 1939 | 1273 | csuscuAfcUfGfAf | 1901 | asUfsaauUfuAfGfcuca | 74.15 | |
| | | gcuaaauuau | | GfuAfgagsusu | | |
| 1940 | 1274 | uscsuaCfuGfAfGf | 1902 | usAfsuaaUfuUfAfgcucA | 75.80 | |
| | | cuaaauuaua | | fgUfagasusu | | |
| 1948 | 1275 | gscsuaAfaUfUfAf | 1903 | gsCfsuggAfuCfUfauaa | 72.80 | |
| | | uagauccagc | | UfuUfagcsusu | | |
| 2301 | 1276 | ascsacAfaGfUfGf | 1904 | gsAfsguuAfgCfAfacacU | 68.69 | |
| | | uugcuaacuc | | fuGfugususu | | |
| 2309 | 1277 | gsusugCfuAfAfCf | 1905 | usCfsacuAfuUfGfaguu | 80.28 | |
| | | ucaauaguga | | AfgCfaacsusu | | |
| 2425 | 1278 | gsasuuUfcUfUfAf | 1906 | asGfsgcgUfcAfCfuuaa | 82.18 | |
| | | agugacgccu | | GfaAfaucsusu | | |
| 2429 | 1279 | uscsuuAfaGfUfGf | 1907 | gsAfsugaGfgCfGfucac | 75.92 | |
| | | acgccucauc | | UfuAfagasusu | | |
| 2431 | 1280 | ususaaGfuGfAfCf | 1908 | usAfsgauGfaGfGfcguc | 77.13 | |
| | | gccucaucua | | AfcUfuaasusu | | |
| 226 | 1281 | csusauAfaGfAfAf | 1909 | gsUfsugaGfgCfUfcuuc | 24.86 | |
| | | gagccucaac | | UfuAfuagsusu | | |
| 227 | 1282 | usasuaAfgAfAfGf | 1910 | gsGfsuugAfgGfCfucuu | 74.25 | |
| | | agccucaacc | | CfuUfauasusu | | |
| 228 | 1283 | asusaaGfaAfGfAf | 1911 | usGfsguuGfaGfGfcucu | 67.88 | |
| | | gccucaacca | | UfcUfuaususu | | |
| 260 | 1284 | ascsaaGfcAfCfGf | 1912 | usAfsgcuUfuUfGfacgu | 20.29 | |
| | | ucaaaagcua | | GfcUfugususu | | |
| 276 | 1285 | csusacAfgAfAfUfc | 1913 | usGfsauaAfaUfAfgauu | 17.45 | |
| | | uauuuauca | | CfuGfuagsusu | | |
| 667 | 1286 | asasaaCfuGfCfAf | 1914 | usUfsguuGfgCfAfgugc | 26.99 | |
| | | cugccaacaa | | AfgUfuuususu | | |
| 668 | 1287 | asasacUfgCfAfCf | 1915 | csUfsuguUfgGfCfagug | 39.83 | |
| | | ugccaacaag | | CfaGfuuususu | | |
| 959 | 1288 | usgsagAfaUfCfAf | 1916 | usUfsuccAfuAfCfuuga | 43.10 | |
| | | aguauggaaa | | UfuCfucasusu | | |
| 1099 | 1289 | asascuGfgUfGfGf | 1917 | csAfscauAfuUfAfaccaC | 46.49 | |
| | | uuaauaugug | | fcAfguususu | | |
| 1100 | 1290 | ascsugGfuGfGfUf | 1918 | usCfsacaUfaUfUfaacc | 30.93 | |
| | | uaauauguga | | AfcCfagususu | | |
| 1590 | 1291 | asusuaUfaCfAfCf | 1919 | usAfsggaAfuAfUfagugU | 40.10 | |
| | | uauauuccua | | faUfaaususu | | |
| 2522 | 1292 | gsusaaAfuUfAfCf | 1920 | usUfscugUfaUfUfggua | 75.52 | |
| | | caauacagaa | | AfuUfuacsusu | | |
| 2576 | 1293 | asascuGfaUfUfAf | 1921 | gsGfsaauAfuGfAfcuaa | 75.22 | |
| | | gucauauucc | | UfcAfguususu | | |
| 2837 | 1294 | ascsuaUfaGfAfAf | 1922 | usAfsagaAfcUfUfauuc | 75.82 | |
| | | uaaguucuua | | UfaUfagususu | | |
| 225 | 1295 | gscsuaUfaAfGfAf | 1923 | usUfsgagGfcUfCfuucu | 16.44 | |
| | | agagccucaa | | UfaUfagcsusu | | |
| 476 | 1296 | asasagGfuCfAfAf | 1924 | asGfsucuUfaAfUfcuug | 16.66 | |
| | | gauuaagacu | | AfcCfuuususu | | |
| 477 | 1297 | asasggUfcAfAfGf | 1925 | usAfsgucUfuAfAfucuu | 25.21 | |
| | | auuaagacua | | GfaCfcuususu | | |
| 484 | 1298 | asgsauUfaAfGfAf | 1926 | usAfsaguUfuUfAfgucu | 21.69 | |
| | | cuaaaacuua | | UfaAfucususu | | |
| 809 | 1299 | ascsaaAfuGfAfAf | 1927 | asUfsaauGfaCfAfcuuc | 50.33 | |
| | | gugucauuau | | AfuUfugususu | | |
| 617 | 1300 | csuscaCfaAfAfUf | 1928 | usGfsggaUfaGfAfaauu | 34.52 | |
| | | uucuauccca | | UfgUfgagsusu | | |
| 666 | 1301 | usasaaAfcUfGfCf | 1929 | usGfsuugGfcAfGfugca | 38.42 | |
| | | acugccaaca | | GfuUfuuasusu | | |
| 669 | 1302 | asascuGfcAfCfUf | 1930 | asCfsuugUfuGfGfcagu | 43.22 | |
| | | gccaacaagu | | GfcAfguususu | | |
| 274 | 1303 | asgscuAfcAfGfAf | 1931 | asUfsaaaUfaGfAfuucu | 19.64 | |
| | | aucuauuuau | | GfuAfgcususu | | |
| 275 | 1304 | gscsuaCfaGfAfAf | 1932 | gsAfsuaaAfuAfGfauuc | 6.59 | |
| | | ucuauuuauc | | UfgUfagcsusu | | |
| 1098 | 1305 | asasaaCfuUfAfUf | 1933 | usAfsuggUfaAfCfaauaA | 14.55 | |
| | | uguuaccaua | | fgUfuuususu | | |
| 1101 | 1306 | gsasacUfgGfUfGf | 1934 | asCfsauaUfuAfAfccac | 47.03 | |
| | | guuaauaugu | | CfaGfuucsusu | | |
| 1413 | 1307 | asascuGfgUfGfGf | 1935 | csAfscauAfuUfAfaccaC | 46.97 | |
| | | uuaauaugug | | fcAfguususu | | |
| 1483 | 1308 | ascsugGfuGfGfUf | 1936 | usCfsacaUfaUfUfaacc | 34.52 | |
| | | uaauauguga | | AfcCfagususu | | |
| 1484 | 1309 | csusggUfgGfUfUf | 1937 | gsUfscacAfuAfUfuaac | 61.54 | |
| | | aauaugugac | | CfaCfcagsusu | | |
| 1485 | 1310 | asusucGfuGfGfGf | 1938 | asUfsuuuGfcGfGfaccc | 35.88 | |
| | | uccgcaaaau | | AfcGfaaususu | | |
| 1600 | 1311 | asusauUfcCfUfAf | 1939 | usAfscuuUfaUfUfguag | 43.99 | |
| | | caauaaagua | | GfaAfuaususu | | |
| 1876 | 1312 | asasuaGfgAfUfAf | 1940 | asAfsaauAfgCfUfauau | 87.40 | |
| | | uagcuauuuu | | CfcUfauususu | | |
| 1933 | 1313 | asgsucCfaCfUfCf | 1941 | usAfsgcuCfaGfUfagag | 80.74 | |
| | | uacugagcua | | UfgGfacususu | | |
| 2158 | 1314 | asusaaUfcCfAfAf | 1942 | asUfsuaaAfaUfUfguug | 82.83 | |
| | | caauuuuaau | | GfaUfuaususu | | |
| 2269 | 1315 | asusauGfuAfAfCf | 1943 | csAfsauaUfuGfAfuguu | 98.34 | |
| | | aucaauauug | | AfcAfuaususu | | |
| 2349 | 1316 | usgsaaCfcCfAfUf | 1944 | asGfsuauCfuCfUfcaug | 72.42 | |
| | | gagagauacu | | GfgUfucasusu | | |
| 2856 | 1317 | uscsuuAfaUfUfUf | 1945 | asGfsugcCfcUfGfuaaa | 75.39 | |
| | | acagggcacu | | UfuAfagasusu | | |
| 2857 | 1318 | csusuaAfuUfUfAf | 1946 | usAfsgugCfcCfUfguaa | 71.79 | |
| | | cagggcacua | | AfuUfaagsusu | | |
| 278 | 1319 | ascsagAfaUfCfUf | 1947 | asUfsugaUfaAfAfuaga | 20.86 | |
| | | auuuaucaau | | UfuCfugususu | | |
| 2692 | 1320 | usgsagGfaUfUfAf | 1948 | usAfsguaUfuCfUfguaa | 80.56 | |
| | | cagaauacua | | UfcCfucasusu | | |
| 757 | 1321 | ususcuUfuAfAfUf | 1949 | asAfsuacUfuAfGfuauu | 48.34 | |
| | | acuaaguauu | | AfaAfgaasusu | | |
| 1379 | 1322 | uscsucCfaAfAfUf | 1950 | asUfsuagUfuAfAfuauu | 43.36 | |
| | | auuaacuaau | | UfgGfagasusu | | |
| 2220 | 1323 | usasgaGfgAfUfGf | 1951 | gsUfsuaaUfuAfCfacau | 84.17 | |
| | | uguaauuaac | | CfcUfcuasusu | | |
| 2102 | 1324 | asasuuUfaAfGfUf | 1952 | asCfsuuuAfgUfCfaacu | 81.56 | |
| | | ugacuaaagu | | UfaAfauususu | | |
| 2690 | 1325 | usgsugAfgGfAfUf | 1953 | gsUfsauuCfuGfUfaauc | 68.42 | |
| | | uacagaauac | | CfuCfacasusu | | |
| 52 | 1326 | ususcaUfaCfAfAf | 1954 | gsAfsguaUfuGfUfguug | 14.61 | |
| | | cacaauacuc | | UfaUfgaasusu | | |
| 1380 | 1327 | csusccAfaAfUfAf | 1955 | asAfsuuaGfuUfAfauau | 38.90 | |
| | | uuaacuaauu | | UfuGfgagsusu | | |
| 1132 | 1328 | csasuaUfcAfCfUf | 1956 | usUfsaguAfuAfUfuagu | 45.45 | |
| | | aauauacuaa | | GfaUfaugsusu | | |
| 1126 | 1329 | asusuaGfuCfAfUf | 1957 | usAfsuuaGfuGfAfuaug | 76.47 | |
| | | aucacuaaua | | AfcUfaaususu | | |
| 281 | 1330 | gsasauCfuAfUfUf | 1958 | gsAfsaauUfgAfUfaaauA | 14.25 | |
| | | uaucaauuuc | | fgAfuucsusu | | |
| 2232 | 1331 | asasuuAfaCfCfAf | 1959 | usUfsagaAfgAfUfauggU | 75.03 | |
| | | uaucuucuaa | | fuAfauususu | | |
| 2219 | 1332 | gsusagAfgGfAfUf | 1960 | usUfsaauUfaCfAfcauc | 75.52 | |
| | | guguaauuaa | | CfuCfuacsusu | | |
| 199 | 1333 | asgsaaAfgUfCfCf | 1961 | gsUfsgagGfuAfUfugga | 37.70 | |
| | | aauaccucac | | CfuUfucususu | | |
| 2093 | 1334 | asascuUfuGfGfUf | 1962 | asAfscuuAfaAfUfuaccA | 112.59 | |
| | | aauuuaaguu | | faAfguususu | | |
| 1378 | 1335 | csuscuCfcAfAfAf | 1963 | usUfsaguUfaAfUfauuu | 40.37 | |
| | | uauuaacuaa | | GfgAfgagsusu | | |
| 2840 | 1336 | asusagAfaUfAfAfg | 1964 | asGfsauaAfgAfAfcuuaU | 75.82 | |
| | | uucuuaucu | | fuCfuaususu | | |
| 1134 | 1337 | usasucAfcUfAfAf | 1965 | usGfsuuaGfuAfUfauua | 100.30 | |
| | | uauacuaaca | | GfuGfauasusu | | |
| 2222 | 1338 | gsasggAfuGfUfGf | 1966 | usGfsguuAfaUfUfacac | 71.03 | |
| | | uaauuaacca | | AfuCfcucsusu | | |
| 2703 | 1339 | gsasauAfcUfAfUf | 1967 | asAfsuuuGfaGfUfuaua | 86.64 | |
| | | aacucaaauu | | GfuAfuucsusu | | |
| 358 | 1340 | csusgcUfaCfAfAf | 1968 | gsAfsucuAfgAfGfguugU | 108.53 | |
| | | ccucuagauc | | faGfcagsusu | | |
| 285 | 1341 | csusauUfuAfUfCf | 1969 | gsAfscagAfaAfUfugauA | 110.13 | |
| | | aauuucuguc | | faAfuagsusu | | |
| 2184 | 1342 | gsasagAfcAfUfGf | 1970 | asUfsauuAfgUfAfacau | 98.95 | |
| | | uuacuaauau | | GfuCfuucsusu | | |
| 1377 | 1343 | uscsucUfcCfAfAf | 1971 | usAfsguuAfaUfAfuuug | 112.58 | |
| | | auauuaacua | | GfaGfagasusu | | |
| 359 | 1344 | usgscuAfcAfAfCf | 1972 | asGfsaucUfaGfAfgguu | 24.14 | |
| | | cucuagaucu | | GfuAfgcasusu | | |
| 194 | 1345 | csasugGfaGfAfAf | 1973 | gsUfsauuGfgAfCfuuuc | 103.75 | |
| | | aguccaauac | | UfcCfaugsusu | | |
| 888 | 1346 | ascsuaUfcAfGfAf | 1974 | gsAfsaugUfaGfAfuucu | 37.58 | |
| | | aucuacauuc | | GfaUfagususu | | |
| 193 | 1347 | uscsauGfgAfGfAf | 1975 | usAfsuugGfaCfUfuucu | 119.63 | |
| | | aaguccaaua | | CfcAfugasusu | | |
| 209 | 1348 | asusacCfuCfAfCf | 1976 | usGfscugAfgCfGfagug | 19.46 | |
| | | ucgcucagca | | AfgGfuaususu | | |
| 210 | 1349 | usasccUfcAfCfUf | 1977 | usAfsgcuGfaGfCfgagu | 30.93 | |
| | | cgcucagcua | | GfaGfguasusu | | |
| 211 | 1350 | ascscuCfaCfUfCf | 1978 | usUfsagcUfgAfGfcgag | 25.70 | |
| | | gcucagcuaa | | UfgAfggususu | | |
| 248 | 1351 | usgsaaAfuGfCfCf | 1979 | usGfscuuGfuUfGfaggc | 9.98 | |
| | | ucaacaagca | | AfuUfucasusu | | |
| 249 | 1352 | gsasaaUfgCfCfUf | 1980 | usUfsgcuUfgUfUfgagg | 9.20 | |
| | | caacaagcaa | | CfaUfuucsusu | | |
| 250 | 1353 | asasauGfcCfUfCf | 1981 | usGfsugcUfuGfUfugag | 26.58 | |
| | | aacaagcaca | | GfcAfuuususu | | |
| 251 | 1354 | asasugCfcUfCfAf | 1982 | usCfsgugCfuUfGfuuga | 24.12 | |
| | | acaagcacga | | GfgCfauususu | | |
| 252 | 1355 | asusgcCfuCfAfAf | 1983 | usAfscguGfcUfUfguug | 21.87 | |
| | | caagcacgua | | AfgGfcaususu | | |
| 253 | 1356 | usgsccUfcAfAfCf | 1984 | usGfsacgUfgCfUfuguu | 41.26 | |
| | | aagcacguca | | GfaGfgcasusu | | |
| 606 | 1357 | cscsucAfaCfAfUf | 1985 | usUfsuguGfaGfCfcaug | 22.54 | |
| | | ggcucacaaa | | UfuGfaggsusu | | |
| 607 | 1358 | csuscaAfcAfUfGf | 1986 | usUfsuugUfgAfGfccau | 42.98 | |
| | | gcucacaaaa | | GfuUfgagsusu | | |
| 608 | 1359 | uscsaaCfaUfGfGf | 1987 | usAfsuuuGfuGfAfgcca | 17.49 | |
| | | cucacaaaua | | UfgUfugasusu | | |
| 609 | 1360 | csasacAfuGfGfCf | 1988 | usAfsauuUfgUfGfagcc | 22.40 | |
| | | ucacaaauua | | AfuGfuugsusu | | |
| 203 | 1361 | asgsucCfaAfUfAf | 1989 | usCfsgagUfgAfGfguau | 13.06 | |
| | | ccucacucga | | UfgGfacususu | | |
| 204 | 1362 | gsusccAfaUfAfCf | 1990 | usGfscgaGfuGfAfggua | 9.32 | |
| | | cucacucgca | | UfuGfgacsusu | | |
| 206 | 1363 | cscsaaUfaCfCfUf | 1991 | usGfsagcGfaGfUfgagg | 13.74 | |
| | | cacucgcuca | | UfaUfuggsusu | | |
| 207 | 1364 | csasauAfcCfUfCf | 1992 | usUfsgagCfgAfGfugag | 17.47 | |
| | | acucgcucaa | | GfuAfuugsusu | | |
| 223 | 1365 | csasgcUfaUfAfAf | 1993 | usAfsggcUfcUfUfcuua | 25.67 | |
| | | gaagagccua | | UfaGfcugsusu | | |
| 230 | 1366 | asasgaAfgAfGfCf | 1994 | usAfsuggUfuGfAfggcu | 7.13 | |
| | | cucaaccaua | | CfuUfcuususu | | |
| 300 | 1367 | usgsucUfcAfUfCf | 1995 | usAfscauAfuUfAfagau | 13.20 | |
| | | uuaauaugua | | GfaGfacasusu | | |
| 301 | 1368 | gsuscuCfaUfCfUf | 1996 | usGfsacaUfaUfUfaaga | 5.31 | |
| | | uaauauguca | | UfgAfgacsusu | | |
| 302 | 1369 | uscsucAfuCfUfUf | 1997 | usAfsgacAfuAfUfuaagA | 58.49 | |
| | | aauaugucua | | fuGfagasusu | | |
| 303 | 1370 | csuscaUfcUfUfAf | 1998 | usGfsagaCfaUfAfuuaa | 43.43 | |
| | | auaugucuca | | GfaUfgagsusu | | |
| 306 | 1371 | asuscuUfaAfUfAf | 1999 | usCfsaagAfgAfCfauau | 10.43 | |
| | | ugucucuuga | | UfaAfgaususu | | |
| 307 | 1372 | uscsuuAfaUfAfUf | 2000 | usGfscaaGfaGfAfcaua | 26.83 | |
| | | gucucuugca | | UfuAfagasusu | | |
| 308 | 1373 | csusuaAfuAfUfGf | 2001 | usAfsgcaAfgAfGfacauA | 17.58 | |
| | | ucucuugcua | | fuUfaagsusu | | |
| 309 | 1374 | ususaaUfaUfGfUf | 2002 | usCfsagcAfaGfAfgaca | 7.96 | |
| | | cucuugcuga | | UfaUfuaasusu | | |
| 310 | 1375 | usasauAfuGfUfCf | 2003 | usUfscagCfaAfGfagac | 10.05 | |
| | | ucuugcugaa | | AfuAfuuasusu | | |
| 311 | 1376 | asasuaUfgUfCfUf | 2004 | usAfsucaGfcAfAfgagaC | 14.83 | |
| | | cuugcugaua | | faUfauususu | | |
| 312 | 1377 | asusauGfuCfUfCf | 2005 | usGfsaucAfgCfAfagagA | 8.29 | |
| | | uugcugauca | | fcAfuaususu | | |
| 314 | 1378 | asusguCfuCfUfUf | 2006 | usCfsagaUfcAfGfcaag | 14.03 | |
| | | gcugaucuga | | AfgAfcaususu | | |
| 315 | 1379 | usgsucUfcUfUfGf | 2007 | usAfscagAfuCfAfgcaaG | 7.80 | |
| | | cugaucugua | | faGfacasusu | | |
| 321 | 1380 | ususgcUfgAfUfCf | 2008 | usGfsaugAfuAfCfagau | 13.01 | |
| | | uguaucauca | | CfaGfcaasusu | | |
| 322 | 1381 | usgscuGfaUfCfUf | 2009 | usCfsgauGfaUfAfcaga | 27.97 | |
| | | guaucaucga | | UfcAfgcasusu | | |
| 323 | 1382 | gscsugAfuCfUfGf | 2010 | usAfscgaUfgAfUfacagA | 11.84 | |
| | | uaucaucgua | | fuCfagcsusu | | |
| 324 | 1383 | csusgaUfcUfGfUf | 2011 | usCfsacgAfuGfAfuaca | 49.29 | |
| | | aucaucguga | | GfaUfcagsusu | | |
| 325 | 1384 | usgsauCfuGfUfAf | 2012 | usUfscacGfaUfGfauac | 21.57 | |
| | | ucaucgugaa | | AfgAfucasusu | | |
| 326 | 1385 | gsasucUfgUfAfUf | 2013 | usAfsucaCfgAfUfgaua | 15.23 | |
| | | caucgugaua | | CfaGfaucsusu | | |
| 327 | 1386 | asuscuGfuAfUfCf | 2014 | usCfsaucAfcGfAfugau | 61.52 | |
| | | aucgugauga | | AfcAfgaususu | | |
| 328 | 1387 | uscsugUfaUfCfAf | 2015 | usGfscauCfaCfGfauga | 31.79 | |
| | | ucgugaugca | | UfaCfagasusu | | |
| 331 | 1388 | gsusauCfaUfCfGf | 2016 | usGfsaagCfaUfCfacga | 4.38 | |
| | | ugaugcuuca | | UfgAfuacsusu | | |
| 334 | 1389 | uscsauCfgUfGfAf | 2017 | usAfsgagAfaGfCfauca | 7.79 | |
| | | ugcuucucua | | CfgAfugasusu | | |
| 335 | 1390 | csasucGfuGfAfUf | 2018 | usCfsagaGfaAfGfcauc | 19.31 | |
| | | gcuucucuga | | AfcGfaugsusu | | |
| 336 | 1391 | asuscgUfgAfUfGf | 2019 | usUfscagAfgAfAfgcauC | 41.61 | |
| | | cuucucugaa | | faCfgaususu | | |
| 337 | 1392 | uscsguGfaUfGfCf | 2020 | usUfsucaGfaGfAfagca | 20.26 | |
| | | uucucugaaa | | UfcAfcgasusu | | |
| 338 | 1393 | csgsugAfuGfCfUf | 2021 | usCfsuucAfgAfGfaagcA | 23.91 | |
| | | ucucugaaga | | fuCfacgsusu | | |
| 349 | 1394 | uscsugAfaGfUfUf | 2022 | usUfsuguAfgCfAfgaacU | 54.86 | |
| | | cugcuacaaa | | fuCfagasusu | | |
| 350 | 1395 | csusgaAfgUfUfCf | 2023 | usGfsuugUfaGfCfagaa | 15.79 | |
| | | ugcuacaaca | | CfuUfcagsusu | | |
| 351 | 1396 | usgsaaGfuUfCfUf | 2024 | usGfsguuGfuAfGfcaga | 31.70 | |
| | | gcuacaacca | | AfcUfucasusu | | |
| 356 | 1397 | ususcuGfcUfAfCf | 2025 | usCfsuagAfgGfUfugua | 64.92 | |
| | | aaccucuaga | | GfcAfgaasusu | | |
| 360 | 1398 | gscsuaCfaAfCfCf | 2026 | usAfsgauCfuAfGfaggu | 5.09 | |
| | | ucuagaucua | | UfgUfagcsusu | | |
| 399 | 1399 | asasauCfuGfUfCf | 2027 | usGfscauGfgGfAfugac | 26.41 | |
| | | aucccaugca | | AfgAfuuususu | | |
| 400 | 1400 | asasucUfgUfCfAf | 2028 | usUfsgcaUfgGfGfauga | 15.50 | |
| | | ucccaugcaa | | CfaGfauususu | | |
| 451 | 1401 | cscsugAfgUfAfGf | 2029 | usGfsaaaCfuCfUfucua | 9.12 | |
| | | aagaguuuca | | CfuCfaggsusu | | |
| 470 | 1402 | ususguGfaAfAfAf | 2030 | usAfsucuUfgAfCfcuuu | 7.11 | |
| | | ggucaagaua | | UfcAfcaasusu | | |
| 471 | 1403 | usgsugAfaAfAfGfg | 2031 | usAfsaucUfuGfAfccuu | 6.80 | |
| | | ucaagauua | | UfuCfacasusu | | |
| 475 | 1404 | asasaaGfgUfCfAf | 2032 | usUfscuuAfaUfCfuuga | 8.77 | |
| | | agauuaagaa | | CfcUfuuususu | | |
| 478 | 1405 | asgsguCfaAfGfAf | 2033 | usUfsaguCfuUfAfaucu | 7.32 | |
| | | uuaagacuaa | | UfgAfccususu | | |
| 480 | 1406 | gsuscaAfgAfUfUf | 2034 | usUfsuuaGfuCfUfuaau | 7.86 | |
| | | aagacuaaaa | | CfuUfgacsusu | | |
| 481 | 1407 | uscsaaGfaUfUfAf | 2035 | usUfsuuuAfgUfCfuuaa | 12.43 | |
| | | agacuaaaaa | | UfcUfugasusu | | |
| 482 | 1408 | csasagAfuUfAfAfg | 2036 | usGfsuuuUfaGfUfcuua | 18.50 | |
| | | acuaaaaca | | AfuCfuugsusu | | |
| 483 | 1409 | asasgaUfuAfAfGf | 2037 | usAfsguuUfuAfGfucuu | 15.38 | |
| | | acuaaaacua | | AfaUfcuususu | | |
| 683 | 1410 | csasagUfuCfAfCf | 2038 | usAfsuauAfuGfAfagugA | 31.18 | |
| | | uucauauaua | | faCfuugsusu | | |
| 684 | 1411 | asasguUfcAfCfUf | 2039 | usUfsauaUfaUfGfaagu | 25.45 | |
| | | ucauauauaa | | GfaAfcuususu | | |
| 715 | 1412 | ascsucUfuUfUfGf | 2040 | usUfsauuCfaCfCfucaa | 16.80 | |
| | | aggugaauaa | | AfaGfagususu | | |
| 716 | 1413 | csuscuUfuUfGfAf | 2041 | usAfsuauUfcAfCfcuca | 23.25 | |
| | | ggugaauaua | | AfaAfgagsusu | | |
| 717 | 1414 | uscsuuUfuGfAfGf | 2042 | usUfsauaUfuCfAfccuc | 25.44 | |
| | | gugaauauaa | | AfaAfagasusu | | |
| 786 | 1415 | uscsacCfaAfGfUf | 2043 | usUfsacuUfuGfAfuacu | 25.53 | |
| | | aucaaaguaa | | UfgGfugasusu | | |
| 788 | 1416 | ascscaAfgUfAfUf | 2044 | usAfsuuaCfuUfUfgaua | 36.86 | |
| | | caaaguaaua | | CfuUfggususu | | |
| 789 | 1417 | cscsaaGfuAfUfCf | 2045 | usUfsauuAfcUfUfugau | 28.62 | |
| | | aaaguaauaa | | AfcUfuggsusu | | |
| 810 | 1418 | csasaaUfgAfAfGf | 2046 | usAfsuaaUfgAfCfacuu | 31.63 | |
| | | ugucauuaua | | CfaUfuugsusu | | |
| 811 | 1419 | asasauGfaAfGfUf | 2047 | usAfsauaAfuGfAfcacu | 33.58 | |
| | | gucauuauua | | UfcAfuuususu | | |
| 812 | 1420 | asasugAfaGfUfGf | 2048 | usGfsaauAfaUfGfacac | 19.89 | |
| | | ucauuauuca | | UfuCfauususu | | |
| 264 | 1421 | gscsacGfuCfAfAf | 2049 | usCfsuguAfgCfUfuuug | 11.50 | |
| | | aagcuacaga | | AfcGfugcsusu | | |
| 521 | 1422 | asuscuGfuUfGfGf | 2050 | usUfsuacAfaGfAfucca | 8.24 | |
| | | aucuuguaaa | | AfcAfgaususu | | |
| 522 | 1423 | uscsugUfuGfGfAf | 2051 | usUfsuuaCfaAfGfaucc | 11.62 | |
| | | ucuuguaaaa | | AfaCfagasusu | | |
| 523 | 1424 | csusguUfgGfAfUf | 2052 | usGfsuuuAfcAfAfgauc | 14.71 | |
| | | cuuguaaaca | | CfaAfcagsusu | | |
| 524 | 1425 | usgsuuGfgAfUfCf | 2053 | usUfsguuUfaCfAfagau | 12.06 | |
| | | uuguaaacaa | | CfcAfacasusu | | |
| 525 | 1426 | gsusugGfaUfCfUf | 2054 | usAfsuguUfuAfCfaaga | 9.62 | |
| | | uguaaacaua | | UfcCfaacsusu | | |
| 593 | 1427 | csasacUfgUfUfGf | 2055 | usUfsgagGfaAfAfucaa | 13.10 | |
| | | auuuccucaa | | CfaGfuugsusu | | |
| 610 | 1428 | asascaUfgGfCfUf | 2056 | usAfsaauUfuGfUfgagc | 30.69 | |
| | | cacaaauuua | | CfaUfguususu | | |
| 618 | 1429 | uscsacAfaAfUfUf | 2057 | usUfsgggAfuAfGfaaau | 27.28 | |
| | | ucuaucccaa | | UfuGfugasusu | | |
| 623 | 1430 | asasuuUfcUfAfUf | 2058 | usAfsgauUfuGfGfgaua | 25.60 | |
| | | cccaaaucua | | GfaAfauususu | | |
| 624 | 1431 | asusuuCfuAfUfCf | 2059 | usAfsagaUfuUfGfggau | 24.02 | |
| | | ccaaaucuua | | AfgAfaaususu | | |
| 718 | 1432 | csusuuUfgAfGfGf | 2060 | usUfsuauAfuUfCfaccu | 20.87 | |
| | | ugaauauaaa | | CfaAfaagsusu | | |
| 776 | 1433 | ususucAfgGfUfCf | 2061 | usCfsuugGfuGfAfagac | 19.06 | |
| | | uucaccaaga | | CfuGfaaasusu | | |
| 777 | 1434 | ususcaGfgUfCfUf | 2062 | usAfscuuGfgUfGfaaga | 26.49 | |
| | | ucaccaagua | | CfcUfgaasusu | | |
| 778 | 1435 | uscsagGfuCfUfUf | 2063 | usUfsacuUfgGfUfgaag | 25.87 | |
| | | caccaaguaa | | AfcCfugasusu | | |
| 779 | 1436 | csasggUfcUfUfCf | 2064 | usAfsuacUfuGfGfugaa | 21.41 | |
| | | accaaguaua | | GfaCfcugsusu | | |
| 780 | 1437 | asgsguCfuUfCfAf | 2065 | usGfsauaCfuUfGfguga | 22.37 | |
| | | ccaaguauca | | AfgAfccususu | | |
| 813 | 1438 | asusgaAfgUfGfUf | 2066 | usUfsgaaUfaAfUfgaca | 28.69 | |
| | | cauuauucaa | | CfuUfcaususu | | |
| 839 | 1439 | csascuGfaCfUfCf | 2067 | usAfsgauGfuGfAfggag | 35.17 | |
| | | cucacaucua | | UfcAfgugsusu | | |
| 926 | 1440 | ususucUfaUfGfCf | 2068 | usGfsuuaAfuGfUfggca | 26.63 | |
| | | cacauuaaca | | UfaGfaaasusu | | |
| 49 | 1441 | gsascuUfcAfUfAf | 2069 | usAfsuugUfgUfUfguau | 6.45 | |
| | | caacacaaua | | GfaAfgucsusu | | |
| 54 | 1442 | csasuaCfaAfCfAf | 2070 | usAfsgagUfaUfUfgugu | 4.36 | |
| | | caauacucua | | UfgUfaugsusu | | |
| 55 | 1443 | asusacAfaCfAfCf | 2071 | usUfsagaGfuAfUfugug | 12.72 | |
| | | aauacucuaa | | UfuGfuaususu | | |
| 109 | 1444 | gsasagAfcAfGfUf | 2072 | usUfsaugAfgAfUfaacu | 14.05 | |
| | | uaucucauaa | | GfuCfuucsusu | | |
| 110 | 1445 | asasgaCfaGfUfUf | 2073 | usAfsuauGfaGfAfuaac | 16.93 | |
| | | aucucauaua | | UfgUfcuususu | | |
| 113 | 1446 | ascsagUfuAfUfCf | 2074 | usCfsaaaUfaUfGfagau | 30.62 | |
| | | ucauauuuga | | AfaCfugususu | | |
| 115 | 1447 | asgsuuAfuCfUfCf | 2075 | usGfsccaAfaUfAfugagA | 43.67 | |
| | | auauuuggca | | fuAfacususu | | |
| 195 | 1448 | asusggAfgAfAfAfg | 2076 | usGfsuauUfgGfAfcuuu | 20.55 | |
| | | uccaauaca | | CfuCfcaususu | | |
| 231 | 1449 | asgsaaGfaGfCfCf | 2077 | usAfsaugGfuUfGfaggc | 7.51 | |
| | | ucaaccauua | | UfcUfucususu | | |
| 232 | 1450 | gsasagAfgCfCfUf | 2078 | usCfsaauGfgUfUfgagg | 13.80 | |
| | | caaccauuga | | CfuCfuucsusu | | |
| 233 | 1451 | asasgaGfcCfUfCf | 2079 | usUfscaaUfgGfUfugag | 46.35 | |
| | | aaccauugaa | | GfcUfcuususu | | |
| 234 | 1452 | asgsagCfcUfCfAf | 2080 | usUfsucaAfuGfGfuuga | 7.26 | |
| | | accauugaaa | | GfgCfucususu | | |
| 237 | 1453 | gscscuCfaAfCfCf | 2081 | usCfsauuUfcAfAfuggu | 18.31 | |
| | | auugaaauga | | UfgAfggcsusu | | |
| 240 | 1454 | uscsaaCfcAfUfUf | 2082 | usAfsggcAfuUfUfcaau | 39.82 | |
| | | gaaaugccua | | GfgUfugasusu | | |
| 244 | 1455 | cscsauUfgAfAfAf | 2083 | usGfsuugAfgGfCfauuu | 15.39 | |
| | | ugccucaaca | | CfaAfuggsusu | | |
| 296 | 1456 | ususucUfgUfCfUf | 2084 | usAfsuuaAfgAfUfgagaC | 9.04 | |
| | | caucuuaaua | | faGfaaasusu | | |
| 304 | 1457 | uscsauCfuUfAfAf | 2085 | usAfsgagAfcAfUfauuaA | 9.97 | |
| | | uaugucucua | | fgAfugasusu | | |
| 305 | 1458 | csasucUfuAfAfUf | 2086 | usAfsagaGfaCfAfuauu | 14.37 | |
| | | augucucuua | | AfaGfaugsusu | | |
| 316 | 1459 | gsuscuCfuUfGfCf | 2087 | usUfsacaGfaUfCfagca | 5.37 | |
| | | ugaucuguaa | | AfgAfgacsusu | | |
| 317 | 1460 | uscsucUfuGfCfUf | 2088 | usAfsuacAfgAfUfcagcA | 22.39 | |
| | | gaucuguaua | | faGfagasusu | | |
| 318 | 1461 | csuscuUfgCfUfGf | 2089 | usGfsauaCfaGfAfucag | 43.38 | |
| | | aucuguauca | | CfaAfgagsusu | | |
| 319 | 1462 | uscsuuGfcUfGfAf | 2090 | usUfsgauAfcAfGfauca | 26.81 | |
| | | ucuguaucaa | | GfcAfagasusu | | |
| 320 | 1463 | csusugCfuGfAfUf | 2091 | usAfsugaUfaCfAfgaucA | 8.46 | |
| | | cuguaucaua | | fgCfaagsusu | | |
| 341 | 1464 | gsasugCfuUfCfUf | 2092 | usGfsaacUfuCfAfgaga | 6.97 | |
| | | cugaaguuca | | AfgCfaucsusu | | |
| 342 | 1465 | asusgcUfuCfUfCf | 2093 | usAfsgaaCfuUfCfagagA | 4.60 | |
| | | ugaaguucua | | faGfcaususu | | |
| 347 | 1466 | uscsucUfgAfAfGf | 2094 | usGfsuagCfaGfAfacuu | 10.37 | |
| | | uucugcuaca | | CfaGfagasusu | | |
| 352 | 1467 | gsasagUfuCfUfGf | 2095 | usAfsgguUfgUfAfgcagA | 7.63 | |
| | | cuacaaccua | | faCfuucsusu | | |
| 362 | 1468 | usascaAfcCfUfCf | 2096 | usGfscagAfuCfUfagag | 15.45 | |
| | | uagaucugca | | GfuUfguasusu | | |
| 402 | 1469 | uscsugUfcAfUfCf | 2097 | usUfscugCfaUfGfggau | 39.43 | |
| | | ccaugcagaa | | GfaCfagasusu | | |
| 446 | 1470 | csascuUfcCfUfGf | 2098 | usUfscuuCfuAfCfucag | 58.12 | |
| | | aguagaagaa | | GfaAfgugsusu | | |
| 450 | 1471 | uscscuGfaGfUfAf | 2099 | usAfsaacUfcUfUfcuac | 8.69 | |
| | | gaagaguuua | | UfcAfggasusu | | |
| 452 | 1472 | csusgaGfuAfGfAf | 2100 | usAfsgaaAfcUfCfuucuA | 8.71 | |
| | | agaguuucua | | fcUfcagsusu | | |
| 479 | 1473 | gsgsucAfaGfAfUf | 2101 | usUfsuagUfcUfUfaauc | 5.45 | |
| | | uaagacuaaa | | UfuGfaccsusu | | |
| 528 | 1474 | gsgsauCfuUfGfUf | 2102 | usUfsucaUfgUfUfuaca | 7.43 | |
| | | aaacaugaaa | | AfgAfuccsusu | | |
| 595 | 1475 | ascsugUfuGfAfUf | 2103 | usGfsuugAfgGfAfaauc | 11.93 | |
| | | uuccucaaca | | AfaCfagususu | | |
| 598 | 1476 | gsusugAfuUfUfCf | 2104 | usCfsaugUfuGfAfggaa | 17.59 | |
| | | cucaacauga | | AfuCfaacsusu | | |
| 600 | 1477 | usgsauUfuCfCfUf | 2105 | usGfsccaUfgUfUfgagg | 17.78 | |
| | | caacauggca | | AfaAfucasusu | | |
| 601 | 1478 | gsasuuUfcCfUfCf | 2106 | usAfsgccAfuGfUfugag | 17.53 | |
| | | aacauggcua | | GfaAfaucsusu | | |
| 602 | 1479 | asusuuCfcUfCfAf | 2107 | usGfsagcCfaUfGfuuga | 22.33 | |
| | | acauggcuca | | GfgAfaaususu | | |
| 604 | 1480 | ususccUfcAfAfCf | 2108 | usGfsugaGfcCfAfuguu | 30.93 | |
| | | auggcucaca | | GfaGfgaasusu | | |
| 605 | 1481 | uscscuCfaAfCfAf | 2109 | usUfsgugAfgCfCfaugu | 23.37 | |
| | | uggcucacaa | | UfgAfggasusu | | |
| 671 | 1482 | csusgcAfcUfGfCf | 2110 | usAfsacuUfgUfUfggca | 39.21 | |
| | | caacaaguua | | GfuGfcagsusu | | |
| 672 | 1483 | usgscaCfuGfCfCf | 2111 | usGfsaacUfuGfUfuggc | 15.09 | |
| | | aacaaguuca | | AfgUfgcasusu | | |
| 674 | 1484 | csascuGfcCfAfAf | 2112 | usGfsugaAfcUfUfguug | 23.73 | |
| | | caaguucaca | | GfcAfgugsusu | | |
| 675 | 1485 | ascsugCfcAfAfCf | 2113 | usAfsgugAfaCfUfuguu | 23.69 | |
| | | aaguucacua | | GfgCfagususu | | |
| 676 | 1486 | csusgcCfaAfCfAf | 2114 | usAfsaguGfaAfCfuugu | 25.06 | |
| | | aguucacuua | | UfgGfcagsusu | | |
| 679 | 1487 | cscsaaCfaAfGfUf | 2115 | usAfsugaAfgUfGfaacu | 23.71 | |
| | | ucacuucaua | | UfgUfuggsusu | | |
| 680 | 1488 | csasacAfaGfUfUf | 2116 | usUfsaugAfaGfUfgaac | 22.11 | |
| | | cacuucauaa | | UfuGfuugsusu | | |
| 681 | 1489 | asascaAfgUfUfCf | 2117 | usAfsuauGfaAfGfugaa | 39.38 | |
| | | acuucauaua | | CfuUfguususu | | |
| 682 | 1490 | ascsaaGfuUfCfAf | 2118 | usUfsauaUfgAfAfgugaA | 24.74 | |
| | | cuucauauaa | | fcUfugususu | | |
| 690 | 1491 | ascsuuCfaUfAfUf | 2119 | usAfsugcUfuUfAfuaua | 20.55 | |
| | | auaaagcaua | | UfgAfagususu | | |
| 782 | 1492 | gsuscuUfcAfCfCf | 2120 | usUfsugaUfaCfUfuggu | 20.44 | |
| | | aaguaucaaa | | GfaAfgacsusu | | |
| 783 | 1493 | uscsuuCfaCfCfAf | 2121 | usUfsuugAfuAfCfuugg | 22.64 | |
| | | aguaucaaaa | | UfgAfagasusu | | |
| 784 | 1494 | csusucAfcCfAfAf | 2122 | usCfsuuuGfaUfAfcuug | 31.18 | |
| | | guaucaaaga | | GfuGfaagsusu | | |
| 785 | 1495 | ususcaCfcAfAfGf | 2123 | usAfscuuUfgAfUfacuu | 29.83 | |
| | | uaucaaagua | | GfgUfgaasusu | | |
| 787 | 1496 | csasccAfaGfUfAf | 2124 | usUfsuacUfuUfGfauac | 24.33 | |
| | | ucaaaguaaa | | UfuGfgugsusu | | |
| 790 | 1497 | csasagUfaUfCfAf | 2125 | usUfsuauUfaCfUfuuga | 23.38 | |
| | | aaguaauaaa | | UfaCfuugsusu | | |
| 791 | 1498 | asasguAfuCfAfAf | 2126 | usGfsuuaUfuAfCfuuug | 25.42 | |
| | | aguaauaaca | | AfuAfcuususu | | |
| 792 | 1499 | asgsuaUfcAfAfAfg | 2127 | usUfsguuAfuUfAfcuuu | 27.13 | |
| | | uaauaacaa | | GfaUfacususu | | |
| 803 | 1500 | asasuaAfcAfCfAf | 2128 | usCfsacuUfcAfUfuugu | 35.55 | |
| | | aaugaaguga | | GfuUfauususu | | |
| 838 | 1501 | cscsacUfgAfCfUf | 2129 | usGfsaugUfgAfGfgagu | 33.03 | |
| | | ccucacauca | | CfaGfuggsusu | | |
| 848 | 1502 | csuscaCfaUfCfUf | 2130 | usUfsaagAfuAfAfcagaU | 33.27 | |
| | | guuaucuuaa | | fgUfgagsusu | | |
| 849 | 1503 | uscsacAfuCfUfGf | 2131 | usAfsuaaGfaUfAfacagA | 33.76 | |
| | | uuaucuuaua | | fuGfugasusu | | |
| 850 | 1504 | csascaUfcUfGfUf | 2132 | usAfsauaAfgAfUfaacaG | 38.28 | |
| | | uaucuuauua | | faUfgugsusu | | |
| 872 | 1505 | asgsaaCfuAfUfUf | 2133 | usGfsuuaCfuAfCfaaau | 29.51 | |
| | | uguaguaaca | | AfgUfucususu | | |
| 873 | 1506 | gsasacUfaUfUfUf | 2134 | usAfsguuAfcUfAfcaaaU | 24.49 | |
| | | guaguaacua | | faGfuucsusu | | |
| 876 | 1507 | csusauUfuGfUfAf | 2135 | usGfsauaGfuUfAfcuac | 23.75 | |
| | | guaacuauca | | AfaAfuagsusu | | |
| 877 | 1508 | usasuuUfgUfAfGf | 2136 | usUfsgauAfgUfUfacua | 34.23 | |
| | | uaacuaucaa | | CfaAfauasusu | | |
| 878 | 1509 | asusuuGfuAfGfUf | 2137 | usCfsugaUfaGfUfuacu | 33.05 | |
| | | aacuaucaga | | AfcAfaaususu | | |
| 882 | 1510 | gsusagUfaAfCfUf | 2138 | usGfsauuCfuGfAfuagu | 24.64 | |
| | | aucagaauca | | UfaCfuacsusu | | |
| 883 | 1511 | usasguAfaCfUfAf | 2139 | usAfsgauUfcUfGfauag | 26.59 | |
| | | ucagaaucua | | UfuAfcuasusu | | |
| 886 | 1512 | usasacUfaUfCfAf | 2140 | usUfsguaGfaUfUfcuga | 34.51 | |
| | | gaaucuacaa | | UfaGfuuasusu | | |
| 889 | 1513 | csusauCfaGfAfAf | 2141 | usGfsaauGfuAfGfauuc | 36.36 | |
| | | ucuacauuca | | UfgAfuagsusu | | |
| 950 | 1514 | usasaaGfuUfGfAf | 2142 | usUfsgauUfcUfCfauca | 31.38 | |
| | | ugagaaucaa | | AfcUfuuasusu | | |
| 951 | 1515 | asasagUfuGfAfUf | 2143 | usUfsugaUfuCfUfcauc | 23.33 | |
| | | gagaaucaaa | | AfaCfuuususu | | |
| 952 | 1516 | asasguUfgAfUfGf | 2144 | usCfsuugAfuUfCfucau | 41.41 | |
| | | agaaucaaga | | CfaAfcuususu | | |
| 953 | 1517 | asgsuuGfaUfGfAf | 2145 | usAfscuuGfaUfUfcuca | 45.77 | |
| | | gaaucaagua | | UfcAfacususu | | |
| 978 | 1518 | asgsuaAfgGfCfCf | 2146 | usUfsaagAfgUfAfuggcC | 32.91 | |
| | | auacucuuaa | | fuUfacususu | | |
| 979 | 1519 | gsusaaGfgCfCfAf | 2147 | usGfsuaaGfaGfUfaugg | 33.92 | |
| | | uacucuuaca | | CfcUfuacsusu | | |
| 980 | 1520 | usasagGfcCfAfUf | 2148 | usUfsguaAfgAfGfuaug | 29.92 | |
| | | acucuuacaa | | GfcCfuuasusu | | |
| 983 | 1521 | gsgsccAfuAfCfUf | 2149 | usUfsuauGfuAfAfgagu | 35.66 | |
| | | cuuacauaaa | | AfuGfgccsusu | | |
| 984 | 1522 | gscscaUfaCfUfCf | 2150 | usAfsuuaUfgUfAfagagU | 32.52 | |
| | | uuacauaaua | | faUfggcsusu | | |
| 1066 | 1523 | asasucUfgUfUfCf | 2151 | usUfsaugUfcUfUfagaa | 22.71 | |
| | | uaagacauaa | | CfaGfauususu | | |
| 1068 | 1524 | uscsugUfuCfUfAf | 2152 | usCfsauaUfgUfCfuuag | 29.19 | |
| | | agacauauga | | AfaCfagasusu | | |
| 1071 | 1525 | gsusucUfaAfGfAf | 2153 | usGfsaucAfuAfUfgucu | 26.90 | |
| | | cauaugauca | | UfaGfaacsusu | | |
| 1072 | 1526 | ususcuAfaGfAfCf | 2154 | usUfsgauCfaUfAfuguc | 30.92 | |
| | | auaugaucaa | | UfuAfgaasusu | | |
| 1073 | 1527 | uscsuaAfgAfCfAf | 2155 | usUfsugaUfcAfUfaugu | 31.98 | |
| | | uaugaucaaa | | CfuUfagasusu | | |
| 1077 | 1528 | asgsacAfuAfUfGf | 2156 | usUfscugUfuGfAfucau | 39.81 | |
| | | aucaacagaa | | AfuGfucususu | | |
| 1078 | 1529 | gsascaUfaUfGfAf | 2157 | usAfsucuGfuUfGfauca | 41.36 | |
| | | ucaacagaua | | UfaUfgucsusu | | |
| 1079 | 1530 | ascsauAfuGfAfUf | 2158 | usCfsaucUfgUfUfgauc | 94.78 | |
| | | caacagauga | | AfuAfugususu | | |
| 1082 | 1531 | usasugAfuCfAfAf | 2159 | usUfscucAfuCfUfguug | 24.50 | |
| | | cagaugagaa | | AfuCfauasusu | | |
| 1083 | 1532 | asusgaUfcAfAfCf | 2160 | usUfsucuCfaUfCfuguu | 34.08 | |
| | | agaugagaaa | | GfaUfcaususu | | |
| 1118 | 1533 | ascsagUfgAfGfAf | 2161 | usUfsaugAfcUfAfaucu | 30.60 | |
| | | uuagucauaa | | CfaCfugususu | | |
| 1119 | 1534 | csasguGfaGfAfUf | 2162 | usAfsuauGfaCfUfaauc | 37.29 | |
| | | uagucauaua | | UfcAfcugsusu | | |
| 1121 | 1535 | gsusgaGfaUfUfAf | 2163 | usUfsgauAfuGfAfcuaa | 51.77 | |
| | | gucauaucaa | | UfcUfcacsusu | | |
| 1205 | 1536 | usasccUfaGfCfUf | 2164 | usAfsguaUfgGfUfaagc | 28.15 | |
| | | uaccauacua | | UfaGfguasusu | | |
| 1206 | 1537 | ascscuAfgCfUfUf | 2165 | usUfsaguAfuGfGfuaag | 35.04 | |
| | | accauacuaa | | CfuAfggususu | | |
| 1207 | 1538 | cscsuaGfcUfUfAf | 2166 | usAfsuagUfaUfGfguaa | 48.17 | |
| | | ccauacuaua | | GfcUfaggsusu | | |
| 1208 | 1539 | csusagCfuUfAfCf | 2167 | usUfsauaGfuAfUfggua | 40.91 | |
| | | cauacuauaa | | AfgCfuagsusu | | |
| 1210 | 1540 | asgscuUfaCfCfAf | 2168 | usGfsauaUfaGfUfaugg | 27.54 | |
| | | uacuauauca | | UfaAfgcususu | | |
| 1375 | 1541 | uscsucUfcUfCfCf | 2169 | usUfsuaaUfaUfUfugga | 32.25 | |
| | | aaauauuaaa | | GfaGfagasusu | | |
| 1417 | 1542 | asusgaAfcUfUfGf | 2170 | usAfsuggGfcCfAfacaa | 40.22 | |
| | | uuggcccaua | | GfuUfcaususu | | |
| 1418 | 1543 | usgsaaCfuUfGfUf | 2171 | usGfsaugGfgCfCfaaca | 53.77 | |
| | | uggcccauca | | AfgUfucasusu | | |
| 1419 | 1544 | gsasacUfuGfUfUf | 2172 | usAfsgauGfgGfCfcaac | 37.67 | |
| | | ggcccaucua | | AfaGfuucsusu | | |
| 1420 | 1545 | asascuUfgUfUfGf | 2173 | usUfsagaUfgGfGfccaa | 64.61 | |
| | | gcccaucuaa | | CfaAfguususu | | |
| 1422 | 1546 | csusugUfuGfGfCf | 2174 | usAfsauaGfaUfGfggcc | 57.75 | |
| | | ccaucuauua | | AfaCfaagsusu | | |
| 1424 | 1547 | usgsuuGfgCfCfCf | 2175 | usGfsuaaUfaGfAfuggg | 66.15 | |
| | | aucuauuaca | | CfcAfacasusu | | |
| 1956 | 1548 | asusagAfuCfCfAf | 2176 | usUfsagcAfuAfGfcuggA | 74.96 | |
| | | gcuaugcuaa | | fuCfuaususu | | |
| 1957 | 1549 | usasgaUfcCfAfGf | 2177 | usAfsuagCfaUfAfgcug | 57.39 | |
| | | cuaugcuaua | | GfaUfcuasusu | | |
| 1958 | 1550 | asgsauCfcAfGfCf | 2178 | usAfsauaGfcAfUfagcu | 65.32 | |
| | | uaugcuauua | | GfgAfucususu | | |
| 2025 | 1551 | uscsagAfaCfAfUf | 2179 | usUfsauuGfgAfAfgaug | 73.83 | |
| | | cuuccaauaa | | UfuCfugasusu | | |
| 2030 | 1552 | ascsauCfuUfCfCf | 2180 | usUfsgagUfuAfUfuggaA | 67.72 | |
| | | aauaacucaa | | fgAfugususu | | |
| 2087 | 1553 | uscsacCfaAfAfCf | 2181 | usAfsuuaCfcAfAfaguu | 82.44 | |
| | | uuugguaaua | | UfgGfugasusu | | |
| 2088 | 1554 | csasccAfaAfCfUf | 2182 | usAfsauuAfcCfAfaagu | 78.70 | |
| | | uugguaauua | | UfuGfgugsusu | | |
| 2094 | 1555 | ascsuuUfgGfUfAf | 2183 | usAfsacuUfaAfAfuuac | 103.75 | |
| | | auuuaaguua | | CfaAfagususu | | |
| 2099 | 1556 | gsgsuaAfuUfUfAf | 2184 | usUfsaguCfaAfCfuuaa | 66.60 | |
| | | aguugacuaa | | AfuUfaccsusu | | |
| 2145 | 1557 | ascsacCfuAfUfAf | 2185 | usAfsuuaUfgCfAfguauA | 76.44 | |
| | | cugcauaaua | | fgGfugususu | | |
| 2146 | 1558 | csasccUfaUfAfCf | 2186 | usGfsauuAfuGfCfagua | 80.08 | |
| | | ugcauaauca | | UfaGfgugsusu | | |
| 2147 | 1559 | ascscuAfuAfCfUf | 2187 | usGfsgauUfaUfGfcagu | 87.40 | |
| | | gcauaaucca | | AfuAfggususu | | |
| 2148 | 1560 | cscsuaUfaCfUfGf | 2188 | usUfsggaUfuAfUfgcag | 89.46 | |
| | | cauaauccaa | | UfaUfaggsusu | | |
| 2150 | 1561 | usasuaCfuGfCfAf | 2189 | usGfsuugGfaUfUfaugc | 51.64 | |
| | | uaauccaaca | | AfgUfauasusu | | |
| 2151 | 1562 | asusacUfgCfAfUf | 2190 | usUfsguuGfgAfUfuaug | 51.05 | |
| | | aauccaacaa | | CfaGfuaususu | | |
| 2152 | 1563 | usascuGfcAfUfAf | 2191 | usUfsuguUfgGfAfuuau | 70.35 | |
| | | auccaacaaa | | GfcAfguasusu | | |
| 2153 | 1564 | ascsugCfaUfAfAf | 2192 | usAfsuugUfuGfGfauua | 48.18 | |
| | | uccaacaaua | | UfgCfagususu | | |
| 2182 | 1565 | ususgaAfgAfCfAf | 2193 | usUfsuagUfaAfCfaugu | 64.02 | |
| | | uguuacuaaa | | CfuUfcaasusu | | |
| 2183 | 1566 | usgsaaGfaCfAfUf | 2194 | usAfsuuaGfuAfAfcaug | 58.98 | |
| | | guuacuaaua | | UfcUfucasusu | | |
| 2188 | 1567 | ascsauGfuUfAfCf | 2195 | usGfsuuaUfaUfUfagua | 66.46 | |
| | | uaauauaaca | | AfcAfugususu | | |
| 2216 | 1568 | asgsagUfaGfAfGf | 2196 | usUfsuacAfcAfUfccuc | 78.32 | |
| | | gauguguaaa | | UfaCfucususu | | |
| 2228 | 1569 | gsusguAfaUfUfAf | 2197 | usAfsgauAfuGfGfuuaa | 76.05 | |
| | | accauaucua | | UfuAfcacsusu | | |
| 2281 | 1570 | asasuaUfuGfAfCf | 2198 | usGfsaaaCfcAfAfggucA | 58.26 | |
| | | cuugguuuca | | faUfauususu | | |
| 2282 | 1571 | asusauUfgAfCfCf | 2199 | usAfsgaaAfcCfAfagguC | 55.68 | |
| | | uugguuucua | | faAfuaususu | | |
| 2284 | 1572 | asusugAfcCfUfUf | 2200 | usUfsaagAfaAfCfcaag | 66.52 | |
| | | gguuucuuaa | | GfuCfaaususu | | |
| 2288 | 1573 | ascscuUfgGfUfUf | 2201 | usUfsgugUfaAfGfaaac | 59.09 | |
| | | ucuuacacaa | | CfaAfggususu | | |
| 2293 | 1574 | gsgsuuUfcUfUfAf | 2202 | usAfscacUfuGfUfguaa | 48.08 | |
| | | cacaagugua | | GfaAfaccsusu | | |
| 2295 | 1575 | ususucUfuAfCfAf | 2203 | usCfsaacAfcUfUfgugu | 57.57 | |
| | | caaguguuga | | AfaGfaaasusu | | |
| 2297 | 1576 | uscsuuAfcAfCfAf | 2204 | usAfsgcaAfcAfCfuuguG | 62.30 | |
| | | aguguugcua | | fuAfagasusu | | |
| 2303 | 1577 | ascsaaGfuGfUfUf | 2205 | usUfsgagUfuAfGfcaac | 77.19 | |
| | | gcuaacucaa | | AfcUfugususu | | |
| 2306 | 1578 | asgsugUfuGfCfUf | 2206 | usUfsauuGfaGfUfuagc | 49.68 | |
| | | aacucaauaa | | AfaCfacususu | | |
| 2307 | 1579 | gsusguUfgCfUfAf | 2207 | usCfsuauUfgAfGfuuag | 53.61 | |
| | | acucaauaga | | CfaAfcacsusu | | |
| 2308 | 1580 | usgsuuGfcUfAfAf | 2208 | usAfscuaUfuGfAfguua | 61.78 | |
| | | cucaauagua | | GfcAfacasusu | | |
| 2310 | 1581 | ususgcUfaAfCfUf | 2209 | usUfscacUfaUfUfgagu | 61.07 | |
| | | caauagugaa | | UfaGfcaasusu | | |
| 2311 | 1582 | usgscuAfaCfUfCf | 2210 | usUfsucaCfuAfUfugag | 63.20 | |
| | | aauagugaaa | | UfuAfgcasusu | | |
| 2315 | 1583 | asascuCfaAfUfAf | 2211 | usCfsuccUfuCfAfcuau | 61.31 | |
| | | gugaaggaga | | UfgAfguususu | | |
| 2317 | 1584 | csuscaAfuAfGfUf | 2212 | usGfsucuCfcUfUfcacu | 77.80 | |
| | | gaaggagaca | | AfuUfgagsusu | | |
| 2380 | 1585 | usgsgaAfaGfUfGf | 2213 | usUfsgaaCfcAfAfacac | 86.81 | |
| | | uuugguucaa | | UfuUfccasusu | | |
| 2389 | 1586 | ususugGfuUfCfAf | 2214 | usAfsgauAfuCfCfcugaA | 67.12 | |
| | | gggauaucua | | fcCfaaasusu | | |
| 2390 | 1587 | ususggUfuCfAfGf | 2215 | usCfsagaUfaUfCfccug | 70.65 | |
| | | ggauaucuga | | AfaCfcaasusu | | |
| 2420 | 1588 | gscsagAfgAfUfUf | 2216 | usCfsacuUfaAfGfaaau | 66.05 | |
| | | ucuuaaguga | | CfuCfugcsusu | | |
| 2432 | 1589 | usasagUfgAfCfGf | 2217 | usUfsagaUfgAfGfgcgu | 58.20 | |
| | | ccucaucuaa | | CfaCfuuasusu | | |
| 2434 | 1590 | asgsugAfcGfCfCf | 2218 | usUfsguaGfaUfGfaggc | 53.76 | |
| | | ucaucuacaa | | GfuCfacususu | | |
| 2437 | 1591 | gsascgCfcUfCfAf | 2219 | usGfscuuGfuAfGfauga | 62.67 | |
| | | ucuacaagca | | GfgCfgucsusu | | |
| 2443 | 1592 | uscsauCfuAfCfAf | 2220 | usUfsuucCfaGfCfuugu | 66.34 | |
| | | agcuggaaaa | | AfgAfugasusu | | |
| 2601 | 1593 | usascaCfuGfUfUf | 2221 | usAfsuugUfaAfCfaaacA | 70.82 | |
| | | uguuacaaua | | fgUfguasusu | | |
| 2681 | 1594 | asuscuUfaUfUfCf | 2222 | usAfsuccUfcAfCfagaa | 68.99 | |
| | | ugugaggaua | | UfaAfgaususu | | |
| 2697 | 1595 | asusuaCfaGfAfAf | 2223 | usGfsuuaUfaGfUfauuc | 67.69 | |
| | | uacuauaaca | | UfgUfaaususu | | |
| 2700 | 1596 | ascsagAfaUfAfCf | 2224 | usUfsgagUfuAfUfagua | 61.44 | |
| | | uauaacucaa | | UfuCfugususu | | |
| 2947 | 1597 | asasauCfaGfAfAf | 2225 | usAfsuauAfgUfGfauuc | 46.88 | |
| | | ucacuauaua | | UfgAfuuususu | | |
| 17 | 1598 | asgsucAfgAfAfAfa | 2226 | usCfsuggGfgAfGfuuuu | 42.21 | |
| | | cuccccaga | | CfuGfacususu | | |
| 18 | 1599 | gsuscaGfaAfAfAf | 2227 | usGfscugGfgGfAfguuu | 48.17 | |
| | | cuccccagca | | UfcUfgacsusu | | |
| 19 | 1600 | uscsagAfaAfAfCf | 2228 | usAfsgcuGfgGfGfaguu | 56.58 | |
| | | uccccagcua | | UfuCfugasusu | | |
| 20 | 1601 | csasgaAfaAfCfUf | 2229 | usUfsagcUfgGfGfgagu | 58.57 | |
| | | ccccagcuaa | | UfuUfcugsusu | | |
| 21 | 1602 | asgsaaAfaCfUfCf | 2230 | usUfsuagCfuGfGfggag | 42.63 | |
| | | cccagcuaaa | | UfuUfucususu | | |
| 45 | 1603 | gsusaaGfaCfUfUf | 2231 | usUfsguuGfuAfUfgaag | 15.40 | |
| | | cauacaacaa | | UfcUfuacsusu | | |
| 47 | 1604 | asasgaCfuUfCfAf | 2232 | usUfsgugUfuGfUfauga | 10.28 | |
| | | uacaacacaa | | AfgUfcuususu | | |
| 48 | 1605 | asgsacUfuCfAfUf | 2233 | usUfsuguGfuUfGfuaug | 12.31 | |
| | | acaacacaaa | | AfaGfucususu | | |
| 91 | 1606 | usgsccAfaGfGfCf | 2234 | usUfsuuuAfgGfUfagcc | 21.14 | |
| | | uaccuaaaaa | | UfuGfgcasusu | | |
| 94 | 1607 | csasagGfcUfAfCf | 2235 | usUfsucuUfuUfAfggua | 29.38 | |
| | | cuaaaagaaa | | GfcCfuugsusu | | |
| 95 | 1608 | asasggCfuAfCfCf | 2236 | usCfsuucUfuUfUfaggu | 27.61 | |
| | | uaaaagaaga | | AfgCfcuususu | | |
| 96 | 1609 | asgsgcUfaCfCfUf | 2237 | usUfscuuCfuUfUfuagg | 40.43 | |
| | | aaaagaagaa | | UfaGfccususu | | |
| 97 | 1610 | gsgscuAfcCfUfAf | 2238 | usGfsucuUfcUfUfuuag | 58.44 | |
| | | aaagaagaca | | GfuAfgccsusu | | |
| 98 | 1611 | gscsuaCfcUfAfAf | 2239 | usUfsgucUfuCfUfuuua | 33.27 | |
| | | aagaagacaa | | GfgUfagcsusu | | |
| 99 | 1612 | csusacCfuAfAfAf | 2240 | usCfsuguCfuUfCfuuuu | 27.04 | |
| | | agaagacaga | | AfgGfuagsusu | | |
| 100 | 1613 | usasccUfaAfAfAf | 2241 | usAfscugUfcUfUfcuuu | 47.89 | |
| | | gaagacagua | | UfaGfguasusu | | |
| 101 | 1614 | ascscuAfaAfAfGf | 2242 | usAfsacuGfuCfUfucuu | 60.92 | |
| | | aagacaguua | | UfuAfggususu | | |
| 102 | 1615 | cscsuaAfaAfGfAf | 2243 | usUfsaacUfgUfCfuucu | 31.31 | |
| | | agacaguuaa | | UfuUfaggsusu | | |
| 103 | 1616 | csusaaAfaGfAfAf | 2244 | usAfsuaaCfuGfUfcuuc | 29.35 | |
| | | gacaguuaua | | UfuUfuagsusu | | |
| 104 | 1617 | usasaaAfgAfAfGf | 2245 | usGfsauaAfcUfGfucuu | 25.09 | |
| | | acaguuauca | | CfuUfuuasusu | | |
| 105 | 1618 | asasaaGfaAfGfAf | 2246 | usAfsgauAfaCfUfgucu | 7.28 | |
| | | caguuaucua | | UfcUfuuususu | | |
| 106 | 1619 | asasagAfaGfAfCf | 2247 | usGfsagaUfaAfCfuguc | 7.51 | |
| | | aguuaucuca | | UfuCfuuususu | | |
| 107 | 1620 | asasgaAfgAfCfAfg | 2248 | usUfsgagAfuAfAfcuguC | 17.79 | |
| | | uuaucucaa | | fuUfcuususu | | |
| 108 | 1621 | asgsaaGfaCfAfGf | 2249 | usAfsugaGfaUfAfacug | 8.17 | |
| | | uuaucucaua | | UfcUfucususu | | |
| 111 | 1622 | asgsacAfgUfUfAf | 2250 | usAfsauaUfgAfGfauaa | 12.33 | |
| | | ucucauauua | | CfuGfucususu | | |
| 112 | 1623 | gsascaGfuUfAfUf | 2251 | usAfsaauAfuGfAfgauaA | 14.76 | |
| | | cucauauuua | | fcUfgucsusu | | |
| 114 | 1624 | csasguUfaUfCfUf | 2252 | usCfscaaAfuAfUfgaga | 18.68 | |
| | | cauauuugga | | UfaAfcugsusu | | |
| 161 | 1625 | csusuaAfaAfCfUf | 2253 | usGfsaagUfcUfGfaagu | 10.58 | |
| | | ucagacuuca | | UfuUfaagsusu | | |
| 163 | 1626 | usasaaAfcUfUfCf | 2254 | usAfsggaAfgUfCfugaaG | 30.49 | |
| | | agacuuccua | | fuUfuuasusu | | |
| 181 | 1627 | gsusccUfgCfUfGf | 2255 | usCfscauGfaUfAfccag | 48.12 | |
| | | guaucaugga | | CfaGfgacsusu | | |
| 182 | 1628 | uscscuGfcUfGfGf | 2256 | usUfsccaUfgAfUfacca | 41.16 | |
| | | uaucauggaa | | GfcAfggasusu | | |
| 183 | 1629 | cscsugCfuGfGfUf | 2257 | usCfsuccAfuGfAfuacc | 14.47 | |
| | | aucauggaga | | AfgCfaggsusu | | |
| 184 | 1630 | csusgcUfgGfUfAf | 2258 | usUfscucCfaUfGfauac | 37.87 | |
| | | ucauggagaa | | CfaGfcagsusu | | |
| 185 | 1631 | usgscuGfgUfAfUf | 2259 | usUfsucuCfcAfUfgaua | 19.30 | |
| | | cauggagaaa | | CfcAfgcasusu | | |
| 186 | 1632 | gscsugGfuAfUfCf | 2260 | usUfsuucUfcCfAfugau | 15.06 | |
| | | auggagaaaa | | AfcCfagcsusu | | |
| 187 | 1633 | csusggUfaUfCfAf | 2261 | usCfsuuuCfuCfCfauga | 14.24 | |
| | | uggagaaaga | | UfaCfcagsusu | | |
| 188 | 1634 | usgsguAfuCfAfUf | 2262 | usAfscuuUfcUfCfcaug | 15.76 | |
| | | ggagaaagua | | AfuAfccasusu | | |
| 189 | 1635 | gsgsuaUfcAfUfGf | 2263 | usGfsacuUfuCfUfccau | 28.42 | |
| | | gagaaaguca | | GfaUfaccsusu | | |
| 197 | 1636 | gsgsagAfaAfGfUf | 2264 | usAfsgguAfuUfGfgacu | 10.97 | |
| | | ccaauaccua | | UfuCfuccsusu | | |
| 198 | 1637 | gsasgaAfaGfUfCf | 2265 | usGfsaggUfaUfUfggac | 10.78 | |
| | | caauaccuca | | UfuUfcucsusu | | |
| 200 | 1638 | gsasaaGfuCfCfAf | 2266 | usGfsugaGfgUfAfuugg | 25.41 | |
| | | auaccucaca | | AfcUfuucsusu | | |
| 201 | 1639 | asasagUfcCfAfAf | 2267 | usAfsgugAfgGfUfauug | 32.42 | |
| | | uaccucacua | | GfaCfuuususu | | |
| 205 | 1640 | uscscaAfuAfCfCf | 2268 | usAfsgcgAfgUfGfagguA | 17.83 | |
| | | ucacucgcua | | fuUfggasusu | | |
| 212 | 1641 | cscsucAfcUfCfGf | 2269 | usAfsuagCfuGfAfgcga | 20.09 | |
| | | cucagcuaua | | GfuGfaggsusu | | |
| 213 | 1642 | csuscaCfuCfGfCf | 2270 | usUfsauaGfcUfGfagcg | 8.81 | |
| | | ucagcuauaa | | AfgUfgagsusu | | |
| 214 | 1643 | uscsacUfcGfCfUf | 2271 | usUfsuauAfgCfUfgagc | 37.53 | |
| | | cagcuauaaa | | GfaGfugasusu | | |
| 215 | 1644 | csascuCfgCfUfCf | 2272 | usCfsuuaUfaGfCfugag | 28.71 | |
| | | agcuauaaga | | CfgAfgugsusu | | |
| 217 | 1645 | csuscgCfuCfAfGf | 2273 | usUfsucuUfaUfAfgcug | 13.58 | |
| | | cuauaagaaa | | AfgCfgagsusu | | |
| 221 | 1646 | csuscaGfcUfAfUf | 2274 | usGfscucUfuCfUfuaua | 50.17 | |
| | | aagaagagca | | GfcUfgagsusu | | |
| 222 | 1647 | uscsagCfuAfUfAf | 2275 | usGfsgcuCfuUfCfuuau | 31.37 | |
| | | agaagagcca | | AfgCfugasusu | | |
| 224 | 1648 | asgscuAfuAfAfGf | 2276 | usGfsaggCfuCfUfucuu | 46.09 | |
| | | aagagccuca | | AfuAfgcususu | | |
| 229 | 1649 | usasagAfaGfAfGf | 2277 | usUfsgguUfgAfGfgcuc | 19.57 | |
| | | ccucaaccaa | | UfuCfuuasusu | | |
| 254 | 1650 | gscscuCfaAfCfAf | 2278 | usUfsgacGfuGfCfuugu | 11.02 | |
| | | agcacgucaa | | UfgAfggcsusu | | |
| 255 | 1651 | cscsucAfaCfAfAf | 2279 | usUfsugaCfgUfGfcuug | 17.96 | |
| | | gcacgucaaa | | UfuGfaggsusu | | |
| 256 | 1652 | csuscaAfcAfAfGf | 2280 | usUfsuugAfcGfUfgcuu | 65.11 | |
| | | cacgucaaaa | | GfuUfgagsusu | | |
| 257 | 1653 | uscsaaCfaAfGfCf | 2281 | usUfsuuuGfaCfGfugcu | 20.27 | |
| | | acgucaaaaa | | UfgUfugasusu | | |
| 290 | 1654 | usasucAfaUfUfUf | 2282 | usAfsugaGfaCfAfgaaa | 8.85 | |
| | | cugucucaua | | UfuGfauasusu | | |
| 291 | 1655 | asuscaAfuUfUfCf | 2283 | usGfsaugAfgAfCfagaaA | 9.15 | |
| | | ugucucauca | | fuUfgaususu | | |
| 292 | 1656 | uscsaaUfuUfCfUf | 2284 | usAfsgauGfaGfAfcagaA | 14.18 | |
| | | gucucaucua | | faUfugasusu | | |
| 293 | 1657 | csasauUfuCfUfGf | 2285 | usAfsagaUfgAfGfacagA | 5.39 | |
| | | ucucaucuua | | faAfuugsusu | | |
| 297 | 1658 | ususcuGfuCfUfCf | 2286 | usUfsauuAfaGfAfugagA | 9.52 | |
| | | aucuuaauaa | | fcAfgaasusu | | |
| 298 | 1659 | uscsugUfcUfCfAf | 2287 | usAfsuauUfaAfGfauga | 7.54 | |
| | | ucuuaauaua | | GfaCfagasusu | | |
| 299 | 1660 | csusguCfuCfAfUf | 2288 | usCfsauaUfuAfAfgaugA | 7.55 | |
| | | cuuaauauga | | fgAfcagsusu | | |
| 313 | 1661 | usasugUfcUfCfUf | 2289 | usAfsgauCfaGfCfaaga | 6.78 | |
| | | ugcugaucua | | GfaCfauasusu | | |
| 346 | 1662 | ususcuCfuGfAfAf | 2290 | usUfsagcAfgAfAfcuucA | 27.15 | |
| | | guucugcuaa | | fgAfgaasusu | | |
| 348 | 1663 | csuscuGfaAfGfUf | 2291 | usUfsguaGfcAfGfaacu | 28.69 | |
| | | ucugcuacaa | | UfcAfgagsusu | | |
| 353 | 1664 | asasguUfcUfGfCf | 2292 | usGfsaggUfuGfUfagca | 6.45 | |
| | | uacaaccuca | | GfaAfcuususu | | |
| 354 | 1665 | asgsuuCfuGfCfUf | 2293 | usAfsgagGfuUfGfuagc | 7.03 | |
| | | acaaccucua | | AfgAfacususu | | |
| 355 | 1666 | gsusucUfgCfUfAf | 2294 | usUfsagaGfgUfUfguag | 13.17 | |
| | | caaccucuaa | | CfaGfaacsusu | | |
| 357 | 1667 | uscsugCfuAfCfAf | 2295 | usUfscuaGfaGfGfuugu | 22.60 | |
| | | accucuagaa | | AfgCfagasusu | | |
| 361 | 1668 | csusacAfaCfCfUf | 2296 | usCfsagaUfcUfAfgaggU | 7.39 | |
| | | cuagaucuga | | fuGfuagsusu | | |
| 363 | 1669 | ascsaaCfcUfCfUf | 2297 | usUfsgcaGfaUfCfuaga | 12.75 | |
| | | agaucugcaa | | GfgUfugususu | | |
| 381 | 1670 | gscsuuGfcCfAfCf | 2298 | usUfsaagCfuGfAfugug | 4.78 | |
| | | aucagcuuaa | | GfcAfagcsusu | | |
| 382 | 1671 | csusugCfcAfCfAf | 2299 | usUfsuaaGfcUfGfaugu | 4.86 | |
| | | ucagcuuaaa | | GfgCfaagsusu | | |
| 383 | 1672 | ususgcCfaCfAfUf | 2300 | usUfsuuaAfgCfUfgaug | 5.63 | |
| | | cagcuuaaaa | | UfgGfcaasusu | | |
| 387 | 1673 | csascaUfcAfGfCf | 2301 | usAfsgauUfuUfAfagcu | 6.15 | |
| | | uuaaaaucua | | GfaUfgugsusu | | |
| 388 | 1674 | ascsauCfaGfCfUf | 2302 | usCfsagaUfuUfUfaagc | 5.44 | |
| | | uaaaaucuga | | UfgAfugususu | | |
| 389 | 1675 | csasucAfgCfUfUf | 2303 | usAfscagAfuUfUfuaag | 8.02 | |
| | | aaaaucugua | | CfuGfaugsusu | | |
| 390 | 1676 | asuscaGfcUfUfAf | 2304 | usGfsacaGfaUfUfuuaa | 28.60 | |
| | | aaaucuguca | | GfcUfgaususu | | |
| 391 | 1677 | uscsagCfuUfAfAf | 2305 | usUfsgacAfgAfUfuuuaA | 6.69 | |
| | | aaucugucaa | | fgCfugasusu | | |
| 392 | 1678 | csasgcUfuAfAfAf | 2306 | usAfsugaCfaGfAfuuuu | 5.88 | |
| | | aucugucaua | | AfaGfcugsusu | | |
| 393 | 1679 | asgscuUfaAfAfAf | 2307 | usGfsaugAfcAfGfauuu | 12.00 | |
| | | ucugucauca | | UfaAfgcususu | | |
| 394 | 1680 | gscsuuAfaAfAfUf | 2308 | usGfsgauGfaCfAfgauu | 10.23 | |
| | | cugucaucca | | UfuAfagcsusu | | |
| 438 | 1681 | usasacAfgAfCfCf | 2309 | usUfscagGfaAfGfuggu | 15.16 | |
| | | acuuccugaa | | CfuGfuuasusu | | |
| 440 | 1682 | ascsagAfcCfAfCf | 2310 | usAfscucAfgGfAfagugG | 13.77 | |
| | | uuccugagua | | fuCfugususu | | |
| 441 | 1683 | csasgaCfcAfCfUf | 2311 | usUfsacuCfaGfGfaagu | 15.01 | |
| | | uccugaguaa | | GfgUfcugsusu | | |
| 442 | 1684 | asgsacCfaCfUfUf | 2312 | usCfsuacUfcAfGfgaag | 13.69 | |
| | | ccugaguaga | | UfgGfucususu | | |
| 443 | 1685 | gsasccAfcUfUfCf | 2313 | usUfscuaCfuCfAfggaa | 14.56 | |
| | | cugaguagaa | | GfuGfgucsusu | | |
| 449 | 1686 | ususccUfgAfGfUf | 2314 | usAfsacuCfuUfCfuacu | 10.17 | |
| | | agaagaguua | | CfaGfgaasusu | | |
| 469 | 1687 | ususugUfgAfAfAf | 2315 | usUfscuuGfaCfCfuuuu | 10.08 | |
| | | aggucaagaa | | CfaCfaaasusu | | |
| 472 | 1688 | gsusgaAfaAfGfGf | 2316 | usUfsaauCfuUfGfaccu | 5.65 | |
| | | ucaagauuaa | | UfuUfcacsusu | | |
| 473 | 1689 | usgsaaAfaGfGfUf | 2317 | usUfsuaaUfcUfUfgacc | 6.20 | |
| | | caagauuaaa | | UfuUfucasusu | | |
| 474 | 1690 | gsasaaAfgGfUfCf | 2318 | usCfsuuaAfuCfUfugac | 12.45 | |
| | | aagauuaaga | | CfuUfuucsusu | | |
| 526 | 1691 | ususggAfuCfUfUf | 2319 | usCfsaugUfuUfAfcaag | 9.46 | |
| | | guaaacauga | | AfuCfcaasusu | | |
| 527 | 1692 | usgsgaUfcUfUfGf | 2320 | usUfscauGfuUfUfacaa | 11.04 | |
| | | uaaacaugaa | | GfaUfccasusu | | |
| 531 | 1693 | uscsuuGfuAfAfAf | 2321 | usCfsuuuUfcAfUfguuu | 16.13 | |
| | | caugaaaaga | | AfcAfagasusu | | |
| 532 | 1694 | csusugUfaAfAfCf | 2322 | usCfscuuUfuCfAfuguu | 12.04 | |
| | | augaaaagga | | UfaCfaagsusu | | |
| 533 | 1695 | ususguAfaAfCfAf | 2323 | usCfsccuUfuUfCfaugu | 12.99 | |
| | | ugaaaaggga | | UfuAfcaasusu | | |
| 537 | 1696 | asasacAfuGfAfAf | 2324 | usAfsaagCfcCfUfuuuc | 9.15 | |
| | | aagggcuuua | | AfuGfuuususu | | |
| 538 | 1697 | asascaUfgAfAfAfa | 2325 | usUfsaaaGfcCfCfuuuu | 11.42 | |
| | | gggcuuuaa | | CfaUfguususu | | |
| 628 | 1698 | csusauCfcCfAfAf | 2326 | usAfsgaaAfaGfAfuuug | 16.43 | |
| | | aucuuuucua | | GfgAfuagsusu | | |
| 630 | 1699 | asusccCfaAfAfUf | 2327 | usUfscagAfaAfAfgauuU | 21.96 | |
| | | cuuuucugaa | | fgGfgaususu | | |
| 631 | 1700 | uscsccAfaAfUfCf | 2328 | usUfsucaGfaAfAfagau | 32.77 | |
| | | uuuucugaaa | | UfuGfggasusu | | |
| 639 | 1701 | csusuuUfcUfGfAf | 2329 | usUfscuuCfaUfCfuuca | 23.42 | |
| | | agaugaagaa | | GfaAfaagsusu | | |
| 640 | 1702 | ususuuCfuGfAfAf | 2330 | usCfsucuUfcAfUfcuuc | 16.65 | |
| | | gaugaagaga | | AfgAfaaasusu | | |
| 641 | 1703 | ususucUfgAfAfGf | 2331 | usAfscucUfuCfAfucuu | 16.55 | |
| | | augaagagua | | CfaGfaaasusu | | |
| 642 | 1704 | ususcuGfaAfGfAf | 2332 | usAfsacuCfuUfCfaucu | 18.04 | |
| | | ugaagaguua | | UfcAfgaasusu | | |
| 643 | 1705 | uscsugAfaGfAfUf | 2333 | usAfsaacUfcUfUfcauc | 17.05 | |
| | | gaagaguuua | | UfuCfagasusu | | |
| 644 | 1706 | csusgaAfgAfUfGf | 2334 | usUfsaaaCfuCfUfucau | 21.95 | |
| | | aagaguuuaa | | CfuUfcagsusu | | |
| 645 | 1707 | usgsaaGfaUfGfAf | 2335 | usCfsuaaAfcUfCfuuca | 22.83 | |
| | | agaguuuaga | | UfcUfucasusu | | |
| 646 | 1708 | gsasagAfuGfAfAfg | 2336 | usAfscuaAfaCfUfcuuc | 26.39 | |
| | | aguuuagua | | AfuCfuucsusu | | |
| 670 | 1709 | ascsugCfaCfUfGf | 2337 | usAfscuuGfuUfGfgcag | 23.93 | |
| | | ccaacaagua | | UfgCfagususu | | |
| 673 | 1710 | gscsacUfgCfCfAf | 2338 | usUfsgaaCfuUfGfuugg | 23.52 | |
| | | acaaguucaa | | CfaGfugcsusu | | |
| 677 | 1711 | usgsccAfaCfAfAfg | 2339 | usGfsaagUfgAfAfcuug | 19.93 | |
| | | uucacuuca | | UfuGfgcasusu | | |
| 687 | 1712 | ususcaCfuUfCfAf | 2340 | usCfsuuuAfuAfUfauga | 26.21 | |
| | | uauauaaaga | | AfgUfgaasusu | | |
| 688 | 1713 | uscsacUfuCfAfUf | 2341 | usGfscuuUfaUfAfuaug | 24.69 | |
| | | auauaaagca | | AfaGfugasusu | | |
| 689 | 1714 | csascuUfcAfUfAf | 2342 | usUfsgcuUfuAfUfauau | 30.20 | |
| | | uauaaagcaa | | GfaAfgugsusu | | |
| 711 | 1715 | ususuuAfcUfCfUf | 2343 | usCfsaccUfcAfAfaaga | 32.63 | |
| | | uuugagguga | | GfuAfaaasusu | | |
| 712 | 1716 | ususuaCfuCfUfUf | 2344 | usUfscacCfuCfAfaaag | 21.59 | |
| | | uugaggugaa | | AfgUfaaasusu | | |
| 713 | 1717 | ususacUfcUfUfUf | 2345 | usUfsucaCfcUfCfaaaa | 20.40 | |
| | | ugaggugaaa | | GfaGfuaasusu | | |
| 714 | 1718 | usascuCfuUfUfUf | 2346 | usAfsuucAfcCfUfcaaa | 19.61 | |
| | | gaggugaaua | | AfgAfguasusu | | |
| 781 | 1719 | gsgsucUfuCfAfCf | 2347 | usUfsgauAfcUfUfggugA | 21.71 | |
| | | caaguaucaa | | faGfaccsusu | | |
| 793 | 1720 | gsusauCfaAfAfGf | 2348 | usGfsuguUfaUfUfacuu | 15.87 | |
| | | uaauaacaca | | UfgAfuacsusu | | |
| 797 | 1721 | csasaaGfuAfAfUf | 2349 | usAfsuuuGfuGfUfuauu | 33.02 | |
| | | aacacaaaua | | AfcUfuugsusu | | |
| 800 | 1722 | asgsuaAfuAfAfCf | 2350 | usUfsucaUfuUfGfuguu | 30.01 | |
| | | acaaaugaaa | | AfuUfacususu | | |
| 842 | 1723 | usgsacUfcCfUfCf | 2351 | usAfsacaGfaUfGfugag | 26.50 | |
| | | acaucuguua | | GfaGfucasusu | | |
| 844 | 1724 | ascsucCfuCfAfCf | 2352 | usAfsuaaCfaGfAfugug | 26.91 | |
| | | aucuguuaua | | AfgGfagususu | | |
| 845 | 1725 | csusccUfcAfCfAf | 2353 | usGfsauaAfcAfGfaugu | 28.04 | |
| | | ucuguuauca | | GfaGfgagsusu | | |
| 846 | 1726 | uscscuCfaCfAfUf | 2354 | usAfsgauAfaCfAfgaugU | 31.11 | |
| | | cuguuaucua | | fgAfggasusu | | |
| 928 | 1727 | uscsuaUfgCfCfAf | 2355 | usAfsuguUfaAfUfguggC | 32.80 | |
| | | cauuaacaua | | faUfagasusu | | |
| 949 | 1728 | ususaaAfgUfUfGf | 2356 | usGfsauuCfuCfAfucaa | 23.97 | |
| | | augagaauca | | CfuUfuaasusu | | |
| 957 | 1729 | gsasugAfgAfAfUfc | 2357 | usCfscauAfcUfUfgauu | 28.03 | |
| | | aaguaugga | | CfuCfaucsusu | | |
| 958 | 1730 | asusgaGfaAfUfCf | 2358 | usUfsccaUfaCfUfugau | 31.29 | |
| | | aaguauggaa | | UfcUfcaususu | | |
| 960 | 1731 | gsasgaAfuCfAfAfg | 2359 | usUfsuucCfaUfAfcuug | 26.65 | |
| | | uauggaaaa | | AfuUfcucsusu | | |
| 961 | 1732 | asgsaaUfcAfAfGf | 2360 | usUfsuuuCfcAfUfacuu | 24.43 | |
| | | uauggaaaaa | | GfaUfucususu | | |
| 962 | 1733 | gsasauCfaAfGfUf | 2361 | usCfsuuuUfcCfAfuacu | 26.08 | |
| | | auggaaaaga | | UfgAfuucsusu | | |
| 963 | 1734 | asasucAfaGfUfAf | 2362 | usAfscuuUfuCfCfauac | 29.27 | |
| | | uggaaaagua | | UfuGfauususu | | |
| 964 | 1735 | asuscaAfgUfAfUf | 2363 | usUfsacuUfuUfCfcaua | 29.69 | |
| | | ggaaaaguaa | | CfuUfgaususu | | |
| 965 | 1736 | uscsaaGfuAfUfGf | 2364 | usUfsuacUfuUfUfccau | 29.12 | |
| | | gaaaaguaaa | | AfcUfugasusu | | |
| 966 | 1737 | csasagUfaUfGfGf | 2365 | usCfsuuaCfuUfUfucca | 32.99 | |
| | | aaaaguaaga | | UfaCfuugsusu | | |
| 967 | 1738 | asasguAfuGfGfAf | 2366 | usCfscuuAfcUfUfuucc | 43.38 | |
| | | aaaguaagga | | AfuAfcuususu | | |
| 968 | 1739 | asgsuaUfgGfAfAf | 2367 | usGfsccuUfaCfUfuuuc | 43.54 | |
| | | aaguaaggca | | CfaUfacususu | | |
| 969 | 1740 | gsusauGfgAfAfAf | 2368 | usGfsgccUfuAfCfuuuu | 29.74 | |
| | | aguaaggcca | | CfcAfuacsusu | | |
| 972 | 1741 | usgsgaAfaAfGfUf | 2369 | usUfsaugGfcCfUfuacu | 41.00 | |
| | | aaggccauaa | | UfuUfccasusu | | |
| 981 | 1742 | asasggCfcAfUfAf | 2370 | usAfsuguAfaGfAfguau | 36.69 | |
| | | cucuuacaua | | GfgCfcuususu | | |
| 985 | 1743 | cscsauAfcUfCfUf | 2371 | usUfsauuAfuGfUfaaga | 28.57 | |
| | | uacauaauaa | | GfuAfuggsusu | | |
| 1022 | 1744 | ususuuCfaAfAfGf | 2372 | usUfscugUfgAfUfucuu | 20.86 | |
| | | aaucacagaa | | UfgAfaaasusu | | |
| 1023 | 1745 | ususucAfaAfGfAf | 2373 | usUfsucuGfuGfAfuucu | 27.92 | |
| | | aucacagaaa | | UfuGfaaasusu | | |
| 1024 | 1746 | ususcaAfaGfAfAf | 2374 | usAfsuucUfgUfGfauuc | 26.51 | |
| | | ucacagaaua | | UfuUfgaasusu | | |
| 1031 | 1747 | asasucAfcAfGfAf | 2375 | usUfsacuAfgAfAfuucu | 29.31 | |
| | | auucuaguaa | | GfuGfauususu | | |
| 1032 | 1748 | asuscaCfaGfAfAf | 2376 | usGfsuacUfaGfAfauuc | 26.62 | |
| | | uucuaguaca | | UfgUfgaususu | | |
| 1061 | 1749 | uscsauAfaAfUfCf | 2377 | usCfsuuaGfaAfCfagau | 56.38 | |
| | | uguucuaaga | | UfuAfugasusu | | |
| 1062 | 1750 | csasuaAfaUfCfUf | 2378 | usUfscuuAfgAfAfcagaU | 37.11 | |
| | | guucuaagaa | | fuUfaugsusu | | |
| 1063 | 1751 | asusaaAfuCfUfGf | 2379 | usGfsucuUfaGfAfacag | 27.70 | |
| | | uucuaagaca | | AfuUfuaususu | | |
| 1064 | 1752 | usasaaUfcUfGfUf | 2380 | usUfsgucUfuAfGfaaca | 19.64 | |
| | | ucuaagacaa | | GfaUfuuasusu | | |
| 1067 | 1753 | asuscuGfuUfCfUf | 2381 | usAfsuauGfuCfUfuaga | 22.01 | |
| | | aagacauaua | | AfcAfgaususu | | |
| 1069 | 1754 | csusguUfcUfAfAf | 2382 | usUfscauAfuGfUfcuua | 25.10 | |
| | | gacauaugaa | | GfaAfcagsusu | | |
| 1070 | 1755 | usgsuuCfuAfAfGf | 2383 | usAfsucaUfaUfGfucuu | 26.79 | |
| | | acauaugaua | | AfgAfacasusu | | |
| 1074 | 1756 | csusaaGfaCfAfUf | 2384 | usGfsuugAfuCfAfuaug | 25.29 | |
| | | augaucaaca | | UfcUfuagsusu | | |
| 1075 | 1757 | usasagAfcAfUfAf | 2385 | usUfsguuGfaUfCfauau | 33.55 | |
| | | ugaucaacaa | | GfuCfuuasusu | | |
| 1080 | 1758 | csasuaUfgAfUfCf | 2386 | usUfscauCfuGfUfugau | 30.58 | |
| | | aacagaugaa | | CfaUfaugsusu | | |
| 1081 | 1759 | asusauGfaUfCfAf | 2387 | usCfsucaUfcUfGfuuga | 33.83 | |
| | | acagaugaga | | UfcAfuaususu | | |
| 1084 | 1760 | usgsauCfaAfCfAf | 2388 | usGfsuucUfcAfUfcugu | 29.23 | |
| | | gaugagaaca | | UfgAfucasusu | | |
| 1086 | 1761 | asuscaAfcAfGfAf | 2389 | usCfsaguUfcUfCfaucu | 32.48 | |
| | | ugagaacuga | | GfuUfgaususu | | |
| 1087 | 1762 | uscsaaCfaGfAfUf | 2390 | usCfscagUfuCfUfcauc | 28.02 | |
| | | gagaacugga | | UfgUfugasusu | | |
| 1120 | 1763 | asgsugAfgAfUfUf | 2391 | usGfsauaUfgAfCfuaau | 27.61 | |
| | | agucauauca | | CfuCfacususu | | |
| 1122 | 1764 | usgsagAfuUfAfGf | 2392 | usGfsugaUfaUfGfacua | 32.43 | |
| | | ucauaucaca | | AfuCfucasusu | | |
| 1136 | 1765 | uscsacUfaAfUfAf | 2393 | usUfsuguUfaGfUfauau | 27.28 | |
| | | uacuaacaaa | | UfaGfugasusu | | |
| 1178 | 1766 | ascsugUfaGfUfGf | 2394 | usCfsagaUfaAfUfucac | 33.31 | |
| | | aauuaucuga | | UfaCfagususu | | |
| 1179 | 1767 | csusguAfgUfGfAf | 2395 | usUfscagAfuAfAfuucaC | 30.55 | |
| | | auuaucugaa | | fuAfcagsusu | | |
| 1231 | 1768 | gsgsaaUfcAfUfGf | 2396 | usUfsuaaGfgUfUfucau | 29.75 | |
| | | aaaccuuaaa | | GfaUfuccsusu | | |
| 1232 | 1769 | gsasauCfaUfGfAf | 2397 | usCfsuuaAfgGfUfuuca | 25.68 | |
| | | aaccuuaaga | | UfgAfuucsusu | | |
| 1233 | 1770 | asasucAfuGfAfAf | 2398 | usUfscuuAfaGfGfuuuc | 28.47 | |
| | | accuuaagaa | | AfuGfauususu | | |
| 1234 | 1771 | asuscaUfgAfAfAf | 2399 | usGfsucuUfaAfGfguuu | 28.39 | |
| | | ccuuaagaca | | CfaUfgaususu | | |
| 1235 | 1772 | uscsauGfaAfAfCf | 2400 | usAfsgucUfuAfAfgguuU | 19.37 | |
| | | cuuaagacua | | fcAfugasusu | | |
| 1236 | 1773 | csasugAfaAfCfCf | 2401 | usAfsaguCfuUfAfaggu | 22.25 | |
| | | uuaagacuua | | UfuCfaugsusu | | |
| 1239 | 1774 | gsasaaCfcUfUfAf | 2402 | usCfsugaAfgUfCfuuaa | 24.33 | |
| | | agacuucaga | | GfgUfuucsusu | | |
| 1240 | 1775 | asasacCfuUfAfAf | 2403 | usUfscugAfaGfUfcuua | 17.79 | |
| | | gacuucagaa | | AfgGfuuususu | | |
| 1266 | 1776 | gscsagGfuUfGfUf | 2404 | usGfsaauGfgAfAfgacaA | 17.13 | |
| | | cuuccauuca | | fcCfugcsusu | | |
| 1267 | 1777 | csasggUfuGfUfCf | 2405 | usGfsgaaUfgGfAfagacA | 27.98 | |
| | | uuccauucca | | faCfcugsusu | | |
| 1268 | 1778 | asgsguUfgUfCfUf | 2406 | usUfsggaAfuGfGfaaga | 26.98 | |
| | | uccauuccaa | | CfaAfccususu | | |
| 1286 | 1779 | gscscuAfaCfAfUf | 2407 | usCfsugcAfuUfGfgaug | 30.83 | |
| | | ccaaugcaga | | UfuAfggcsusu | | |
| 1287 | 1780 | cscsuaAfcAfUfCf | 2408 | usCfscugCfaUfUfggau | 64.20 | |
| | | caaugcagga | | GfuUfaggsusu | | |
| 1288 | 1781 | csusaaCfaUfCfCf | 2409 | usGfsccuGfcAfUfugga | 44.20 | |
| | | aaugcaggca | | UfgUfuagsusu | | |
| 1289 | 1782 | usasacAfuCfCfAf | 2410 | usUfsgccUfgCfAfuuggA | 32.14 | |
| | | augcaggcaa | | fuGfuuasusu | | |
| 1290 | 1783 | asascaUfcCfAfAf | 2411 | usUfsugcCfuGfCfauug | 27.11 | |
| | | ugcaggcaaa | | GfaUfguususu | | |
| 1291 | 1784 | ascsauCfcAfAfUf | 2412 | usCfsuugCfcUfGfcauu | 38.29 | |
| | | gcaggcaaga | | GfgAfugususu | | |
| 1292 | 1785 | csasucCfaAfUfGf | 2413 | usCfscuuGfcCfUfgcau | 35.27 | |
| | | caggcaagga | | UfgGfaugsusu | | |
| 1293 | 1786 | asusccAfaUfGfCf | 2414 | usUfsccuUfgCfCfugca | 40.03 | |
| | | aggcaaggaa | | UfuGfgaususu | | |
| 1294 | 1787 | uscscaAfuGfCfAf | 2415 | usUfsuccUfuGfCfcugc | 31.21 | |
| | | ggcaaggaaa | | AfuUfggasusu | | |
| 1295 | 1788 | cscsaaUfgCfAfGf | 2416 | usUfsuucCfuUfGfccug | 25.76 | |
| | | gcaaggaaaa | | CfaUfuggsusu | | |
| 1296 | 1789 | csasauGfcAfGfGf | 2417 | usUfsuuuCfcUfUfgccu | 28.84 | |
| | | caaggaaaaa | | GfcAfuugsusu | | |
| 1297 | 1790 | asasugCfaGfGfCf | 2418 | usAfsuuuUfcCfUfugcc | 35.56 | |
| | | aaggaaaaua | | UfgCfauususu | | |
| 1304 | 1791 | gscsaaGfgAfAfAfa | 2419 | usAfsucuUfuUfAfuuuu | 39.65 | |
| | | uaaaagaua | | CfcUfugcsusu | | |
| 1312 | 1792 | asasuaAfaAfGfAf | 2420 | usCfsacuGfgAfAfaucu | 37.10 | |
| | | uuuccaguga | | UfuUfauususu | | |
| 1313 | 1793 | asusaaAfaGfAfUf | 2421 | usUfscacUfgGfAfaauc | 24.38 | |
| | | uuccagugaa | | UfuUfuaususu | | |
| 1314 | 1794 | usasaaAfgAfUfUf | 2422 | usGfsucaCfuGfGfaaau | 26.42 | |
| | | uccagugaca | | CfuUfuuasusu | | |
| 1315 | 1795 | asasaaGfaUfUfUf | 2423 | usUfsgucAfcUfGfgaaa | 26.96 | |
| | | ccagugacaa | | UfcUfuuususu | | |
| 1366 | 1796 | usasuaUfgAfAfUf | 2424 | usGfsgagAfgAfGfaauu | 28.41 | |
| | | ucucucucca | | CfaUfauasusu | | |
| 1367 | 1797 | asusauGfaAfUfUf | 2425 | usUfsggaGfaGfAfgaau | 19.93 | |
| | | cucucuccaa | | UfcAfuaususu | | |
| 1368 | 1798 | usasugAfaUfUfCf | 2426 | usUfsuggAfgAfGfagaaU | 18.78 | |
| | | ucucuccaaa | | fuCfauasusu | | |
| 1369 | 1799 | asusgaAfuUfCfUf | 2427 | usUfsuugGfaGfAfgaga | 33.99 | |
| | | cucuccaaaa | | AfuUfcaususu | | |
| 1370 | 1800 | usgsaaUfuCfUfCf | 2428 | usAfsuuuGfgAfGfagagA | 17.29 | |
| | | ucuccaaaua | | faUfucasusu | | |
| 1371 | 1801 | gsasauUfcUfCfUf | 2429 | usUfsauuUfgGfAfgaga | 24.77 | |
| | | cuccaaauaa | | GfaAfuucsusu | | |
| 1372 | 1802 | asasuuCfuCfUfCf | 2430 | usAfsuauUfuGfGfagag | 28.98 | |
| | | uccaaauaua | | AfgAfauususu | | |
| 1373 | 1803 | asusucUfcUfCfUf | 2431 | usAfsauaUfuUfGfgaga | 24.84 | |
| | | ccaaauauua | | GfaGfaaususu | | |
| 1412 | 1804 | ususgaAfaUfGfAf | 2432 | usCfscaaCfaAfGfuuca | 45.46 | |
| | | acuuguugga | | UfuUfcaasusu | | |
| 1414 | 1805 | gsasaaUfgAfAfCf | 2433 | usGfsgccAfaCfAfaguu | 49.27 | |
| | | uuguuggcca | | CfaUfuucsusu | | |
| 1415 | 1806 | asasauGfaAfCfUf | 2434 | usGfsggcCfaAfCfaagu | 51.78 | |
| | | uguuggccca | | UfcAfuuususu | | |
| 1416 | 1807 | asasugAfaCfUfUf | 2435 | usUfsgggCfcAfAfcaagU | 52.44 | |
| | | guuggcccaa | | fuCfauususu | | |
| 1421 | 1808 | ascsuuGfuUfGfGf | 2436 | usAfsuagAfuGfGfgccaA | 46.46 | |
| | | cccaucuaua | | fcAfagususu | | |
| 1423 | 1809 | ususguUfgGfCfCf | 2437 | usUfsaauAfgAfUfgggcC | 44.51 | |
| | | caucuauuaa | | faAfcaasusu | | |
| 1426 | 1810 | ususggCfcCfAfUf | 2438 | usAfsuguAfaUfAfgaugG | 61.92 | |
| | | cuauuacaua | | fgCfcaasusu | | |
| 1430 | 1811 | cscscaUfcUfAfUf | 2439 | usGfsuagAfuGfUfaaua | 27.96 | |
| | | uacaucuaca | | GfaUfgggsusu | | |
| 1431 | 1812 | cscsauCfuAfUfUf | 2440 | usUfsguaGfaUfGfuaau | 37.37 | |
| | | acaucuacaa | | AfgAfuggsusu | | |
| 1432 | 1813 | csasucUfaUfUfAf | 2441 | usCfsuguAfgAfUfguaa | 33.11 | |
| | | caucuacaga | | UfaGfaugsusu | | |
| 1433 | 1814 | asuscuAfuUfAfCf | 2442 | usGfscugUfaGfAfugua | 51.68 | |
| | | aucuacagca | | AfuAfgaususu | | |
| 1434 | 1815 | uscsuaUfuAfCfAf | 2443 | usAfsgcuGfuAfGfaugu | 46.70 | |
| | | ucuacagcua | | AfaUfagasusu | | |
| 1435 | 1816 | csusauUfaCfAfUf | 2444 | usCfsagcUfgUfAfgaug | 35.99 | |
| | | cuacagcuga | | UfaAfuagsusu | | |
| 1436 | 1817 | usasuuAfcAfUfCf | 2445 | usUfscagCfuGfUfagau | 74.77 | |
| | | uacagcugaa | | GfuAfauasusu | | |
| 1437 | 1818 | asusuaCfaUfCfUf | 2446 | usGfsucaGfcUfGfuaga | 43.34 | |
| | | acagcugaca | | UfgUfaaususu | | |
| 1438 | 1819 | ususacAfuCfUfAf | 2447 | usGfsgucAfgCfUfguagA | 39.40 | |
| | | cagcugacca | | fuGfuaasusu | | |
| 1439 | 1820 | usascaUfcUfAfCf | 2448 | usGfsgguCfaGfCfugua | 60.23 | |
| | | agcugaccca | | GfaUfguasusu | | |
| 1440 | 1821 | ascsauCfuAfCfAf | 2449 | usAfsgggUfcAfGfcuguA | 55.82 | |
| | | gcugacccua | | fgAfugususu | | |
| 1441 | 1822 | csasucUfaCfAfGf | 2450 | usAfsaggGfuCfAfgcugU | 49.19 | |
| | | cugacccuua | | faGfaugsusu | | |
| 1442 | 1823 | asuscuAfcAfGfCf | 2451 | usCfsaagGfgUfCfagcu | 50.31 | |
| | | ugacccuuga | | GfuAfgaususu | | |
| 1466 | 1824 | gsgsggUfuAfGfGf | 2452 | usGfsucaGfcUfCfcccu | 40.53 | |
| | | ggagcugaca | | AfaCfcccsusu | | |
| 1467 | 1825 | gsgsguUfaGfGfGf | 2453 | usUfsgucAfgCfUfcccc | 44.52 | |
| | | gagcugacaa | | UfaAfcccsusu | | |
| 1468 | 1826 | gsgsuuAfgGfGfGf | 2454 | usUfsuguCfaGfCfuccc | 37.16 | |
| | | agcugacaaa | | CfuAfaccsusu | | |
| 1469 | 1827 | gsusuaGfgGfGfAf | 2455 | usAfsuugUfcAfGfcucc | 25.04 | |
| | | gcugacaaua | | CfcUfaacsusu | | |
| 1498 | 1828 | csasaaAfuCfUfUf | 2456 | usUfsaggUfaGfUfuaag | 36.59 | |
| | | aacuaccuaa | | AfuUfuugsusu | | |
| 1501 | 1829 | asasucUfuAfAfCf | 2457 | usUfsauuAfgGfUfaguu | 38.49 | |
| | | uaccuaauaa | | AfaGfauususu | | |
| 1502 | 1830 | asuscuUfaAfCfUf | 2458 | usCfsuauUfaGfGfuagu | 32.27 | |
| | | accuaauaga | | UfaAfgaususu | | |
| 1505 | 1831 | ususaaCfuAfCfCf | 2459 | usAfsggcUfaUfUfagguA | 29.74 | |
| | | uaauagccua | | fgUfuaasusu | | |
| 1506 | 1832 | usasacUfaCfCfUf | 2460 | usUfsaggCfuAfUfuagg | 29.56 | |
| | | aauagccuaa | | UfaGfuuasusu | | |
| 1508 | 1833 | ascsuaCfcUfAfAf | 2461 | usAfsguaGfgCfUfauua | 33.87 | |
| | | uagccuacua | | GfgUfagususu | | |
| 1526 | 1834 | asusugAfcCfAfUf | 2462 | usGfsuaaGfgUfUfuaug | 31.60 | |
| | | aaaccuuaca | | GfuCfaaususu | | |
| 1527 | 1835 | ususgaCfcAfUfAf | 2463 | usAfsguaAfgGfUfuuau | 28.07 | |
| | | aaccuuacua | | GfgUfcaasusu | | |
| 1528 | 1836 | usgsacCfaUfAfAf | 2464 | usCfsaguAfaGfGfuuua | 27.44 | |
| | | accuuacuga | | UfgGfucasusu | | |
| 1529 | 1837 | gsasccAfuAfAfAfc | 2465 | usUfscagUfaAfGfguuu | 34.35 | |
| | | cuuacugaa | | AfuGfgucsusu | | |
| 1530 | 1838 | ascscaUfaAfAfCf | 2466 | usAfsucaGfuAfAfgguu | 32.28 | |
| | | cuuacugaua | | UfaUfggususu | | |
| 1531 | 1839 | cscsauAfaAfCfCf | 2467 | usUfsaucAfgUfAfagguU | 43.98 | |
| | | uuacugauaa | | fuAfuggsusu | | |
| 1532 | 1840 | csasuaAfaCfCfUf | 2468 | usUfsuauCfaGfUfaagg | 36.41 | |
| | | uacugauaaa | | UfuUfaugsusu | | |
| 1533 | 1841 | asusaaAfcCfUfUf | 2469 | usGfsuuaUfcAfGfuaag | 41.03 | |
| | | acugauaaca | | GfuUfuaususu | | |
| 1543 | 1842 | csusgaUfaAfCfAf | 2470 | usUfsacuGfuUfUfaugu | 45.77 | |
| | | uaaacaguaa | | UfaUfcagsusu | | |
| 1674 | 1843 | uscsauUfaAfAfUf | 2471 | usCfsccaCfuUfCfcauu | 33.95 | |
| | | ggaaguggga | | UfaAfugasusu | | |
| 1675 | 1844 | csasuuAfaAfUfGf | 2472 | usAfscccAfcUfUfccau | 29.44 | |
| | | gaagugggua | | UfuAfaugsusu | | |
| 1706 | 1845 | ususcaUfuCfUfCf | 2473 | usGfsaagAfcAfAfugagA | 40.93 | |
| | | auugucuuca | | faUfgaasusu | | |
| 1707 | 1846 | uscsauUfcUfCfAf | 2474 | usUfsgaaGfaCfAfauga | 38.43 | |
| | | uugucuucaa | | GfaAfugasusu | | |
| 1708 | 1847 | csasuuCfuCfAfUf | 2475 | usGfsugaAfgAfCfaaugA | 42.63 | |
| | | ugucuucaca | | fgAfaugsusu | | |
| 1711 | 1848 | uscsucAfuUfGfUf | 2476 | usAfsaugUfgAfAfgacaA | 45.77 | |
| | | cuucacauua | | fuGfagasusu | | |
| 1782 | 1849 | gsuscuUfgUfCfUf | 2477 | usAfscacCfcCfUfaaga | 36.00 | |
| | | uaggggugua | | CfaAfgacsusu | | |
| 1783 | 1850 | uscsuuGfuCfUfUf | 2478 | usCfsacaCfcCfCfuaag | 41.37 | |
| | | agggguguga | | AfcAfagasusu | | |
| 1817 | 1851 | asusauUfcAfUfGf | 2479 | usCfscacUfuAfUfacau | 49.18 | |
| | | uauaagugga | | GfaAfuaususu | | |
| 1822 | 1852 | csasugUfaUfAfAf | 2480 | usAfsgggUfcCfAfcuuaU | 57.50 | |
| | | guggacccua | | faCfaugsusu | | |
| 2000 | 1853 | asusuuUfcAfAfUf | 2481 | usAfsgagGfaGfAfaauu | 37.60 | |
| | | uucuccucua | | GfaAfaaususu | | |
| 2001 | 1854 | ususuuCfaAfUfUf | 2482 | usCfsagaGfgAfGfaaau | 41.61 | |
| | | ucuccucuga | | UfgAfaaasusu | | |
| 2002 | 1855 | ususucAfaUfUfUf | 2483 | usUfscagAfgGfAfgaaaU | 44.25 | |
| | | cuccucugaa | | fuGfaaasusu | | |
| 116 | 1856 | gsusuaUfcUfCfAf | 2484 | usAfsgccAfaAfUfaugaG | 25.28 | |
| | | uauuuggcua | | faUfaacsusu | | |
| 261 | 1857 | csasagCfaCfGfUf | 2485 | usUfsagcUfuUfUfgacg | 10.02 | |
| | | caaaagcuaa | | UfgCfuugsusu | | |
| 263 | 1858 | asgscaCfgUfCfAf | 2486 | usUfsguaGfcUfUfuuga | 18.61 | |
| | | aaagcuacaa | | CfgUfgcususu | | |
| 267 | 1859 | csgsucAfaAfAfGf | 2487 | usAfsuucUfgUfAfgcuu | 9.80 | |
| | | cuacagaaua | | UfuGfacgsusu | | |
| 268 | 1860 | gsuscaAfaAfGfCf | 2488 | usGfsauuCfuGfUfagcu | 6.35 | |
| | | uacagaauca | | UfuUfgacsusu | | |
| 534 | 1861 | usgsuaAfaCfAfUf | 2489 | usGfscccUfuUfUfcaug | 9.98 | |
| | | gaaaagggca | | UfuUfacasusu | | |
| 535 | 1862 | gsusaaAfcAfUfGf | 2490 | usAfsgccCfuUfUfucau | 14.77 | |
| | | aaaagggcua | | GfuUfuacsusu | | |
| 1089 | 1863 | asascaGfaUfGfAf | 2491 | usCfsaccAfgUfUfcuca | 41.56 | |
| | | gaacugguga | | UfcUfguususu | | |
| 1090 | 1864 | ascsagAfuGfAfGf | 2492 | usCfscacCfaGfUfucuc | 32.85 | |
| | | aacuggugga | | AfuCfugususu | | |
| 1091 | 1865 | csasgaUfgAfGfAf | 2493 | usAfsccaCfcAfGfuucu | 24.91 | |
| | | acugguggua | | CfaUfcugsusu | | |
| 1092 | 1866 | asgsauGfaGfAfAf | 2494 | usAfsaccAfcCfAfguucU | 20.21 | |
| | | cuggugguua | | fcAfucususu | | |
| 1093 | 1867 | gsasugAfgAfAfCfu | 2495 | usUfsaacCfaCfCfaguu | 18.42 | |
| | | ggugguuaa | | CfuCfaucsusu | | |
| 1094 | 1868 | asusgaGfaAfCfUf | 2496 | usUfsuaaCfcAfCfcagu | 23.41 | |
| | | ggugguuaaa | | UfcUfcaususu | | |
| 1095 | 1869 | usgsagAfaCfUfGf | 2497 | usAfsuuaAfcCfAfccagU | 23.27 | |
| | | gugguuaaua | | fuCfucasusu | | |
| 1096 | 1870 | gsasgaAfcUfGfGf | 2498 | usUfsauuAfaCfCfacca | 24.74 | |
| | | ugguuaauaa | | GfuUfcucsusu | | |
| 1097 | 1871 | asgsaaCfuGfGfUf | 2499 | usAfsuauUfaAfCfcacc | 27.87 | |
| | | gguuaauaua | | AfgUfucususu | | |
| 1228 | 1872 | ususugGfaAfUfCf | 2500 | usAfsgguUfuCfAfugau | 20.34 | |
| | | augaaaccua | | UfcCfaaasusu | | |
| 1229 | 1873 | ususggAfaUfCfAf | 2501 | usAfsaggUfuUfCfauga | 16.53 | |
| | | ugaaaccuua | | UfuCfcaasusu | | |
| 1614 | 1874 | asasguAfaGfCfUf | 2502 | usAfsuuuUfcUfCfuagc | 37.67 | |
| | | agagaaaaua | | UfuAfcuususu | | |
| 1615 | 1875 | asgsuaAfgCfUfAf | 2503 | usCfsauuUfuCfUfcuag | 32.79 | |
| | | gagaaaauga | | CfuUfacususu | | |
| 1616 | 1876 | gsusaaGfcUfAfGf | 2504 | usAfscauUfuUfCfucua | 34.14 | |
| | | agaaaaugua | | GfcUfuacsusu | | |
| 1673 | 1877 | ususcaUfuAfAfAf | 2505 | usCfscacUfuCfCfauuu | 31.62 | |
| | | uggaagugga | | AfaUfgaasusu | | |
| 1701 | 1878 | asasguCfuUfCfAf | 2506 | usCfsaauGfaGfAfauga | 51.66 | |
| | | uucucauuga | | AfgAfcuususu | | |
| 1702 | 1879 | asgsucUfuCfAfUf | 2507 | usAfscaaUfgAfGfaaugA | 48.04 | |
| | | ucucauugua | | faGfacususu | | |
| 1703 | 1880 | gsuscuUfcAfUfUf | 2508 | usGfsacaAfuGfAfgaau | 49.89 | |
| | | cucauuguca | | GfaAfgacsusu | | |
| 1704 | 1881 | uscsuuCfaUfUfCf | 2509 | usAfsgacAfaUfGfagaaU | 37.01 | |
| | | ucauugucua | | fgAfagasusu | | |
| 1784 | 1882 | csusugUfcUfUfAf | 2510 | usCfscacAfcCfCfcuaa | 46.42 | |
| | | ggggugugga | | GfaCfaagsusu | | |
| 1818 | 1883 | usasuuCfaUfGfUf | 2511 | usUfsccaCfuUfAfuaca | 51.32 | |
| | | auaaguggaa | | UfgAfauasusu | | |
| 1821 | 1884 | uscsauGfuAfUfAf | 2512 | usGfsgguCfcAfCfuuau | 54.84 | |
| | | aguggaccca | | AfcAfugasusu | | |
| 470 | 1402 | ususguGfaAfAfAf | 2513 | vpUsAfsucuUfgAfCfcu | 8.30 | 0.59 |
| | | ggucaagaua | | uuUfcAfcaasusu | | |
| 471 | 1403 | usgsugAfaAfAfGfg | 2514 | vpUsAfsaucUfuGfAfcc | 9.30 | 2.91 |
| | | ucaagauua | | uuUfuCfacasusu | | |
| 475 | 1404 | asasaaGfgUfCfAf | 2515 | vpUsUfscuuAfaUfCfuu | 13.70 | 21.27 |
| | | agauuaagaa | | gaCfcUfuuususu | | |
| 450 | 1471 | uscscuGfaGfUfAf | 2516 | vpUsAfsaacUfcUfUfcu | 10.00 | 12.81 |
| | | gaagaguuua | | acUfcAfggasusu | | |
| 452 | 1472 | csusgaGfuAfGfAf | 2517 | vpUsAfsgaaAfcUfCfuuc | 8.20 | 0.27 |
| | | agaguuucua | | uAfcUfcagsusu | | |
| 528 | 1474 | gsgsauCfuUfGfUf | 2518 | vpUsUfsucaUfgUfUfua | 11.10 | 5.04 |
| | | aaacaugaaa | | caAfgAfuccsusu | | |
| 381 | 1670 | gscsuuGfcCfAfCf | 2519 | vpUsUfsaagCfuGfAfug | 7.60 | 9.74 |
| | | aucagcuuaa | | ugGfcAfagcsusu | | |
| 382 | 1671 | csusugCfcAfCfAf | 2520 | vpUsUfsuaaGfcUfGfau | 7.50 | 3.44 |
| | | ucagcuuaaa | | guGfgCfaagsusu | | |
| 383 | 1672 | ususgcCfaCfAfUf | 2521 | vpUsUfsuuaAfgCfUfga | 8.40 | 2.73 |
| | | cagcuuaaaa | | ugUfgGfcaasusu | | |
| 472 | 1688 | gsusgaAfaAfGfGf | 2522 | vpUsUfsaauCfuUfGfac | 8.20 | 0.06 |
| | | ucaagauuaa | | cuUfuUfcacsusu | | |

TABLE 1C
provides exemplary siRNAs with the unmodified sequences of the sense strand
and the antisense strand.
Column 1 shows the beginning position of the target sequence in PLN_human_#_
NM_002667.5 (SEQ ID NO: 2523). The target sequence is 19 nucleotide long
starting from the beginning position. For example, row 1 shows that the
beginning position of the target sequence is 202, which means that the target
sequence is the sequence corresponding to positions 202-220 of PLN_human_#_
NM_002667.5 (SEQ ID NO: 2523). The sequences of the sense strands and
antisense strands are shown from the 5′ end to the 3′ end.

| | New | | New | |
|---|---|---|---|---|
| Position in | SEQ ID | | SEQ ID | |
| transcript | NO: | Sense strand | NO: | Antisense strand |
| 202 | 2524 | AAGUCCAAUACCUCACUCG | 2644 | CGAGUGAGGUAUUGGACUU |
| 1209 | 2525 | UAGCUUACCAUACUAUAUC | 2645 | GAUAUAGUAUGGUAAGCUA |
| 1211 | 2526 | GCUUACCAUACUAUAUCUU | 2646 | AAGAUAUAGUAUGGUAAGC |
| 1481 | 2527 | GACAAUUCGUGGGUCCGCA | 2647 | UGCGGACCCACGAAUUGUC |
| 2424 | 2528 | AGAUUUCUUAAGUGACGCC | 2648 | GGCGUCACUUAAGAAAUCU |
| 2430 | 2529 | CUUAAGUGACGCCUCAUCU | 2649 | AGAUGAGGCGUCACUUAAG |
| 42 | 2530 | CCCGUAAGACUUCAUACAA | 2650 | UUGUAUGAAGUCUUACGGG |
| 219 | 2531 | CGCUCAGCUAUAAGAAGAG | 2651 | CUCUUCUUAUAGCUGAGCG |
| 329 | 2532 | CUGUAUCAUCGUGAUGCUU | 2652 | AAGCAUCACGAUGAUACAG |
| 330 | 2533 | UGUAUCAUCGUGAUGCUUC | 2653 | GAAGCAUCACGAUGAUACA |
| 1130 | 2534 | GUCAUAUCACUAAUAUACU | 2654 | AGUAUAUUAGUGAUAUGAC |
| 1202 | 2535 | AGUUACCUAGCUUACCAUA | 2655 | UAUGGUAAGCUAGGUAACU |
| 1503 | 2536 | UCUUAACUACCUAAUAGCC | 2656 | GGCUAUUAGGUAGUUAAGA |
| 1504 | 2537 | CUUAACUACCUAAUAGCCU | 2657 | AGGCUAUUAGGUAGUUAAG |
| 1900 | 2538 | CUCUAUCAACCAAAUGGUA | 2658 | UACCAUUUGGUUGAUAGAG |
| 1906 | 2539 | CAACCAAAUGGUAAGCAUC | 2659 | GAUGCUUACCAUUUGGUUG |
| 1939 | 2540 | CUCUACUGAGCUAAAUUAU | 2660 | AUAAUUUAGCUCAGUAGAG |
| 1940 | 2541 | UCUACUGAGCUAAAUUAUA | 2661 | UAUAAUUUAGCUCAGUAGA |
| 1948 | 2542 | GCUAAAUUAUAGAUCCAGC | 2662 | GCUGGAUCUAUAAUUUAGC |
| 2301 | 2543 | ACACAAGUGUUGCUAACUC | 2663 | GAGUUAGCAACACUUGUGU |
| 2309 | 2544 | GUUGCUAACUCAAUAGUGA | 2664 | UCACUAUUGAGUUAGCAAC |
| 2425 | 2545 | GAUUUCUUAAGUGACGCCU | 2665 | AGGCGUCACUUAAGAAAUC |
| 2429 | 2546 | UCUUAAGUGACGCCUCAUC | 2666 | GAUGAGGCGUCACUUAAGA |
| 2431 | 2547 | UUAAGUGACGCCUCAUCUA | 2667 | UAGAUGAGGCGUCACUUAA |
| 226 | 2548 | CUAUAAGAAGAGCCUCAAC | 2668 | GUUGAGGCUCUUCUUAUAG |
| 227 | 2549 | UAUAAGAAGAGCCUCAACC | 2669 | GGUUGAGGCUCUUCUUAUA |
| 228 | 2550 | AUAAGAAGAGCCUCAACCA | 2670 | UGGUUGAGGCUCUUCUUAU |
| 260 | 2551 | ACAAGCACGUCAAAAGCUA | 2671 | UAGCUUUUGACGUGCUUGU |
| 276 | 2552 | CUACAGAAUCUAUUUAUCA | 2672 | UGAUAAAUAGAUUCUGUAG |
| 667 | 2553 | AAAACUGCACUGCCAACAA | 2673 | UUGUUGGCAGUGCAGUUUU |
| 668 | 2554 | AAACUGCACUGCCAACAAG | 2674 | CUUGUUGGCAGUGCAGUUU |
| 959 | 2555 | UGAGAAUCAAGUAUGGAAA | 2675 | UUUCCAUACUUGAUUCUCA |
| 1099 | 2556 | AACUGGUGGUUAAUAUGUG | 2676 | CACAUAUUAACCACCAGUU |
| 1100 | 2557 | ACUGGUGGUUAAUAUGUGA | 2677 | UCACAUAUUAACCACCAGU |
| 1590 | 2558 | AUUAUACACUAUAUUCCUA | 2678 | UAGGAAUAUAGUGUAUAAU |
| 2522 | 2559 | GUAAAUUACCAAUACAGAA | 2679 | UUCUGUAUUGGUAAUUUAC |
| 2576 | 2560 | AACUGAUUAGUCAUAUUCC | 2680 | GGAAUAUGACUAAUCAGUU |
| 2837 | 2561 | ACUAUAGAAUAAGUUCUUA | 2681 | UAAGAACUUAUUCUAUAGU |
| 225 | 2562 | GCUAUAAGAAGAGCCUCAA | 2682 | UUGAGGCUCUUCUUAUAGC |
| 476 | 2563 | AAAGGUCAAGAUUAAGACU | 2683 | AGUCUUAAUCUUGACCUUU |
| 477 | 2564 | AAGGUCAAGAUUAAGACUA | 2684 | UAGUCUUAAUCUUGACCUU |
| 484 | 2565 | AGAUUAAGACUAAAACUUA | 2685 | UAAGUUUUAGUCUUAAUCU |
| 809 | 2566 | ACAAAUGAAGUGUCAUUAU | 2686 | AUAAUGACACUUCAUUUGU |
| 617 | 2567 | CUCACAAAUUUCUAUCCCA | 2687 | UGGGAUAGAAAUUUGUGAG |
| 666 | 2568 | UAAAACUGCACUGCCAACA | 2688 | UGUUGGCAGUGCAGUUUUA |
| 669 | 2569 | AACUGCACUGCCAACAAGU | 2689 | ACUUGUUGGCAGUGCAGUU |
| 274 | 2570 | AGCUACAGAAUCUAUUUAU | 2690 | AUAAAUAGAUUCUGUAGCU |
| 275 | 2571 | GCUACAGAAUCUAUUUAUC | 2691 | GAUAAAUAGAUUCUGUAGC |
| 1098 | 2572 | AAAACUUAUUGUUACCAUA | 2692 | UAUGGUAACAAUAAGUUUU |
| 1101 | 2573 | GAACUGGUGGUUAAUAUGU | 2693 | ACAUAUUAACCACCAGUUC |
| 1413 | 2574 | AACUGGUGGUUAAUAUGUG | 2694 | CACAUAUUAACCACCAGUU |
| 1483 | 2575 | ACUGGUGGUUAAUAUGUGA | 2695 | UCACAUAUUAACCACCAGU |
| 1484 | 2576 | CUGGUGGUUAAUAUGUGAC | 2696 | GUCACAUAUUAACCACCAG |
| 1485 | 2577 | AUUCGUGGGUCCGCAAAAU | 2697 | AUUUUGCGGACCCACGAAU |
| 1600 | 2578 | AUAUUCCUACAAUAAAGUA | 2698 | UACUUUAUUGUAGGAAUAU |
| 1876 | 2579 | AAUAGGAUAUAGCUAUUUU | 2699 | AAAAUAGCUAUAUCCUAUU |
| 1933 | 2580 | AGUCCACUCUACUGAGCUA | 2700 | UAGCUCAGUAGAGUGGACU |
| 2158 | 2581 | AUAAUCCAACAAUUUUAAU | 2701 | AUUAAAAUUGUUGGAUUAU |
| 2269 | 2582 | AUAUGUAACAUCAAUAUUG | 2702 | CAAUAUUGAUGUUACAUAU |
| 2349 | 2583 | UGAACCCAUGAGAGAUACU | 2703 | AGUAUCUCUCAUGGGUUCA |
| 2856 | 2584 | UCUUAAUUUACAGGGCACU | 2704 | AGUGCCCUGUAAAUUAAGA |
| 2857 | 2585 | CUUAAUUUACAGGGCACUA | 2705 | UAGUGCCCUGUAAAUUAAG |
| 278 | 2586 | ACAGAAUCUAUUUAUCAAU | 2706 | AUUGAUAAAUAGAUUCUGU |
| 2692 | 2587 | UGAGGAUUACAGAAUACUA | 2707 | UAGUAUUCUGUAAUCCUCA |
| 757 | 2588 | UUCUUUAAUACUAAGUAUU | 2708 | AAUACUUAGUAUUAAAGAA |
| 1379 | 2589 | UCUCCAAAUAUUAACUAAU | 2709 | AUUAGUUAAUAUUUGGAGA |
| 2220 | 2590 | UAGAGGAUGUGUAAUUAAC | 2710 | GUUAAUUACACAUCCUCUA |
| 2102 | 2591 | AAUUUAAGUUGACUAAAGU | 2711 | ACUUUAGUCAACUUAAAUU |
| 2690 | 2592 | UGUGAGGAUUACAGAAUAC | 2712 | GUAUUCUGUAAUCCUCACA |
| 52 | 2593 | UUCAUACAACACAAUACUC | 2713 | GAGUAUUGUGUUGUAUGAA |
| 1380 | 2594 | CUCCAAAUAUUAACUAAUU | 2714 | AAUUAGUUAAUAUUUGGAG |
| 1132 | 2595 | CAUAUCACUAAUAUACUAA | 2715 | UUAGUAUAUUAGUGAUAUG |
| 1126 | 2596 | AUUAGUCAUAUCACUAAUA | 2716 | UAUUAGUGAUAUGACUAAU |
| 281 | 2597 | GAAUCUAUUUAUCAAUUUC | 2717 | GAAAUUGAUAAAUAGAUUC |
| 2232 | 2598 | AAUUAACCAUAUCUUCUAA | 2718 | UUAGAAGAUAUGGUUAAUU |
| 2219 | 2599 | GUAGAGGAUGUGUAAUUAA | 2719 | UUAAUUACACAUCCUCUAC |
| 199 | 2600 | AGAAAGUCCAAUACCUCAC | 2720 | GUGAGGUAUUGGACUUUCU |
| 2093 | 2601 | AACUUUGGUAAUUUAAGUU | 2721 | AACUUAAAUUACCAAAGUU |
| 1378 | 2602 | CUCUCCAAAUAUUAACUAA | 2722 | UUAGUUAAUAUUUGGAGAG |
| 2840 | 2603 | AUAGAAUAAGUUCUUAUCU | 2723 | AGAUAAGAACUUAUUCUAU |
| 1134 | 2604 | UAUCACUAAUAUACUAACA | 2724 | UGUUAGUAUAUUAGUGAUA |
| 2222 | 2605 | GAGGAUGUGUAAUUAACCA | 2725 | UGGUUAAUUACACAUCCUC |
| 2703 | 2606 | GAAUACUAUAACUCAAAUU | 2726 | AAUUUGAGUUAUAGUAUUC |
| 358 | 2607 | CUGCUACAACCUCUAGAUC | 2727 | GAUCUAGAGGUUGUAGCAG |
| 285 | 2608 | CUAUUUAUCAAUUUCUGUC | 2728 | GACAGAAAUUGAUAAAUAG |
| 2184 | 2609 | GAAGACAUGUUACUAAUAU | 2729 | AUAUUAGUAACAUGUCUUC |
| 1377 | 2610 | UCUCUCCAAAUAUUAACUA | 2730 | UAGUUAAUAUUUGGAGAGA |
| 359 | 2611 | UGCUACAACCUCUAGAUCU | 2731 | AGAUCUAGAGGUUGUAGCA |
| 194 | 2612 | CAUGGAGAAAGUCCAAUAC | 2732 | GUAUUGGACUUUCUCCAUG |
| 888 | 2613 | ACUAUCAGAAUCUACAUUC | 2733 | GAAUGUAGAUUCUGAUAGU |
| 193 | 2614 | UCAUGGAGAAAGUCCAAUA | 2734 | UAUUGGACUUUCUCCAUGA |
| 116 | 2615 | GUUAUCUCAUAUUUGGCUA | 2735 | UAGCCAAAUAUGAGAUAAC |
| 261 | 2616 | CAAGCACGUCAAAAGCUAA | 2736 | UUAGCUUUUGACGUGCUUG |
| 263 | 2617 | AGCACGUCAAAAGCUACAA | 2737 | UUGUAGCUUUUGACGUGCU |
| 267 | 2618 | CGUCAAAAGCUACAGAAUA | 2738 | UAUUCUGUAGCUUUUGACG |
| 268 | 2619 | GUCAAAAGCUACAGAAUCA | 2739 | UGAUUCUGUAGCUUUUGAC |
| 534 | 2620 | UGUAAACAUGAAAAGGGCA | 2740 | UGCCCUUUUCAUGUUUACA |
| 535 | 2621 | GUAAACAUGAAAAGGGCUA | 2741 | UAGCCCUUUUCAUGUUUAC |
| 1089 | 2622 | AACAGAUGAGAACUGGUGA | 2742 | UCACCAGUUCUCAUCUGUU |
| 1090 | 2623 | ACAGAUGAGAACUGGUGGA | 2743 | UCCACCAGUUCUCAUCUGU |
| 1091 | 2624 | CAGAUGAGAACUGGUGGUA | 2744 | UACCACCAGUUCUCAUCUG |
| 1092 | 2625 | AGAUGAGAACUGGUGGUUA | 2745 | UAACCACCAGUUCUCAUCU |
| 1093 | 2626 | GAUGAGAACUGGUGGUUAA | 2746 | UUAACCACCAGUUCUCAUC |
| 1094 | 2627 | AUGAGAACUGGUGGUUAAA | 2747 | UUUAACCACCAGUUCUCAU |
| 1095 | 2628 | UGAGAACUGGUGGUUAAUA | 2748 | UAUUAACCACCAGUUCUCA |
| 1096 | 2629 | GAGAACUGGUGGUUAAUAA | 2749 | UUAUUAACCACCAGUUCUC |
| 1097 | 2630 | AGAACUGGUGGUUAAUAUA | 2750 | UAUAUUAACCACCAGUUCU |
| 1228 | 2631 | UUUGGAAUCAUGAAACCUA | 2751 | UAGGUUUCAUGAUUCCAAA |
| 1229 | 2632 | UUGGAAUCAUGAAACCUUA | 2752 | UAAGGUUUCAUGAUUCCAA |
| 1614 | 2633 | AAGUAAGCUAGAGAAAAUA | 2753 | UAUUUUCUCUAGCUUACUU |
| 1615 | 2634 | AGUAAGCUAGAGAAAAUGA | 2754 | UCAUUUUCUCUAGCUUACU |
| 1616 | 2635 | GUAAGCUAGAGAAAAUGUA | 2755 | UACAUUUUCUCUAGCUUAC |
| 1673 | 2636 | UUCAUUAAAUGGAAGUGGA | 2756 | UCCACUUCCAUUUAAUGAA |
| 1701 | 2637 | AAGUCUUCAUUCUCAUUGA | 2757 | UCAAUGAGAAUGAAGACUU |
| 1702 | 2638 | AGUCUUCAUUCUCAUUGUA | 2758 | UACAAUGAGAAUGAAGACU |
| 1703 | 2639 | GUCUUCAUUCUCAUUGUCA | 2759 | UGACAAUGAGAAUGAAGAC |
| 1704 | 2640 | UCUUCAUUCUCAUUGUCUA | 2760 | UAGACAAUGAGAAUGAAGA |
| 1784 | 2641 | CUUGUCUUAGGGGUGUGGA | 2761 | UCCACACCCCUAAGACAAG |
| 1818 | 2642 | UAUUCAUGUAUAAGUGGAA | 2762 | UUCCACUUAUACAUGAAUA |
| 1821 | 2643 | UCAUGUAUAAGUGGACCCA | 2763 | UGGGUCCACUUAUACAUGA |

TABLE 2
List of abbreviations of nucleotides used in the nucleic
acid sequences disclosed herein, e.g., in Table 1B

| Abbreviation | Nucleotide(s) |
|---|---|
| A | adenosine-3′-phosphate |
| Af | 2′-deoxy-2′-fluoro-adenosine-3′-phosphate |
| Afs | 2′-deoxy-2′-fluoro-adenosine-3′-phosphorothioate |
| As | adenosine-3′-phosphorothioate |
| C | cytidine-3′-phosphate |
| Cf | 2′-deoxy-2′-fluoro-cytidine-3′-phosphate |
| Cfs | 2′-deoxy-2′-fluoro-cytidine-3′-phosphorothioate |
| Cs | cytidine-3′-phosphorothioate |
| G | guanosine-3′-phosphate |
| Gf | 2′-deoxy-2′-fluoro-guanosine-3′-phosphate |
| Gfs | 2′-deoxy-2′-fluoro-guanosine-3′-phosphorothioate |
| Gs | guanosine-3′-phosphorothioate |
| U | uridine-3′-phosphate |
| Uf | 2′-deoxy-2′-fluoro-uridine-3′-phosphate |
| Ufs | 2′-deoxy-2′-fluoro-uridine-3′-phosphorothioate |
| Us | uridine-3′-phosphorothioate |
| a | 2′-O-methyl-adenosine-3′-phosphate |
| as | 2′-O-methyl-adenosine-3′-phosphorothioate |
| c | 2′-O-methyl-cytidine-3′-phosphate |
| cs | 2′-O-methyl-cytidine-3′-phosphorothioate |
| g | 2′-O-methyl-guanosine-3′-phosphate |
| gs | 2′-O-methyl-guanosine-3′-phosphorothioate |
| u | 2′-O-methyl-uridine-3′-phosphate |
| us | 2′-O-methyl-uridine-3′-phosphorothioate |
| T | deoxythymidine-3′-phosphate |
| vpUs | 5′-vinylphosphonate-2′-O-methyl-uridine-3′- |
| | phosphorothioate |

TABLE 3
Exemplary sequences of PLN mRNA transcript

| SEQ ID | |
|---|---|
| NO: | Sequence |
| 2523 | NM_002667.5 Homo sapiens phospholamban (PLN), mRNA: |
| | ATAAACTGGGTGACAGAGTCAGAAAACTCCCCAGCTAAACACCCGTAAGACTTCA |
| | TACAACACAATACTCTATACTGTGATGATCACAGCTGCCAAGGCTACCTAAAAGAA |
| | GACAGTTATCTCATATTTGGCTGCCAGCTTTTTATCTTTCTCTCGACCACTTAAAAC |
| | TTCAGACTTCCTGTCCTGCTGGTATCATGGAGAAAGTCCAATACCTCACTCGCTCA |
| | GCTATAAGAAGAGCCTCAACCATTGAAATGCCTCAACAAGCACGTCAAAAGCTACA |
| | GAATCTATTTATCAATTTCTGTCTCATCTTAATATGTCTCTTGCTGATCTGTATCATC |
| | GTGATGCTTCTCTGAAGTTCTGCTACAACCTCTAGATCTGCAGCTTGCCACATCAG |
| | CTTAAAATCTGTCATCCCATGCAGACAGGAAAACAATATTGTATAACAGACCACTT |
| | CCTGAGTAGAAGAGTTTCTTTGTGAAAAGGTCAAGATTAAGACTAAAACTTATTGTT |
| | ACCATATGTATTCATCTGTTGGATCTTGTAAACATGAAAAGGGCTTTATTTTCAAAA |
| | ATTAACTTCAAAATAAGTGTATAAAATGCAACTGTTGATTTCCTCAACATGGCTCAC |
| | AAATTTCTATCCCAAATCTTTTCTGAAGATGAAGAGTTTAGTTTTAAAACTGCACTG |
| | CCAACAAGTTCACTTCATATATAAAGCATTATTTTTACTCTTTTGAGGTGAATATAAT |
| | TTATATTACAATGTAAAAGCTTCTTTAATACTAAGTATTTTTCAGGTCTTCACCAAGT |
| | ATCAAAGTAATAACACAAATGAAGTGTCATTATTCAAAATAGTCCACTGACTCCTCA |
| | CATCTGTTATCTTATTATAAAGAACTATTTGTAGTAACTATCAGAATCTACATTCTAA |
| | AACAGAAATTGTATTTTTTCTATGCCACATTAACATCTTTTAAAGTTGATGAGAATCA |
| | AGTATGGAAAAGTAAGGCCATACTCTTACATAATAAAATTCCTTTTAAGTAATTTTTT |
| | CAAAGAATCACAGAATTCTAGTACATGTAGGTAAATCATAAATCTGTTCTAAGACAT |
| | ATGATCAACAGATGAGAACTGGTGGTTAATATGTGACAGTGAGATTAGTCATATCA |
| | CTAATATACTAACAACAGAATCTAATCTTCATTTAAGGCACTGTAGTGAATTATCTG |
| | AGCTAGAGTTACCTAGCTTACCATACTATATCTTTGGAATCATGAAACCTTAAGACT |
| | TCAGAATGATTTTGCAGGTTGTCTTCCATTCCAGCCTAACATCCAATGCAGGCAAG |
| | GAAAATAAAAGATTTCCAGTGACAGAAAAATATATTATCTCAAGTATTTTTTAAAAAT |
| | ATATGAATTCTCTCTCCAAATATTAACTAATTATTAGATTATATTTTGAAATGAACTT |
| | GTTGGCCCATCTATTACATCTACAGCTGACCCTTGAACATGGGGGTTAGGGGAGC |
| | TGACAATTCGTGGGTCCGCAAAATCTTAACTACCTAATAGCCTACTATTGACCATA |
| | AACCTTACTGATAACATAAACAGTAAATTAACACATATTTTGCGTGTTATATGTATTA |
| | TACACTATATTCCTACAATAAAGTAAGCTAGAGAAAATGTTATTTAGAAAATCATAA |
| | GAAAGAGAAAATATATTTACTATTCATTAAATGGAAGTGGGTCAACATAAAAGTCTT |
| | CATTCTCATTGTCTTCACATTGAGTAGGCAGAGGAGGAGAAAGATGGGGAGGAAG |
| | AGAAGGCGTTGGTCTTGCAGTCTTGTCTTAGGGGTGTGGGGAGTGGGGGAAAGA |
| | ATATTCATGTATAAGTGGACCCTTGCAATTCAAGCCCTTGTTGTTCAAGGGTCAAC |
| | TGTAATAGGATATAGCTATTTTTCTTCCTCTATCAACCAAATGGTAAGCATCTATTTT |
| | GCAGTCCACTCTACTGAGCTAAATTATAGATCCAGCTATGCTATTTATAATTATTTT |
| | CTTGATGAATAAATTTTCAATTTCTCCTCTGACCATTTCAGAACATCTTCCAATAACT |
| | CATAAAACAACTGAAGTAAAATTGAGTGCTGGAAAATATATTCACCAAACTTTGGTA |
| | ATTTAAGTTGACTAAAGTTTAAAATTAAGTCTAAAATAGTTTACACCTATACTGCATA |
| | ATCCAACAATTTTAATTTCAGTTGAAGACATGTTACTAATATAACTATTATTAAAAGA |
| | GTAGAGGATGTGTAATTAACCATATCTTCTAAAACATGGTTACTAAAAGAATATGTA |
| | ACATCAATATTGACCTTGGTTTCTTACACAAGTGTTGCTAACTCAATAGTGAAGGA |
| | GACACTATTAAATTTTCTGAACCCATGAGAGATACTAGAGATGGGGAGTGGAAAGT |
| | GTTTGGTTCAGGGATATCTGAAGAACAGAAGGGCAGAGATTTCTTAAGTGACGCC |
| | TCATCTACAAGCTGGAAATTCCTAAAAACAAGTAGAAAGCTTATAAACAACAGGTG |
| | ATACACTCACCTCACTGGTTTTAGTAAATTACCAATACAGAAAGTATCCCTAGTCTT |
| | AAAAACAAGTGGAAAATTTGAACTGATTAGTCATATTCCTTTGATTACACTGTTTGT |
| | TACAATATTTTTCTCAGTAAACAGAAATAACTAATTTTTTTGTTCTTCATTCTTTGATA |
| | GAAATTAAAATCTTATTCTGTGAGGATTACAGAATACTATAACTCAAATTATAAAGT |
| | AGAATAAACTCTTTAAATAATTATTCTTCATCATAAAGTGTAAAGAATAAGATATAAG |
| | AAAACAATTTATTTTTAAAATTTAATATACTAAATGCTCAAATATGTTCTACTATAGA |
| | ATAAGTTCTTATCTTAATTTACAGGGCACTAAAAACAATTTTAAAATGCTTAATGTTG |
| | CCTTTTATATTTTAATTGGTTAAGAATATATATTTGTTTAATGCAAATCAGAATCACT |
| | ATATTAAAATGAATGTTCTTGAAAACTCA |

Table 4 provides targeting positions with SEQ ID NO: 2523 (NM_002667.5) for an oligonucleotide of the present disclosure. In some embodiments, an oligonucleotide targets a sequence corresponding to positions selected from Table 4. In some embodiments, an oligonucleotide targets a sequence corresponding to positions selected from 470-488, 471-489, 475-493, 450-468, 452-470, 528-546, 381-399, 382-400, 383-401, and 472-490 of SEQ ID NO: 2523 (NM_{M}002667.5).

```
TABLE 4
Targeting positions
17-35
18-36
19-37
20-38
21-39
42-60
45-63
47-65
48-66
49-67
52-70
54-72
55-73
  91-109
  94-112
  95-113
  96-114
  97-115
  98-116
  99-117
100-118
101-119
102-120
103-121
104-122
105-123
106-124
107-125
108-126
109-127
110-128
111-129
112-130
113-131
114-132
115-133
116-134
161-179
163-181
181-199
182-200
183-201
184-202
185-203
186-204
187-205
188-206
189-207
193-211
194-212
195-213
197-215
198-216
199-217
200-218
201-219
202-220
203-221
204-222
205-223
206-224
207-225
209-227
210-228
211-229
212-230
213-231
214-232
215-233
217-235
219-237
221-239
222-240
223-241
224-242
225-243
226-244
227-245
228-246
229-247
230-248
231-249
232-250
233-251
234-252
237-255
240-258
244-262
248-266
249-267
250-268
251-269
252-270
253-271
254-272
255-273
256-274
257-275
260-278
261-279
263-281
264-282
267-285
268-286
274-292
275-293
276-294
278-296
281-299
285-303
290-308
291-309
292-310
293-311
296-314
297-315
298-316
299-317
300-318
301-319
302-320
303-321
304-322
305-323
306-324
307-325
308-326
309-327
310-328
311-329
312-330
313-331
314-332
315-333
316-334
317-335
318-336
319-337
320-338
321-339
322-340
323-341
324-342
325-343
326-344
327-345
328-346
329-347
330-348
331-349
334-352
335-353
336-354
337-355
338-356
341-359
342-360
346-364
347-365
348-366
349-367
350-368
351-369
352-370
353-371
354-372
355-373
356-374
357-375
358-376
359-377
360-378
361-379
362-380
363-381
381-399
382-400
383-401
387-405
388-406
389-407
390-408
391-409
392-410
393-411
394-412
399-417
400-418
402-420
438-456
440-458
441-459
442-460
443-461
446-464
449-467
450-468
451-469
452-470
469-487
470-488
471-489
472-490
473-491
474-492
475-493
476-494
477-495
478-496
479-497
480-498
481-499
482-500
483-501
484-502
521-539
522-540
523-541
524-542
525-543
526-544
527-545
528-546
531-549
532-550
533-551
534-552
535-553
537-555
538-556
593-611
595-613
598-616
600-618
601-619
602-620
604-622
605-623
606-624
607-625
608-626
609-627
610-628
617-635
618-636
623-641
624-642
628-646
630-648
631-649
639-657
640-658
641-659
642-660
643-661
644-662
645-663
646-664
666-684
667-685
668-686
669-687
670-688
671-689
672-690
673-691
674-692
675-693
676-694
677-695
679-697
680-698
681-699
682-700
683-701
684-702
687-705
688-706
689-707
690-708
711-729
712-730
713-731
714-732
715-733
716-734
717-735
718-736
757-775
776-794
777-795
778-796
779-797
780-798
781-799
782-800
783-801
784-802
785-803
786-804
787-805
788-806
789-807
790-808
791-809
792-810
793-811
797-815
800-818
803-821
809-827
810-828
811-829
812-830
813-831
838-856
839-857
842-860
844-862
845-863
846-864
848-866
849-867
850-868
872-890
873-891
876-894
877-895
878-896
882-900
883-901
886-904
888-906
889-907
926-944
928-946
949-967
950-968
951-969
952-970
953-971
957-975
958-976
959-977
960-978
961-979
962-980
963-981
964-982
965-983
966-984
967-985
968-986
969-987
972-990
978-996
979-997
980-998
981-999
  983-1001
  984-1002
  985-1003
1022-1040
1023-1041
1024-1042
1031-1049
1032-1050
1061-1079
1062-1080
1063-1081
1064-1082
1066-1084
1067-1085
1068-1086
1069-1087
1070-1088
1071-1089
1072-1090
1073-1091
1074-1092
1075-1093
1077-1095
1078-1096
1079-1097
1080-1098
1081-1099
1082-1100
1083-1101
1084-1102
1086-1104
1087-1105
1089-1107
1090-1108
1091-1109
1092-1110
1093-1111
1094-1112
1095-1113
1096-1114
1097-1115
1098-1116
1099-1117
1100-1118
1101-1119
1118-1136
1119-1137
1120-1138
1121-1139
1122-1140
1126-1144
1130-1148
1132-1150
1134-1152
1136-1154
1178-1196
1179-1197
1202-1220
1205-1223
1206-1224
1207-1225
1208-1226
1209-1227
1210-1228
1211-1229
1228-1246
1229-1247
1231-1249
1232-1250
1233-1251
1234-1252
1235-1253
1236-1254
1239-1257
1240-1258
1266-1284
1267-1285
1268-1286
1286-1304
1287-1305
1288-1306
1289-1307
1290-1308
1291-1309
1292-1310
1293-1311
1294-1312
1295-1313
1296-1314
1297-1315
1304-1322
1312-1330
1313-1331
1314-1332
1315-1333
1366-1384
1367-1385
1368-1386
1369-1387
1370-1388
1371-1389
1372-1390
1373-1391
1375-1393
1377-1395
1378-1396
1379-1397
1380-1398
1412-1430
1413-1431
1414-1432
1415-1433
1416-1434
1417-1435
1418-1436
1419-1437
1420-1438
1421-1439
1422-1440
1423-1441
1424-1442
1426-1444
1430-1448
1431-1449
1432-1450
1433-1451
1434-1452
1435-1453
1436-1454
1437-1455
1438-1456
1439-1457
1440-1458
1441-1459
1442-1460
1466-1484
1467-1485
1468-1486
1469-1487
1481-1499
1483-1501
1484-1502
1485-1503
1498-1516
1501-1519
1502-1520
1503-1521
1504-1522
1505-1523
1506-1524
1508-1526
1526-1544
1527-1545
1528-1546
1529-1547
1530-1548
1531-1549
1532-1550
1533-1551
1543-1561
1590-1608
1600-1618
1614-1632
1615-1633
1616-1634
1673-1691
1674-1692
1675-1693
1701-1719
1702-1720
1703-1721
1704-1722
1706-1724
1707-1725
1708-1726
1711-1729
1782-1800
1783-1801
1784-1802
1817-1835
1818-1836
1821-1839
1822-1840
1876-1894
1900-1918
1906-1924
1933-1951
1939-1957
1940-1958
1948-1966
1956-1974
1957-1975
1958-1976
2000-2018
2001-2019
2002-2020
2025-2043
2030-2048
2087-2105
2088-2106
2093-2111
2094-2112
2099-2117
2102-2120
2145-2163
2146-2164
2147-2165
2148-2166
2150-2168
2151-2169
2152-2170
2153-2171
2158-2176
2182-2200
2183-2201
2184-2202
2188-2206
2216-2234
2219-2237
2220-2238
2222-2240
2228-2246
2232-2250
2269-2287
2281-2299
2282-2300
2284-2302
2288-2306
2293-2311
2295-2313
2297-2315
2301-2319
2303-2321
2306-2324
2307-2325
2308-2326
2309-2327
2310-2328
2311-2329
2315-2333
2317-2335
2349-2367
2380-2398
2389-2407
2390-2408
2420-2438
2424-2442
2425-2443
2429-2447
2430-2448
2431-2449
2432-2450
2434-2452
2437-2455
2443-2461
2522-2540
2576-2594
2601-2619
2681-2699
2690-2708
2692-2710
2697-2715
2700-2718
2703-2721
2837-2855
2840-2858
2856-2874
2857-2875
2947-2965
```

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. An RNAi oligonucleotide comprising a sense strand and an antisense strand, wherein the sense strand and the antisense strand form a duplex, wherein the sense strand and antisense strands are selected from the group consisting of:

| SEQ ID | | SEQ | |
|---|---|---|---|
| NO: | Sense strand | ID NO: | Antisense strand |
| 1402 | ususguGfaAfAfAfggucaagaua | 2030 | usAfsucuUfgAfCfcuuuUfcAfcaasusu |
| 1403 | usgsugAfaAfAfGfgucaagauua | 2031 | usAfsaucUfuGfAfccuuUfuCfacasusu |
| 1404 | asasaaGfgUfCfAfagauuaagaa | 2032 | usUfscuuAfaUfCfuugaCfcUfuuususu |
| 1471 | uscscuGfaGfUfAfgaagaguuua | 2099 | usAfsaacUfcUfUfcuacUfcAfggasusu |
| 1472 | csusgaGfuAfGfAfagaguuucua | 2100 | usAfsgaaAfcUfCfuucuAfcUfcagsusu |
| 1474 | gsgsauCfuUfGfUfaaacaugaaa | 2102 | usUfsucaUfgUfUfuacaAfgAfuccsusu |
| 1670 | gscsuuGfcCfAfCfaucagcuuaa | 2298 | usUfsaagCfuGfAfugugGfcAfagcsusu |
| 1671 | csusugCfcAfCfAfucagcuuaaa | 2299 | usUfsuaaGfcUfGfauguGfgCfaagsusu |
| 1672 | ususgcCfaCfAfUfcagcuuaaaa | 2300 | usUfsuuaAfgCfUfgaugUfgGfcaasusu |
| 1688 | gsusgaAfaAfGfGfucaagauuaa | 2316 | usUfsaauCfuUfGfaccuUfuUfcacsusu |
| 1402 | ususguGfaAfAfAfggucaagaua | 2513 | vpUsAfsucuUfgAfCfcuuuUfcAfcaasusu |
| 1403 | usgsugAfaAfAfGfgucaagauua | 2514 | vpUsAfsaucUfuGfAfccuuUfuCfacasusu |
| 1404 | asasaaGfgUfCfAfagauuaagaa | 2515 | vpUsUfscuuAfaUfCfuugaCfcUfuuususu |
| 1471 | uscscuGfaGfUfAfgaagaguuua | 2516 | vpUsAfsaacUfcUfUfcuacUfcAfggasusu |
| 1472 | csusgaGfuAfGfAfagaguuucua | 2517 | vpUsAfsgaaAfcUfCfuucuAfcUfcagsusu |
| 1474 | gsgsauCfuUfGfUfaaacaugaaa | 2518 | vpUsUfsucaUfgUfUfuacaAfgAfuccsusu |
| 1670 | gscsuuGfcCfAfCfaucagcuuaa | 2519 | vpUsUfsaagCfuGfAfugugGfcAfagcsusu |
| 1671 | csusugCfcAfCfAfucagcuuaaa | 2520 | vpUsUfsuaaGfcUfGfauguGfgCfaagsusu |
| 1672 | ususgcCfaCfAfUfcagcuuaaaa | 2521 | vpUsUfsuuaAfgCfUfgaugUfgGfcaasusu |
| 1688 | gsusgaAfaAfGfGfucaagauuaa | 2522 | vpUsUfsaauCfuUfGfaccuUfuUfcacsusu |

wherein:
Af represents 2′-deoxy-2′-fluoro-adenosine-3′-phosphate;
Afs represents 2′-deoxy-2′-fluoro-adenosine-3′-phosphorothioate;
Cf represents 2′-deoxy-2′-fluoro-cytidine-3′-phosphate;
Gf represents 2′-deoxy-2′-fluoro-guanosine-3′-phosphate;
Uf represents 2′-deoxy-2′-fluoro-uridine-3′-phosphate;
Ufs represents 2′-deoxy-2′-fluoro-uridine-3′-phosphorothioate;
a represents 2′-O-methyl-adenosine-3′-phosphate;
as represents 2′-O-methyl-adenosine-3′-phosphorothioate;
c represents 2′-O-methyl-cytidine-3′-phosphate;
cs represents 2′-O-methyl-cytidine-3′-phosphorothioate;
g represents 2′-O-methyl-guanosine-3′-phosphate;
gs represents 2′-O-methyl-guanosine-3′-phosphorothioate;
u represents 2′-O-methyl-uridine-3′-phosphate;
us represents 2′-O-methyl-uridine-3′-phosphorothioate; and
vpUs represents 5′-vinylphosphonate-2′-O-methyl-uridine-3′-phosphorothioate.

2. The RNAi oligonucleotide of claim 1, wherein the sense strand is ususguGfaAfAfAfggucaagaua (SEQ ID NO: 1402), and the antisense strand is usAfsucuUfgAfCfcuuuUfcAfcaasusu (SEQ ID NO: 2030).

3. The RNAi oligonucleotide of claim 1, wherein the sense strand is usgsugAfaAfAfGfgucaagauua (SEQ ID NO: 1403), and the antisense strand is usAfsaucUfuGfAfccuuUfuCfacasusu (SEQ ID NO: 2031).

4. The RNAi oligonucleotide of claim 1, wherein the sense strand is asasaaGfgUfCfAfagauuaagaa(SEQ ID NO: 1404), and the antisense strand is usUfscuuAfaUfCfuugaCfcUfuuususu (SEQ ID NO: 2032).

5. The RNAi oligonucleotide of claim 1, wherein the sense strand is uscscuGfaGfUfAfgaagaguuua (SEQ ID NO: 1471), and the antisense strand is usAfsaacUfcUfUfcuacUfcAfggasusu (SEQ ID NO: 2099).

6. The RNAi oligonucleotide of claim 1, wherein the sense strand is csusgaGfuAfGfAfagaguuucua (SEQ ID NO: 1472), and the antisense strand is usAfsgaaAfcUfCfuucuAfcUfcagsusu (SEQ ID NO: 2100).

7. The RNAi oligonucleotide of claim 1, wherein the sense strand is gsgsauCfuUfGfUfaaacaugaaa(SEQ ID NO: 1474), and the antisense strand is usUfsucaUfgUfUfuacaAfgAfuccsusu (SEQ ID NO: 2102).

8. The RNAi oligonucleotide of claim 1, wherein the sense strand is gscsuuGfcCfAfCfaucagcuuaa(SEQ ID NO: 1670), and the antisense strand is usUfsaagCfuGfAfugugGfcAfagcsusu (SEQ ID NO: 2298).

9. The RNAi oligonucleotide of claim 1, wherein the sense strand is csusugCfcAfCfAfucagcuuaaa (SEQ ID NO: 1671), and the antisense strand is usUfsuaaGfcUfGfauguGfgCfaagsusu (SEQ ID NO: 2299).

10. The RNAi oligonucleotide of claim 1, wherein the sense strand is ususgcCfaCfAfUfcagcuuaaaa(SEQ ID NO: 1672), and the antisense strand is usUfsuuaAfgCfUfgaugUfgGfcaasusu (SEQ ID NO: 2300).

11. The RNAi oligonucleotide of claim 1, wherein the sense strand is gsusgaAfaAfGfGfucaagauuaa (SEQ ID NO: 1688), and the antisense strand is usUfsaauCfuUfGfaccuUfuUfcacsusu (SEQ ID NO: 2316).

12. The RNAi oligonucleotide of claim 1, wherein the sense strand is ususguGfaAfAfAfggucaagaua (SEQ ID NO: 1402), and the antisense strand is vpUsAfsucuUfgAfCfcuuuUfcAfcaasusu (SEQ ID NO: 2513).

13. The RNAi oligonucleotide of claim 1, wherein
the sense strand is usgsugAfaAfAfGfgucaagauua (SEQ ID NO: 1403), and the antisense strand is vpUsAfsaucUfuGfAfccuuUfuCfacasusu (SEQ ID NO: 2514).

14. The RNAi oligonucleotide of claim 1, wherein the sense strand is asasaaGfgUfCfAfagauuaagaa(SEQ ID NO: 1404), and the antisense strand is vpUsUfscuuAfaUfCfuugaCfcUfuuususu (SEQ ID NO: 2515).

15. The RNAi oligonucleotide of claim 1, wherein the sense strand is uscscuGfaGfUfAfgaagaguuua (SEQ ID NO: 1471), and the antisense strand is vpUsAfsaacUfcUfUfcuacUfcAfggasusu (SEQ ID NO: 2516).

16. The RNAi oligonucleotide of claim 1, wherein the sense strand is csusgaGfuAfGfAfagaguuucua (SEQ ID NO: 1472), and the antisense strand is vpUsAfsgaaAfcUfCfuucuAfcUfcagsusu (SEQ ID NO: 2517).

17. The RNAi oligonucleotide of claim 1, wherein the sense strand is gsgsauCfuUfGfUfaaacaugaaa (SEQ ID NO: 1474), and the antisense strand is vpUsUfsucaUfgUfUfuacaAfgAfuccsusu (SEQ ID NO: 2518).

18. The RNAi oligonucleotide of claim 1, wherein the sense strand is gscsuuGfcCfAfCfaucagcuuaa (SEQ ID NO: 1670), and the antisense strand is vpUsUfsaagCfuGfAfugugGfcAfagcsusu (SEQ ID NO: 2519).

19. The RNAi oligonucleotide of claim 1, wherein the sense strand is csusugCfcAfCfAfucagcuuaaa (SEQ ID NO: 1671), and the antisense strand is vpUsUfsuaaGfcUfGfauguGfgCfaagsusu (SEQ ID NO: 2520).

20. The RNAi oligonucleotide of claim 1, wherein the sense strand is ususgcCfaCfAfUfcagcuuaaaa(SEQ ID NO: 1672), and the antisense strand is vpUsUfsuuaAfgCfUfgaugUfgGfcaasusu (SEQ ID NO: 2521).

21. The RNAi oligonucleotide of claim 1, wherein the sense strand is gsusgaAfaAfGfGfucaagauuaa (SEQ ID NO: 1688), and the antisense strand is vpUsUfsaauCfuUfGfaccuUfuUfcacsusu (SEQ ID NO: 2522).

22. A pharmaceutical composition comprising the RNAi oligonucleotide of claim 1, or a pharmaceutically acceptable salt thereof, and a pharmaceutically acceptable carrier or excipient.

23. A method for inhibiting Phospholamban (PLN) expression or treating a PLN related disorder in a subject, the method comprising administering to the subject an effective amount of the RNAi oligonucleotide of claim 1, or a pharmaceutically acceptable salt thereof.
